(19) United States
(12) Patent Application Publication
(10) Pub. No.: US 2024/0336627 A1
(43) Pub. Date: Oct. 10, 2024

(54) NOVEL COMPOUNDS FOR THE DIAGNOSIS OF TDP-43 PROTEINOPATHIES

(71) Applicant: AC IMMUNE SA, Lausanne (CH)

(72) Inventors: Heiko KROTH, Ecublens (CH); Vincent DARMENCY, Yens (CH); Nicolas DREYFUS, Morges (CH); Cédric BOUDOU, Lausanne (CH)

(21) Appl. No.: 18/579,771

(22) PCT Filed: Jul. 15, 2022
(86) PCT No.: PCT/EP2022/069873

§ 371 Date: Jan. 16, 2024

(30) Foreign Application Priority Data
Jul. 16, 2021 (EP) 21186037.4

(51) Int. Cl. C07D 513/04; A61K 51/04

(52) U.S. Cl. CPC C07D 513/04; A61K 51/0455; A61K 51/0459

(57) ABSTRACT

The present invention relates to compounds which are suitable for imaging TDP-43 (Transactive response (TAR) DNA binding protein 43 kDa) aggregates. The compounds can be used, for example, for diagnosing a disease, disorder or abnormality associated with TDP-43 aggregates or a TDP-43 proteinopathy, such as amyotrophic lateral sclerosis (ALS), Alzheimer's disease (AD), Frontotemporal dementia (FTD) and limbic-predominant age-related TDP-43 encephalopathy (LATE).

### FIELD OF THE INVENTION

The present invention relates to compounds which are suitable for imaging TDP-43 (Transactive response (TAR) DNA binding protein 43 kDa) aggregates. Said compounds can be used, for example, for diagnosing a disease, disorder or abnormality associated with TDP-43 aggregates or a TDP-43 proteinopathy, such as amyotrophic lateral sclerosis (ALS), Alzheimer's disease (AD), Frontotemporal dementia (FTD) and limbic-predominant age-related TDP-43 encephalopathy (LATE). The present invention invention also relates to processes for the preparation of said compounds, diagnostic compositions comprising said compounds, methods of using said compounds, kits comprising said compounds and their uses thereof.

### BACKGROUND OF THE INVENTION

Age-associated brain disorders characterized by pathological aggregation of proteins in the CNS (proteinopathies) and peripheral organs represent one of the leading causes of disability and mortality in the world. The best characterized protein that forms extracellular aggregates is amyloid beta (Abeta) in Alzheimer's disease (AD) and Abeta-related disorders. Other disease-associated, aggregation-prone proteins leading to neurodegeneration include but are not limited to Tau, alpha-synuclein (a-syn), huntingtin, fused in sarcoma (FUS), dipeptide repeat proteins (DPRs) produced by unconventional translation of the C9orf72 repeat expansion, superoxide dismutase 1 (SOD1), and TDP-43. Diseases involving TDP-43 aggregates are generally referred to as TDP-43 proteinopathies and include, but are not limited to, amyotrophic lateral sclerosis (ALS) and frontotemporal lobar degeneration (FTLD), including frontotemporal lobar dementia with TDP-43 pathology (FTLD-TDP, Frontotemporal lobar degeneration with TDP-43 inclusions) and limbic-predominant age-related TDP-43 encephalopathy (LATE).

### TDP-43 Introduction

Transactive response (TAR) DNA binding protein 43 kDa (TDP-43) is a 414-amino acid protein encoded by the TARDBP gene on chromosome 1p36.2 (ALS10). TARDBP is comprised of six exons (exon 1 is non-coding; exons 2-6 are protein-coding). TDP-43 belongs to the family of heterogeneous ribonucleoprotein (hnRNP) RNA binding proteins (Wang et al. Trends in Molecular Medicine, Vol. 14, No. 11, 2008, 479-485; Lagier-Tourenne et al., Human Molecular Genetics, 2010, Vol. 19, Review Issue 1 R46-R64). TDP-43 contains five functional domains (FIG. 1 in Warraich et al., The International Journal of Biochemistry & Cell Biology, 42 (2010) 1606-1609): two RNA recognition motifs (RRM1 and RRM2), which have two highly conserved hexameric ribonucleoprotein 2 (RNP2) and octameric ribonucleioprotein 1 (RNP1) regions, a nuclear export signal (NES) and a nuclear localization signal (NLS) enabling it to shuttle between the nucleus and the cytoplasm transporting bound mRNA, and a glycine rich domain at the C-terminal, which mediates protein-protein interactions. TDP-43 is involved in multiple aspects of RNA processing, including transcription, splicing, transport, and stabilization (Buratti and Baralle, FEBS Journal, 277 (2010) 2268-2281). It is a highly conserved, ubiquitously expressed protein with a tightly autoregulated expression level that shuttles continuously between the nucleus and cytoplasm, but is normally localized predominantly to the nucleus. In 2006, TDP-43 was identified as the protein that accumulates in the vast majority of cases of frontotemporal lobar degeneration (FTLD) with tau-negative, ubiquitin-positive inclusions (then referred to as FTLD-TDP), and in most cases of amyotrophic lateral sclerosis (ALS) (Arai et al. Biochemical and Biophysical Research Communications, 351 (2006) 602-611; Neumann et al. Science, 314, (2006), 130-133).

Thirty-eight negative-dominant mutations in TDP-43 have been identified in sporadic and familial ALS patients as well as in patients with inherited FTD (K263E, N267S), mainly located in the glycine rich domain (FIG. 1; Lagier-Tourenne and Cleveland, Cell, 136, 2009, 1001-1004). TDP-43 is inherently aggregation-prone, as shown by sedimentation assays, and this propensity is increased by some ALS-associated TARDBP mutations (Ticozzi et al., CNS Neurol Disord Drug Targets, 2010, 9(3), 285-296).

### TDP-43 in Neurodegeneration

TDP-43 aggregates have been identified in a growing list of pathological conditions (Lagier-Tourenne et al., Human Molecular Genetics, 2010, Vol. 19, Review Issue 1 R46-R64), including but not limited to: frontotemporal dementia (sporadic or familial with or without motor-neuron disease (MND), with progranulin (GRN) mutation, with TARDBP mutation, with valosine-containing protein (VCP) mutation, linked to chromosome 9p, corticobasal degeneration, frontotemporal lobar degeneration with ubiquitin-positive inclusions, argyrophilic grain disease, Pick's disease and the like), amyotrophic lateral sclerosis (sporadic ALS, with TARDBP mutation, with ANG mutation), Alzheimer's disease (sporadic and familial), Down syndrome, familial British dementia, polyglutamine diseases (Huntington's disease and SCA3), hippocampal sclerosis dementia and myophaties (sporadic inclusion body myositis, inclusion body myopathy with VCP mutation, oculo-pharyngeal muscular dystrophy with rimmed vacuoles, myofibrillar myopathies with MYOT or DES mutation).

Aggregated TDP-43 from patient brains shows a number of abnormal modifications, including hyperphosphorylation, ubiquitination, acetylation and C-terminal fragments through proteolytic cleavage (Arai et al. Biochemical and Biophysical Research Communications, 351 (2006) 602-611; Neumann et al. Science, 314, (2006), 130-133; Neumann et al. Acta Neuropathol, (2009) 117: 137-149; Hasegawa et al. Annals of Neurology, 2008, Vol 64 No 1, 60-70; Cohen et al., Nat Commun.; 2015, 6: 5845). Another characteristic feature of TDP-43 pathology is redistribution and accumulation of TDP-43 from nucleus to cytoplasm. The hallmark lesions of FTLD-TDP are neuronal and glial cytoplasmic inclusions (neuronal cytoplasmic inclusions (NCI) and glial cytoplasmic inclusions (GCI), respectively) and dystrophic neurites (DN) that are immunoreactive for TDP-43, as well as ubiquitin and p62, but negative for other neurodegenerative disease-related proteins. Differences in inclusion morphology and tissue distribution thereof are associated with specific mutations and/or clinical representations. Four types of TDP-43 pathology are described so far by histological methods (Mackenzie and Neumann, J. Neurochem., (2016), 138 (Suppl. 1), 54-70). FTLD-TDP type A cases are characterized by abundant short DN and compact oval or crescentic NCI, predominantly in layer II of the neocortex (FIG. 2f in Mackenzie et al., J. Neurochem., 2016, 138 (Suppl. 1), 54-70). Cases with this pathology usually present clinically with either behavioral-variant frontotemporal dementia (bvFTD) or nonfluent/agrammatic variants of Primary Progressive Aphasia (nfvPPA) and are associated with progranulin (GRN) mutations. Type B cases show moderate numbers of compact or granular NCI in both superficial and deep cortical layers with relatively few DN and Nil (FIG. 2g in Mackenzie et al., J. Neurochem., 2016, 138 (Suppl. 1), 54-70). Most cases with coappearence of FTD and ALS symptoms are found to have FTLD-TDP type B pathology. Type C cases have an abundance of long tortuous neurites, predominantly in the superficial cortical laminae, with few or no NCI (FIG. 2j in Mackenzie et al., J. Neurochem., 2016, 138 (Suppl. 1), 54-70). This pathology is particularly found in cases presenting with svPPA (semantic variant of primary progressive aphasia). FTLD-TDP type D displays with abundant lentiform neuronal intranuclear inclusions (Nil) and short DN in the neocortex with only rare NCI (FIG. 2k in Mackenzie et al., J. Neurochem., 2016, 138 (Suppl. 1), 54-70). This pattern of pathology is only found in cases with VCP in association with inclusion body myositis.

### TDP-43 in FTD

Frontotemporal dementia (FTD) is a clinical term that covers a wide spectrum of disorders based on the degeneration of frontal and temporal lobes—a pathological feature termed frontotemporal lobar degeneration (FTLD). FTD is the second most abundant cause of early degenerative dementias in the age group below 65 years (Le Ber, Revue Neurologique, 169 (2013), 811-819). FTD is presented by several syndromes including bvFTD which is characterized by changes in personality and behavior; semantic dementia (SD) and progressive nonfluent aphasia (PNFA) characterized by changes in the language function; corticobasal syndrome (CBS), progressive supranuclear palsy syndrome and motor neuron disease (FTD-MND) characterized by movement disfunction. Diagnosis of these syndromes is complicated and final conclusion can only be achieved through postmortem tissue analysis based on immunohistochemistry to detect aggregated protein and description of the affected brain regions. In terms of pathological, proteinaceaous inclusions, about 45% of cases show pathological accumulation of misfolded Tau, 45% of cases have pathological TDP-43 and a smaller subgroup has aggregates of FUS and other proteins. FTLD-TDP is a pathology term describing FTD cases with TDP-43 pathology found predominantly as cytoplasmic or neuritic protein aggregates in neurons and glia containing misfolded, insoluble, phosphorylated and truncated TDP-43.

### TDP-43 in ALS

Amyotrophic lateral sclerosis (ALS) is an adult-onset neurodegenerative disorder characterized by the premature loss of upper and lower motor neurons. The progression of ALS is marked by fatal paralysis and respiratory failure with a disease course from diagnosis to death of 1 to 5 years. In most cases of sporadic ALS the neuropathology is characterized by abnormal cytoplasmic accumulations of TDP-43 in neurons and glia of the primary motor cortex, brainstem motor nuclei, spinal cord and the associated white matter tracts. ALS with dementia involves accumulation of TDP-43 in extramotor neocortex and hippocampus. The role of phosphorylation of TDP-43 in ALS patients has been explored with the help of phospho-specific antibodies that strongly bind to nuclear and cytoplasmic TDP-43 inclusions. Amino acids S379, S403, S404, S409, and S410 have been identified as the major sites of phosphorylation of TDP-43 (Hasegawa et al., Ann Neurol., 2008; 64: 60-70; Neumann et al., Acta Neuropathol., 2009, 117: 137-149).

### TDP-43 in LATE

Limbic-predominant age-related TDP-43 encephalopathy (LATE) neuropathological change (LATE-NC) is defined by a stereotypical TDP-43 proteinopathy in older adults, with or without coexisting hippocampal sclerosis pathology. LATE-NC is a common TDP-43 proteinopathy, associated with an amnestic dementia syndrome that mimicked Alzheimer's-type dementia in retrospective autopsy studies. LATE is distinguished from frontotemporal lobar degeneration with TDP-43 pathology based on its epidemiology (LATE generally affects older subjects), and relatively restricted neuroanatomical distribution of TDP-43 proteinopathy. There is no molecule-specific biomarker for LATE. A discovery of a TDP-43 PET tracer may enable accurate, potentially earlier diagnosis as well as monitoring of disease progression to facilitate longitudinal drug efficacy measurements in patients during clinical trials (including as a potential exclusion criterion for Alzheimer's disease clinical trials) and longitudinal studies of the clinical and pathological progression of LATE (Nelson et al., Brain, 2019, Vol. 142; issue 6, 1503-1527).

### TDP-43 in AD and Other Diseases

TDP-43 pathology occurs in up to 57% of brains of patients with Alzheimer's disease (Josephs K A et al., Acta Neuropathol., 2014; 127(6): 811-824; Josephs K A et al., Acta Neuropathol., 2014; 127(3): 441-450; McAleese et al., Brain Pathol., 2017 July; 27(4): 472-479). TDP-43 aggregation is associated with cognitive decline, memory loss and medial temporal atrophy in AD. TDP-43 positive patients are 10-fold more likely to be cognitively impaired at death compared to TDP-43 negative subjects. It appears that TDP-43 represents a secondary or independent pathology that shares overlapping features with AD by targeting the medial temporal lobe. Pathologic TDP-43 follows a stereotypical pattern of deposition that was captured by the TDP-43 in AD (TAD) staging scheme: TDP-43 first deposits in the amygdala (stage 1) followed by hippocampus, limbic, temporal, and finally frontostriatum (stage V) (Josephs K A et al., Acta Neuropathol., 2014; 127(6): 811-824; Josephs K A et al. Acta Neuropathol., 2014; 127(3): 441-450).

### Diagnostics in FTD and ALS

The diagnosis of FTD based on clinical manifestations is insufficient since the clinical representation can overlap with other diseases, in particular, in the earlier stages. Therefore, the development of sensitive and specific biomarkers allowing the differentiation between types of pathology within the FTD spectrum is an urgent task. Such tools will allow better detecting and understanding the specific type of pathology causing neurodegeneration. Eventually this will lead to the development of diagnostic biomarkers enabling more efficient and precise patient selection for longitudinal monitoring in clinical studies, supporting the development of novel therapeutics for ALS and FTD.

A number of approaches aim at development of biochemical biomarkers to distinguish different types of FTD pathology. Some studies showed that TDP-43 concentration is increased in cerebrospinal fluid (CSF) of clinically defined FTD or FTD-MND populations, although there is a significant overlap with control or AD subjects and it remains unclear if such an approach will prove clinically useful (Foulds et al., Acta Neuropathol., 2008, 116: 141-146; Steinacker et al., Arch. Neurol., 2008; 65(11): 1481-1487). Levels of total Tau or Thr181-phosphorylated Tau do not discriminate FTLD-Tau from control. A possible diagnostic tool for the differentiation of FTLD-Tau and FTLD-TDP is the reduced CSF p-Tau181 to Tau ratio below a value of 0.37 (Hu et al., Neurology., 2013; 81(22): 1945-1952). Another study showed that CSF phosphorylated Tau levels are positively associated with cerebral Tau burden in FTD and might help to distinguish TDP-43 proteinopathy from tauopathy (Irwin et al., Ann. Neurol., 2017 August; 82(2):247-258).

In parallel to biochemical biomarkers the development of imaging biomarkers will enable early and specific detection of the pathology in FTD and ALS. The ability to image TDP-43 deposition in the brain will be a substantial achievement for diagnosis and drug development for FTD, ALS and other neurodegenerative disorders. Progressive TDP-43 accumulation in the CNS is associated with disease progression and represents an obvious target for development of novel therapeutics and diagnostic tools to study pharmacodynamics and disease progression. Given the relative novelty of TDP-43 as a target, the development of a PET-tracer targeting this protein is at its beginning. However, most of the compound's reported so far are not specific for TDP-43 and no direct binding to the target was demonstrated for any of these compounds.

A number of challenges are associated with the development of a TDP-43-specific PET-tracer including low abundance and heterogenic distribution of the target in the patient's brain as well as the lack of reference compounds. In order to reduce background signal interference resulting from non-specific, off-target binding and to reduce dosing requirements, TDP-43 imaging compounds should bind with high affinity and selectivity to the target. For imaging of TDP-43 aggregates associated with neurological disorders such as FTD and ALS, imaging compounds need to penetrate the blood brain barrier and pass into the relevant regions of the brain. For targeting intracellular amyloid-like inclusions such as TDP-43 aggregates, cell permeability is a further requirement of imaging compounds. A further prerequisite in order to avoid accumulation of the compound in the tissue, which may result in increased risk of unwanted side effects, is a fast compound wash-out from the brain (or other target organ).

It was an object of the present invention to provide compounds which are able to bind to TDP-43 aggregates. In particular, the compounds of the present invention should be useful for identification and differentiation of patients and patient groups with TDP-43 proteinopathies (such as FTD, FTLD-TDP, LATE and ALS) and for differentiating TDP-43 proteinopathies from other proteinopathies.

The present inventors have surprisingly found that compounds having the formula (I) can recognize and bind to TDP-43 aggregates.

### SUMMARY OF THE INVENTION

The present invention is summarized in the appended claims. In particular, the present invention refers to a compound having the formula (I)

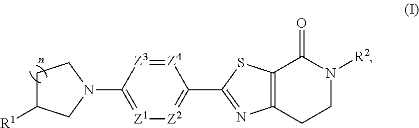

or a detectably labelled compound, stereoisomer, polymorph, racemic mixture, tautomer, pharmaceutically acceptable salt, prodrug, hydrate, or solvate thereof, or mixtures thereof;
wherein
Z¹, Z², Z³ and Z⁴ are each selected from CH and N, wherein
   if Z¹ is N, Z² is CH;
if Z² is N, Z¹ is CH;
if Z³ is N, Z⁴ is CH;
if Z⁴ is N, Z³ is CH;
n is 1 or 2;
R¹ is H or F; and
R² is
   a 5- or 6-membered carbocyclic ring which can be optionally substituted with F, NH₂, CN and/or CH₃,
   a 5- or 6-membered heterocycloalkyl ring which can be optionally substituted with F, NH₂, CN and/or CH₃, wherein the 5- or 6-membered heterocycloalkyl ring contains one or more heteroatoms selected from N, O and S,
   a 5-membered heteroaryl ring which can be optionally substituted with F, NH₂, CN and/or CH₃, wherein the 5-membered heteroaryl ring contains one or more heteroatoms selected from N, O and S, or
   a 6-membered heteroaryl ring which can be optionally substituted with F, NH₂, CN and/or CH₃, wherein the 6-membered heteroaryl ring contains one heteroatom selected from O and S, or two or more heteroatoms selected from O, N and S.

In another aspect, the present invention provides a diagnostic composition comprising a compound according to the definition of a compound of formula (I), or subformulae thereof, as defined herein, and optionally at least one physiologically acceptable carrier, diluent, adjuvant and/or excipient. Said compounds can be used for imaging of TDP-43 aggregates, particularly wherein the imaging is conducted by positron emission tomography or for diagnosing a disease, disorder or abnormality associated with TDP-43 aggregates, particularly wherein the diagnosis is conducted by positron emission tomography.

In another aspect, the invention provides a compound according to the definition of a compound of formula (I), or subformulae thereof, which can be used in the following methods:
A method of imaging a disease, disorder or abnormality associated with TDP-43 aggregates, in a subject;
A method of positron emission tomography (PET) imaging of TDP-43 aggregates in a tissue of a subject;
A method for the detection and optionally quantification of TDP-43 aggregates in a tissue of a subject;
A method of the diagnostic imaging of the brain of a subject;
A method of collecting data for the diagnosis of a disease, disorder or abnormality associated with TDP-43 aggregates or for the diagnosis of a TDP-43 proteinopathy;
A method of collecting data for determining a predisposition to a disease, disorder or abnormality associated with TDP-43 aggregates or a TDP-43 proteinopathy;
A method of collecting data for monitoring the progression of a disease, disorder or abnormality associated with TDP-43 aggregates or for monitoring the progression of a TDP-43 proteinopathy in a patient; and
A method of collecting data for predicting responsiveness of a patient suffering from a disease, disorder or abnormality associated with TDP-43 aggregates to a treatment with a medicament.

In another aspect, the invention provides a compound according to the definition of a compound of formula (I), or subformulae thereof, that can also be used as a TDP-43 aggregates' biomarker or a TDP-43 proteinopathy biomarker, as a TDP-43 proteinopathy diagnostic agent or diagnostic tool or as an in vitro analytical reference or an in vitro screening tool.

Another aspect of the present invention provides a method of preparing a compound according to the definition of a compound of formula (I), or subformulae thereof.

In yet another aspect, the present invention relates to kit for preparing a radiopharmaceutical preparation, said kit comprising a compound according to the definition of a compound of formula (I), or subformulae thereof.

### BRIEF DESCRIPTION OF THE FIGURES

FIG. 1: Specific binding (non-specific binding subtracted from total) to recombinant TDP-43 aggregates of [³H]-Compound 1 and [³H]-Compound 3 with increasing concentrations using a one-site specific binding model in GraphPad Prism.

FIG. 2: Micro-autoradiography staining with [³H]-Compound 1 on pTDP-43 aggregates in FTLD-TDP brain sections.

Co-localization of silver grains (black dots, left panels) and fluorescent signals from pTDP-43 antibody staining (right panels) in frontal cortex brain sections from FTLD-TDP donors incubated with [³H]-Compound 1 (60 nM). Accumulation of silver grains on pTDP-43 aggregates (arrows) was found.

### DEFINITIONS

Unless defined otherwise, within the meaning of the present application the following definitions apply, and, when appropriate, a term used in the singular will also include the plural and vice versa: Compounds of the invention may have one or more optically active carbons that can exist as racemates and racemic mixtures, stereoisomers (including diastereomeric mixtures and individual diastereomers, enantiomeric mixtures and single enantiomers, mixtures of conformers and single conformers), tautomers, atropoisomers, and rotamers. All isomeric forms are included in the present invention. Compounds described in this specification containing olefinic double bonds include E and Z geometric isomers. Also included in this invention are all salt forms, such as pharmaceutically acceptable salts, polymorphs, hydrates, solvates, prodrugs, and mixtures thereof. Unless specified otherwise, the terms “compound of formula (I)” or “compound of the (present) invention” refer to a “compound of formula (I), or a detectably labelled compound, stereoisomer, polymorph, racemic mixture, tautomer, pharmaceutically acceptable salt, prodrug, hydrate, or solvate thereof, or mixtures thereof”. Unless specified otherwise, the terms “compound of formula (I)” or “compound of the (present) invention” refers to compounds of formula (I), and subformulae thereof, and and isotopically labelled compounds (including, but not limited to ¹⁸F and ³H substitutions). The terms “compound of formula (I)” or “compound of the (present) invention” refers to a compound as defined in any one of embodiments mentioned herein below.

The term “polymorphs” refers to the various crystalline structures of the compounds of the invention. This may include, but is not limited to, crystal morphologies (and amorphous materials) and all crystal lattice forms. Salts can also be crystalline and may exist as more than one polymorph.

Solvates, hydrates as well as anhydrous forms of the salt are also encompassed by the invention. The solvent included in the solvates is not particularly limited and can be any pharmaceutically acceptable solvent. Examples include C₁₋₄ alcohols (such as methanol or ethanol).

“Pharmaceutically acceptable salts” are defined as derivatives of the compounds of the present invention wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compound of formula (I) can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18^{th} ed., Mack Publishing Company, Easton, P A, 1990, p. 1445, the disclosure of which is hereby incorporated by reference. Typically, the pharmaceutically acceptable salts are salts of amine residues in the compounds of the present invention.

The compound of the invention can also be provided in the form of a prodrug, namely a compound which is metabolized in vivo to the active metabolite.

The “patients” or “subjects” in the present invention are typically animals, particularly mammals, more particularly humans and mice. Even more particularly humans.

A “diagnostic composition” is defined in the present invention as a composition comprising the compound of the invention in a form suitable for administration to a patient, wherein the patient is e.g. a mammal such as a human.

“TDP-43 aggregates” are TDP-43-positive multimeric rich assemblies of TDP-43. They can be found in intracellular deposits in a range of diseases termed TDP-43 proteinopathies, in particular in amyotrophic lateral sclerosis (ALS), Alzheimer's disease (AD), Frontotemporal dementia (FTD) and limbic-predominant age-related TDP-43 encephalopathy (LATE). TDP-43 aggregates can be found in the following morphologies: compact oval or crescentic neuronal cytoplasmic inclusions (NCI), lentiform neuronal intranuclear inclusions (Nil), glial cytoplasmic inclusions (GCI), dystrophic neurites (DN) and long tortous neurites. In pathological aggregates TDP-43 often displays a substantial increase in post-translational modifications such as phosphorylation, ubiquitination, acetylation, sumoylation and proteolytic cleavage to generate C-terminal fragments.

The “preclinical state” of disease is defined as the phase of disease where disease-associated changes on the molecular level are not leading to overt clinical representation in the patient.

The “clinical state” of a disease is defined as the phase of a disease where disease-associated changes on the molecular level led to overt clinical representation in the patient.

The terms “diagnosing” or “diagnosis” generally refer to the process or act of recognizing, deciding on or concluding on a disease or condition in a patient on the basis of symptoms and signs and/or from results of a diagnostic procedure. This method should be conducted with a plurality of subjects.

The average amount of the compound bound to the TDP-43 aggregates, if any, is then calculated and defined as a range.

A “normal control value” is determined by conducting the respective method with a plurality of healthy subjects, measuring the amount of the compound bound to the TDP-43 aggregates, if any, for each healthy subject and calculating an average thereof.

A “healthy control subject” or “healthy subject” is a person showing no clinical evidence of neurodegenerative disease. Said person needs to meet the following criteria:
Males and females' subjects which are healthy with no clinically relevant findings upon physical examination.
No family history of TDP-43 proteinopathy, TDP-43 aggregates formation, or other early-onset neurological diseases associated with dementia.
No personal history of clinically significant neurologic and/or psychiatric disorders.
No clinical signs or symptoms of a current neurological deficit such as cognitive impairment or motor deficit.

A “preclinical control value” is determined by conducting the respective method with a plurality of subjects who are in a preclinical state, measuring the amount of the compound bound to the TDP-43 aggregates, if any, for each subject and calculating an average thereof.

A “clinical control value” is determined by conducting the respective method with a plurality of subjects who are in a clinical state, measuring the amount of the compound bound to the TDP-43 aggregates, if any, for each subject and calculating an average thereof.

The terms “predicting” or “prediction” generally refer to an advance declaration, indication or foretelling of a disease or condition in a patient not having a disease, disorder or abnormality. For example, a prediction of a disease, disorder or abnormality in a patient may indicate a probability, chance or risk that the patient will contract the disease, disorder or abnormality, for example within a certain time period or by a certain age.

Detectable labels include suitable isotopes such as radioisotopes, in particular positron emitters or gamma emitters, and include ²H, ³H, ¹⁸F, ¹²³I, ¹²⁴I, ¹²⁵I, ¹³¹I, ¹¹C, ¹³N, ¹⁵O, ^{99m}Tc and ⁷⁷Br, preferably ²H, ³H, ¹C, ¹³N, ¹⁵O, and ¹⁸F, more preferably ²H, ³H and ¹⁸F, even more preferably ³H and ¹⁸F, most preferably ¹⁸F.

The term “Hal”, “halogen” or “halo” means F, Cl, Br or I, particularly Br or I, more particularly Br.

The term “carbocyclic” refers to a 5- or 6-membered carbocyclic ring which is not particularly limited and includes any 5- or 6-membered, saturated or unsaturated carbocyclic ring. Unsaturated carbocyclic rings include, but are not limited to, aromatic rings. Examples of 5- or 6-membered carbocyclic rings include, for instance, phenyl, cyclopentyl, cyclohexyl, cyclopentenyl, and cyclohexenyl. Phenyl being preferred.

The term “heterocyclic ring” refers to a stable 5- or 6-membered heterocyclic ring, is not particularly limited and includes any 5- or 6-membered, saturated or unsaturated heterocyclic ring. Unsaturated heterocyclic rings include, but are not limited to, aromatic rings. The heterocyclic ring contains one or more heteroatoms (for instance, one or two heteroatoms) selected from N, O and S. The heteroatom is/are preferably N or O, more preferably N. Examples of 5- or 6-membered heterocyclic rings include, for instance, pyrrolyl, pyrrolidinyl, furanyl, tetrahydrofuranyl, thiophenyl, imidazolidinyl, pyrazolidinyl, imidazolyl, pyrazolyl, oxathiolidinyl, isoxthiolidinyl, oxathiolyl, isoxathiolyl, thiazolidinyl, iosthiazolidinyl, thiazolyl, and isothiazolyl.

The term “5- or 6-membered heterocycloalkyl ring” is not particularly limited and includes any 5- or 6-membered, saturated or unsaturated heterocyclic ring which is not aromatic. The heterocycloalkyl ring contains one or more (e.g., one or two) heteroatoms selected from N, O and S. The heteroatom is/are preferably N or O, more preferably N. Examples include, for instance, pyrrolidinyl, tetrahydrofuranyl, imidazolidinyl, pyrazolidinyl, oxathiolidinyl, and isoxthiolidinyl.

The term “5-membered heteroaryl ring” is not particularly limited and includes any 5-membered heteroaromatic ring. The heteroaryl ring contains one or more (e.g., one or two) heteroatoms selected from N, O and S. The heteroatom is/are preferably N or O, more preferably N. Examples include, for instance, pyrrolyl, furanyl, thiophenyl, imidazolyl, pyrazolyl, isoxathiolyl, thiazolidinyl, iosthiazolidinyl, thiazolyl, and isothiazolyl. Pyrazolyl being preferred.

The term “6-membered heteroaryl ring” is not particularly limited and includes any 5-membered heteroaromatic ring. The heteroaryl ring contains one heteroatom selected from O and S, or two or more heteroatoms selected from O, N and S. The heteroatom is/are preferably N or O, more preferably N. Examples include, for instance, pyridinyl, pryidazinyl, pyrimidinyl, and pyrazinyl,

The term “leaving group” (LG) as employed herein is any leaving group and means an atom or group of atoms that can be replaced by another atom or group of atoms. Examples are given, e.g., in Synthesis (1982), p. 85-125, table 2, Carey and Sundberg, Organische Synthese, (1995), pages 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33). Preferably, the “leaving group” (LG) is selected from C₁₋₄ alkyl sulfonate and C₆₋₁₀ aryl sulfonate. More preferably, the Leaving Group (LG) is mesylate, tosylate or nosylate. Even more preferably, the Leaving Group (LG) is mesylate.

The term “detecting” as used herein encompasses quantitative and/or qualitative detection.

### DETAILED DESCRIPTION OF THE INVENTION

Various embodiments of the invention are described herein, it will be recognized that features specified in each embodiment may be combined with other specified features to provide further embodiments of the present invention.

It is understood that all of the definitions which are given with respect to the formula (I) apply to all of the subgenuses thereof, including the formulae (Ia), (Ib), (Ic), (Id), (Ie), (If), (I*), (I-H), (I*-H), (I-F), (I*-F), (II), (II*), (I-C), (I*-C), (Ill), (III*), (IV) and (IV*).

All of the definitions which are given with respect to the formula (I-H) or (I-F) apply to (I*-H) or (I*-F), respectively.

In a first aspect, the present invention relates to a compound having the formula (I)

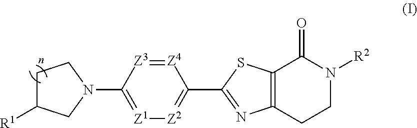

or a detectably labelled compound, stereoisomer, polymorph, racemic mixture, tautomer, pharmaceutically acceptable salt, prodrug, hydrate, or solvate thereof, or mixtures thereof; wherein
Z¹, Z², Z³ and Z⁴ are each selected from CH and N, wherein
   if Z¹ is N, Z² is CH;
   if Z² is N, Z¹ is CH;
   if Z³ is N, Z⁴ is CH;
   if Z⁴ is N, Z³ is CH;
n is 1 or 2;
R⁴ is H or F;
R² is
   a 5- or 6-membered carbocyclic ring which can be optionally substituted with F, NH₂, CN and/or CH₃,
   a 5- or 6-membered heterocycloalkyl ring which can be optionally substituted with F, NH₂, CN and/or CH₃, wherein the 5- or 6-membered heterocycloalkyl ring contains one or more heteroatoms selected from N, O and S,
   a 5-membered heteroaryl ring which can be optionally substituted with F, NH₂, CN and/or CH₃, wherein the 5-membered heteroaryl ring contains one or more heteroatoms selected from N, O and S, or
   a 6-membered heteroaryl ring which can be optionally substituted with F, NH₂, CN and/or CH₃, wherein the 6-membered heteroaryl ring contains one heteroatom selected from O and S, or two or more heteroatoms selected from O, N and S.

The ring

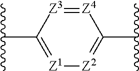

can preferably be selected from

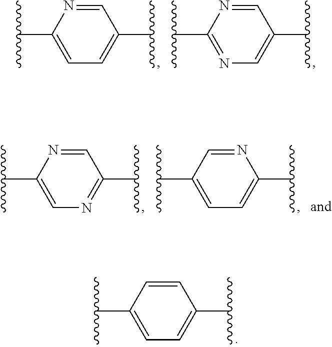

Preferred embodiments of the compound having the formula (I) are

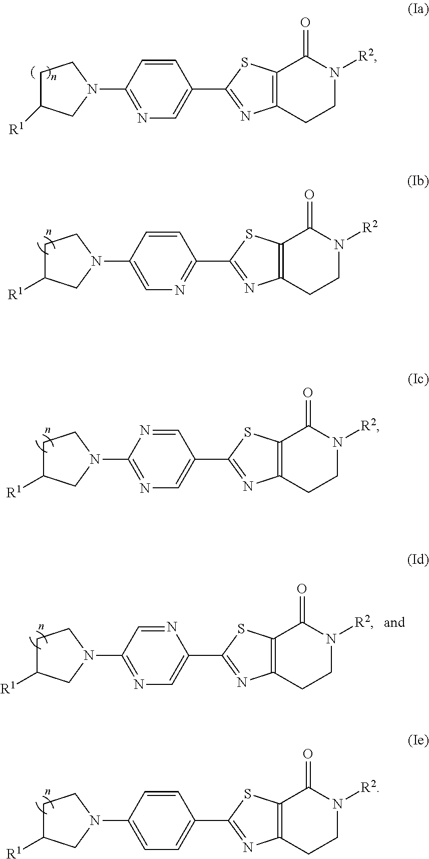

In one embodiment of the first aspect, the present invention relates to a compound having the formula (I*)

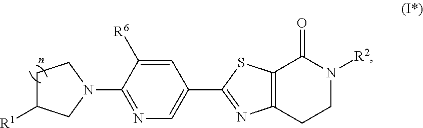

or a detectably labelled compound, stereoisomer, polymorph, racemic mixture, tautomer, pharmaceutically acceptable salt, prodrug, hydrate, or solvate thereof, or mixtures thereof, wherein
n is 1 or 2;
R¹ is H or F;
R² is a 5- or 6-membered carbocyclic or heterocyclic ring which can be optionally substituted with F,
NH₂ and/or CH₃, wherein the heterocyclic ring contains one or more heteroatoms selected from N, O and S; and
R⁶ is H.

The present invention relates to compound of formula (I), wherein n is 1 or 2. In a preferred embodiment n is 1. In another preferred embodiment n is 2.

The present invention relates to a compound of formula (I), wherein R¹ is H or F. In a preferred embodiment R¹ is H. In another preferred embodiment R¹ is F.

The present invention refers to a compound of formula (I), wherein R² is
a 5- or 6-membered carbocyclic ring which can be optionally substituted with F, NH₂, CN and/or CH₃, preferably the optional substituent is F, NH₂, and/or CN,
a 5- or 6-membered heterocycloalkyl ring which can be optionally substituted with F, NH₂, CN and/or CH₃, wherein the 5- or 6-membered heterocycloalkyl ring contains one or more heteroatoms selected from N, O and S, preferably the optional substituent is F, NH₂, and/or CN,
a 5-membered heteroaryl ring which can be optionally substituted with F, NH₂, CN and/or CH₃, wherein the 5-membered heteroaryl ring contains one or more heteroatoms selected from N, O and S, preferably the optional substituent is CN and/or CH₃, or
a 6-membered heteroaryl ring which can be optionally substituted with F, NH₂, CN and/or CH₃ preferably the optional substituent is CN and/or CH₃, wherein the 6-membered heteroaryl ring contains one heteroatom selected from O and S, or two or more heteroatoms selected from O, N and S.

Preferably, R² is
a 5- or 6-membered carbocyclic ring which can be optionally substituted with F, NH₂, CN and/or CH₃,
a 5-membered heteroaryl ring which can be optionally substituted with F, NH₂, CN and/or CH₃, wherein the 5-membered heteroaryl ring contains one or more heteroatoms selected from N, O and S, or
a 6-membered heteroaryl ring which can be optionally substituted with F, NH₂, CN and/or CH₃, wherein the 6-membered heteroaryl ring contains one heteroatom selected from O and S, or two or more heteroatoms selected from O, N and S.

Preferred examples of the ring of R² are given in the Definitions section above. Preferably, R² is phenyl or pyrazolyl, either of which can be optionally substituted with F, NH₂, CN and/or CH₃, e.g., by F, NH₂ and/or CH₃. In one preferred embodiment the phenyl can be optionally substituted by F, NH₂, and/or CN. In one preferred embodiment the pyrazolyl can be optionally substituted by CH₃.

The ring of R² can be optionally substituted with F, NH₂, CN and/or CH₃ (e.g., F, NH₂ and/or CH₃) at any available position. Preferably the 5- or 6-membered carbocyclic or heterocyclic ring is substituted with one or more of F, NH₂, CN and/or CH₃ (e.g., F, NH₂ and/or CH₃).

In one embodiment, the present invention relates to a compound of formula (I) such as (I*), wherein R² is a 5- or 6-membered carbocyclic or heterocyclic ring which can be optionally substituted with F, NH₂ and/or CH₃, wherein the heterocyclic ring contains one or more heteroatoms selected from N, O and S. In a preferred embodiment R² is a 5- or 6-membered carbocyclic or heterocyclic ring which can be optionally substituted with F, NH₂ and/or CH₃ at any available position. Preferably the 5- or 6-membered carbocyclic or heterocyclic ring is substituted with one or more of F, NH₂ and/or CH₃. R⁶ is H.

In a preferred embodiment, the present invention relates to a compound of formula (I), wherein R² is (i)

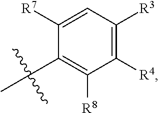

wherein
R³ is F, R⁴ is NH₂, R⁷ is H, and R⁸ is H;
R³ is NH₂, R⁴ is F, R⁷ is H, and R⁸ is H,
R³ is CN, R⁴ is NH₂, R⁷ is H, and R⁸ is H,
R³ is H, R⁴ is NH₂, R⁷ is H, and R⁸ is CN, or
R³ is H, R⁴ is NH₂, R⁷ is H, and R⁸ is F.

In another preferred embodiment, the present invention relates to a compound of formula (I), wherein R² is (ii)

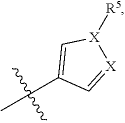

wherein X is N and R⁵ is CH₃.

In another preferred embodiment, the present invention relates to a compound of formula (I), wherein R² is (ii)

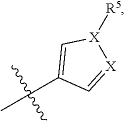

wherein X is N and R⁵ is H.

In one embodiment, the compound of formula (I) is defined as n is 1; R¹ is F; and R² is

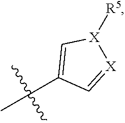

wherein X is N and R⁵ is CH₃ or H, preferably CH₃.

In one embodiment, the compound of formula (I) is defined as n is 1; R¹ is H; and R² is

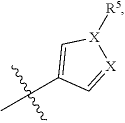

wherein X is N and R⁵ is CH₃ or H, preferably CH₃.

In one embodiment, the compound of formula (I) is defined as n is 2; R¹ is F; and R² is

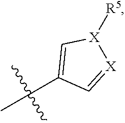

wherein X is N and R⁵ is CH₃ or H, preferably CH₃.

In one embodiment, the compound of formula (I) is defined as n is 2; R¹ is H; and R² is

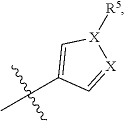

wherein X is N and R⁵ is CH₃ or H, preferably CH₃.

In one embodiment, the compound of formula (I) is defined as n is 2; R¹ is H; and R² is

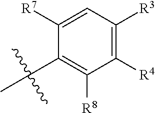

wherein R³ is F, R⁴ is —NH₂, R¹ is H, R⁸ is H.

In one embodiment, the compound of formula (I) is defined as n is 2; R¹ is H; and R² is

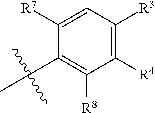

wherein R³ is F, R⁴ is —NH₂, R¹ is H, R⁸ is H.

In one embodiment, the compound of formula (I) is defined as n is 1; R¹ is F; and R² is

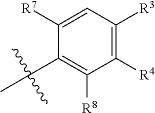

wherein R³ is F, R⁴ is —NH₂, R⁷ is H, R⁸ is H.

In one embodiment, the compound of formula (I) is defined as n is 1; R¹ is H; and R² is

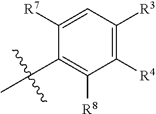

wherein R³ is NH₂, R⁴ is F, R⁷ is H, and R⁸ is H.

In one embodiment, the compound of formula (I) is defined as n is 1; R¹ is H; and R² is

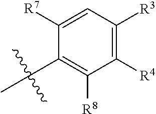

wherein R³ is ON, R⁴ is NH₂, R⁷ is H, and R⁸ is H.

In one embodiment, the compound of formula (I) is defined as n is 1; R¹ is F; and R² is

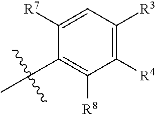

wherein R³ is CN, R⁴ is NH₂, R⁷ is H, and R⁸ is H.

In one embodiment, the compound of formula (I) is defined as n is 1; R¹ is H; and R² is

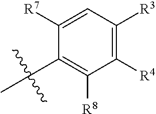

wherein R³ is H, R⁴ is NH₂, R⁷ is H, and R⁸ is CN.

In one embodiment, the compound of formula (I) is defined as n is 1; R¹ is F; and R² is

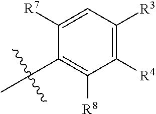

wherein R³ is H, R⁴ is NH₂, R⁷ is H, and R⁸ is CN.

In one embodiment, the compound of formula (I) is defined as n is 1; R¹ is H; and R² is

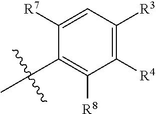

wherein R³ is H, R⁴ is NH₂, R⁷ is H, and R¹ is F.

In one embodiment, the compound of formula (I) is defined as n is 1; R¹ is F; and R² is

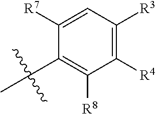

wherein R³ is H, R⁴ is NH₂, R⁷ is H, and R¹ is F.

In all of the above compounds having the formula (I*), R⁶ is H.

Preferred compounds of formula (I) include

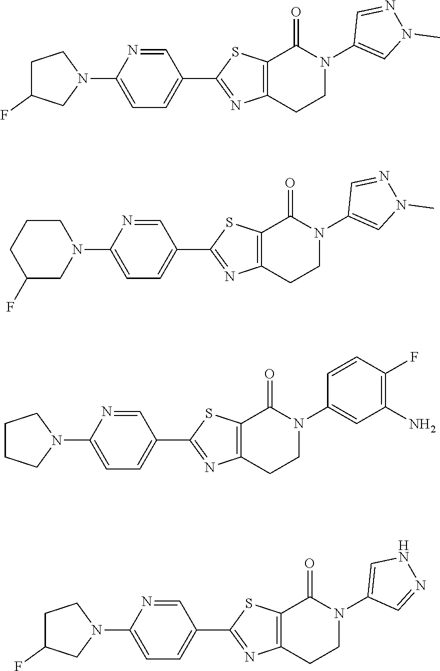 [SMILES: Cn1cc(N2CCc3nc(-c4ccc(N5CCC(F)C5)nc4)sc3C2=O)cn1 ; Cn1cc(N2CCc3nc(-c4ccc(N5CCCC(F)C5)nc4)sc3C2=O)cn1 ; Nc1cc(N2CCc3nc(-c4ccc(N5CCCC5)nc4)sc3C2=O)ccc1F ; O=C1c2sc(-c3ccc(N4CCC(F)C4)nc3)nc2CCN1c1cn[nH]c1]

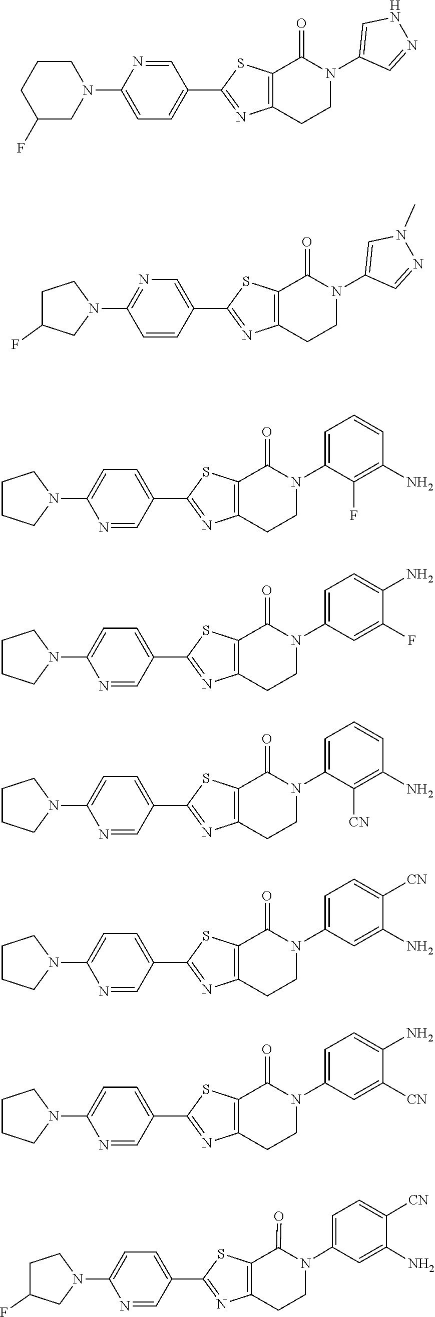

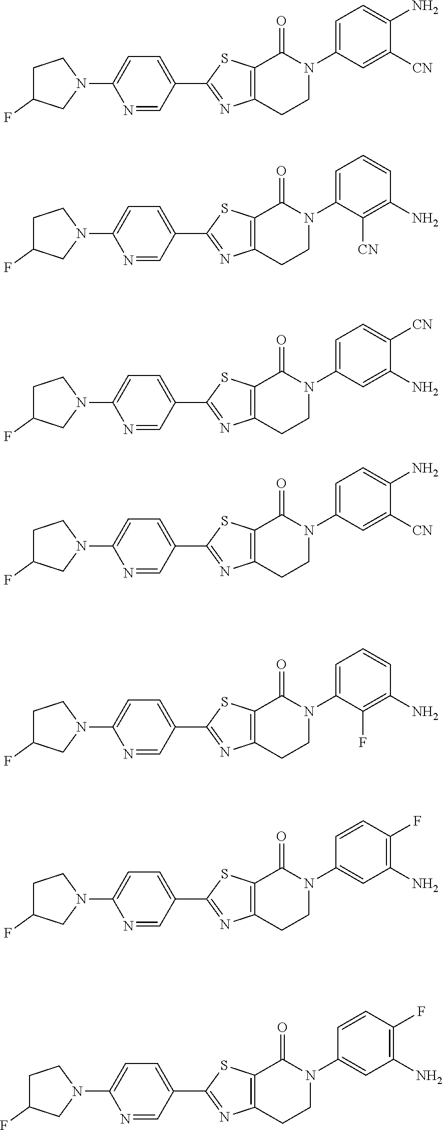

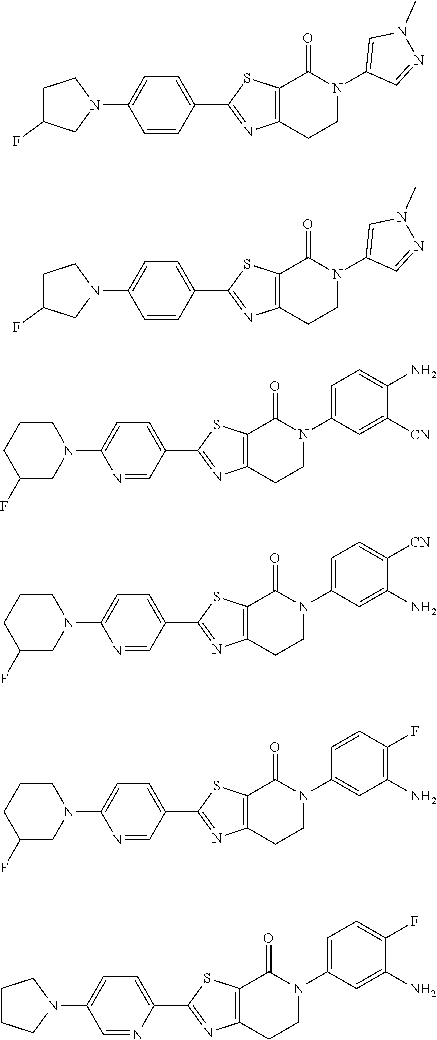 [SMILES: Cn1cc(N2CCc3nc(-c4ccc(N5CCC(F)C5)cc4)sc3C2=O)cn1 ; Cn1cc(N2CCc3nc(-c4ccc(N5CCC(F)C5)cc4)sc3C2=O)cn1 ; N#Cc1cc(N2CCc3nc(-c4ccc(N5CCCC(F)C5)nc4)sc3C2=O)ccc1N ; N#Cc1ccc(N2CCc3nc(-c4ccc(N5CCCC(F)C5)nc4)sc3C2=O)cc1N ; Nc1cc(N2CCc3nc(-c4ccc(N5CCCC(F)C5)nc4)sc3C2=O)ccc1F ; Nc1cc(N2CCc3nc(-c4ccc(N5CCCC5)cn4)sc3C2=O)ccc1F]

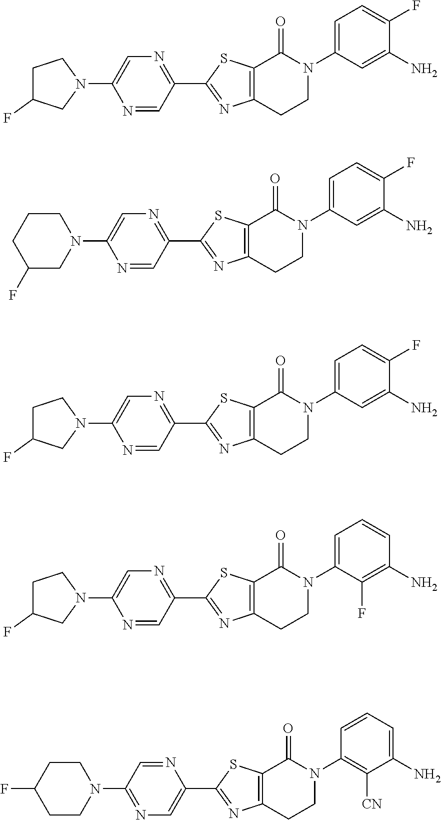 [SMILES: Nc1cc(N2CCc3nc(-c4cnc(N5CCC(F)C5)cn4)sc3C2=O)ccc1F ; Nc1cc(N2CCc3nc(-c4cnc(N5CCCC(F)C5)cn4)sc3C2=O)ccc1F ; Nc1cc(N2CCc3nc(-c4cnc(N5CCC(F)C5)cn4)sc3C2=O)ccc1F ; Nc1cccc(N2CCc3nc(-c4cnc(N5CCC(F)C5)cn4)sc3C2=O)c1F ; N#Cc1c(N)cccc1N1CCc2nc(-c3cnc(N4CCC(F)CC4)cn3)sc2C1=O]

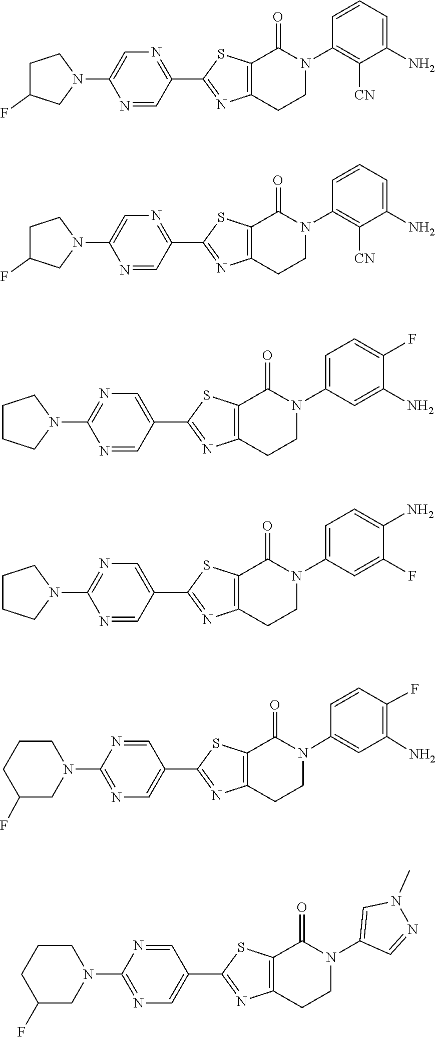 [SMILES: N#Cc1c(N)cccc1N1CCc2nc(-c3cnc(N4CCC(F)C4)cn3)sc2C1=O ; N#Cc1c(N)cccc1N1CCc2nc(-c3cnc(N4CCC(F)C4)cn3)sc2C1=O ; Nc1cc(N2CCc3nc(-c4cnc(N5CCCC5)nc4)sc3C2=O)ccc1F ; Nc1ccc(N2CCc3nc(-c4cnc(N5CCCC5)nc4)sc3C2=O)cc1F ; Nc1cc(N2CCc3nc(-c4cnc(N5CCCC(F)C5)nc4)sc3C2=O)ccc1F ; Cn1cc(N2CCc3nc(-c4cnc(N5CCCC(F)C5)nc4)sc3C2=O)cn1]

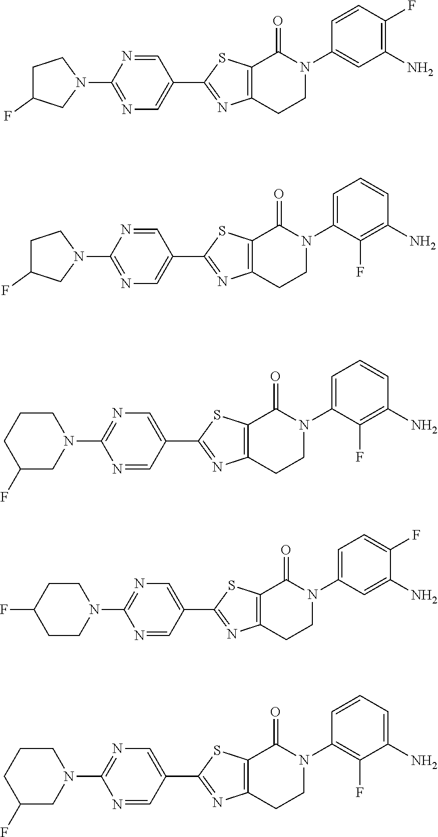 [SMILES: Nc1cc(N2CCc3nc(-c4cnc(N5CCC(F)C5)nc4)sc3C2=O)ccc1F ; Nc1cccc(N2CCc3nc(-c4cnc(N5CCC(F)C5)nc4)sc3C2=O)c1F ; Nc1cccc(N2CCc3nc(-c4cnc(N5CCCC(F)C5)nc4)sc3C2=O)c1F ; Nc1cc(N2CCc3nc(-c4cnc(N5CCC(F)CC5)nc4)sc3C2=O)ccc1F ; Nc1cccc(N2CCc3nc(-c4cnc(N5CCCC(F)C5)nc4)sc3C2=O)c1F]

According to one embodiment, preferred compounds of formula (I) can be selected from

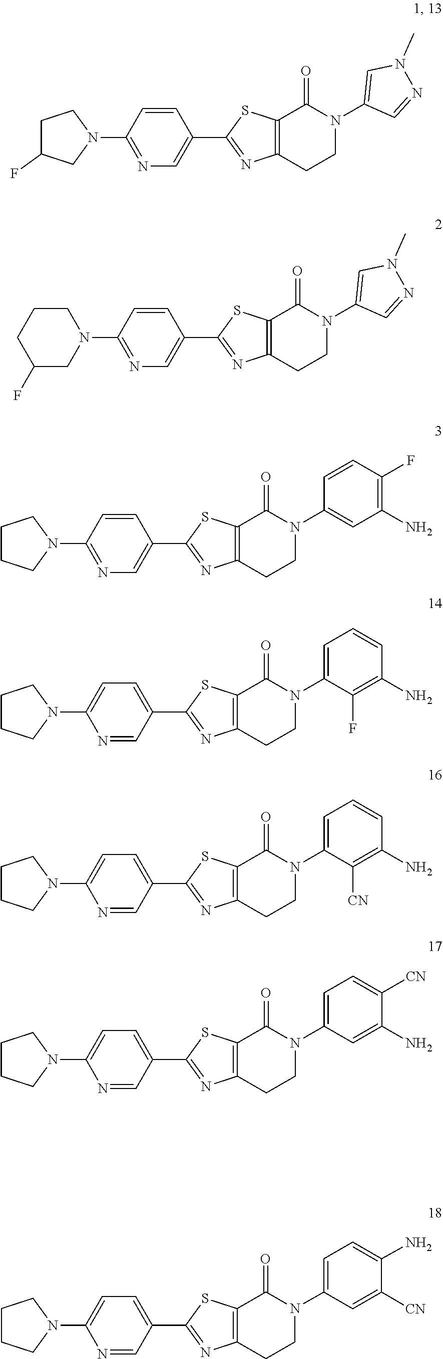

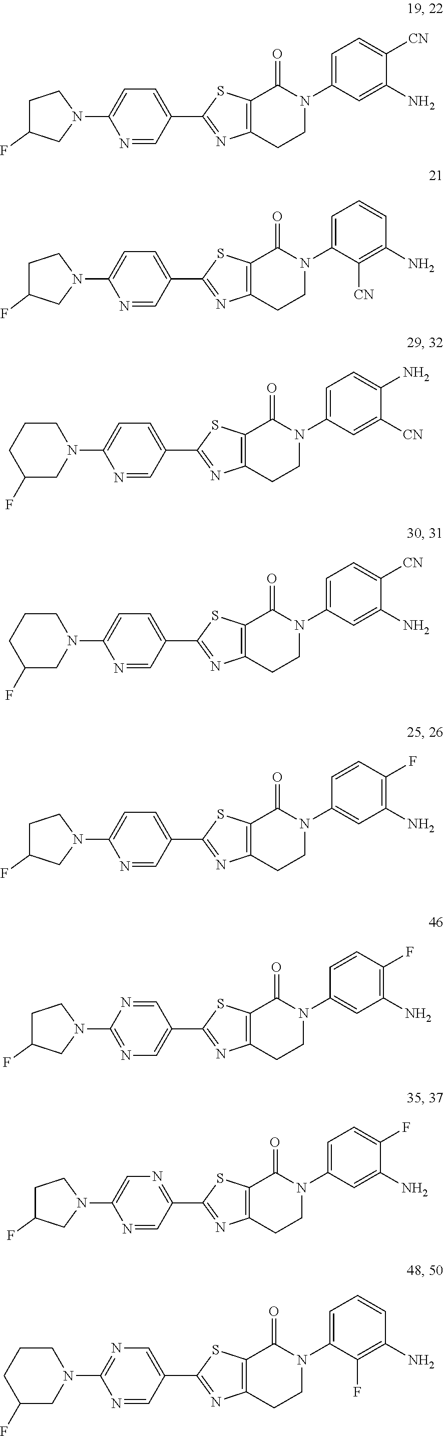

In one embodiment preferred compounds of formula (I) can be selected from the following stereoisomers:

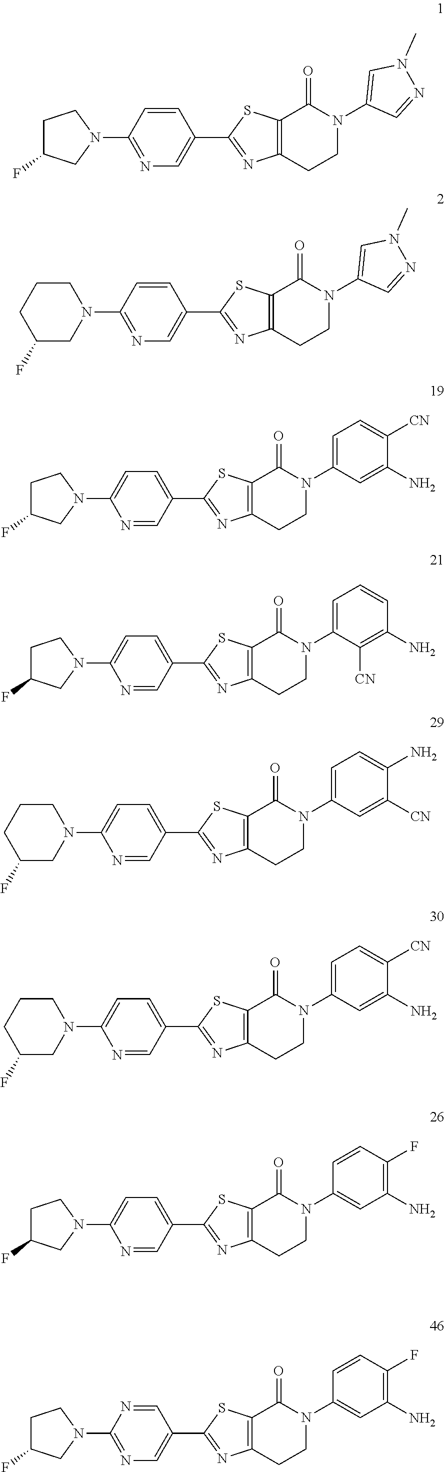

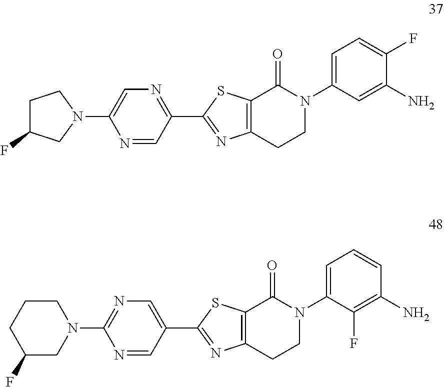 [SMILES: Nc1cc(N2CCc3nc(-c4cnc(N5CC[C@H](F)C5)cn4)sc3C2=O)ccc1F ; Nc1cccc(N2CCc3nc(-c4cnc(N5CCC[C@H](F)C5)nc4)sc3C2=O)c1F]

In one embodiment, the present invention relates to a compound of formula (I), which comprises a detectable label. Preferably, the compound of formula (I) comprises one or more detectable labels.

The type of the detectable label is not specifically limited and will depend on the detection method chosen. Examples of possible detectable labels include isotopes such as radioisotopes (namely radionuclides), in particular, positron emitters or gamma emitters. The detectable label such as the radioisotope, in particular, the positron emitter or gamma emitter, should be present in an amount, which is not identical to the natural amount of the respective isotope. Furthermore, the employed amount should allow detection thereof by the chosen detection method.

In a preferred embodiment, the detectable label is selected from ³H and ¹⁸F, most preferably ¹⁸F. The detectable label can be present at any available position. Typically, the detectable label is a radioactive isotope of one of the atoms which are present in the compound of formula (I). For instance, any reference to “F” in the present invention covers ¹⁹F (stable) or ¹⁸F (detectable label). Any reference to “H” covers ¹H (stable) or ³H (detectable label, so called tritium and represented herein as “T”).

Isotopic variations of the compounds of the invention can generally be prepared by conventional procedures such as by the illustrative methods or by the preparations described in the Examples and Preparative Examples hereinafter using appropriate isotopic variations of suitable reagents, commercially available or prepared by known synthetic techniques. Radioisotopes, in particular positron emitters or gamma emitters, can be included into the compounds of the invention by methods which are usual in the field of organic synthesis. Typically, they will be introduced by using a correspondingly labeled starting material. Illustrative methods of introducing detectable labels are described, for instance, in U.S. Pat. No. 8,932,557 which is incorporated herein by reference. ¹⁸F can be attached at any position which is suitable for attaching a fluorine. ¹⁸F-labeled compounds are particularly suitable for imaging applications such as positron emission tomography (PET). The corresponding compounds which include natural fluorine isotope ⁴⁹F are also of particular interest as they can be used as analytical standards and references during manufacturing, quality control, release, and clinical use of their ¹⁸F-analogs.

In the compounds having the formula (I), ⁴⁸F can be present, for instance, as the F substituent of R² or as R¹. Preferably it is present as R¹.

If ³H is employed as a detectable label it is preferably attached in the form of —CT₃ (T means ³H) at any position at which a CH₃ group can be attached. Substitution with radioisotopes such as ³H may afford certain diagnostic advantages resulting from greater metabolic stability by reducing, for example, defluorination, increasing in vivo half-life or reducing dosage requirements, while keeping or improving the original compound efficacy.

In one embodiment, the present invention relates to Tritium (³H) detectably labeled compounds having the formula (I), as described above, wherein at least one Hydrogen (H) is replaced by a detectable label selected from Tritium (³H). Tritium (³H) detectably labeled compounds having the formula (I) are preferably defined wherein 1 to 3 Hydrogens (H) are replaced by Tritium (³H). Tritium (³H) detectably labeled compounds having the formula (I) are more preferably defined wherein 2 or 3 Hydrogens (H) are replaced by Tritium (³H). Tritium (³H) detectably labeled compounds having the formula (I) are even more preferably defined wherein 3 Hydrogens (H) are replaced by Tritium (³H).

In one embodiment, the present invention provides a compound of formula (I), having a formula (I-T). In particular, the present invention relates to Tritium (³H) detectably labeled compounds having the formula (I-T)

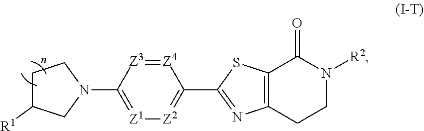

or a stereoisomer, polymorph, racemic mixture, tautomer, pharmaceutically acceptable salt, prodrug, hydrate, or solvate thereof, or mixtures thereof; wherein n, Z¹, Z², Z³, Z⁴, R¹, and R² are as defined herein with respect to compound of formula (I). In one embodiment, at least one of Z¹, Z², Z³, Z⁴ is selected from C-T. In another embodiment, R² is substituted by at least one CT₃ or at least one of the hydrogen atoms in R² is replaced by T.

In one embodiment, the present invention provides a compound of formula (I*), having a formula (I*-T). In particular, the present invention relates to Tritium (³H) detectably labeled compounds having the formula (I*-T)

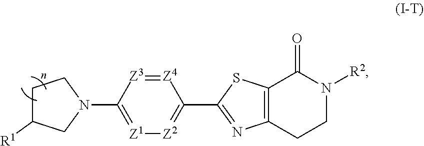

or a stereoisomer, polymorph, racemic mixture, tautomer, pharmaceutically acceptable salt, prodrug, hydrate, or solvate thereof, or mixtures thereof; wherein n, R¹, R² and R⁶ are as defined herein with respect to compound of formula (I*).

In a preferred embodiment, the present invention relates to a compound of formula (I-T) or (I*-T),
wherein
n is 1 or 2; preferably n is 1;
R¹ is H or F;
(with respect to formula (I-T): Z¹, Z², Z³ and Z⁴ are as defined above; with respect to formula (I*-T):
R⁶ is T or H);
T is ³H; and
wherein
R² is

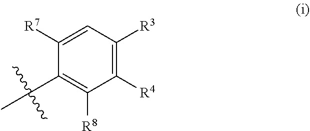

wherein R³ is F, R⁴ is —NH₂, and at least one of R⁷ and R⁶ is T and, if applicable, the other is H;
preferably R⁷ is T; R⁸ is T;
or
R² is

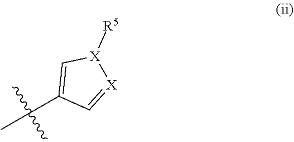

wherein X is N and R⁵ is tritiated CH₃ (CT₃).

In one embodiment, the present invention relates to a compound of formula (I-T) or (I*-T), wherein
R¹ is H;
(with respect to formula (I-T): Z¹, Z², Z³ and Z⁴ are as defined above; with respect to formula (I*-T):
R⁶ is T); and
R² is

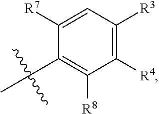

wherein R³ is F; R⁴ is —NH₂; and at least one of R⁷ and R⁸ is T and, if applicable, the other is H.

Preferably R⁷ is T; R⁶ is T.

In one embodiment, the present invention relates to a compound of formula (I-T) or (I*-T) wherein
R¹ is F;
(with respect to formula (I-T): Z¹, Z², Z³ and Z⁴ are as defined above; with respect to formula (I*-T):
R⁶ is H); and
R² is

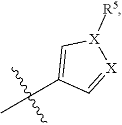

wherein X is N, and R⁵ is tritiated CH₃ (CT₃).

Preferred Tritium (³H) detectably labeled compounds of formula (I-T), according to present invention can be selected from (wherein T means ³H)

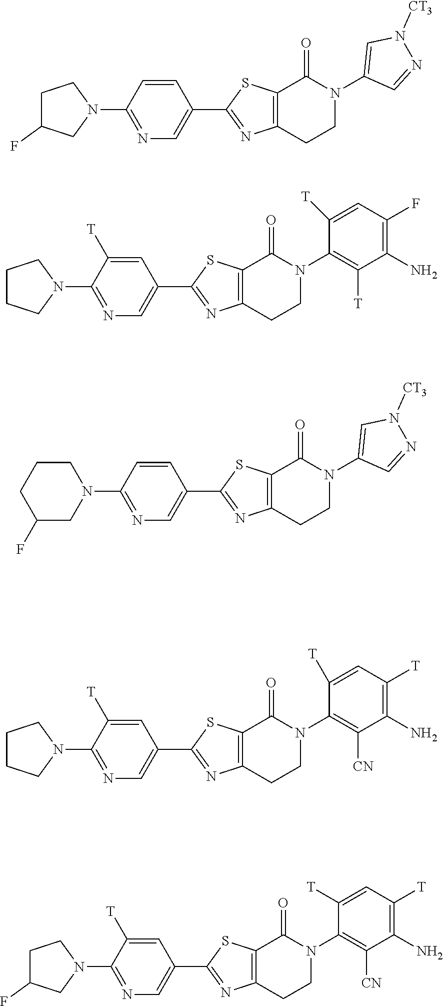

More preferably, the Tritium (³H) detectably labeled compounds of formula (I-T) or (I*-T) according to the present invention can be selected from the stereoisomers such as (wherein T means ³H)

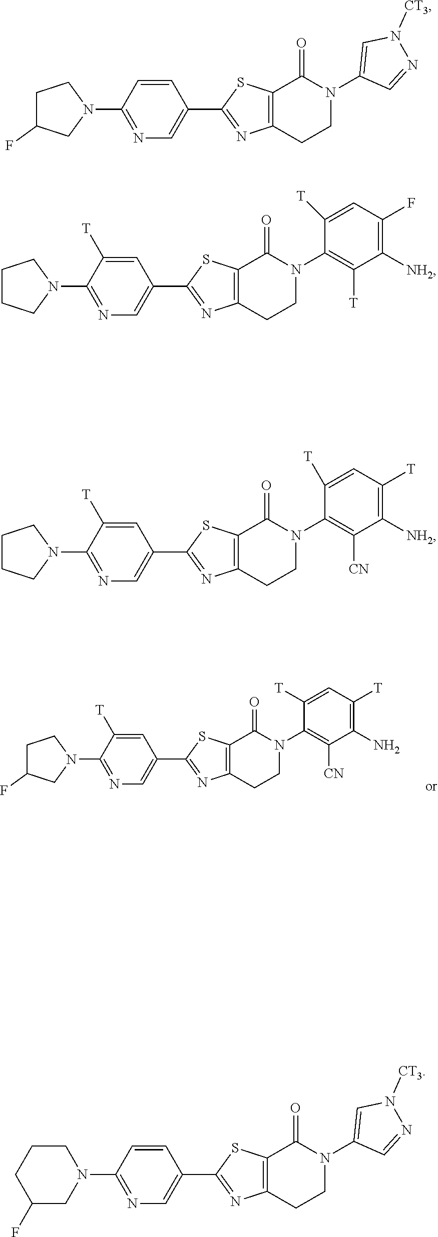

Even more preferably, the Tritium (³H) detectably labeled compound for formula (I-T), according to the present invention can be the stereoisomer (wherein T means ³H)

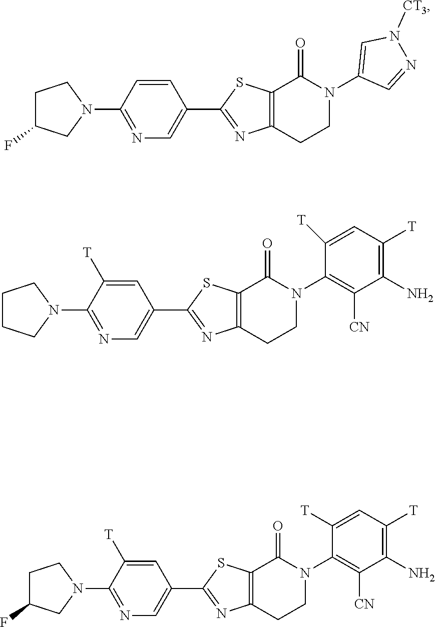

In one embodiment, the present invention provides a ¹⁸F detectably labeled compound of formula (I-F)

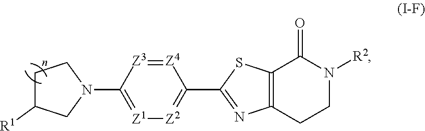

or a stereoisomer, polymorph, racemic mixture, tautomer, pharmaceutically acceptable salt, prodrug, hydrate, or solvate thereof, or mixtures thereof; wherein n, Z¹, Z², Z³, Z⁴, R¹, and R² are as defined herein with respect to compound of formula (I) and at least one F is ¹⁸F.

In one embodiment, the present invention provides a ¹⁸F detectably labeled compound of formula (I*-F)

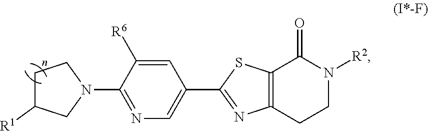

or a stereoisomer, polymorph, racemic mixture, tautomer, pharmaceutically acceptable salt, prodrug, hydrate, or solvate thereof, or mixtures thereof; wherein n, R¹, R² and R⁶ are as defined herein with respect to compound of formula (I*).

In a preferred embodiment, the present invention relates to a compound of formula (I-F), wherein R¹ is ¹⁸F (detectable label).

In a further preferred embodiment,
n is 1 or 2;
R¹ is ¹⁸F (detectable label); and
R² is

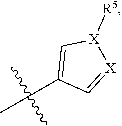

wherein X is N and R⁵ is CH₃ or H; or
R² is

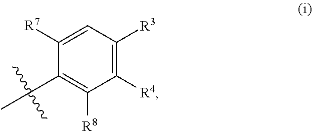

wherein
R³ is F, R⁴ is NH₂, R⁷ is H, and R⁸ is H,
R³ is NH₂, R⁴ is F, R⁷ is H, and R⁸ is H,
R³ is CN, R⁴ is NH₂, R⁷ is H, and R⁸ is H,
R³ is H, R⁴ is NH₂, R⁷ is H, and R⁸ is CN, or
R³ is H, R⁴ is NH₂, R⁷ is H, and R⁸ is F.

In a preferred embodiment, the present invention relates to a compound of formula (I*-F), wherein
n is 1 or 2;
R¹ is ¹⁸F (detectable label);
R² is

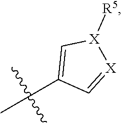

wherein X is N and R⁵ is CH₃; and
R⁶ is H.

In a preferred embodiment n is 1. In another preferred embodiment n is 2.

Preferred ¹⁸F detectably labeled compounds of formula (I-F) according to the present invention can be selected from

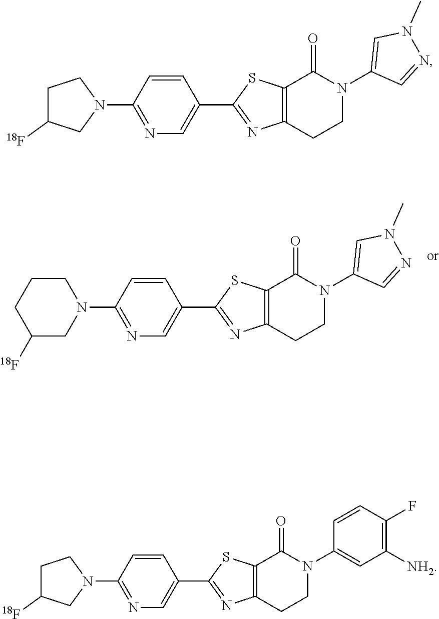 [SMILES: Cn1cc(N2CCc3nc(-c4ccc(N5CCC([18F])C5)nc4)sc3C2=O)cn1 ; Cn1cc(N2CCc3nc(-c4ccc(N5CCCC([18F])C5)nc4)sc3C2=O)cn1 ; Nc1cc(N2CCc3nc(-c4ccc(N5CCC([18F])C5)nc4)sc3C2=O)ccc1F]

More preferably, the ¹⁸F detectably labeled compounds of formula (I-F) is

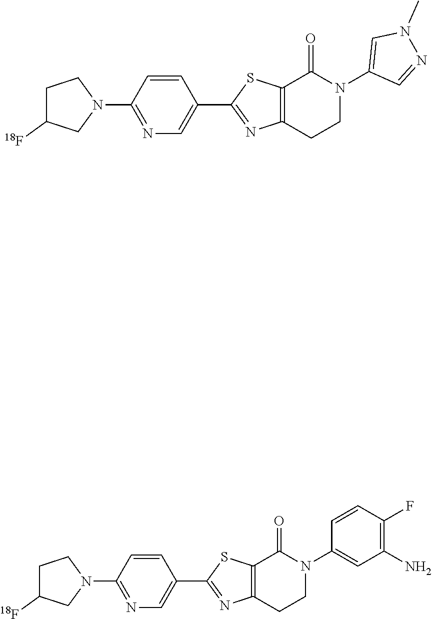 [SMILES: Cn1cc(N2CCc3nc(-c4ccc(N5CCC([18F])C5)nc4)sc3C2=O)cn1 ; Nc1cc(N2CCc3nc(-c4ccc(N5CCC([18F])C5)nc4)sc3C2=O)ccc1F]

Even more preferably, the ¹⁸F detectably labeled compound of formula (I-F) according to the present invention can be the stereoisomer

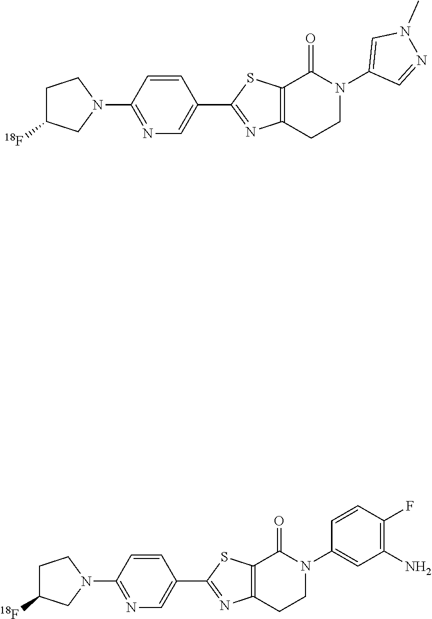 [SMILES: Cn1cc(N2CCc3nc(-c4ccc(N5CC[C@@H]([18F])C5)nc4)sc3C2=O)cn1 ; Nc1cc(N2CCc3nc(-c4ccc(N5CC[C@H]([18F])C5)nc4)sc3C2=O)ccc1F]

### Diagnostic Compositions

In a second aspect, the present invention relates to a diagnostic composition comprising a compound of formula (I), as described above, and optionally at least one physiologically acceptable carrier, diluent, adjuvant and/or excipient.

The compounds of the present invention are particularly suitable for imaging TDP-43 aggregates. The imaging can be conducted in mammals, preferably in humans. The imaging is preferably in vitro imaging, ex vivo imaging, or in vivo imaging. More preferably the imaging is in vivo imaging. Even more preferably, the imaging is brain imaging. The imaging can also be eye/retinal imaging or imaging of tissue of the central nervous system.

The compounds of the present invention are particularly suitable for use in diagnostics. The diagnostics can be conducted for mammals, preferably for humans. The tissue of interest on which the diagnostics is conducted can be brain, tissue of the central nervous system, tissue of the eye (such as retinal tissue) or other tissues, or body fluids such as cerebrospinal fluid (CSF). The tissue is preferably brain tissue.

A “diagnostic composition” is defined in the present invention as a composition comprising one or more compounds of the present invention, in a form suitable for administration to a patient, (e.g., a mammal such as a human), and which is suitable for use in the diagnosis of the specific disease, disorder or abnormality at issue. In one embodiment, the diagnostic composition comprises a detectably labeled compound of the invention as described above and optionally at least one physiologically acceptable carrier, diluent, adjuvant and/or excipient.

Preferred detectably labeled compounds of the invention are of formula (I-T) or (I-F).

The diagnostic composition is suitable for use in the diagnosis of a disease, disorder or abnormality associated with TDP-43 aggregates or a TDP-43 proteinopathy, as defined herein below. Preferably a diagnostic composition further comprises, optionally, a physiologically acceptable excipient, carrier, diluent, or adjuvant. Administration is preferably carried out as defined below. More preferably by injection of the composition as an aqueous solution. The diagnostic composition may optionally contain further ingredients such as buffers; pharmaceutically acceptable solubilizers (e.g., cyclodextrins or surfactants such as Pluronic, Tween or phospholipids); and pharmaceutically acceptable stabilizers or antioxidants (such as ascorbic acid, gentisic acid or para-aminobenzoic acid). The dose of the compound of the invention will vary depending on the exact compound to be administered, the weight of the patient, and other variables as would be apparent to a physician skilled in the art.

While it is possible for the compounds of the invention to be administered alone, it is preferable to formulate them into a diagnostic composition in accordance with standard pharmaceutical practice. Thus, a diagnostic composition which comprises a diagnostically effective amount of a compound of the invention in combination with a pharmaceutically acceptable carrier, diluent, adjuvant and/or excipient is part of the invention. The preferred pharmaceutically acceptable carrier, diluent, adjuvant and/or excipient is one that is physiologically compatible with the diagnostic composition according to the present invention.

Pharmaceutically acceptable excipients are well known in the pharmaceutical art, and are described, for example, in Remington's Pharmaceutical Sciences, 18^{th} Ed. (Alfonso R. Gennaro, ed.; Mack Publishing Company, Easton, P A, 1990). The pharmaceutically acceptable excipient can be selected with regard to the intended route of administration and standard pharmaceutical practice. The excipient must be acceptable in the sense of being not deleterious to the recipient thereof.

Pharmaceutically useful excipients, carriers, adjuvants and diluents that may be used in the formulation of the diagnostic composition of the present invention may comprise, for example, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-β-cyclodextrin, polyvinylpyrrolidone, low melting waxes, and ion exchange resins.

The routes for administration (delivery) of the compounds of the invention include, but are not limited to, one or more of: intravenous, gastrointestinal, intraspinal, intraperitoneal, intramuscular, oral (e. g. as a tablet, capsule, or as an ingestible solution), topical, mucosal (e. g. as a nasal spray or aerosol for inhalation), nasal, parenteral (e. g. by an injectable form), intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. Preferably, the route for administration (delivery) of the compounds of the invention is parenteral.

If the compounds of the present invention (for instance, detectably labeled compounds such as those with a ³H or ¹⁸F detectable label) are administered parenterally, then examples of such routes of administration include one or more of: intravenously, intraarterially, intraperitoneally, intrathecally, intraventricularly, intraurethrally, intrasternally, intracranially, intramuscularly or subcutaneously and/or using infusion techniques. For parenteral administration, the compounds are best used in the form of a sterile aqueous solution which may contain other excipients. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Typically, a physician will determine the actual dosage which will be most suitable for an individual patient. The dose of the compounds of the present invention (for instance, detectably labeled compounds such as those with a ³H or ¹⁸F detectable label) will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

Due to their design and their binding characteristics, the compounds of the present invention, as defined herein, can be use in the diagnosis of diseases, disorders and abnormalities associated with TDP-43 aggregates. The compounds of the present invention are particularly suitable for positron emission tomography imaging of TDP-43 aggregates.

The compounds of the present invention, as disclosed herein, are particularly suitable for use in the diagnosis of diseases, disorders or abnormalities associated with TDP-43 aggregates or the TDP-43 proteinopathy, such as disease, disorder or abnormality selected from, but not limited to, Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) including Frontotemporal lobar dementia TDP-43 or Frontotemporal lobar degeneration with ubiquitin-positive TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (such as nfvPPA), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy, Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (Sporadic inclusion body myositis, Inclusion body myopathy with a mutation in the valosin-containing protein (VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES), Traumatic Brain Injury (TBI), Dementia with Lewy Bodies (DLB) and Parkinson's disease (PD), preferably, the disease, the disorder or the abnormality associated with TDP-43 aggregates or the TDP-43 proteinopathy is selected from Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

In one embodiment, the diseases, disorders or abnormalities associated with TDP-43 aggregates or the TDP-43 proteinopathy is amyotrophic lateral sclerosis (ALS).

In one embodiment, the diagnosis of diseases, disorders or abnormalities associated with TDP-43 aggregates or the TDP-43 proteinopathy is Alzheimer's disease (AD).

In one embodiment, the diagnosis of diseases, disorders or abnormalities associated with TDP-43 aggregates or the TDP-43 proteinopathy is Frontotemporal dementia (FTD) including Frontotemporal lobar dementia TDP-43 or Frontotemporal lobar degeneration with TDP-43 inclusions (FTLD-TDP).

In one embodiment, the diagnosis of diseases, disorders or abnormalities associated with TDP-43 aggregates or the TDP-43 proteinopathy is limbic-predominant age-related TDP-43 encephalopathy (LATE).

### Methods and Uses

In a third aspect, the present invention relates to the methods and uses as listed below
A method of imaging a disease, disorder or abnormality associated with TDP-43 aggregates in a subject;
A method of positron emission tomography (PET) imaging of TDP-43 aggregates in a tissue of a subject;
A method for the detection and optionally quantification of TDP-43 aggregates in a tissue of a subject;
A method of the diagnostic imaging of the brain of a subject;
A method of determining an amount of TDP-43 aggregates in a sample or a specific body part or body area;
A method of diagnosing a disease, disorder or abnormality associated with TDP-43 aggregates or of diagnosing a TDP-43 proteinopathy;
A method of collecting data for the diagnosis of a disease, disorder or abnormality associated with TDP-43 aggregates or for the diagnosis of a TDP-43 proteinopathy;
A method of collecting data for determining a predisposition to a disease, disorder or abnormality associated with TDP-43 aggregates or a TDP-43 proteinopathy;
A method of collecting data for monitoring the progression of a disease, disorder or abnormality associated with TDP-43 aggregates or for monitoring the progression of a TDP-43 proteinopathy in a patient,
A method of collecting data for predicting responsiveness of a patient suffering from a disease, disorder or abnormality associated with TDP-43 aggregates to a treatment with a medicament;
Use of a compound of the invention as a TDP-43 aggregates' biomarker or a TDP-43 proteinopathy biomarker,
Use of a compound of the invention as a TDP-43 proteinopathy diagnostic agent or diagnostic tool,
Use of a compound of the invention as an in vitro analytical reference or an in vitro screening tool.

Any of the compounds of the present invention (e.g. compound of formula (I), (I-T) or (I-F)) can be used in the above summarized methods. Preferably, said compounds are detectably labeled compounds (e.g. such as those with a ³H or ⁹⁸F detectable label).

The methods of the invention can include the step of bringing a sample, a specific body part or a body area suspected to contain TDP-43 aggregates into contact with a compound of the invention.

The body is preferably of a mammal, more preferably of a human, including the full body or partial body area/part of the patient suspected to contain TDP-43 aggregates.

The sample can be selected from tissue or body fluids suspected to contain TDP-43 aggregates, the sample being obtained from the patient. Preferably, the tissue is selected from tissue of the central nervous system (CNS), eye tissue or brain tissue, more preferably brain tissue. Examples of body fluids include cerebrospinal fluid (CSF) or blood. The sample can be obtained from a mammal, more preferably a human. Preferably, the sample is an in vitro sample from a patient.

An in vitro sample or a specific body part or body area obtained from a patient can be brought into contact with a compound of the invention by direct incubation.

In an in vivo method, the specific body part or body area can be brought into contact with a compound of the invention by administering an effective amount of a compound of the invention to the patient. The effective amount of a compound of the invention is an amount which is suitable for allowing the presence or absence of TDP-43 aggregates in the specific body part or body area to be determined using the chosen analytical technique.

The step of allowing the compound of the invention to bind to the TDP-43 aggregates includes allowing sufficient time for said binding to happen. The amount of time required for binding will depend on the type of test (e.g., in vitro or in vivo) and can be determined by a person skilled in the field by routine experiments. In an in vitro method the amount of time will depend on the sample or specific body part or body area and can range, for instance, from about 30 min to about 120 min. In an in vivo method, the amount of time will depend on the time which is required for the compound of the invention to reach the specific body part or body area suspected to contain TDP-43 aggregates. The amount of time should not be too long to avoid washout and/or metabolism of the compound of the invention. The duration can range, for instance, from about 0 min to about 240 min (which is the duration of a PET scan during initial compound characterization (NHP PET and later FiH-study)).

The method of detecting the compound of the invention bound to the TDP-43 aggregates is not particularly limited and depends, among others, on the detectable label, the type of sample, specific body part or body area and whether the method is an in vitro or in vivo method. Possible detection methods include, but are not limited to, a fluorescence imaging technique or a nuclear imaging technique such as positron emission tomography (PET), single photon emission computed tomography (SPECT), magnetic resonance imaging (MRI), and contrast-enhanced magnetic resonance imaging (MRI). The fluorescence imaging technique and/or nuclear imaging technique can be employed for monitoring and/or visualizing the distribution of the compound of the invention within the sample or the body. The imaging system is such to provide an image of bound detectably label such as radioisotopes, in particular positron emitters or gamma emitters, as present in the tested sample, the tested specific body part or the tested body area. Preferably, the compound of the invention bound to the TDP-43 aggregates is detected by an imaging apparatus such as PET or a SPECT scanner. The amount of the compound bound to the TDP-43 can be determined by visual or quantitative analysis, for example, using PET scan images.

In one embodiment, the presence or absence of a compound of the invention bound with the TDP-43 aggregates can be correlated with the presence or absence of TDP-43 aggregates in the sample or specific body part or body area. The correlation can be qualitative or quantitative. In a preferred embodiment, this step comprises:
Determining the amount of the compound of the invention bound to the TDP-43 aggregates;
Correlating the amount of the compound of the invention bound to the TDP-43 aggregates with the amount of TDP-43 aggregates in the sample or specific body part or body area; and
Optionally comparing the amount of the compound bound with the TDP-43 aggregates in the sample or specific body part or body area to a normal control value in a healthy control subject.

The amount of the compound bound with the TDP-43 aggregates can be determined by any appropriate method. A preferred method is positron emission tomography (PET).

In another embodiment, the presence or absence of the compound of the invention bound to TDP-43 aggregates can be correlated with the disease, disorder or abnormality associated with TDP-43 aggregates or with the TDP-43 proteinopathy. The correlation can be qualitative or quantitative. In a preferred embodiment, this step comprises:
Determining the amount of the compound of the invention bound to the TDP-43 aggregates;
Correlating the amount of the compound of the invention bound to the TDP-43 aggregates with the amount of TDP-43 aggregates in the sample or specific body part or body area; and
Optionally comparing the amount of the compound bound with the TDP-43 aggregates in the sample or specific body part or body area to a normal control value in a healthy control subject.

In any of the methods disclosed herein, steps (a) to (c) and, if present, optional step (d) can be repeated at least one time. The repetition of the steps is particularly useful in the method of collecting data for monitoring the progression and the method of collecting data for predicting responsiveness.

In these methods, it may be expedient to monitor the patient over time and repeat the above steps after a certain period of time has elapsed. The time interval before the above-mentioned steps are repeated can be determined by a physician depending on the severity of the disease, disorder or abnormality associated with TDP-43 aggregates or the TDP-43 proteinopathy.

In one embodiment, the present invention relates to a method of detecting a neurological disease, disorder or abnormality associated with TDP-43 aggregates in a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound which is bound to the TDP-43 aggregates.

In one embodiment, the present invention relates to a method (e.g. in vivo or in vitro method) for the detection and optionally quantification of TDP-43 aggregates in a tissue of a subject, the method comprising the steps:
(a) Administering a compound of the invention as disclosed herein; or a diagnostic composition comprising a compound of the invention as disclosed herein, to the subject;
(b) Allowing the compound to bind to the TDP-43 aggregates; and
(c) Detecting and optionally quantifying the compound bound to the TDP-43 aggregates using positron emission tomography.

In one embodiment, the present invention relates to a method of the diagnostic imaging of the brain of a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates by collecting a positron emission tomography (PET) image of the brain of the subject.

### Imaging:

The present invention relates to a method of imaging TDP-43 aggregates using the compounds of the invention. Imaging can be conducted, for example, using any of the above-mentioned methods, particularly by PET.

In one embodiment, the present invention relates to a method of imaging TDP-43 aggregates in a sample or a patient, in particular in a brain or a sample taken from the patient's brain, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates.

In one embodiment, the present invention relates to a method of imaging a disease, disorder or abnormality associated with TDP-43 aggregates in a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates in the brain of the subject.

In one embodiment, the present invention relates to a method of imaging a disease, disorder or abnormality associated with TDP-43 aggregates in a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates.

In one embodiment, the present invention relates to a method of imaging a disease, disorder or abnormality associated with TDP-43 aggregates in a subject, the method comprising the steps:
(a) Administering a compound of the invention; or a diagnostic composition comprising a compound of the invention as disclosed herein, to the subject;
(b) Allowing the compound to bind to the TDP-43 aggregates;
(c) Detecting the compound bound to the TDP-43 aggregates; and
(d) Generating an image representative of the location and/or amount of the compound bound to the TDP-43 aggregates.

In one embodiment, the present invention relates to a method of positron emission tomography (PET) imaging of TDP-43 aggregates in a tissue of a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates by collecting a positron emission tomography (PET) image of the tissue of the subject.

Preferably, the tissue is a tissue of the central nervous system (CNS), an eye tissue or a brain tissue. More preferably, the tissue is brain tissue.

In one embodiment, the present invention relates to a method of imaging TDP-43 aggregates in a sample or a patient, the method comprises the steps:
(a) Bringing a sample, a specific body part or body area suspected to contain TDP-43 aggregates into contact with a compound of the invention; or with a diagnostic composition comprising a compound of the invention as disclosed herein;
(b) Allowing the compound to bind to the TDP-43 aggregates; and
(c) Detecting the compound bound to the TDP-43 aggregates by imaging the sample, the specific body part or the body area with an imaging system.

In one embodiment, the present invention relates to a method for imaging TDP-43 aggregates in an in vitro sample of a patient, the method comprising the steps:
(a) Bringing the in vitro sample suspected to contain TDP-43 aggregated into contact with a compound of the invention; or with a diagnostic composition comprising a compound of the invention as disclosed herein;
(b) Allowing the compound to bind to the TDP-43 aggregates; and
(c) Detecting the compound bound to the TDP-43 aggregates by imaging the in vitro sample with an imaging system.

In one embodiment, the present invention relates to a method of imaging TDP-43 aggregates in a patient or a specific body part or a body area of a patient, the method comprising the steps:
(a) Bringing a sample or a specific body part or body area suspected to contain TDP-43 aggregates into contact with a compound of the invention, preferably with a compound of formula (I-T) or of formula (I-F); or with diagnostic composition comprising a compound of the invention as disclosed herein, preferably a compound of formula (I-T) or of formula (I-F);
(b) Allowing the compound to bind to the TDP-43 aggregates; and
(c) Detecting the compound bound to the TDP-43 aggregates by imaging the sample or the specific body part or the body area of the patient with an imaging system.

The step of imaging the sample, the patient, the specific body part or the body area of the patient with an imaging system includes detecting the compound of the invention bound to the TDP-43 aggregates using an imaging system as disclosed herein. Detecting the compound of the invention bound to the TDP-43 aggregates allows to identify by imaging the distribution of TDP-43 aggregates in the tested sample, the patient, the specific body part or body area. The PET imaging should be conducted when the compound has penetrated the tissue and the compound has bound to the TDP-43 aggregates.

### Determining the Amount of TDP-43 Aggregates:

In one embodiment, the present invention relates to a method of determining the amount of TDP-43 aggregates in a sample, a specific body part or body area suspected to contain TDP-43 aggregates using a compound of the invention.

In one embodiment the present invention provides a method for determining the amount of TDP-43 aggregates in the sample, the specific body part or the body area suspected to contain TDP-43 aggregates, wherein the method comprises the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates;
(d) Determining the amount of compound of the invention bound to the TDP-43 aggregates; and
(e) Optionally calculating the amount of TDP-43 aggregates in the sample, the specific body part or body area.

A radioactive signal is observed when a detectably labelled compound of the invention, which comprises at least one radiolabeled atom (e.g. ³H, ²H, or ¹⁸F), is bound to the TDP-43 aggregates.

Diagnosing: In one embodiment, the present invention relates to a method of diagnosing a disease, disorder or abnormality associated with TDP-43 aggregates or TDP-43 proteinopathy, the method comprising the steps of:
(a) Detecting the compound of the invention bound to the TDP-43 aggregates; and
(b) Correlating the presence or absence of the compound of the invention bound to TDP-43 aggregates with the disease, disorder or abnormality associated with TDP-43 aggregates or with the TDP-43 proteinopathy.

Preferably, the method of diagnosing the disease, disorder or abnormality associated with TDP-43 aggregates or the TDP-43 proteinopathy comprises the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Correlating the presence or absence of the compound of the invention bound to TDP-43 aggregates with the disease, disorder or abnormality associated with TDP-43 aggregates or with the TDP-43 proteinopathy.

In one embodiment, the present invention relates to a method of collecting data for the diagnosis of a disease, disorder or abnormality associated with TDP-43 aggregates or a TDP-43 proteinopathy, the method comprising the following steps:
(a) Bringing a sample or a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Optionally correlating the presence or absence of the compound of the invention bound with the TDP-43 aggregates with the presence or absence of TDP-43 aggregates in the sample or specific body part or body area.

After the sample or a specific body part or body area has been brought into contact with the compound of the present invention, the compound is allowed to bind to the TDP-43 aggregates. The amount of time required for binding will depend on the type of test (e.g., in vitro or in vivo) and can be determined by a person skilled in the field by routine experiments. The compound which has bound to the TDP-43 aggregates can be subsequently detected by any appropriate method. The specific method chosen will depend on the detectable label which has been chosen. Examples of possible methods include, but are not limited to, a fluorescence imaging technique or a nuclear imaging technique such as positron emission tomography (PET), single photon emission computed tomography (SPECT), magnetic resonance imaging (MRI), and contrast-enhanced magnetic resonance imaging (MRI). The fluorescence imaging technique and/or nuclear imaging technique can be employed for monitoring and/or visualizing the distribution of the detectably labelled compound within the sample or a specific body part or body area.

The step of optionally correlating the presence or absence of the compound bound to the TDP-43 aggregates with the presence or absence of TDP-43 aggregates in the sample or specific body part or body area; as mentioned herein above, comprises the steps of
determining the amount of the compound bound to the TDP-43 aggregates;
correlating the amount of the compound bound to the TDP-43 aggregates with the amount of TDP-43 aggregates in the sample or specific body part or body area; and
optionally comparing the amount of the compound bound with the TDP-43 aggregates in the sample or specific body part or body area to a normal control value in a healthy control subject.

The amount of compound bound with the TDP-43 aggregates can be compared to a normal control value which has been determined in a sample or a specific body part or body area of a healthy subject, wherein an increase in the amount of the compound bound with the TDP-43 aggregates compared to a normal control value may indicate that the patient is suffering from or is at risk of developing a disease, disorder or abnormality associated with TDP-43 aggregates.

If the amount of the compound bound with the TDP-43 aggregates is higher than the normal control value, as defined herein, then it can be expected that the patient is suffering from or is likely to suffer from a disease, disorder or abnormality associated with TDP-43 aggregates or TDP-43 proteinopathy.

### Determining a Predisposition:

A further aspect of the present invention relates to a method of collecting data for determining a predisposition to a disease, disorder or abnormality associated with TDP-43 aggregates or a TDP-43 proteinopathy. The method comprises the steps:
(a) Bringing a sample or a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Optionally correlating the presence or absence of the compound of the invention bound with the TDP-43 aggregates with the presence or absence of TDP-43 aggregates in the sample or specific body part or body area.

The step of optionally correlating the presence or absence of the compound bound to the TDP-43 aggregates with the presence or absence of TDP-43 aggregates in the sample or specific body part or body area; as mentioned herein above, comprises the steps of
determining the amount of the compound bound to the TDP-43 aggregates;
correlating the amount of the compound bound to the TDP-43 aggregates with the amount of TDP-43 aggregates in the sample or specific body part or body area; and
optionally comparing the amount of the compound bound with the TDP-43 aggregates in the sample or specific body part or body area to a normal control value in a healthy control subject.

If the amount of the compound bound to the TDP-43 aggregates is higher than a normal control value of a healthy/reference subject this indicates that the patient/subject is suffering from or is at risk of developing a disease, disorder or abnormality associated with TDP-43 aggregates. In particular, if the amount of the compound bound to the TDP-43 aggregates is higher than what expected in a person showing no clinical evidence of neurodegenerative disease, it can be assumed that the patient has a disposition to a disease, disorder or abnormality associated with TDP-43 aggregates or with a TDP-43 proteinopathy.

### Monitoring Disease Progression:

In one embodiment, the present invention relates to a method of monitoring the progression of a disease, disorder or abnormality associated with TDP-43 aggregates or a TDP-43 proteinopathy in a patient. Typically, the patient is or has been undergoing treatment of the disease, disorder or abnormality associated with TDP-43 aggregates or is or with TDP-43 proteinopathy. In particular, the treatment can involve administration of an anti-TDP-43 medicament.

The method of collecting data for monitoring the progression of a disease, disorder or abnormality associated with TDP-43 aggregates or for monitoring the progression of a TDP-43 proteinopathy in a patient comprises the steps:
(a) Bringing a sample, a specific body part or body area suspected to contain TDP-43 aggregates into contact with the compound of the invention;
(b) Allowing the compound of the invention to bind to the TDP-43 aggregates;
(c) Detecting the compound of the invention bound to the TDP-43 aggregates;
(d) Optionally correlating the presence or absence of the compound of the invention bound with the TDP-43 aggregates with the presence or absence of TDP-43 aggregates in the sample or specific body part or body area; and
(e) Optionally repeating steps (a) to (c) and, if present, optional step (d) at least one time.

In order to monitor the progression over time of the disease, disorder or abnormality associated with TDP-43 aggregates or the TDP-43 proteinopathy, steps (a) to (c) and optional step (d) (if present) can be repeated one or more times. Preferably, the steps should be repeated until no further progression of the disease is observed in the patient.

The step of optionally correlating the presence or absence of the compound bound to the TDP-43 aggregates with the presence or absence of TDP-43 aggregates in the sample or specific body part or body area; as mentioned herein above, comprises the steps of
Determining the amount of the compound bound to the TDP-43 aggregates;
Correlating the amount of the compound bound to the TDP-43 aggregates with the amount of TDP-43 aggregates in the sample or specific body part or body area; and
Optionally comparing the amount of the compound bound with the TDP-43 aggregates in the sample or specific body part or body area to a normal control value in a healthy control subject.

In the method of monitoring progression over time the amount of the compound of the invention bound to the TDP-43 aggregates can be optionally compared at various points of time during the treatment, for instance, before and after onset of the treatment and/or at various points of time after the onset of the treatment. A change, especially a decrease, in the amount of the compound of the invention bound to the TDP-43 aggregates may indicate that the disease is not progressing.

### Predicting Responsiveness:

In one embodiment, the present invention relates to a method of predicting responsiveness of a patient suffering from a disease, disorder or abnormality associated with TDP-43 aggregates, or suffering from a TDP-43 proteinopathy to a treatment of said disease, disorder or abnormality associated with TDP-43 aggregates or TDP-43 proteinopathy.

The method can be used to predict the treatment which is most suitable for the patient. In particular, the treatment can involve administration of an anti-TDP-43 medicament.

The method for predicting responsiveness of a patient suffering from a disease, disorder or abnormality associated with TDP-43 aggregates or suffering from a TDP-43 proteinopathy to a treatment of said disease, disorder or abnormality associated with TDP-43 aggregates or TDP-43 proteinopathy can comprise the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates;
(d) Optionally correlating the presence or absence of the compound of the invention bound with the TDP-43 aggregates with the presence or absence of TDP-43 aggregates in the sample or specific body part or body area; and
(e) Optionally repeating steps (a) to (c) and, if present, optional step (d) at least one time.

Typically, the patient is/has been undergoing treatment of the disease, disorder or abnormality associated with TDP-43 aggregates or the TDP-43 proteinopathy. In particular, the treatment can involve administration of a medicament which is suitable for treating the disease, disorder or abnormality associated with TDP-43 aggregates.

The present method allows to predict the responsiveness of a patient to a certain treatment. In one embodiment, the responsiveness can be estimated, e.g., by repeating steps (a) to (c) and, if present, optional step (d) and monitoring the amount of the compound of the invention bound with the TDP-43 aggregates over a period of time during which the patient is undergoing treatment of the disease, disorder or abnormality associated with TDP-43 aggregates or with TDP-43 proteinopathy. If the amount of the compound of the invention bound with the TDP-43 aggregates decreases over time, it can be assumed that the patient is responsive to the treatment. If the amount of the compound bound with the TDP-43 aggregates is essentially constant or increases over time, it can be assumed that the patient is non-responsive to the treatment.

Alternatively, the responsiveness can be estimated by determining the amount of the compound of the invention bound to the TDP-43 aggregates. The amount of the compound bound to the TDP-43 aggregates can be compared to a control value such as a normal control value, a preclinical control value or a clinical control value. The control value may refer to the control value of healthy control subjects. Alternatively, the control value may refer to the control value of subjects known to be responsive to a certain therapy, or to the control value may refer to the control value of subjects known to be non-responsive to a certain therapy. The outcome with respect to responsiveness can either be “responsive” to a certain therapy, “non-responsive” to a certain therapy or “response undetermined” to a certain therapy. Response to the therapy may be different for the respective patients.

The step of optionally correlating the presence or absence of the compound bound to the TDP-43 aggregates with the presence or absence of TDP-43 aggregates in the sample or specific body part or body area; as mentioned herein above, comprises the steps of
Determining the amount of the compound bound to the TDP-43 aggregates;
Correlating the amount of the compound bound to the TDP-43 aggregates with the amount of TDP-43 aggregates in the sample or specific body part or body area; and
Optionally comparing the amount of the compound bound with the TDP-43 aggregates in the sample or specific body part or body area to a normal control value in a healthy control subject.

The control value can be, e.g., a normal control value, a preclinical control value and/or a clinical control value. A “healthy control subject” or “healthy subject” is a person showing no clinical evidence of neurodegenerative disease.

If in any of the above summarized methods the amount of the compound bound with the TDP-43 aggregates is higher than the normal control value, then it can be expected that the patient is suffering from or is likely to suffer from a disease, disorder or abnormality associated with TDP-43 aggregates or a TDP-43 proteinopathy.

Any of the compounds of the present invention can be used in the above summarized methods. Preferably detectably labeled compounds of the present invention, as disclosed herein, are employed in the above summarized methods.

In a fourth aspect, the present invention relates to the use of the compounds of the invention as a TDP-43 aggregates' diagnostic agent or diagnostic tool. In one embodiment, the present invention relates to the use of the compounds of the invention as an in vitro analytical reference or an in vitro screening tool. Said compounds of the invention are also useful in in vivo diagnostic methods. In such instances, the compounds of the invention may be detectably labeled compounds or contain cold isotopes.

In another embodiment, the present invention further relates to the use of the compounds of the present invention, more specifically detectably labelled compounds of the invention as defined herein, as diagnostic biomarkers enabling more efficient and precise patient selection, e.g., for longitudinal monitoring in clinical studies, or for supporting the development of novel therapeutics for treating TDP-43 proteinopathies. In another embodiment, the present invention further relates to the use of the compounds of the present invention, more specifically detectably labelled compounds of the invention as defined herein, as a TDP-43 aggregates' biomarker or a TDP-43 proteinopathy biomarker.

In another embodiment, the compounds of the invention may be employed for research use, in particular, as an analytical tool or reference molecule. Said compounds may also be used in detecting TDP-43 aggregates in vitro or in vivo. The compounds of the invention may be used to stain TDP-43 aggregates. For example, compounds of the invention may be used for histochemical detection in postmortem tissue such as brain tissue. The compounds of the invention are preferably detectably labelled compounds and may be directly or indirectly labelled as discussed herein.

### Kit of Parts

In a fifth aspect, the present invention relates further to a kit for use in one or more of the methods of the invention, wherein the kit comprises a compound of the invention as described herein. The kit typically comprises a container holding the compound of the invention and instructions for using the compound of the invention. Preferably, the kit comprises a compound of formula (I), as disclosed herein. More preferably, the compound of the invention is a detectably labelled compound (e.g. compound of formula (I-T) or (I-F)).

The term “kit” refers in general to any diagnostic kit known in the art. More specifically, the latter term refers to a diagnostic kit as described in Zrein et al., Clin. Diagn. Lab. Immunol., 1998, 5, 45-49.

The dose of the detectably labelled compounds of the present invention will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

In particular, such kits may be useful for performing the methods of the invention (which include, for example, but not limited to, imaging, diagnosing, and monitoring methods), e.g., for diagnosing of a disease, disorder or abnormality associated with TDP-43 aggregates or a TDP-43 proteinopathy.

Such kits may comprise all necessary components for performing the herein provided methods. Typically, each component is stored separately in a single overall packaging. Suitable additional components for inclusion in the kits are, for example, buffers, detectable dyes, laboratory equipment, reaction containers, instructions and the like. Instructions for use may be tailored to the specific method for which the kit is to be employed.

The present invention relates further to a kit for the preparation of a detectably labeled compound of the invention, wherein in particular the detectable label is a radioisotope. Thus, the kit comprises a precursor of the detectably labeled compound of the formula (I) and a labeling agent which reacts with the precursor to introduce a detectable (e.g., radioactive) label. Preferred precursors are compounds of the formulae (II), (III) and (IV). The labeling agent which reacts with the precursor can be an agent which introduces a detectable (e.g., radioactive) label such as ¹⁸F or ³H. The labeling agent can be a ¹⁸F-fluorination agent.

### Method for Preparing a Compound of the Invention

In a sixth aspect, the present invention relates further to a method for preparing a compound of formula (I).

### Cold Isotope Compounds:

In one embodiment, the present invention relates to a method for preparing a compound of formula (I), as described above, the method comprising the step of:

Reacting a compound of formula (II) with R⁹ to provide a compound of formula (I)

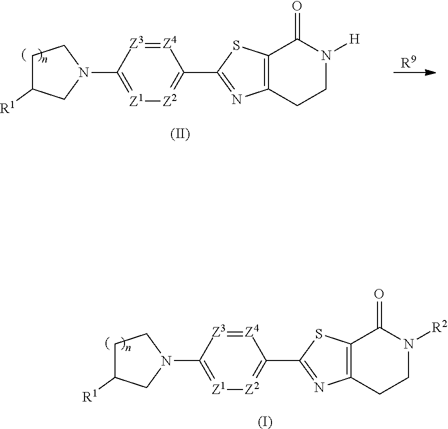

wherein
n, R¹, R², Z¹, Z², Z³, and Z⁴ are as defined above; and
R⁹ is
a 5- or 6-membered carbocyclic ring which can be optionally substituted with F, NH₂, CN and/or CH₃,
a 5- or 6-membered heterocycloalkyl ring which can be optionally substituted with F, NH₂, CN and/or CH₃, wherein the 5- or 6-membered heterocycloalkyl ring contains one or more heteroatoms selected from N, O and S,
a 5-membered heteroaryl ring which can be optionally substituted with F, NH₂, CN and/or CH₃, wherein the 5-membered heteroaryl ring contains one or more heteroatoms selected from N, O and S, or
a 6-membered heteroaryl ring which can be optionally substituted with F, NH₂, CN and/or CH₃, wherein the 6-membered heteroaryl ring contains one heteroatom selected from O and S, or two or more heteroatoms selected from O, N and S,
wherein the 5- or 6-membered carbocyclic ring, the 5- or 6-membered heterocycloalkyl ring, the 5-membered heteroaryl ring or the 6-membered heteroaryl ring is substituted with Br or I.

In a preferred embodiment, a compound having the formula (11) with R⁹ as defined in the following to result in a compound of formula (I-C)
R⁹ is

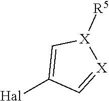

wherein X is N and R¹ is CH₃ or H; and wherein Hal is Br or I, preferably Hal is Br; or
R⁹ is

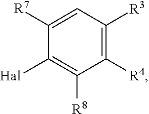

wherein Hal is Br or I, preferably Hal is Br; and
R³ is F, R⁴ is NH₂, R⁷ is H, and R⁸ is H;
R³ is NH₂, R⁴ is F, R⁷ is H, and R⁸ is H,
R³ is CN, R⁴ is NH₂, R⁷ is H, and R⁸ is H,
R³ is H, R⁴ is NH₂, R⁷ is H, and R⁸ is CN, or
R3 is H, R⁴ is NH₂, R⁷ is H, and R⁸ is F.

In one embodiment, the present invention relates to a method for preparing a compound of formula (I*), as described above, the method comprising the step of:

Reacting a compound of formula (II*) with R⁹

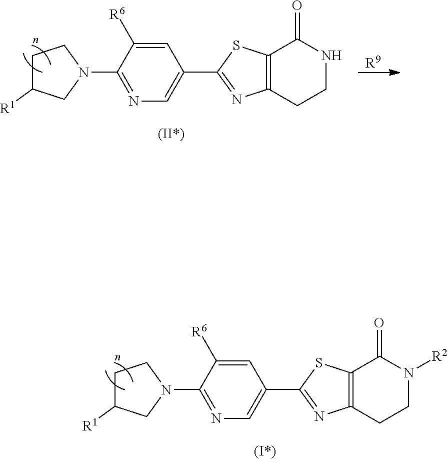

wherein
n is 1 or 2;
R¹ is H or F;
R⁶ is H; and wherein
R⁹ is a 5- or 6-membered carbocyclic or heterocyclic ring which can be optionally substituted with F, NH₂ and/or CH₃, wherein the heterocyclic ring contains one or more heteroatoms selected from N, O and S, wherein the 5- or 6-membered carbocyclic or heterocyclic ring is substituted with Br or I; to provide a compound of formula (I*), wherein R² is as defined herein.

A preferred embodiment of this method comprises the step of:

Reacting a compound having the formula (II) with R⁹

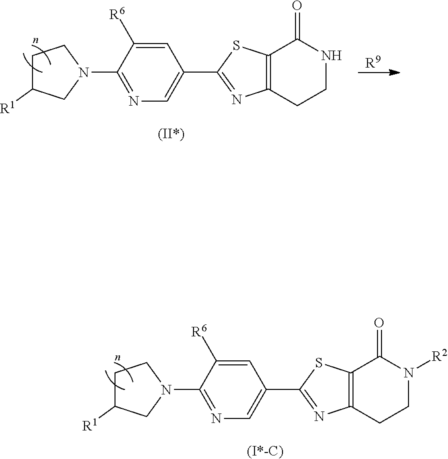

wherein
n is 1 or 2;
R¹ is H or F;
R⁶ is H; and
R⁹ is

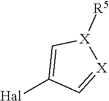

wherein X is N and R⁵ is CH₃; and wherein Hal is Br or I, preferably Hal is Br; or
R⁹ is

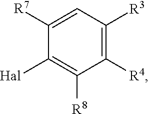

wherein R³ is F; R⁴ is —NH₂; R¹ is H; R⁸ is H; and wherein Hal is Br or I, preferably Hal is Br;
to provide a compound of formula (I-C), wherein
n is 1 or 2;
R¹ is H or F;
R⁶ is H; and
R² is

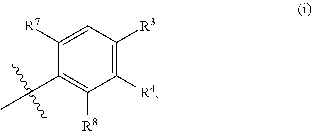

wherein R³ is F, R⁴ is —NH₂, R¹ is H, and R⁸ is H;
or
R² is

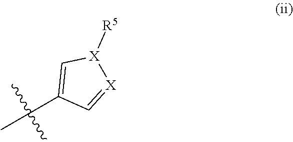

wherein X is N, and R⁵ is CH₃.

The method of reacting the compound having the formula (II) with R⁹ can be conducted by any suitable method. In one option, the reaction can be conducted in the presence of a diamine chelator such as DMEDA, a base such as potassium carbonate, a catalyst such as CuI, and an aprotic solvent such as dioxane.

In one embodiment, the present invention provides a method for preparing the compound of formula (I-C), which is Compound 1, wherein the method comprises the step of reacting a compound of formula (II), which is Prep 1, with R⁹ which is

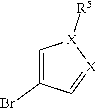

wherein X is N, and R⁵ is CH₃, as shown below:

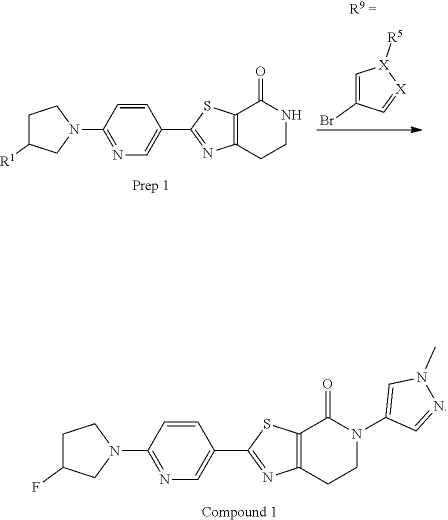

In another embodiment, the present invention provides a method for preparing the compound of formula (I-C), which is Compound 2, wherein the method comprises the step of reacting the compound of formula (II), which is Prep 2, with R⁹ which is

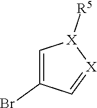

wherein X is N and R⁵ is CH₃, as shown below:

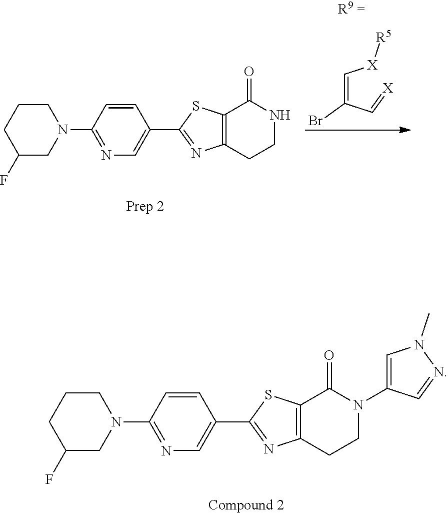

In another embodiment, the present invention provides a method for preparing the compound of formula (I-C), which is Compound 3, wherein the method comprises the step of reacting the compound of formula (II), which is Prep 3, with R⁹ which is

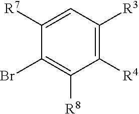

wherein R³ is F, R⁴ is —NH₂, R⁷ is H, R⁸ is H as shown below:

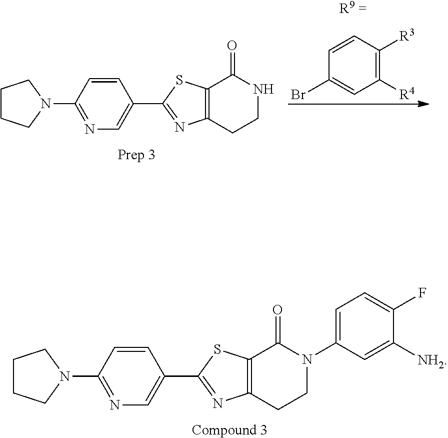

Preferably, the method concerns compounds of the invention having the formula (I) and compounds having the formula (II) that do not include a detectable label.

### Tritium (³H) Detectably Labeled Compounds

In one embodiment, the present invention provides a method for preparing the Tritium (³H) detectably labeled compounds of the invention having the formula (I), said method comprises the step of radiolabeling a precursor of the compound having the formula (I) with a radioisotope, wherein at least one leaving group of the precursor of the compound having the formula (I) is replaced by Tritium (³H).

Tritium (³H) detectably labeled compounds having the formula (I) are preferably defined wherein at least 1 to 3 Hydrogen (H) are each replaced by Tritium (³H). Tritium (³H) detectably labeled compounds having the formula (I) are more preferably defined wherein 2 or 3 Hydrogen (H) are replaced by Tritium (³H). Tritium (³H) detectably labeled compounds having the formula (I) are even more preferably defined wherein 3 Hydrogen (H) are replaced by Tritium (³H).

In another embodiment, the present invention provides a method for preparing a compound of formula (I), said method comprises the step of:

Radiolabeling a precursor compound having the formula (III) with T (i.e., ³H)

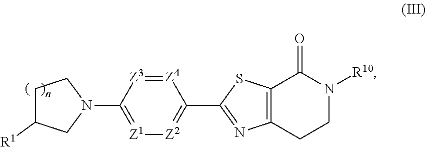

or a stereoisomer, polymorph, racemic mixture, tautomer, pharmaceutically acceptable salt, prodrug, hydrate, or solvate thereof, or mixtures thereof,
wherein
Z¹, Z², Z³ and Z⁴ are each selected from C—Br, C—I, C—H and N, wherein
   if Z¹ is N, Z² is C—Br, C—I, or C—H;
   if Z² is N, Z¹ is C—Br, C—I, or C—H;
   if Z³ is N, Z⁴ is C—Br, C—I, or C—H;
   if Z⁴ is N, Z³ is C—Br, C—I, or C—H;
n is 1 or 2;
R¹ is H or F;
R¹⁰ is
   a 5- or 6-membered carbocyclic ring which can be optionally substituted with Br, I, F, NH₂, CN and/or CH₃,
   a 5- or 6-membered heterocycloalkyl ring which can be optionally substituted with Br, I, F, NH₂, CN and/or CH₃, wherein the 5- or 6-membered heterocycloalkyl ring contains one or more heteroatoms selected from N, O and S,
   a 5-membered heteroaryl ring which can be optionally substituted with Br, I, F, NH₂, CN and/or CH₃, wherein the 5-membered heteroaryl ring contains one or more heteroatoms selected from N, O and S, or
   a 6-membered heteroaryl ring which can be optionally substituted with Br, I, F, NH₂, CN and/or CH₃, wherein the 6-membered heteroaryl ring contains one heteroatom selected from O and S, or two or more heteroatoms selected from O, N and S;
at least one of Z¹, Z², Z³ or Z⁴ is selected from C—Br or C—I, and/or
R¹⁰ comprises Br or I,
wherein the at least one Br or I is replaced by CT₃, or T,
and wherein T is ³H.

In one embodiment, the Br or I of C—Br or C—I is replaced by T or CT₃, preferably by T.

In a preferred embodiment, Z³ is C—Br or C—I and the Br or I is replaced by T or CT₃, preferably by T.

In a further embodiment, R¹⁰ comprises Br or I and Br or I is replaced by T or CT₃.

In another embodiment, R¹⁰ comprises Br or I and the Br or I is replaced by T or CT₃. For instance, R¹⁰ can be

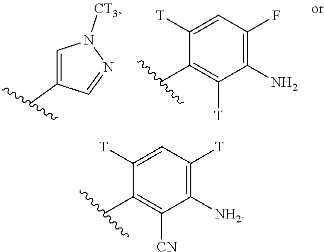

In another embodiment, the present invention provides a method for preparing a compound of formula (I*), said method comprises the step of:

Radiolabeling a precursor compound having the formula (III*) with ³H

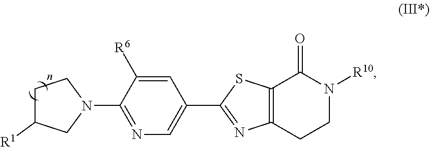

wherein
n is 1 or 2;
R1 is H or F;
R⁶ is Br or H;
R¹⁰ is a 5- or 6-membered carbocyclic or heterocyclic ring which can be optionally substituted with F, Br, NH₂ and/or CH₃, wherein the heterocyclic ring contains one or more heteroatoms selected from N, O and S; and
wherein at least one proton of the 5- or 6-membered carbocyclic or heterocyclic ring is replaced by CT₃, or
wherein at least one Br group is replaced by ³H.

In another embodiment, the present invention provides a method for preparing a compound of formula (I*), said method comprises the step of: Radiolabeling a precursor compound having the formula (IIIa*) with ³H

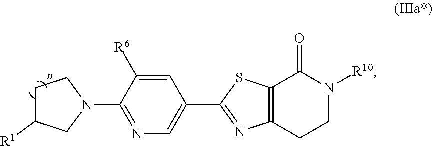

wherein
n is 1 or 2;
R¹ is H or F;
R⁶ is H; and
R¹⁰ is a 5- or 6-membered carbocyclic or heterocyclic ring which can be optionally substituted with F, NH₂ and/or CH₃, wherein the heterocyclic ring contains one or more heteroatoms selected from N, O and S, wherein at least one proton of the 5- or 6-membered carbocyclic or heterocyclic ring is replaced by CT₃.

Preferably, the compound of formula (IIIa) comprises one CT₃ group.

In another embodiment, the present invention provides a method for preparing a compound of formula (I), said method comprises the step of:

Radiolabeling a precursor compound having the formula (IIIb*) with ³H

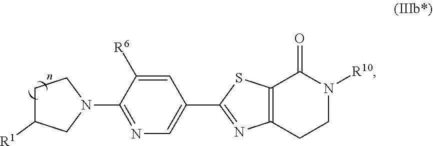

wherein
n is 1 or 2;
R¹ is H or F;
R⁶ is H or Br;
R¹⁰ is a 5- or 6-membered carbocyclic or heterocyclic ring which can be optionally substituted with F, Br, NH₂ and/or CH₃, wherein the heterocyclic ring contains one or more heteroatoms selected from N, O and S; wherein the 5- or 6-membered carbocyclic or heterocyclic ring is substituted with at least one Br; and
wherein at least one of the Br groups is replaced by ³H.

Preferably, the compound of formula (IIIb*) comprises 1 to 3 ³H, and 16 ³H, and 21 ³H. More preferably, the compound of formula (IIIb) comprises 2 or 3 or 16 ³H. Even more preferably, the compound of formula (IIIb*) comprises 16 ³H.

In one embodiment, the present invention provides a method is for preparing a compound of formula (I*-T), the method comprising the step of:
radiolabeling a compound having the formula (III*) with ³H

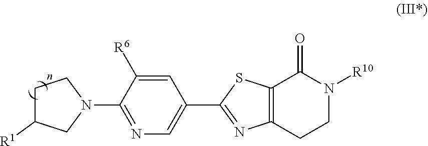

wherein
n is 1 or 2;
R¹ is H or F;
wherein
R¹⁰ is

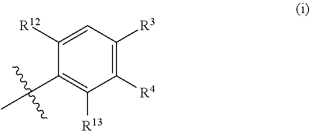

wherein R³ is F, R⁴ is —NH₂, and at least one of R¹² and R¹³ is Br and, if applicable, the other is H; preferably R³ is F, R⁴ is —NH₂, R¹² is Br, R¹³ is Br; and
R⁶ is Br;
or wherein
R¹⁰ is

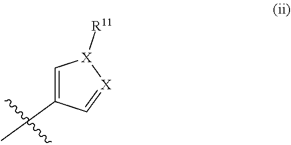

wherein X is N, R¹¹ is H; and
R⁶ is H;
to provide the compound having the formula (I*-T)

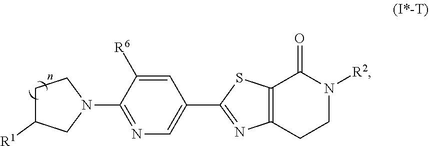

wherein
n and R¹ are as defined above;
T is ³H; and wherein
R² is

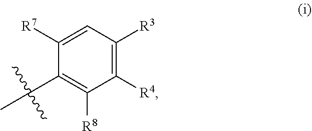

wherein R³ is F, R⁴ is —NH₂, at least one of R¹ and R⁸ is T and, if applicable, the other is H; preferably R⁷ is T, and R⁸ is T; and
R⁶ is T;
or wherein
R² is

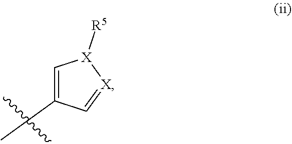

wherein X is N and R⁵ is CT₃; and
R⁶ is H.

In another embodiment, the present invention provides a method for preparing the Tritium (³H) detectably labeled compound having the formula (I-T), the method comprises radiolabeling a precursor having the formula (III*) with ³H

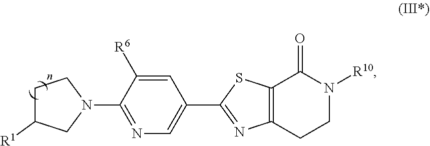

wherein
n is 1 or 2;
R¹ is H or F; and
wherein
   R¹⁰ is

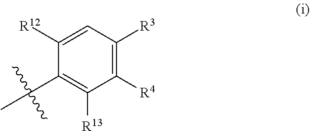

wherein R³ is F; R⁴ is —NH₂; at least one of R¹² and R¹³ is Br and, if applicable, the other is H; preferably R³ is F, R⁴ is —NH₂, R¹² is Br, R¹³ is Br; and
   R⁶ is Br;
or wherein
   R¹⁰ is

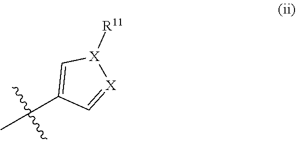

wherein X is N, R¹¹ is H; and
   R⁶ is H;
wherein the Tritium (3H) detectably labeled compound having the formula (I*-T) is

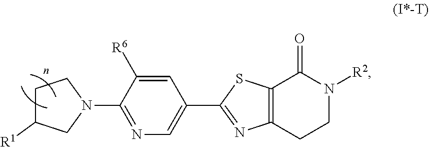

wherein
T means 3H;
n is 1 or 2;
R¹ is H or F; and wherein
   R² is

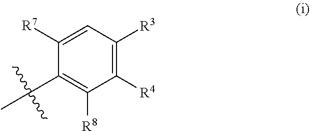

wherein R3 is F, R4 is —NH₂, R¹ is T; R⁸ is T; and
   R⁶ is T;
or
   R² is

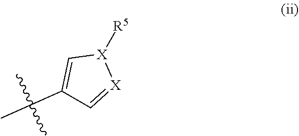

wherein X is N, R⁵ is tritiated CH₃ (CT₃); and
   R⁶ is H.

Preferably, the Tritium (³H) detectably labeled compounds having the formula (I-T), according to the present invention, can be selected from (wherein T means ³H)

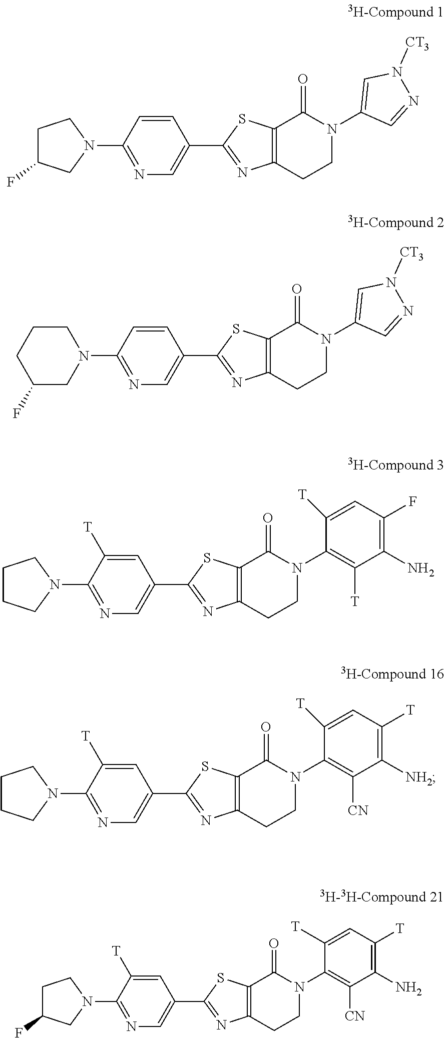

Preferably, the precursors having the formula (III), according to the present invention, can be selected from

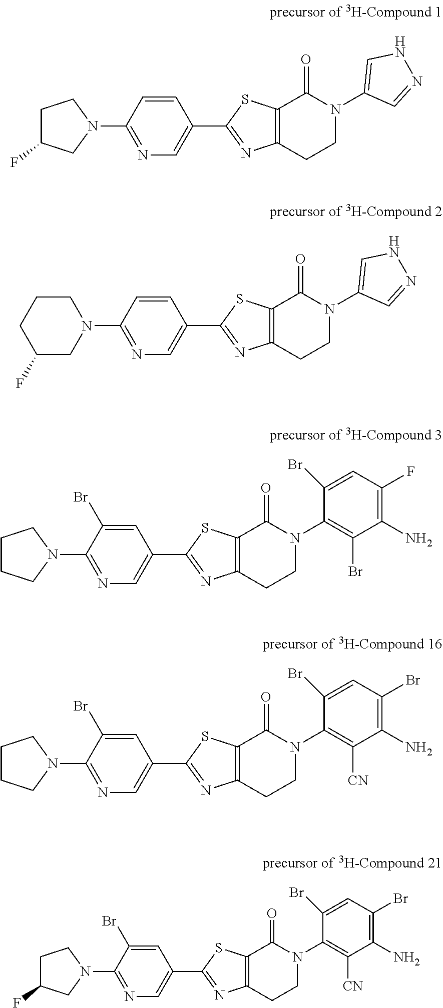 [SMILES: O=C1c2sc(-c3ccc(N4CC[C@@H](F)C4)nc3)nc2CCN1c1cn[nH]c1 ; O=C1c2sc(-c3ccc(N4CCC[C@@H](F)C4)nc3)nc2CCN1c1cn[nH]c1 ; Nc1c(F)cc(Br)c(N2CCc3nc(-c4cnc(N5CCCC5)c(Br)c4)sc3C2=O)c1Br ; N#Cc1c(N)c(Br)cc(Br)c1N1CCc2nc(-c3cnc(N4CCCC4)c(Br)c3)sc2C1=O ; N#Cc1c(N)c(Br)cc(Br)c1N1CCc2nc(-c3cnc(N4CC[C@H](F)C4)c(Br)c3)sc2C1=O]

The methods used for introducing a radioisotope such as 3H are well known in the art and one method is described below.

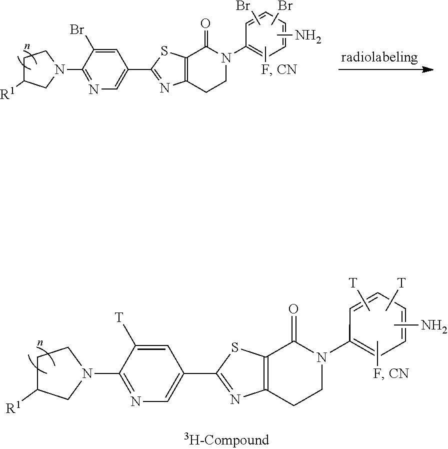

The ³H radiolabeling agent can be tritium gas. The method can be conducted in the presence of a catalyst such as palladium on carbon (Pd/C) or Lindlar's catalyst, a solvent such as N,N-dimethylformamide (DMF) and a base such as N,N-diisopropylethylamine (DIEA).

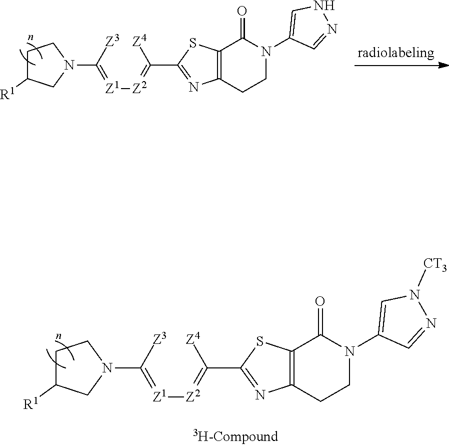

Alternatively, the present invention relates to a method using a CT₃ radiolabeling agent, wherein T is ³H. The CT₃ radiolabeling agent can be ICT₃ (derivative of iodomethane with ³H). The method can be conducted in the presence of a solvent such as dimethylformamide (DMF) and a base such cesium carbonate or sodium hydride.

### Fluorine (¹⁸F) Detectably Labeled Compounds:

In one embodiment, the present invention provides a method for preparing the Fluorine (¹⁸F) detectably labeled compounds of the invention, said method comprises radiolabeling a precursor having the formula (IV) with a radioisotope [⁴⁸F]:

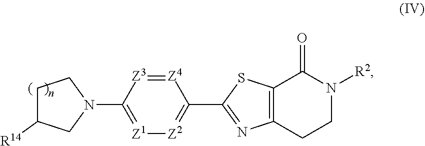

wherein
n, Z¹, Z², Z³, Z⁴ and R² are as defined above, and
R¹⁴ is a leaving group which is replaced by ¹⁸F in the radiolabeling step.

In one embodiment, the present invention provides a method for preparing the Fluorine (¹⁸F) detectably labeled compounds of the invention, said method comprises radiolabeling a precursor having the formula (IV*) with a radioisotope [¹⁸F]:

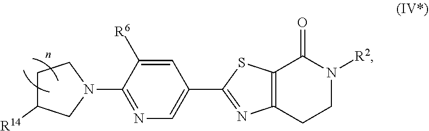

wherein
n is 1 or 2;
R⁶ is H;
R² is a 5- or 6-membered carbocyclic or heterocyclic ring which can be optionally substituted with F,
NH₂ and/or CH₃, wherein the heterocyclic ring contains one or more heteroatoms selected from N, O and S; and
R¹⁴ is a leaving group which is replaced by ¹⁸F in the radiolabeling step.

In another embodiment, the present invention provides a method for preparing the Fluorine (¹⁸F) detectably labeled compounds having the formula (I*-F), said method comprises radiolabeling a precursor compound having the formula (IV*) with ¹⁸F

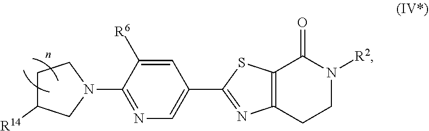

wherein
n is 1 or 2;
R⁶ is H;
R² is

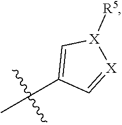

wherein X is N and R⁵ is CH₃; and
R¹⁴ is a leaving group which is replaced by ¹⁸F in the radiolabeling step;
to provide the compound having the formula (I*-F):

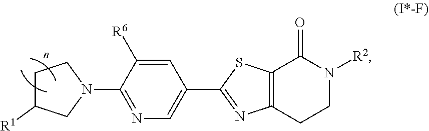

wherein
n, R², and R⁶ are as defined above; and
R¹ is ¹⁸F.

The fluorination can be conducted in the presence of a ¹⁸F-fluorination agent which can be selected from K[¹⁸F], Cs¹⁸F, Na¹⁸F, Rb¹⁸F, Kryptofix[222]K¹⁸F, tetra(C₁₋₆alkyl) ammonium salt of ¹⁸F, and tetrabutylammonium [¹⁸F]fluoride.

Preferably, the Leaving Group (LG) is C₁₋₄ alkyl sulfonate or C₆₋₁₀ aryl sulfonate. More preferably, the Leaving Group (LG) is mesylate, tosylate or nosylate. Even more preferably, the Leaving Group (LG) is mesylate.

Suitable solvents for the ¹⁸F-fluorination step are known to a skilled person. The solvent can be, for example, selected from the group consisting of DMF, DMSO, acetonitrile, DMA, or mixtures thereof.

Preferably, the solvent is acetonitrile or DMSO.

Preferably, the method for preparing the Fluorine (¹⁸F) detectably labeled Compound 1 comprises a radiolabeling step in which the Leaving Group (LG), which in this case is mesylate, of the precursor L1 is replaced with a Fluorine (¹⁸F) in the presence of the ¹⁸F-fluorinating agent, such as K[¹⁸F] or [¹⁸F]TBAF, as shown below:

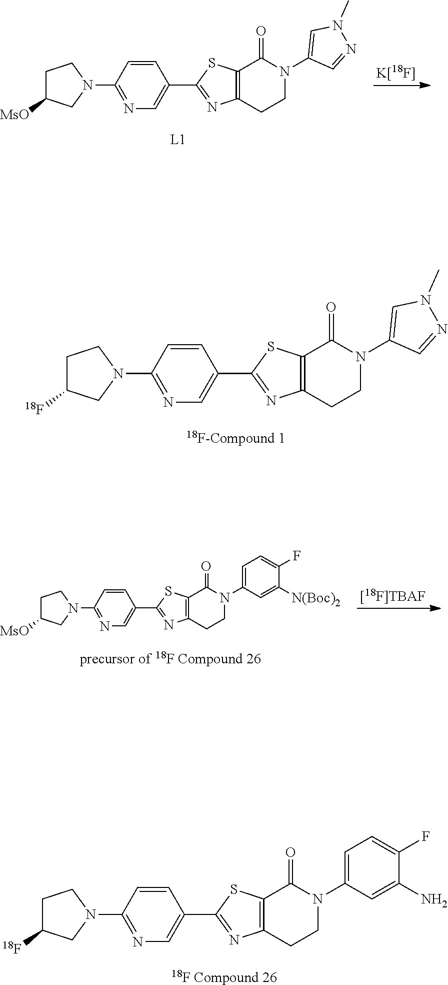

The compounds of the invention can be prepared by one of the general methods shown in the following schemes. These methods are only given for illustrative purposes and should not be construed as limiting.

In a seventh aspect, the present invention relates to precursor compounds of formulae (II), (Ill) and (IV) as disclosed below:
precursor having the formula (II):

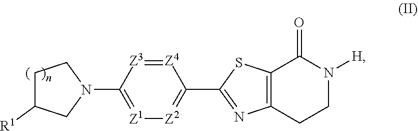

wherein
R¹, n, Z¹, Z², Z³ and Z⁴ are as defined above;
precursor having the formula (III)

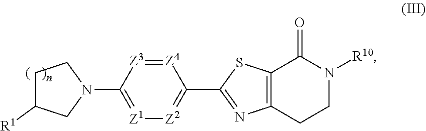

wherein
R¹, R¹⁰, n, Z¹, Z², Z³ and Z⁴ are as defined above.
precursor having the formula (IV)

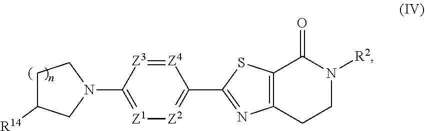

wherein R², n, Z¹, Z², Z³ and Z⁴ are as defined above, and
R¹⁴ is a leaving group LG.

In an embodiment of the seventh aspect, the present invention relates to precursor compounds of formulae (II*), (III*) and (IV*) as disclosed below:
precursor having the formula (II*):

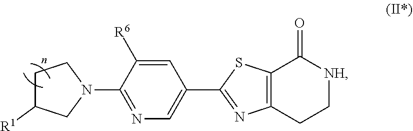

wherein
n is 1 or 2;
R¹ is H or F; and
R⁶ is H;
precursor compound having the formula (IIIa*)

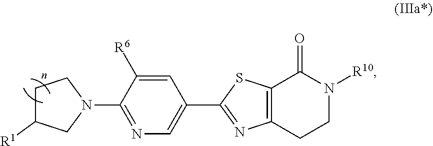

wherein
n is 1 or 2;
R¹ is H or F;
R⁶ is H; and
R¹⁰ is a 5- or 6-membered carbocyclic or heterocyclic ring which can be optionally substituted with F, NH₂ and/or CH₃, wherein the heterocyclic ring contains one or more heteroatoms selected from N, O and S.

Preferably, R¹⁰ is

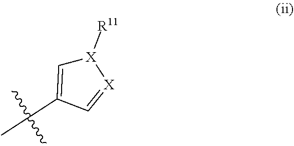

wherein X is N, R¹¹ is H; and
R⁶ is H.
precursor having the formula (IIb*)

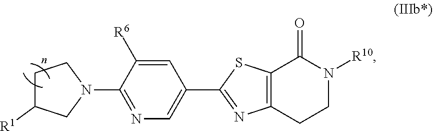

wherein
n is 1 or 2;
R¹ is H or F;
R⁶ is Br or H; and
R¹⁰ is a 5- or 6-membered carbocyclic or heterocyclic ring which can be optionally substituted with F, Br, NH₂ and/or CH₃, wherein the heterocyclic ring contains one or more heteroatoms selected from N, O and S; and
wherein at least one proton of the 5- or 6-membered carbocyclic or heterocyclic ring is substituted with at least one Br.

Preferably, R¹⁰ is

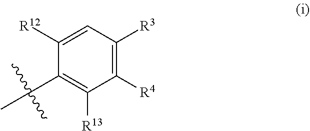

wherein R³ is F; R⁴ is —NH₂; at least one of R¹² and R¹³ is Br and, if applicable, the other is H; more preferably R³ is F, R⁴ is —NH₂, R¹² is Br, R¹³ is Br; and R⁶ is Br;
precursor having the formula (IV*)

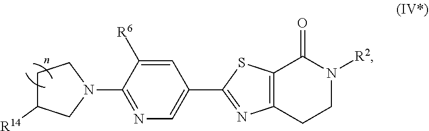

wherein
n is 1 or 2;
R⁶ is H;
R² is a 5- or 6-membered carbocyclic or heterocyclic ring which can be optionally substituted with F, NH₂ and/or CH₃, wherein the heterocyclic ring contains one or more heteroatoms selected from N, O and S; and
R¹⁴ is a leaving group LG.

Preferably, the Leaving Group (LG) is C₁₋₄ alkyl sulfonate or C₆₋₁₀ aryl sulfonate. More preferably, the Leaving Group (LG) is mesylate, tosylate or nosylate. Even more preferably, the Leaving Group (LG) is mesylate.

R¹, R², R³, R⁴, R⁵, R⁶, R⁷, R⁸, R¹⁰, R¹¹, R¹², R¹³, R¹⁴, and n are as described above and include the preferred embodiments.

The precursor compounds having the formulae (11), (111), (IV), (II*), (111*) or (IV*), or the stereoisomer, the polymorph, the racemic mixture, the tautomer, the pharmaceutically acceptable salt, the prodrug, the hydrate, or the solvate thereof or the mixture of the foregoing are part of the invention.

### Abbreviations

DCM Dichloromethane
DIEA N,N-Diisopropylamine
DMEDA 1,2-Dimethylethylenediamine
EtOAc Ethyl Acetate
K₂CO₃ Potassium carbonate
NMR Nuclear magnetic resonance
RT Room Temperature (approx. 25° C.)
TFA Trifluoroacetic acid

### General Synthetic Schemes:

### Synthetic Scheme for the Preparation of (R)-2-(6-(3-fluoropyrrolidin-1-yl)pyridin-3-yl)-5-(pyridin-3-yl)-6,7-dihydrothiazolo[5,4-c]pyridin-4(5H)-one (Compound 1)

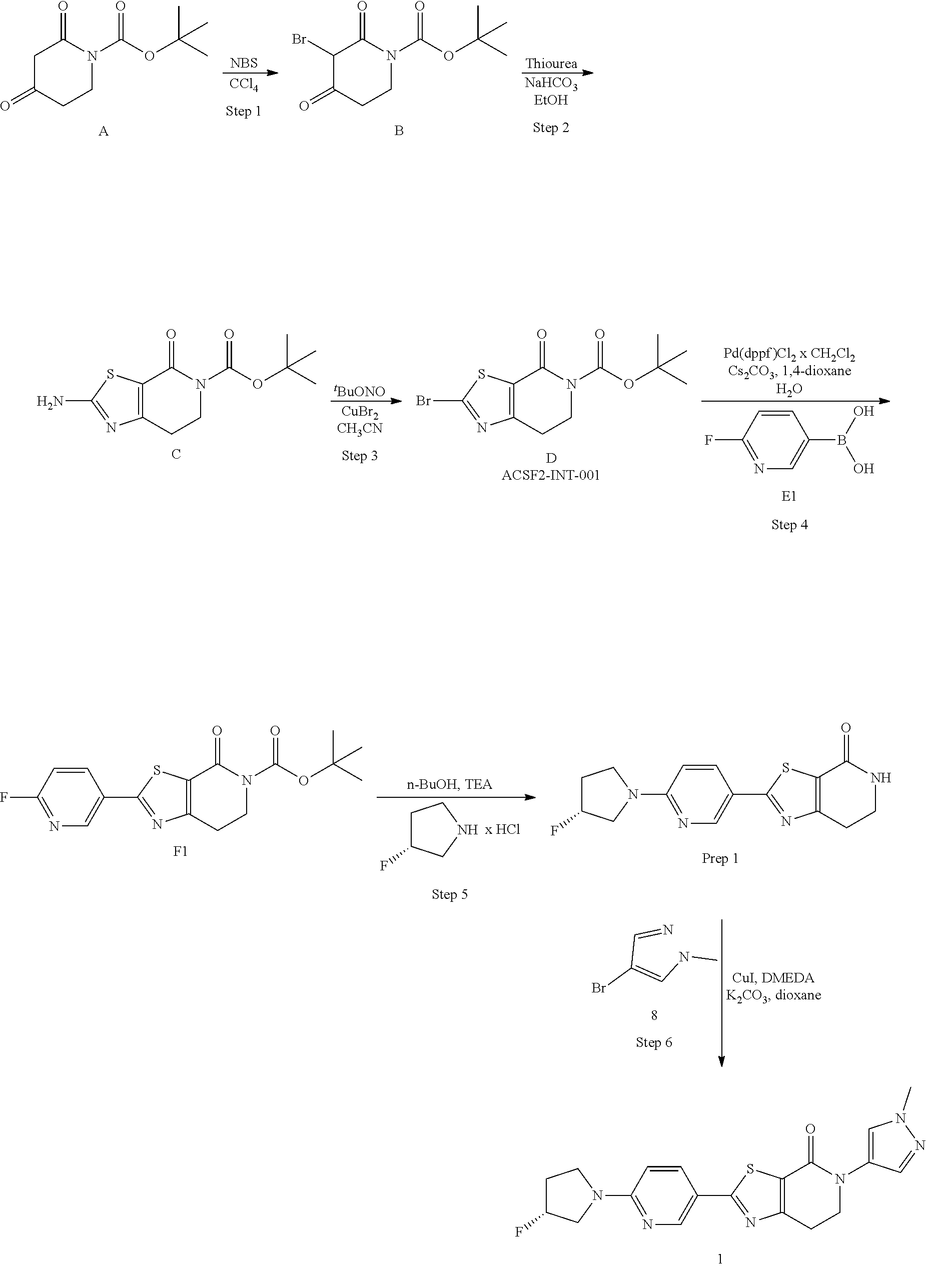

### Synthetic Scheme for the Preparation of (R)-2-(6-(3-fluoropiperidin-1-yl)pyridin-3-yl)-5-(1-methyl-1H-pyrazol-4-yl)-6,7-dihydrothiazolo[5,4-c]pyridin-4(5H)-one (Compound 2)

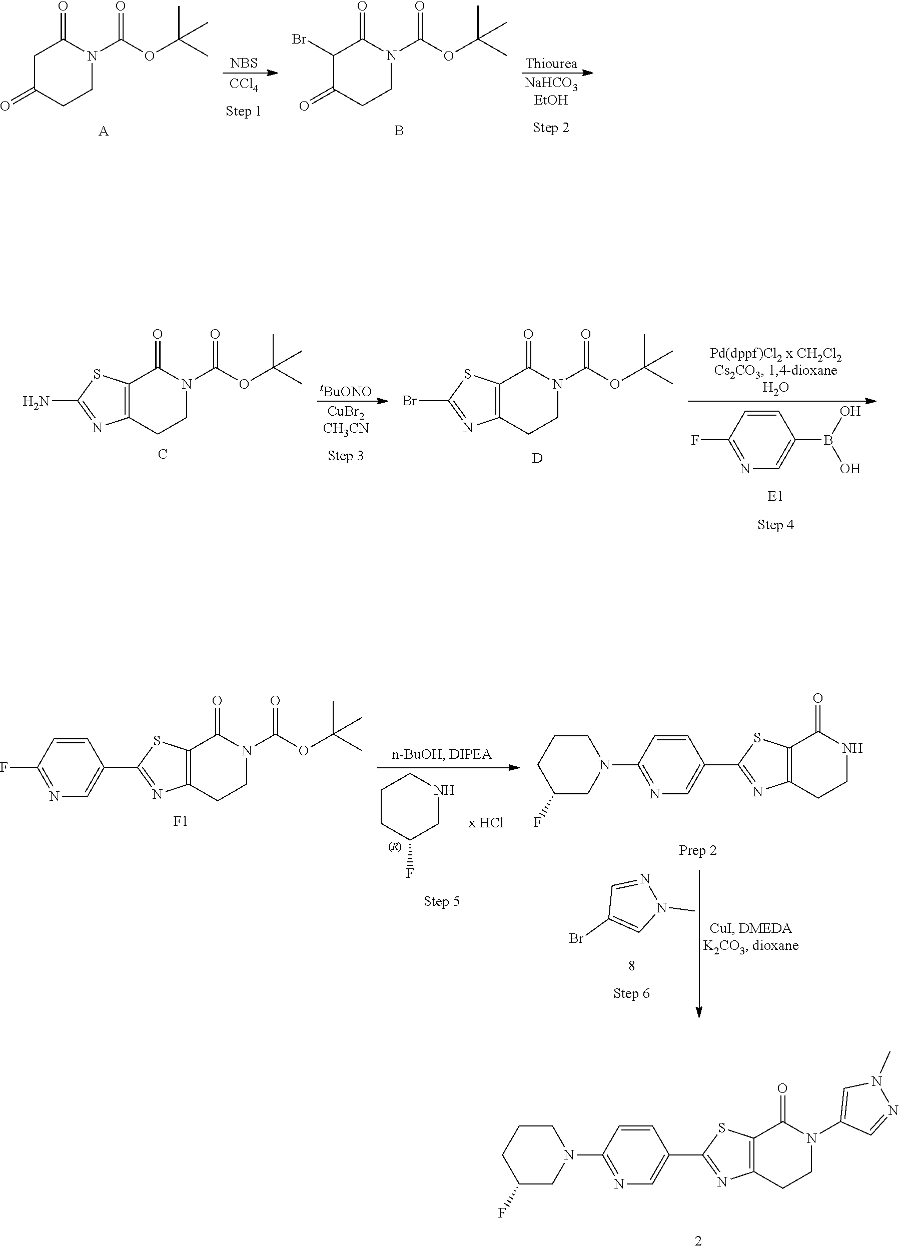

### Synthetic Scheme for the Preparation of (5-(3-amino-4-fluorophenyl)-2-(6-(pyrrolidin-1-yl)pyridin-3-yl)-6,7-dihydrothiazolo[5,4-c]pyridin-4(5H)-one (Compound 3)

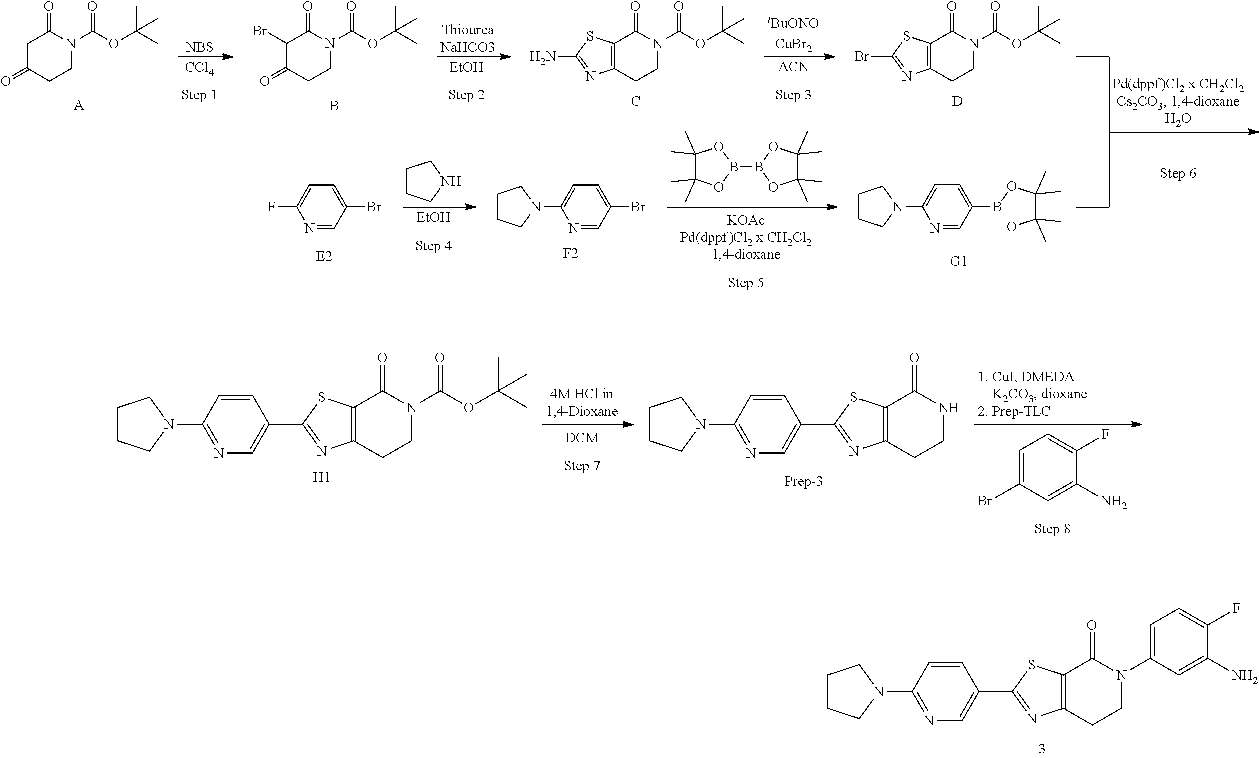

### Synthetic Scheme for the Preparation of [³H] Precursor Compounds

### Synthetic Scheme for the Preparation of (R)-2-(6-(3-fluoropyrrolidin-1-yl)pyridin-3-yl)-5-(1H-pyrazol-4-yl)-6,7-dihydrothiazolo[5,4-c]pyridin-4(5H)-one (precursor of ³H Compound 1)

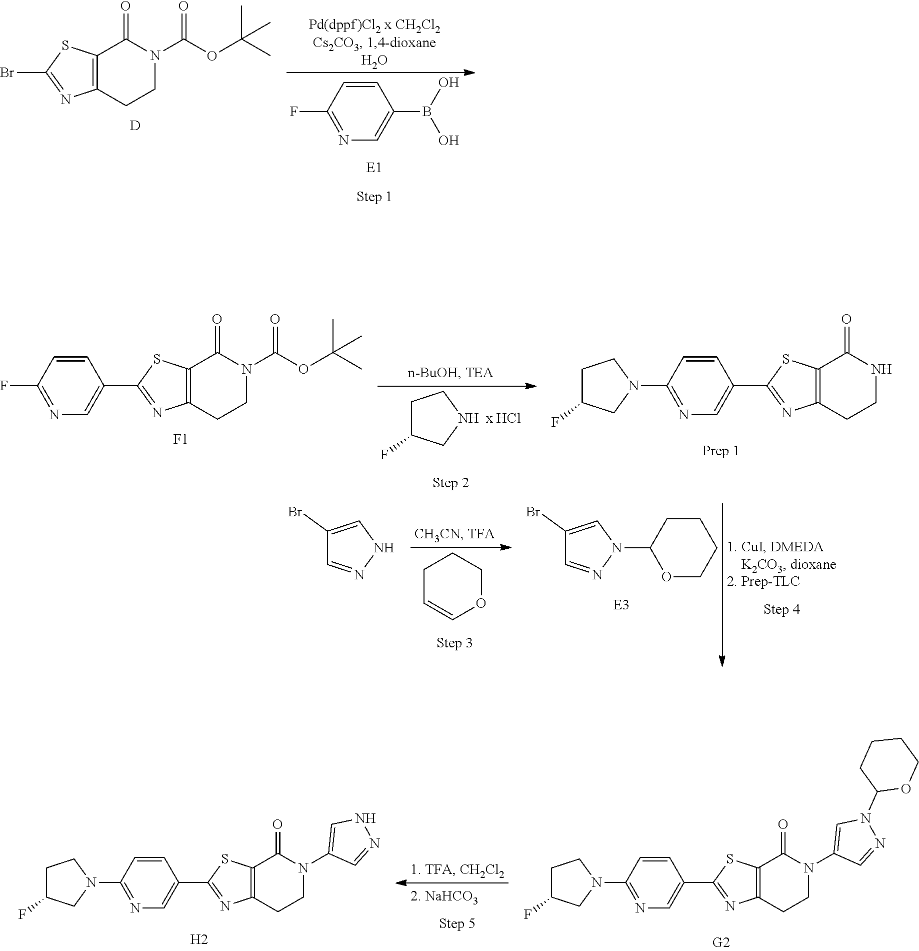

### Synthetic Scheme for the Preparation of (R)-2-(6-(3-fluoropiperidin-1-yl)pyridin-3-yl)-5-(1H-pyrazol-4-yl)-6,7-dihydrothiazolo[5,4-c]pyridin-4(5H)-one (precursor of ³H Compound 2)

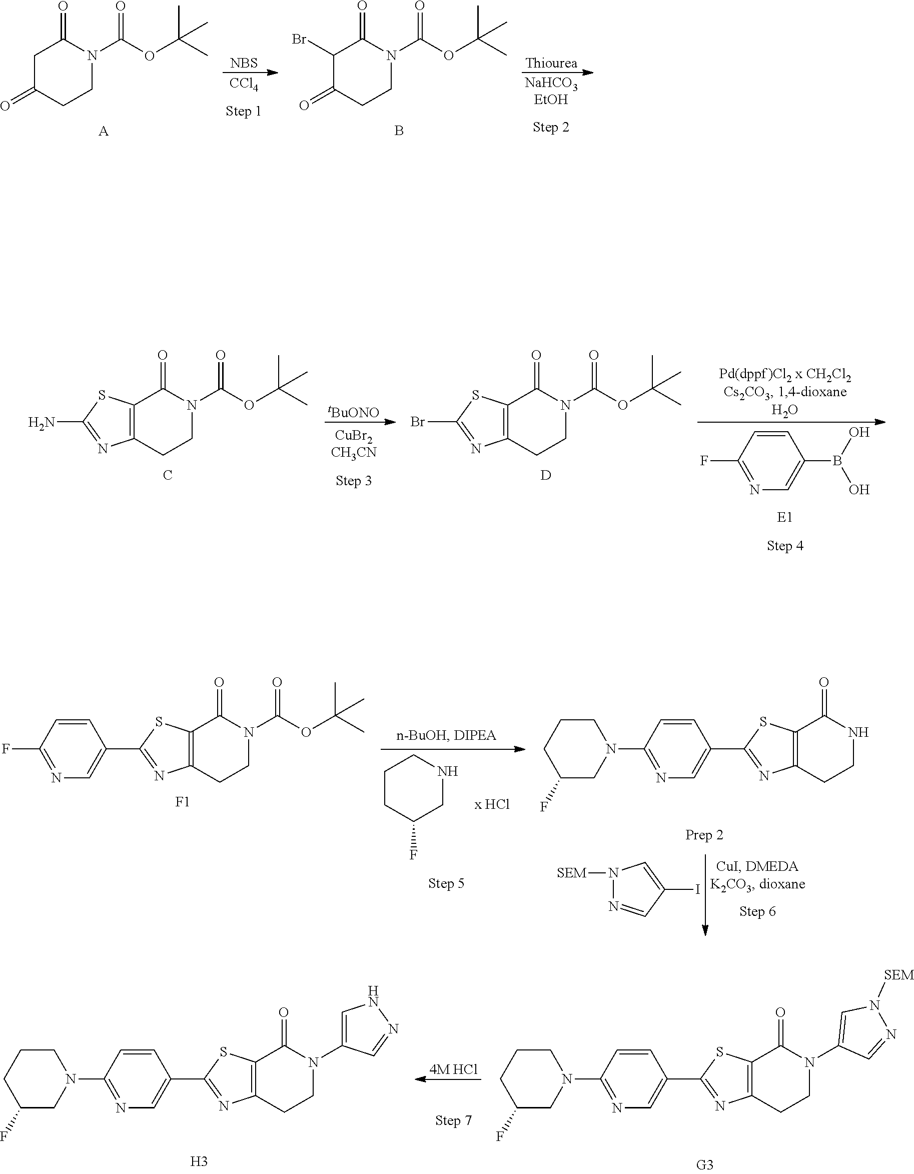

### Synthetic Scheme for the Preparation of 5-(3-amino-2,6-dibromo-4-fluorophenyl)-2-(5-bromo-6-(pyrrolidin-1-yl)pyridin-3-yl)-6,7-dihydrothiazolo[5,4-c]pyridin-4(5H)-one (precursor of ³H Compound 3)

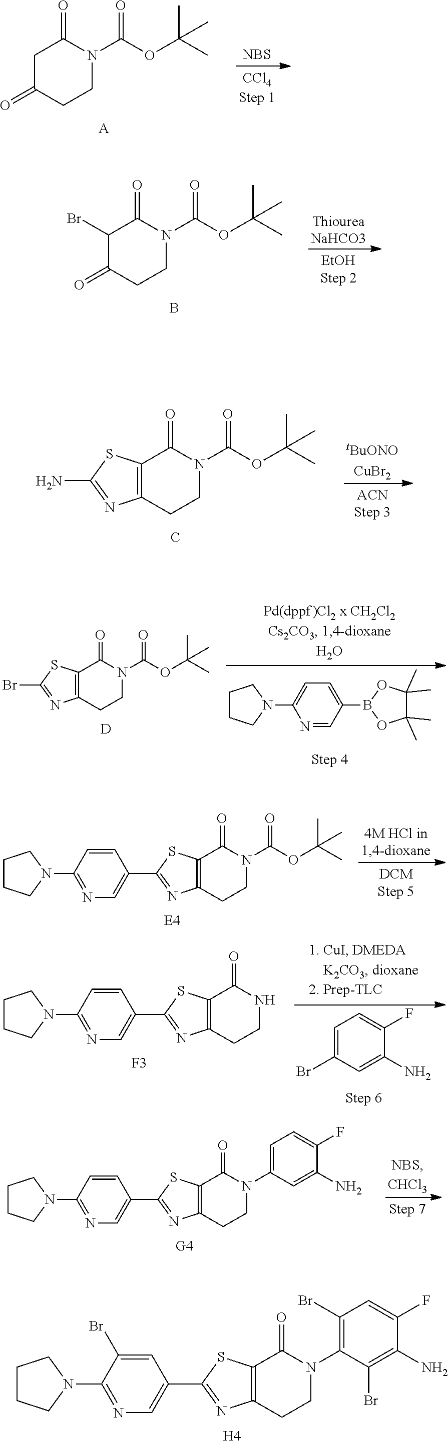

³H labeled compounds can be prepared from a suitable precursor compound containing halogen atoms by catalytic tritiodehalogenation with tritium gas (M. Saljoughian Synthesis (2002), 1781-1801), or from a suitable precursor compound containing a NH moiety by methylation with methyl iodide [³H] (Y. Chen Chemistry 25 (2019):3405-3439). Preferably, the solvents used in the ³H-labeling are DMF or DMA, preferably the solvent is DMF.

### Synthetic Schemes for the Preparation of Precursors for ¹⁸F-Labeling

### Synthetic Scheme for the Preparation of (S)-1-(5-(5-(1-methyl-1H-pyrazol-4-yl)-4-oxo-4,5,6,7-tetrahydrothiazolo[5,4-c]pyridin-2-yl)pyridin-2-yl)pyrrolidin-3-yl methanesulfonate (precursor of ¹⁸F Compound 1)

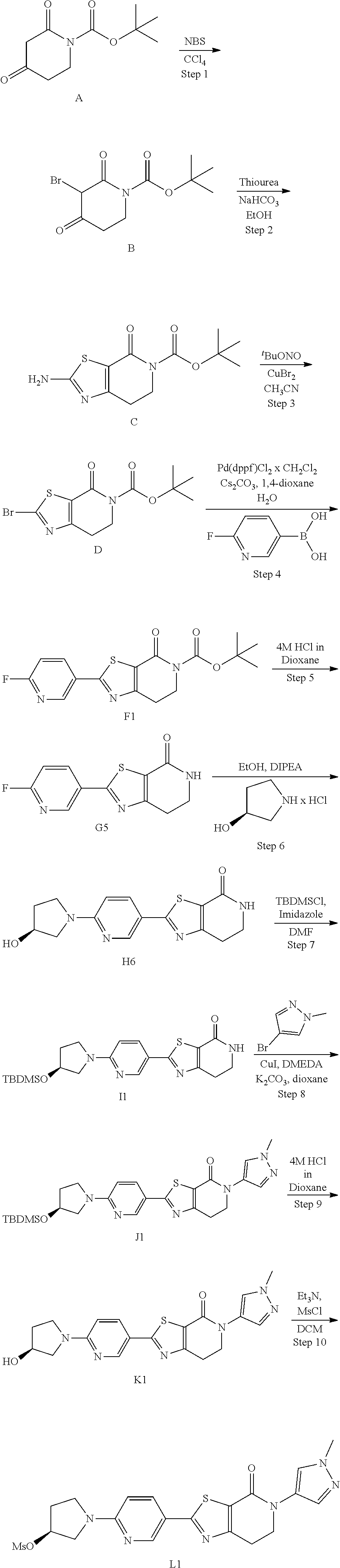

### Synthetic Scheme for the Preparation of (S)-1-(5-(5-(1-methyl-1H-pyrazol-4-yl)-4-oxo-4,5,6,7-tetrahydrothiazolo[5,4-c]pyridin-2-yl)pyridin-2-yl)piperidin-3-yl methanesulfonate (precursor of ¹⁸F Compound 2)

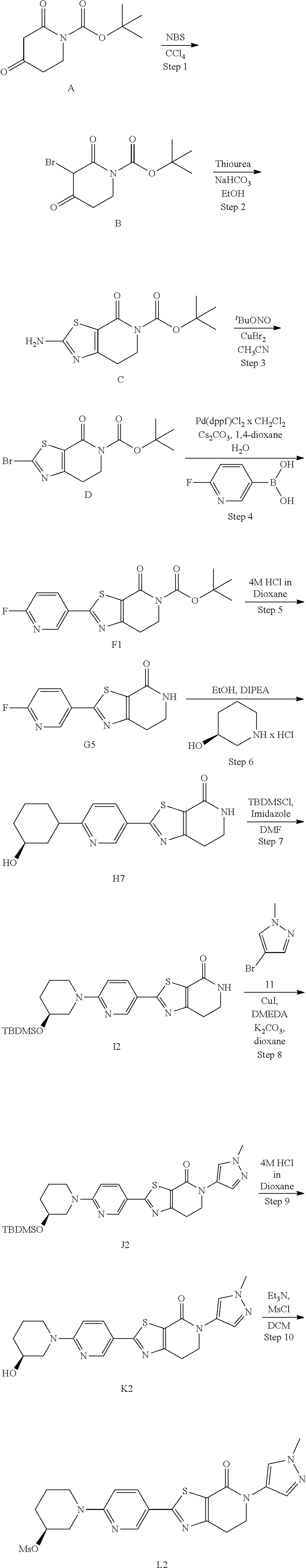

The reactions take place in the presence of a fluorinating agent and typically a solvent.

¹⁸F labeled compounds can be prepared by reacting the precursor compounds containing a LG with an ¹⁸F-fluorinating agent, so that the LG is replaced by ¹⁸F. The ¹⁸F-fluorinating agent can be a tetraalkylammonium salt of ¹⁸F (such as tetra(C₁₋₆ alkyl) ammonium salt of ¹⁸F, e.g., tetrabutylammonium [¹⁸F]fluoride), a tetraalkylphosphonium salt of ¹⁸F (such as tetra(C₁₋₆ alkyl) phosphonium salt of ¹⁸F), K[¹⁸F], Cs¹⁸F, Na¹⁸F, Rb¹⁸F, or Kryptofix[222]K¹⁸F. Preferably, the ¹⁸F-fluorination agent is Cs¹⁸F, K¹⁸F, or tetrabutylammonium [¹⁸F] fluoride. The reagents, solvents and conditions which can be used for the ¹⁸F-fluorination are well-known to a skilled person in the field (L. Cai, S. Lu, V. Pike, Eur. J. Org. Chem. 2008, 2853-2873; J. Fluorine Chem., 27 (1985):177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). Preferably, the solvents used in the ¹⁸F-fluorination are DMF, DMSO, acetonitrile, DMA, or mixtures thereof, preferably the solvent is acetonitrile or DMSO.

Although the reaction is shown above with respect to ¹⁶F as a radioactive label, other radioactive labels can be introduced following similar procedures.

The invention is illustrated by the following examples which, however, should not be construed as limiting.

### EXAMPLES

All reagents and solvents were obtained from commercial sources and were used without further purification. Proton (¹H) NMR spectra were recorded on a Bruker DRX-400 MHz NMR spectrometer or on a Bruker AV-400 MHz NMR spectrometer in deuterated solvents. Mass spectra (MS) were recorded on an Advion CMS mass spectrometer. Chromatography was performed using silica gel (Fluka: Silica gel 60, 0.063-0.2 mm) and suitable solvents as indicated in the specific examples. Flash purification was conducted with a Biotage Isolera One flash purification system using HP-Sil or KP-NH SNAP cartridges (Biotage) and the solvent gradient indicated in the specific examples. Thin layer chromatography (TLC) was carried out on silica gel plates with UV detection.

Although some of the present examples do not indicate that the respective compounds were detectably labeled, it is understood that corresponding detectably labeled compounds are intended and can be easily prepared, e.g., by using detectably labeled starting materials, such as starting materials containing C(³H)₃, (¹¹C)H₃ or ¹⁸F.

### Example 1 Synthesis of (R)-2-(6-(3-fluoropyrrolidin-1-yl)pyridin-3-yl)-5-(1-methyl-1H-pyrazol-4-yl)-6,7-dihydrothiazolo[5,4-c]pyridin-4(5H)-one (1) Compound 1

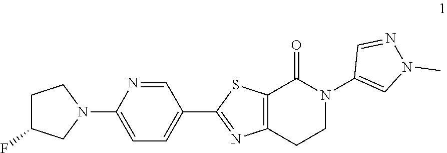 [SMILES: Cn1cc(N2CCc3nc(-c4ccc(N5CC[C@@H](F)C5)nc4)sc3C2=O)cn1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%). ¹H NMR (500 MHz, DMSO-d₆): δ 11.89 (s, 1H), 3.74 (t, 2H), 2.68 (t, 2H), 1.44 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%). ¹H NMR (500 MHz, DMSO-d₆): δ 8.08 (s, 2H), 3.89 (t, 2H), 2.75 (t, 2H), 1.45 (s, 9H) LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper(II)-bromide (6.5 g, 29.43 mmol) was added to the above mixture and it was allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered, and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%). ¹H NMR (500 MHz DMSO-d₆): δ 4.12 (t, 2H), 3.10 (t, 2H), 1.50 (s, 9H). LCMS (ESI) 279 m/z [M+H-C₄H₈]⁺.

### Step-4: Synthesis of tert-butyl 2-(6-fluoropyridin-3-yl)-4-oxo-6,7-dihydrothiazolo[5,4-c]pyridine-5(4H)-carboxylate (F1)

Water (4.0 mL) and 1,4-dioxane (16 mL) were combined and degassed by passing a stream of nitrogen through the mixture. Then [1,1′-bis(diphenyl-phosphino)ferrocene]-dichloropalladium(II), complex with dichloromethane (0.245 g, 0.3 mmol), tert-butyl 2-bromo-4-oxo-6,7-dihydrothiazolo[5,4-c]pyridine-5(4H)-carboxylate D (1.0 g, 3.0 mmol), (6-fluoropyridin-3-yl)boronic acid E1 (0.507 g, 3.6 mmol) and cesium carbonate (2.9 g, 9.0 mmol) were added, and the reaction mixture was heated at 80° C. in an oil bath for 12 hours. The reaction mixture was diluted with two runs of ethyl acetate (80 mL) and water (80 mL). The organic phase was separated, dried over Na₂SO₄, filtered, and the solvents were removed under reduced pressure. The residue was purified by column chromatography on silica gel (100 to 200 mesh) using ethyl acetate/n-hexane gradient (0/100->10/90->20/80) to obtain the title compound F1 as a white solid (0.5 g, 50%). ¹H NMR (500 MHz, DMSO-d₆): δ 8.92 (d, 1H), 8.60 (m, 1H), 7.40 (dd, 1H), 4.09 (t, 2H), 3.17 (t, 2H), 1.50 (s, 9H). LCMS (ESI) 293.94 m/z [M+H-C₄H₈]⁺.

### Step-5: Synthesis of (R)-2-(6-(3-fluoropyrrolidin-1-yl)pyridin-3-yl)-6,7-dihydrothiazolo[5,4-c]pyridin-4(5H)-one (Prep 1)

Title compound F1 (0.3 g, 0.86 mmol), (R)-3-fluoropyrrolidine hydrochloric acid salt (0.213 g, 1.7 mmol) and triethylamine (0.33 mL, 2.36 mmol) were suspended in n-butanol (8 mL) using a microwave vial. The sealed vial was then heated at 160° C. for 1 hour using a CEM microwave. The reaction mixture was concentrated under reduced pressure. The residue obtained was suspended in water (20 mL) and filtered over Whatmann filter paper. The solid was washed with water (20 mL), followed by n-hexane (20 mL) and dried under reduced pressure to obtain the title compound Prep 1 as an off-white solid (0.27 g, 98%). ¹H NMR (500 MHz, DMSO-d₆): δ 8.75 (d, 1H), 8.06 (dd, 1H), 7.81 (s, 1H), 6.64 (d, 1H), 5.48 (d, 1H), 3.90-3.70 (m, 3H), 3.51-3.48 (m, 3H), 2.98 (t, 2H), 2.34-2.13 (m, 2H). LCMS (ESI) 319.00 m/z [M+H]⁺.

### Step-6: Synthesis of (R)-2-(6-(3-fluoropyrrolidin-1-yl)pyridin-3-yl)-5-(1-methyl-1H-pyrazol-4-yl)-6,7-dihydrothiazolo[5,4-c]pyridin-4(5H)-one (1)

Commercial reagent 4-bromo-1-methyl-1H-pyrazole 8 (0.57 g, 3.54 mmol), the title compound Prep 1 (0.375 g, 1.18 mmol), copper(I)-iodide (0.022 g, 0.1157 mmol), N,N′-dimethylethylenediamine (0.2 mL, 2.0 mmol) and potassium carbonate (0.49 g, 3.55 mmol) were suspended in 1,4-dioxane (10 mL). The reaction mixture was heated at about 120° C. in an oil bath for 48 hours. The reaction mixture was diluted with ethyl acetate (100 mL) and washed with water (40 mL) and brine (40 mL). The organic phase was separated, dried over Na₂SO₄, filtered and the solvents were removed under reduced pressure. The residue obtained was purified by column chromatography on silica gel (180 g, 100 to 200 mesh) using dichloromethane/methanol gradient (100/0->99/1->98/2->97.5/2.5) to afford a mixture of title compound 1 and title compound Prep 1. The mixture was further purified by preparative TLC plates using dichloromethane/methanol (93/7) as a mobile phase to obtain the title compound 1 as a yellow solid (0.2 g, 39%). ⁴H NMR (500 MHz, DMSO-d₆): δ 8.75 (d, 1H), 8.07 (dd, 1H), 8.09 (dd, 1H), 8.04 (s, 1H), 7.68 (s, 1H), 6.64 (d, 1H), 5.47 (d, 1H), 4.07 (t, 2H), 3.83 (t, 2H), 3.72 (dd, 2H), 3.64 (dd, 1H), 3.50 (td, 1H), 3.21 (t, 2H), 2.24 (m, 2H). MS (ESI) 399.38 m/z [M+H]⁺. HPLC: 99.87% [Ret. Time: 5.87 min, Eclipse Plus C18].

### Example 2 Synthesis of (R)-2-(6-(3-fluoropiperidin-1-yl)pyridin-3-yl)-5-(1-methyl-1H-pyrazol-4-yl)-6,7-dihydrothiazolo[5,4-c]pyridin-4(5H)-one Compound 2

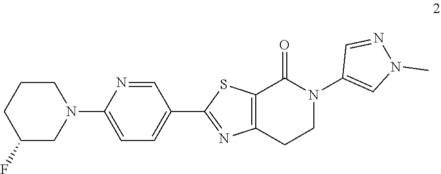 [SMILES: Cn1cc(N2CCc3nc(-c4ccc(N5CCC[C@@H](F)C5)nc4)sc3C2=O)cn1]

Step-1: Synthesis of tert-butyl 3-bromo-2,4-dioxopiperidine-1-carboxylate (B) Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%). ¹H NMR (500 MHz, DMSO-d₆): ¹H NMR (500 MHz, DMSO-d₆): δ 11.89 (s, 1H), 3.74 (t, 2H), 2.68 (t, 2H), 1.44 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) was dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered, and the solvents were removed under reduced pressure. The solid obtained was recrystallized from ethanol to obtain the title compound C as a white solid (6.6 g, 71%). ¹H NMR (500 MHz, DMSO-d₆): δ 8.08 (s, 2H), 3.89 (t, 2H), 2.75 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper(II)-bromide (6.5 g, 29.43 mmol) was added to the above mixture and allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%). ¹H NMR (500 MHz DMSO-d₆): δ 4.12 (t, 2H), 3.10 (t, 2H), 1.50 (s, 9H). LCMS (ESI) 279 m/z [M+H-C₄H₈]⁺.

### Step-4: Synthesis of tert-butyl 2-(6-fluoropyridin-3-yl)-4-oxo-6,7-dihydrothiazolo[5,4-c]pyridine-5(4H)-carboxylate (F1)

Water (4.0 mL) and 1,4-dioxane (16 mL) were combined and degassed by passing a stream of nitrogen through the mixture. Then [1,1′-bis(diphenyl-phosphino)ferrocene]-dichloropalladium(II), complex with dichloromethane (0.245 g, 0.3 mmol), tert-butyl 2-bromo-4-oxo-6,7-dihydrothiazolo[5,4-c]pyridine-5(4H)-carboxylate D (1.0 g, 3.0 mmol), (6-fluoropyridin-3-yl)boronic acid E1 (0.507 g, 3.6 mmol) and cesium carbonate (2.9 g, 9.0 mmol) were added, and the reaction mixture was heated at 80° C. in an oil bath for 12 hours. The reaction mixture was diluted with two runs of ethyl acetate (80 mL) and water (80 mL). The organic phase was separated, dried over Na₂SO₄, filtered, and the solvents were removed under reduced pressure. The residue was purified by column chromatography on silica gel (100 to 200 mesh) using ethyl acetate/n-hexane gradient (0/100->10/90->20/80) to obtain the title compound F1 as a white solid (0.5 g, 50%). ¹H NMR (500 MHz, DMSO-d₆): δ 8.92 (d, 1H), 8.60 (m, 1H), 7.40 (dd, 1H), 4.09 (t, 2H), 3.17 (t, 2H), 1.50 (s, 9H). LCMS (ESI) 293.94 m/z [M+H-C₄H₈]⁺.

### Step-5: Synthesis of (R)-2-(6-(3-fluoropiperidin-1-yl)pyridin-3-yl)-6,7-dihydrothiazolo[5,4-c]pyridin-4(5H)-one (Prep 2)

Title compound F1 (0.55 g, 1.5 mmol), (R)-3-fluoropiperidine hydrochloric acid salt (0.328 g, 2.4 mmol), DIPEA (0.79 mL, 4.5 mmol) were suspended in n-butanol (6.6 mL) using a microwave vial. The sealed vial was then heated at 160° C. for 45 minutes. using a CEM microwave. The reaction mixture was concentrated under reduced pressure. The residue obtained was suspended in water (20 mL) and filtered over Whatmann filter paper. The solid was washed with water (20 mL), followed by n-hexane (20 mL) and dried under reduced pressure to obtain the title compound Prep 2 as a green solid (0.33 g, 63%). ¹H NMR (500 MHz, DMSO-d₆): δ 8.7 (d, 1H), 8.02 (dd, 1H), 7.81 (s, 1H), 6.98 (d, 1H), 4.80 (d, 1H), 4.10-4.06 (m, 1H), 3.94-3.90 (m, 1H), 3.76-3.67 (m, 1H), 3.51-3.48 (m, 2H), 3.44-3.99 (m, 1H), 3.94-3.90 (m, 1H), 2.98 (t, 2H), 1.97-1.86 (m, 2H), 1.77-1.74 (m, 1H), 1.57-1.54 (m, 1H). LCMS: 95% [Ret. Time: 7.0 min, Hypersil BDS C18], (ESI) 332.9 m/z [M+H]⁺.

### Step-6: Synthesis of (R)-2-(6-(3-fluoropiperidin-1-yl)pyridin-3-yl)-5-(1-methyl-1H-pyrazol-4-yl)-6,7-dihydrothiazolo[5,4-c]pyridin-4(5H)-one (2)

Commercial reagent 4-bromo-1-methyl-1H-pyrazole 8 (0.218 g, 1.35 mmol), the title compound Prep 2 (0.18 g, 0.54 mmol), copper(I)-iodide (0.01 g, 0.05 mmol), N,N′-dimethylethylenediamine (0.095 mL, 0.9 mmol) and potassium carbonate (0.186 g, 1.35 mmol) were suspended in 1,4-dioxane (6 mL). The reaction mixture was heated at about 120° C. in an oil bath for 36 hours. The reaction mixture was concentrated under reduced pressure. The residue obtained was purified by column chromatography on silica gel (180 g, 100 to 200 mesh) using dichloromethane/methanol gradient (100/0->99/1->98/2->97.5/2.5) to afford a mixture of title compound 2 and title compound Prep 2. The mixture was further purified by preparative TLC plates using dichloromethane/methanol (93/7) as a mobile phase to obtain the title compound 2 as a yellow solid (0.065 g, 29%). ¹H NMR (500 MHz, DMSO-d₆): δ 8.73 (d, 1H), 8.05-8.02 (m, 2H), 7.67 (s, 1H), 8.04 (s, 1H), 6.99 (d, 1H), 4.81 (d, 1H), 4.10-4.05 (m, 3H), 4.00-3.90 (m, 1H), 3.83 (s, 3H), 3.45-3.39 (m, 1H), 3.21 (t, 2H), 1.97-1.87 (m, 2H), 1.80-1.73 (m, 1H), 1.60-1.53 (m, 1H). MS (ESI) 413.35 m/z [M+H]⁺. HPLC: 97.4% [Ret. Time: 6.89 min, Eclipse Plus C18].

### Example 3 Synthesis of (5-(3-amino-4-fluorophenyl)-2-(6-(pyrrolidin-1-yl)pyridin-3-yl)-6,7-dihydrothiazolo[5,4-c]pyridin-4(5H)-one Compound 3

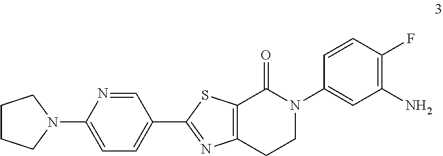 [SMILES: Nc1cc(N2CCc3nc(-c4ccc(N5CCCC5)nc4)sc3C2=O)ccc1F]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%). ¹H NMR (500 MHz, DMSO-d₆): δ 11.89 (s, 1H), 3.74 (t, 2H), 2.68 (t, 2H), 1.44 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%). ¹H NMR (500 MHz, DMSO-d₆): δ 8.08 (s, 2H), 3.89 (t, 2H), 2.75 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-Butyl nitrite (4.3 mL, 36.4 mmol) was added to the above solution and stirring was continued at −10° C. for 1 hour. Copper(II)-bromide (6.5 g, 29.43 mmol) was added to the above mixture and allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%). ¹H NMR (500 MHz DMSO-d₆): δ 4.12 (t, 2H), 3.10 (t, 2H), 1.50 (s, 9H). LCMS (ESI) 279 m/z [M+H-C₄H₈]⁺.

### Step-4: Synthesis of 5-bromo-2-(pyrrolidin-1-yl) pyridine (F2)

5-Bromo-2-fluoropyridine E2 (1.5 g, 8.5 mmol) and pyrrolidine (3.6 g, 50.62 mmol) were dissolved in ethanol (8 mL) using a microwave vial. The sealed vial was then heated at 120° C. for 1 hour using a CEM microwave. The reaction mixture was concentrated under reduced pressure to afford crude product. The resultant crude product was suspended in water (15 mL) and filtered over Whatmann filter paper. The solid was washed with water (2×15 mL) and dried under reduced pressure to afford the title compound F2 as an off-white solid (1.9 g, 98%). ¹H NMR (500 MHz, DMSO-d₆): δ 8.10 (d, 1H), 7.61 (dd, 1H), 6.42 (d, 1H), 3.34-3.33 (m, 4H), 1.94-1.91 (m, 4H). LCMS (ESI) 228.99 m/z [M+H]⁺.

### Step-5: Synthesis of 2-(pyrrolidin-1-yl)-5-(4,4,5,5-tetramethyl-1,3,2-dioxaborolan-2-yl) pyridine (G1)

1,4-Dioxane (25 mL) was degassed by passing a stream of nitrogen through the mixture. Then [1,1′-bis(diphenyl-phosphino)ferrocene]-dichloropalladium(II), complex with dichloromethane (0.683 g, 0.83 mmol), 5-bromo-2-(pyrrolidin-1-yl)pyridine F2 (1.4 g, 4.2 mmol), 4,4,4′,4′,5,5,5′,5′-octamethyl-2,2′-bi(1,3,2-dioxaborolane) (4.25 g, 16.73 mmol) and potassium acetate (1.64 g, 16.73 mmol) were added, and the reaction mixture was heated at 100° C. in an oil bath for 3 hours. After completion of the reaction, solvent was removed from the reaction mixture under reduced pressure. The residue was washed with 3 runs of n-hexane (100 mL). The reaction mixture was diluted with two runs of ethyl acetate (80 mL) and water (80 mL). The organic phase was dried over Na₂SO₄, filtered and the solvents were removed under reduced pressure to afford a pale-yellow sticky solid G1 (5.7 g, crude). The solid was used in step as such without further purification. LCMS (ESI) 275.22 m/z [M+H]⁺.

### Step-6: Synthesis of tert-butyl 4-oxo-2-(6-(pyrrolidin-1-yl)pyridin-3-yl)-6,7-dihydrothiazolo[5,4-c]pyridine-5(4H)-carboxylate (H1)

Water (10 mL) and 1,4-dioxane (40 mL) were combined and degassed by passing a stream of nitrogen through the mixture. Then [1,1′-bis(diphenyl-phosphino)ferrocene]-dichloropalladium(II), complex with dichloromethane (0.342 g, 0.4 mmol), tert-butyl 2-bromo-4-oxo-6,7-dihydrothiazolo[5,4-c]pyridine-5(4H)-carboxylate D (1.4 g, 4.2 mmol), 2-(pyrrolidin-1-yl)-5-(4,4,5,5-tetramethyl-1,3,2-dioxaborolan-2-yl)pyridine G1 (1.73 g, 6.3 mmol) and cesium carbonate (4.1 g, 12.6 mmol) were added, and the reaction mixture was heated at 100° C. in an oil bath for 5 hours. The reaction mixture was diluted with two runs of ethyl acetate (80 mL) and water (80 mL). The organic phase was separated, dried over Na₂SO₄, filtered and the solvents were removed under reduced pressure. The residue was purified by column chromatography on basic silica gel (60 to 120 mesh) using ethyl acetate/n-hexane gradient (0/100->10/90->20/80->30/70->40/60) to obtain the title compound H1 as a pale-yellow solid (0.8 g, 48%). ¹H NMR (500 MHz, DMSO-d₆): δ 8.77 (d, 1H), 7.97 (dd, 1H), 6.40 (d, 1H), 4.14 (t, 2H), 3.54 (s, 4H), 3.10 (t, 2H), 2.04 (m, 4H), 1.57 (s, 9H). LCMS (APCI) 401.26 m/z [M+H]⁺.

### Step-7: Synthesis of 2-(6-(pyrrolidin-1-yl)pyridin-3-yl)-6,7-dihydrothiazolo[5,4-c]pyridin-4(5H)-one (Prep 3)

Title compound H1 (0.34 g, 0.99 mmol) was dissolved in DCM (8 mL) and cooled to 0° C. in an ice bath with stirring. 4M HCl in 1,4-dioxane (4.0 mL) was added to the above solution and stirring was continued at room temperature for 4 hours. After completion of the reaction, solvent was removed under reduced pressure. The residue obtained was dissolved in ice cold water and basified with aqueous saturated sodium bicarbonate solution to pH 8 to 9, the precipitated solid was filtered and dried to afford the title compound Prep 3 as a pale-yellow solid (0.25 g, 83%). ¹H NMR (500 MHz, DMSO-d₆): δ 8.70 (d, 1H), 8.00 (dd, 1H), 7.80 (s, 1H), 6.55 (d, 1H), 3.50-3.40 (m, 6H), 2.97 (t, 2H), 1.97-1.92 (m, 4H). LCMS (ESI) 301.08 m/z [M+H]⁺.

### Step-8: Synthesis of 5-(3-amino-4-fluorophenyl)-2-(6-(pyrrolidin-1-yl)pyridin-3-yl)-6,7-dihydrothiazolo[5,4-c]pyridin-4(5H)-one (3)

Commercial reagent 5-bromo-2-fluoroaniline (0.095 g, 0.5 mmol), the title compound Prep 3 (0.05 g, 0.166 mmol), copper (I)-iodide (0.003 g, 0.0166 mmol), N,N′-dimethylethylenediamine (0.03 mL, 0.28 mmol) and potassium carbonate (0.046 g, 0.33 mmol) were suspended in 1,4-dioxane (3 mL). The reaction mixture was heated at about 120° C. in an oil bath for 24 hours. The reaction mixture was concentrated under reduced pressure to afford a crude product. The residue obtained was purified by column chromatography on silica gel (80 g, 100 to 200 mesh) using dichloromethane/methanol gradient (100/0->98/2) to afford the title compound 3 as a yellow solid (80 mg, 73.4%). ¹H NMR (500 MHz, DMSO-d6): δ 8.73 (d, 1H), 8.04 (dd, 1H), 7.02-6.98 (dd, 1H), 6.76-6.74 (dd, 1H), 6.58 (d, 1H), 6.50-6.48 (m, 1H), 5.24 (d, 1H), 3.98 (t, 2H), 3.47 (s, 4H), 3.17 (t, 2H), 1.97 (s, 4H). LCMS: 96.7% [Ret. Time: 8.6 min, Hypersil BDS C18], (ESI) 410 m/z [M+H]⁺.

### Example 4 Synthesis of (R)-2-(6-(3-fluoropyrrolidin-1-yl)pyridin-3-yl)-5-(1H-pyrazol-4-yl)-6,7-dihydrothiazolo[5,4-c]pyridin-4(5H)-one (precursor of ³H Compound 1)

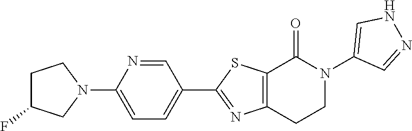 [SMILES: O=C1c2sc(-c3ccc(N4CC[C@@H](F)C4)nc3)nc2CCN1c1cn[nH]c1]

### Step-1: Synthesis of tert-butyl 2-(6-fluoropyridin-3-yl)-4-oxo-6,7-dihydrothiazolo[5,4-c]pyridine-5(4H)-carboxylate (F1)

Water (2.2 mL) and 1,4-dioxane (9.5 mL) were combined and degassed by sonication while a stream of argon was passed through the mixture. Then [1,1′-bis(diphenyl-phosphino)ferrocene]-dichloropalladium(II) complex with dichloromethane (0.019 g, 0.022 mmoil), tert-butyl 2-bromo-4-oxo-6,7-dihydrothiazolo[5,4-c]pyridine-5(4H)-carboxylate D (0.15 g, 0.45 mmol), (6-fluoropyridin-3-yl)boronic acid (0.078 g, 0.55 mmol) and cesium carbonate (0.319 g, 0.9 mmol) were added, and the reaction mixture was heated at about 85° C. in a sand bath for 5 hours. The reaction mixture of two runs was diluted with ethyl acetate (200 mL) and water (80 mL). The organic phase was separated, dried over Na₂SO₄, filtered and the solvents were removed under reduced pressure. The residue was purified by chromatography on silica (50 g HP-Ultra) using a Biotage Isolera system employing an ethyl acetate/n-heptane gradient (5/95->10/90->20/80->30/70->40/60) to afford the title compound F1 as a white solid (0.225 g, 71%). ¹H NMR (400 MHz, DMSO-d₆): δ 8.92 (d, 1H), 8.60 (ddd, 1H), 7.41 (dd, 1H), 4.10 (t, 2H), 3.17 (t, 2H), 1.50 (s, 9H). LCMS (ESI) 293.94 m/z [M+H-C₄H₈]⁺.

### Step-2: Synthesis of (R)-2-(6-(3-fluoropyrrolidin-1-yl)pyridin-3-yl)-6,7-dihydrothiazolo[5,4-c]pyridin-4(5H)-one (Prep 1)

Title compound F1 (0.1125 g, 0.3225 mmol), (R)-3-fluoropyrrolidine hydrochloric acid salt (0.122 g, 0.97 mmol), and N,N′-diisopropylethylamine (0.25 mL, 1.45 mmol) were suspended in n-butanol (10 mL) using a microwave vial. The sealed vial was then heated at 160° C. for 1 hour using a Biotage Initiator microwave. The reaction mixture of two runs was diluted with ethyl acetate/methanol (200 mL; 9/1) and washed with water (80 mL) and brine (80 mL). The organic phase was separated, dried over Na₂SO₄, filtered and the solvents were removed under reduced pressure. The residue was purified by chromatography on silica (25 g HP-Ultra) using a Biotage Isolera system employing dichloromethane/methanol gradient (100/0->95/5->93/7->90/10) to afford the title compound Prep 1 as an off-white solid (0.151 g, 73%). ¹H NMR (400 MHz, DMSO-d₆): δ 8.73 (d, 1H), 8.06 (dd, 1H), 7.81 (s, 1H), 6.64 (d, 1H), 5.48 (d, 1H), 3.85-3.61 (m, 4H), 3.50 (t, 2H), 2.99 (t, 2H), 2.32-2.17 (m, 2H). LCMS (ESI) 319.00 m/z [M+H]⁺.

### Step-3: Synthesis of 4-bromo-1-(tetrahydro-2H-pyran-2-yl)-1H-pyrazole (E3)

Commercial reagent 4-bromo-1H-pyrazole (1.47 g, 10 mmol) was dissolved in acetonitrile (10 mL) and 3,4-dihydro-2H-pyran (2.39 mL, 26 mmol) was added. After the addition of trifluoroacetic acid (0.05 mL, 0.69 mmol), the reaction mixture was heated at 90° C. for 2 hours using a Biotage Initiator microwave. The reaction mixture was diluted with ethyl acetate (200 mL) and washed with water (80 mL) and brine (80 mL). The organic phase was separated, dried over Na₂SO₄, filtered and the solvents were removed under reduced pressure. The residue was purified by chromatography on silica (100 g HP-Ultra) using a Biotage Isolera system employing an ethyl acetate/n-heptane gradient (5/95->10/90->20/80->30/70->40/60) to afford a pale-yellow liquid. Title compound was further purified by chromatography on silica (40 g Interchim) using a Biotage Isolera system employing an ethyl acetate/n-heptane gradient (5/95->10/90->20/80->30/70) to afford the title compound E3 as a colorless liquid (0.989 g, 42%). ¹H NMR (400 MHz, CDCl₃): δ 7.56 (s, 1H), 7.43 (s, 1H), 5.29-5.26 (m, 1H), 3.99-3.94 (m, 1H), 3.65-3.58 (m, 1H), 2.02-1.92 (m, 3H), 1.64-1.52 (m, 3H).

### Step-4: Synthesis of 2-(6-((R)-3-fluoropyrrolidin-1-yl)pyridin-3-yl)-5-(1-(tetrahydro-2H-pyran-2-yl)-1H-pyrazol-4-yl)-6,7-dihydrothiazolo[5,4-c]pyridin-4(5H)-one (G2)

The title compound Prep 1 (0.05 g, 0.157 mmol), title compound E3 (0.109 g, 0.472 mmol), copper(I) iodide (0.003 g, 0.0157 mmol), N,N′-dimethylethylenediamine (0.025 mL, 0.27 mmol) and potassium carbonate (0.043 g, 0.314 mmol) were suspended in 1,4-dioxane (9 mL). The reaction mixture was heated at about 120° C. in a sand bath for 24 hours. The reaction mixture was diluted with ethyl acetate (100 mL) and washed with water (40 mL) and brine (40 mL). The organic phase was separated, dried over Na₂SO₄, filtered and the solvents were removed under reduced pressure. The residue was purified by chromatography on silica (25 g HP-Ultra) using a Biotage Isolera system employing a dichloromethane/methanol gradient (100/0->95/5->95/15) to afford a mixture of title compound G2 and title compound Prep 1. The mixture was further purified by preparative TLC plates using dichloromethane/methanol (90/10) as a mobile phase to afford the title compound G2 as a yellow solid (0.0384 g, 52%). ¹H NMR (400 MHz, DMSO-d₆): δ 8.76 (d, 1H), 8.19 (s, 1H), 8.09 (dd, 1H), 7.81 (s, 1H), 6.65 (d, 1H), 5.48 (d, 1H), 5.41-5.38 (m, 1H), 4.10 (t, 2H), 3.95-3.91 (m, 1H), 3.87-3.61 (m, 4H), 3.55-3.48 (m, 1H), 3.23 (t, 2H), 2.34-2.26 (m, 2H), 2.10-2.05 (m, 1H), 1.97-1.89 (m, 2H), 1.72-1.64 (m, 1H), 1.57-1.52 (m, 2H). LCMS (ESI) 469.15 m/z [M+H]⁺

### Step-5: Synthesis of (R)-2-(6-(3-fluoropyrrolidin-1-yl)pyridin-3-yl)-5-(1H-pyrazol-4-yl)-6,7-dihydrothiazolo[5,4-c]pyridin-4(5H)-one (H2)

Title compound G2 (0.0384 g, 0.082 mmol) was dissolved in dichloromethane (4 mL) and trifluoroacetic acid (2 mL) was added. The reaction mixture was stirred at room temperature for 18 hours. The solvent was evaporated under reduced pressure, the residue was dissolved in methanol (5 mL), and the solvent was evaporated under reduced pressure. The residue was dissolved in ethyl acetate (80 mL) and saturated NaHCO₃ (30 mL) was added. The organic phase was separated, washed with brine (30 mL), dried over Na₂SO₄, filtered and the solvents were removed under reduced pressure. The residue was purified by chromatography on silica (25 g HP Ultra) using a Biotage Isolera system employing a dichloromethane/methanol gradient (100/0->95/5->90/10->80/20) to afford the title compound H2 as a yellow solid (0.0188 g, 60%). ¹H NMR (400 MHz, DMSO-d₆): δ 12.77 (br-s, 1H), 8.76 (d, 1H), 8.08 (dd, 1H), 8.03-7.76 (br-m, 2H), 6.65 (d, 1H), 5.49 (d, 1H), 4.10 (t, 1H), 3.88-3.62 (m, 3H), 3.55-3.47 (m, 1H), 3.22 (t, 2H), 2.34-2.26 (m, 2H). LCMS (ESI) 385.17 m/z [M+H]⁺.

### Example 5 Synthesis of (R)-2-(6-(3-fluoropiperidin-1-yl)pyridin-3-yl)-5-(1H-pyrazol-4-yl)-6,7-dihydrothiazolo[5,4-c]pyridin-4(5H)-one (precursor of ³H Compound 2)

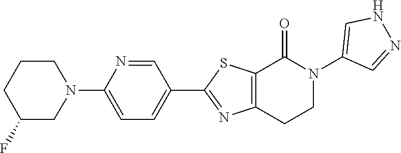 [SMILES: O=C1c2sc(-c3ccc(N4CCC[C@@H](F)C4)nc3)nc2CCN1c1cn[nH]c1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%). ¹H NMR (500 MHz, DMSO-d₆): δ 11.89 (s, 1H), 3.74 (t, 2H), 2.68 (t, 2H), 1.44 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%). ¹H NMR (500 MHz, DMSO-d₆): δ 8.10 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hours. Copper (II)-bromide (6.5 g, 29.43 mmol) was added to the above mixture and it was allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered, and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%). ¹H NMR (500 MHz DMSO-d₆): δ 4.12 (t, 2H), 3.10 (t, 2H), 1.50 (s, 9H). LCMS (ESI) 279 m/z [M+H-C₄H8]⁺.

### Step-4: Synthesis of tert-butyl 2-(6-fluoropyridin-3-yl)-4-oxo-6,7-dihydrothiazolo[5,4-c]pyridine-5(4H)-carboxylate (F1)

Water (4.0 mL) and 1,4-dioxane (16 mL) were combined and degassed by passing a stream of nitrogen through the mixture. Then [1,1′-bis(diphenyl-phosphino)ferrocene]-dichloropalladium(II) complex with dichloromethane (0.245 g, 0.3 mmol), tert-butyl 2-bromo-4-oxo-6,7-dihydrothiazolo[5,4-c]pyridine-5(4H)-carboxylate D (1.0 g, 3.0 mmol), (6-fluoropyridin-3-yl)boronic acid (0.507 g, 3.6 mmol) and cesium carbonate (2.9 g, 9.0 mmol) were added, and the reaction mixture was heated at 80° C. in an oil bath for 12 hours. The reaction mixture was diluted with two runs of ethyl acetate (80 mL) and water (80 mL). The organic phase was separated, dried over Na₂SO₄, filtered and the solvents were removed under reduced pressure. The residue was purified by column chromatography on silica gel (100 to 200 mesh) using ethyl acetate/n-hexane gradient (0/100->10/90->20/80) to obtain the title compound F1 as a white solid (0.5 g, 50%). ¹H NMR (500 MHz, DMSO-d₆): δ 8.92 (d, 1H), 8.60 (m, 1H), 7.40 (dd, 1H), 4.09 (t, 2H), 3.17 (t, 2H), 1.50 (s, 9H). LCMS (ESI) 293.94 m/z [M+H-C₄H₈]⁺.

### Step-5: Synthesis of (R)-2-(6-(3-fluoropiperidin-1-yl)pyridin-3-yl)-6,7-dihydrothiazolo[5,4-c]pyridin-4(5H)-one (Prep 2)

Title compound F1 (0.55 g, 1.5 mmol), (R)-3-fluoropiperidine hydrochloric acid salt (0.328 g, 2.4 mmol), N,N-diisopropylethylamine (DIPEA) (0.79 mL, 4.5 mmol) were suspended in n-butanol (6.6 mL) using a microwave vial. The sealed vial was then heated at 160° C. for 45 minutes. using a CEM microwave. The reaction mixture was concentrated under reduced pressure. The residue obtained was suspended in water (20 mL) and filtered over Whatmann filter paper. The solid was washed with water (20 mL), followed by n-hexane (20 mL) and dried under reduced pressure to obtain the title compound Prep 2 as a green solid (0.33 g, 63%). ¹H NMR (500 MHz, DMSO-d₆): δ 8.70 (d, 1H), 8.02 (dd, 1H), 7.81 (s, 1H), 6.98 (d, 1H), 4.80 (d, 1H), 4.10-4.06 (m, 1H), 3.94-3.90 (m, 1H), 3.76-3.67 (m, 1H), 3.51-3.48 (m, 2H), 3.44-3.99 (m, 1H), 3.94-3.90 (m, 1H), 2.98 (t, 2H), 1.97-1.86 (m, 2H), 1.77-1.74 (m, 1H), 1.57-1.54 (m, 1H). LCMS: 95% [Ret. Time: 7.0 min, Hypersil BDS C18], (ESI) 332.9 m/z [M+H]⁺.

### Step-6: Synthesis of (R)-2-(6-(3-fluoropiperidin-1-yl)pyridin-3-yl)-5-(1-methyl-1H-pyrazol-4-yl)-6,7-dihydrothiazolo[5,4-c]pyridin-4(5H)-one (G3)

4-Iodo-1-((2-(trimethylsilyl)ethoxy)methyl)-1H-pyrazole (0.194 g, 0.6 mmol), the title compound Prep 2 (0.1 g, 0.3 mmol), copper(I)-iodide (0.03 g, 0.15 mmol), N,N′-dimethylethylenediamine (0.03 mL, 0.3 mmol) and potassium carbonate (0.08 g, 0.6 mmol) were suspended in 1,4-dioxane (10 mL). The reaction mixture was heated at about 120° C. in an oil bath for 24 hours. The reaction mixture was concentrated under reduced pressure. The residue obtained was purified by column chromatography on basic silica gel (100 g, 100 to 200 mesh) using dichloromethane/methanol gradient (100/0->98/2->97/3) to afford the title compound G3 as a yellow solid (0.12 g, 75%). ¹H NMR (500 MHz, DMSO-d₆): δ 8.73 (d, 1H), 8.22 (s, 1H), 8.05 (dd, 1H), 7.83 (s, 1H), 6.99 (d, 1H), 5.40 (s, 2H), 4.81 (d, 1H), 4.10-4.05 (m, 3H), 3.94 (d, 1H), 3.76-3.68 (m, 1H), 3.54 (t, 2H), 3.42 (t, 1H), 3.22 (t, 2H), 1.99-1.94 (m, 2H), 1.89-1.75 (m, 1H), 1.56-1.53 (m, 1H), 0.85 (t, 2H), −30.39 (s, 9H). MS (ESI) 529.3 m/z [M+H]⁺.

### Step-7: Synthesis of (R)-2-(6-(3-fluoropiperidin-1-yl)pyridin-3-yl)-5-(1H-pyrazol-4-yl)-6,7-dihydrothiazolo[5,4-c]pyridin-4(5H)-one (H3)

Title compound G3 (0.12 g, 0.23 mmol) was dissolved in dichloromethane (4 mL) and cooled to 0° C. in an ice bath with stirring. 4M HCl in 1,4-dioxane (2.0 mL) was added to the above solution and stirring was continued at room temperature for 4 hours. After completion of the reaction, solvent was removed under reduced pressure. The residue obtained was dissolved in ice cold water and basified with aqueous saturated sodium bicarbonate solution to pH 8 to 9, the precipitated solid was filtered. The solid was further purified by column chromatography on silica gel (100 g, 100 to 200 mesh) using dichloromethane/methanol gradient (100/0->98/2->97/3) to afford the title compound H3 as a yellow solid (0.06 g, 55%). ¹H NMR (500 MHz, DMSO-d₆): δ 12.79 (s, 1H), 8.73 (d, 1H), 8.04 (dd, 2H), 7.79 (d, 1H), 6.99 (d, 1H), 4.88-4.72 (m, 1H), 4.13-4.06 (m, 3H), 3.97-3.91 (m, 1H), 3.77-3.66 (m, 1H), 3.45-3.40 (m, 1H), 3.21 (t, 2H), 1.98-1.87 (m, 2H), 1.79-1.72 (m, 1H), 1.59-1.53 (m, 1H). MS (ESI) 399.22 m/z [M+H]⁺. HPLC: 98.2% [Ret. Time: 7.65 min, Hypersil BDS C18].

### Example 6 Synthesis of 5-(3-amino-2,6-dibromo-4-fluorophenyl)-2-(5-bromo-6-(pyrrolidin-1-yl)pyridin-3-yl)-6,7-dihydrothiazolo[5,4-c]pyridin-4(5H)-one (precursor of ³H Compound 3)

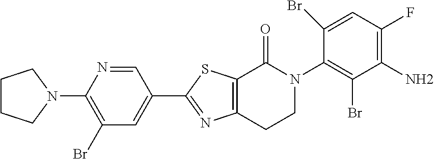 [SMILES: Nc1c(F)cc(Br)c(N2CCc3nc(-c4cnc(N5CCCC5)c(Br)c4)sc3C2=O)c1Br]

Step-1: Synthesis of tert-butyl 3-bromo-2,4-dioxopiperidine-1-carboxylate (B) Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%). ¹H NMR (500 MHz, DMSO-d₆): δ 8.10 (s, 2H), 3.89 (t, 2H), 2.76 (t, 2H), 1.45 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%). ¹H NMR (500 MHz, DMSO-d₆): δ 8.10 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper (II)-bromide (6.5 g, 29.43 mmol) was added to the above mixture and allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%). ¹H NMR (500 MHz DMSO-d₆): δ 4.12 (t, 2H), 3.10 (t, 2H), 1.50 (s, 9H). LCMS (ESI) 279 m/z [M+H-C₄H₈]⁺.

### Step-4: Synthesis of tert-butyl 4-oxo-2-(6-(pyrrolidin-1-yl)pyridin-3-yl)-6,7-dihydrothiazolo[5,4-c]pyridine-5(4H)-carboxylate (E4)

Water (10 mL) and 1,4-dioxane (40 mL) were combined and degassed by passing a stream of nitrogen through the mixture. Then [1,1′-bis(diphenyl-phosphino)ferrocene]-ichloropalladium(II), complex with dichloromethane (0.342 g, 0.4 mmol), tert-butyl 2-bromo-4-oxo-6,7-dihydrothiazolo[5,4-c]pyridine-5(4H)-carboxylate D (1.4 g, 4.2 mmol), 2-(pyrrolidin-1-yl)-5-(4,4,5,5-tetramethyl-1,3,2-dioxaborolan-2-yl)pyridine (1.73 g, 6.3 mmol) and cesium carbonate (4.1 g, 12.6 mmol) were added, and the reaction mixture was heated at 100° C. in an oil bath for 5 hours. The reaction mixture was diluted with two runs of ethyl acetate (80 mL) and water (80 mL). The organic phase was separated, dried over Na₂SO₄, filtered and the solvents were removed under reduced pressure. The residue was purified by column chromatography on basic silica gel (60 to 120 mesh) using ethyl acetate/n-hexane gradient (0/100->10/90->20/80->30/70->40/60) to obtain the title compound E4 as a pale-yellow solid (0.8 g, 48%). ¹H NMR (500 MHz, DMSO-d₆): δ 8.77 (d, 1H), 7.97 (dd, 1H), 6.40 (d, 1H), 4.14 (t, 2H), 3.54 (s, 4H), 3.10 (t, 2H), 2.04 (m, 4H), 1.57 (s, 9H). LCMS (APCI) 401.26 m/z [M+H]⁺.

### Step-5: Synthesis of 2-(6-(pyrrolidin-1-yl)pyridin-3-yl)-6,7-dihydrothiazolo[5,4-c]pyridin-4(5H)-one (F3)

Title compound E4 (0.34 g, 0.99 mmol) was dissolved in dichloromethane (8 mL) and cooled to 0° C. in an ice bath with stirring. 4M HCl in 1,4-dioxane (4.0 mL) was added to the above solution and stirring was continued at room temperature for 4 hours. After completion of the reaction, solvent was removed under reduced pressure. The residue obtained was dissolved in ice cold water and basified with aqueous saturated sodium bicarbonate solution to pH 8 to 9, the precipitated solid was filtered and dried to afford the title compound F3 as a pale-yellow solid (0.25 g, 83%). ¹H NMR (500 MHz, DMSO-d₆): δ 8.70 (d, 1H), 8.00 (dd, 1H), 7.80 (s, 1H), 6.55 (d, 1H), 3.50-3.40 (m, 6H), 2.97 (t, 2H), 1.97-1.92 (m, 4H). LCMS (ESI) 301.08 m/z [M+H]⁺.

### Step-6: Synthesis of 5-(3-amino-4-fluorophenyl)-2-(6-(pyrrolidin-1-yl)pyridin-3-yl)-6,7-dihydrothiazolo[5,4-c]pyridin-4(5H)-one (G4)

Commercial reagent 5-bromo-2-fluoroaniline (0.095 g, 0.5 mmol), the title compound F3 (0.05 g, 0.166 mmol), copper (I)-iodide (0.003 g, 0.0166 mmol), N,N′-dimethylethylenediamine (0.03 mL, 0.28 mmol) and potassium carbonate (0.046 g, 0.33 mmol) were suspended in 1,4-dioxane (3 mL). The reaction mixture was heated at about 120° C. in an oil bath for 24 hours. The reaction mixture was concentrated under reduced pressure to afford a crude product. The residue obtained was purified by column chromatography on silica gel (80 g, 100 to 200 mesh) using dichloromethane/methanol gradient (100/0->98/2) to afford the title compound G4 as a yellow solid (80 mg, 73%). ¹H NMR (500 MHz, DMSO-d6): δ 8.73 (d, 1H), 8.04 (dd, 1H), 7.02-6.98 (dd, 1H), 6.76-6.74 (dd, 1H), 6.58 (d, 1H), 6.50-6.48 (m, 1H), 5.24 (d, 1H), 3.98 (t, 2H), 3.47 (s, 4H), 3.17 (t, 2H), 1.97 (s, 4H). LCMS: 96.7% [Ret. Time: 8.6 min, Hypersil BDS C18], (ESI) 410 m/z [M+H]⁺.

### Step-7: Synthesis of 5-(3-amino-2,6-dibromo-4-fluorophenyl)-2-(5-bromo-6-(pyrrolidin-1-yl)pyridin-3-yl)-6,7-dihydrothiazolo[5,4-c]pyridin-4(5H)-one (H4)

Title compound G4 (90 g, 0.22 mmol) was dissolved in chloroform (15 mL) and cooled to 0 to 5° C. To the above solution, N-bromo succinimide (0.15 g, 0.44 mmol) was added portionwise and stirring was continued at 5 to 20° C. for 1 hour. The solvent was removed from the reaction mixture under reduced pressure. The residue obtained was purified by column chromatography on silica gel (60 g, 100 to 200 mesh) using dichloromethane/methanol gradient (100/0->99/1->98/2) to afford compound H4 as a yellow solid (0.035 g, 25%). ¹H NMR (500 MHz, DMSO-d6): δ 8.72 (d, 1H), 8.31 (d, 1H), 7.59 (d, 1H), 5.74 (s, 2H), 3.89 (t, 2H), 3.73 (t, 4H), 3.30-3.39 (m, 2H), 1.92-1.89 (m, 4H). LCMS: 97% [Ret. Time: 12.84 min, Hypersil BDS C18], (ESI) 645.6 m/z [M+2H]⁺.

### Example 7 Synthesis of (S)-1-(5-(5-(1-methyl-1H-pyrazol-4-yl)-4-oxo-4,5,6,7-tetrahydrothiazolo[5,4-c]pyridin-2-yl)pyridin-2-yl)pyrrolidin-3-yl methanesulfonate (precursor of ¹⁸F Compound 1)

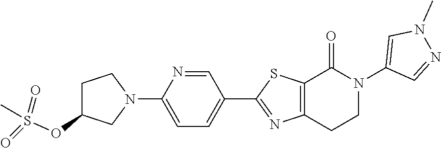 [SMILES: Cn1cc(N2CCc3nc(-c4ccc(N5CC[C@H](OS(C)(=O)=O)C5)nc4)sc3C2=O)cn1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%). ¹H NMR (500 MHz, DMSO-d₆): δ 11.89 (s, 1H), 3.74 (t, 2H), 2.68 (t, 2H), 1.44 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%). ¹H NMR (500 MHz, DMSO-d₆): δ 8.10 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper (II)-bromide (6.5 g, 29.43 mmol) was added to the above mixture and allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered, and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%). ¹H NMR (500 MHz DMSO-d₆): δ 4.12 (t, 2H), 3.10 (t, 2H), 1.50 (s, 9H). LCMS (ESI) 279 m/z [M+H-C₄H₃]⁺.

### Step-4: Synthesis of tert-butyl 4-oxo-2-(6-(pyrrolidin-1-yl)pyridin-3-yl)-6,7-dihydrothiazolo[5,4-c]pyridine-5(4H)-carboxylate (F1)

Water (10 mL) and 1,4-dioxane (40 mL) were combined and degassed by passing a stream of nitrogen through the mixture. Then [1,1′-bis(diphenyl-phosphino)ferrocene]-dichloropalladium(II) complex with dichloromethane (0.342 g, 0.4 mmol), tert-butyl 2-bromo-4-oxo-6,7-dihydrothiazolo[5,4-c]pyridine-5(4H)-carboxylate D (1.4 g, 4.2 mmol), 6-fluropyridine-3-boronic acid (1.73 g, 6.3 mmol) and cesium carbonate (4.1 g, 12.6 mmol) were added, and the reaction mixture was heated at 100° C. in an oil bath for 5 hours. The reaction mixture was diluted with two runs of ethyl acetate (80 mL) and water (80 mL). The organic phase was separated, dried over Na₂SO₄, filtered and the solvents were removed under reduced pressure. The residue was purified by column chromatography on basic silica gel (60 to 120 mesh) using ethyl acetate/n-hexane gradient (0/100->10/90->20/80->30/70->40/60) to obtain the title compound F1 as a pale-yellow solid (0.8 g, 48%). ¹H NMR (500 MHz, DMSO-d₆): δ 8.77 (d, 1H), 7.97 (dd, 1H), 6.40 (d, 1H), 4.14 (t, 2H), 3.54 (s, 4H), 3.10 (t, 2H), 2.04 (m, 4H), 1.57 (s, 9H). LCMS (APCI) 401.26 m/z [M+H]⁺.

### Step-5: Synthesis of 2-(6-fluoropyridin-3-yl)-6,7-dihydrothiazolo[5,4-c]pyridin-4(5H)-one (G5)

Title compound F1 (1.14 g, 3.2 mmol) was dissolved in dichloromethane (20 mL) and cooled to 0° C. in an ice bath with stirring. 4M HCl in 1,4-dioxane (10 mL) was added to the above solution and stirring was continued at room temperature for 16 hours. After completion of the reaction, solvent was removed under reduced pressure. The residue obtained was dissolved in ice cold water and basified with aqueous saturated sodium bicarbonate solution to pH 8 to 9, the precipitated solid was filtered and dried to afford the title compound G5 as a white solid (0.66 g, 80%) without further purification. LCMS (ESI) 250.15 m/z [M+H]⁺

### Step-6: Synthesis of (S)-2-(6-(3-hydroxypyrrolidin-1-yl)pyridin-3-yl)-6,7-dihydrothiazolo[5,4-c]pyridin-4(5H)-one (H6)

Title compound G5 (0.66 g, 2.65 mmol), (S)-pyrrolidin-3-ol hydrochloride salt (0.49 g, 3.97 mmol), diisopropylethylamine (1.41 mL, 7.9 mmol) were suspended in ethanol (7.3 mL) using a microwave vial. The sealed vial was then heated at 120° C. for 1.5 hours using a CEM microwave. The reaction mixture was concentrated under reduced pressure. The residue obtained was suspended in water (20 mL) and filtered over Whatmann filter paper. The solid was washed with water (20 mL), followed by n-hexane (20 mL) and dried under reduced pressure to obtain the title compound H6 as a pale-yellow solid (0.7 g, 83%). ¹H NMR (500 MHz, DMSO-d₆): δ 8.70 (d, 1H), 8.00 (dd, 1H), 7.78 (s, 1H), 6.55 (d, 1H), 5.02 (d, 1H), 4.41 (d, 1H), 3.56-3.47 (m, 5H), 2.97 (t, 2H), 2.06-1.98 (m, 2H). LCMS (ESI) 317.15 m/z [M+H]⁺.

### Step-7: Synthesis of (S)-2-(6-(3-((tert-butyldimethylsilyl) oxy) pyrrolidin-1-yl) pyridin-3-yl)-6,7-dihydrothiazolo[5,4-c]pyridin-4(5H)-one (11)

Title compound H6 (0.95 g, 3.0 mmol) and imidazole (1.84 g, 27 mmol) were dissolved in dimethylformamide (25 mL) under stirring at 0° C. in an ice bath. Tert-butyl dimethyl silyl chloride (1.81 g, 12 mmol) was added and stirring was continued at RT for 16 hours. The reaction mixture was quenched with ice cold water (100 mL), the precipitated solid was filtered over Whatmann filter paper and dried to obtain the title compound 11 as a pale-yellow solid (1.0 g, 78%). ¹H NMR (500 MHz, DMSO-d6): δ 8.70 (d, 1H), 8.01 (dd, 1H), 7.80 (s, 1H), 6.58 (d, 1H), 4.59 (s, 1H), 3.66-3.62 (m, 2H), 3.57-3.47 (m, 3H), 2.97 (t, 2H), 2.10 (dd, 1H), 2.11-1.91 (m, 2H), 0.82 (s, 9H), 0.09 (d, 6H). LCMS: (ESI) 637.44 m/z [M+H]⁺.

### Step-8: Synthesis of (S)-2-(6-(3-((tert-butyldimethylsilyl) oxy) pyrrolidin-1-yl) pyridin-3-yl)-5-(1-methyl-1H-pyrazol-4-yl)-6,7-dihydrothiazolo[5,4-c]pyridin-4(5H)-one (J1)

Title compound 11 (1.02 g, 2.4 mmol), 4-bromo-1-methyl-1H-pyrazole (1.14 g, 7.1 mmol), copper (I)-iodide (0.053 g, 0.28 mmol), N, N′-dimethylethylenediamine (0.42 mL, 4.1 mmol) and potassium carbonate (0.98 g, 7.1 mmol) were suspended in 1,4-dioxane (70 mL). The reaction mixture was heated at about 120° C. in an oil bath for 24 hours. The reaction mixture was concentrated under reduced pressure. The residue obtained was purified by column chromatography on basic silica gel (150 g, 100 to 200 mesh) using dichloromethane/methanol gradient (100/0->99/1->98/2->97/3->95/5) to afford the title compound J1 as a yellow solid (660 mg, 54%). ¹H NMR (500 MHz, DMSO-d6): δ 8.69 (d, 1H), 7.99 (dd, 2H), 7.63 (s, 1H), 6.56 (d, 1H), 4.55 (s, 1H), 4.02 (t, 2H), 3.79 (s, 3H), 3.62-3.47 (m, 3H), 3.32 (d, 1H), 3.16 (s, 2H), 2.07 (t, 1H), 1.88 (s, 1H), 0.82 (s, 9H), 0.06 (d, 6H). LCMS: (ESI) 511.37 m/z [M+H]⁺.

### Step-9: Synthesis of (S)-2-(6-(3-hydroxypyrrolidin-1-yl) pyridin-3-yl)-5-(1-methyl-1H-pyrazol-4-yl)-6,7-dihydrothiazolo[5,4-c]pyridin-4(5H)-one (K1)

Title compound J1 (0.65 g, 1.27 mmol) was dissolved in dichloromethane (13 mL) and cooled to 0° C. in an ice bath with stirring. 4M HCl in 1,4-dioxane (6.5 mL) was added to the above solution and stirring was continued at room temperature for 4 hours. After completion of the reaction, solvent was removed under reduced pressure. The residue obtained was dissolved in ice cold water and basified with aqueous saturated sodium bicarbonate solution to pH 8 to 9, the precipitated solid was filtered and dried to afford the title compound K1 as a pale yellow solid (0.5 g, 99%). ¹H NMR (500 MHz, DMSO-d₆): δ 8.69 (d, 1H), 7.99 (dd, 2H), 7.63 (s, 1H), 6.53 (d, 1H), 4.99 (s, 1H), 4.37 (s, 1H), 4.02 (t, 2H), 3.79 (s, 3H), 3.51 (dd, 3H), 3.16 (t, 2H), 2.00 (q, 1H), 1.89 (s, 1H). LCMS (ESI) 397.39 m/z [M+H]⁺

### Step-10: Synthesis of (S)-1-(5-(5-(1-methyl-1H-pyrazol-4-yl)-4-oxo-4, 5, 6, 7-tetrahydrothiazolo [5,4-c]pyridin-2-yl)pyridin-2-yl)pyrrolidin-3-yl methanesulfonate (L1)

Title compound K1 (0.68 g, 1.7 mmol) and triethylamine (14.3 mL, 103 mmol) were suspended in dichloromethane (90 mL) with stirring. The mixture was cooled to 0° C. in an ice bath. Methane sulfonyl chloride (4.0 mL, 51.4 mmol) and stirring was continued at room temperature for 24 hours. The reaction mixture was quenched with cold water (150 mL) and extracted with two runs of dichloromethane (150 mL). The organic phase was separated, dried over Na₂SO₄, filtered and the solvents were removed under reduced pressure. The residue obtained was purified by column chromatography on silica gel (400 g, 230-400 mesh) using dichloromethane/methanol gradient (100/0->99/1->98/2) to obtain the title compound L1 as a pale-yellow solid (0.1 g, 12.3%). ¹H NMR (500 MHz, DMSO-d₆): δ 8.76 (d, 1H), 8.09 (dd, 1H), 8.04 (s, 1H), 7.68 (s, 1H), 6.65 (d, 1H), 5.45 (s, 1H), 4.07 (t, 2H), 3.83 (s, 4H), 3.77 (dd, 1H), 3.70 (t, 1H), 3.56-3.52 (m, 1H), 3.28 (s, 3H), 3.27-3.23 (m, 2H). LCMS (ESI) 475.3 m/z [M+H]⁺. HPLC: 96.9% [Ret. Time: 6.08 min, Eclipse plus C18].

### Example 8 Synthesis of (S)-1-(5-(5-(1-methyl-1H-pyrazol-4-yl)-4-oxo-4,5,6,7-tetrahydrothiazolo[5,4-c]pyridin-2-yl)pyridin-2-yl)piperidin-3-yl methanesulfonate (precursor of ¹⁸F Compound 2)

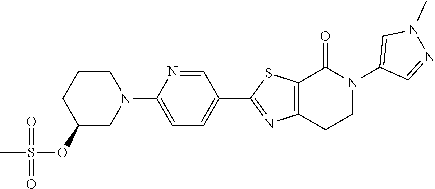 [SMILES: Cn1cc(N2CCc3nc(-c4ccc(N5CCC[C@H](OS(C)(=O)=O)C5)nc4)sc3C2=O)cn1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%). ¹H NMR (500 MHz, DMSO-d₆): δ 11.89 (s, 1H), 3.74 (t, 2H), 2.68 (t, 2H), 1.44 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) was dissolved in ethanol (160 mL) and heated in an oil bath at 80° C. for 2.5 h. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%). ¹H NMR (500 MHz, DMSO-d₆): δ 8.10 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper (II)-bromide (6.5 g, 29.43 mmol) was added to the above mixture, and it was allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%). ¹H NMR (500 MHz DMSO-d₆): δ 4.12 (t, 2H), 3.10 (t, 2H), 1.50 (s, 9H). LCMS (ESI) 279 m/z [M+H-C₄H₈]⁺.

### Step-4: Synthesis of tert-butyl 4-oxo-2-(6-(pyrrolidin-1-yl)pyridin-3-yl)-6,7-dihydrothiazolo[5,4-c]pyridine-5(4H)-carboxylate (F1)

Water (10 mL) and 1,4-dioxane (40 mL) were combined and degassed by passing a stream of nitrogen through the mixture. Then [1,1′-bis(diphenyl-phosphino)ferrocene]-dichloropalladium(II) complex with dichloromethane (0.342 g, 0.4 mmol), tert-butyl 2-bromo-4-oxo-6,7-dihydrothiazolo[5,4-c]pyridine-5(4H)-carboxylate D (1.4 g, 4.2 mmol), 6-fluropyridine-3-boronic acid (1.73 g, 6.3 mmol) and cesium carbonate (4.1 g, 12.6 mmol) were added, and the reaction mixture was heated at 100° C. in an oil bath for 5 hours. The reaction mixture was diluted with two runs of ethyl acetate (80 mL) and water (80 mL). The organic phase was separated, dried over Na₂SO₄, filtered, and the solvents were removed under reduced pressure. The residue was purified by column chromatography on basic silica gel (60 to 120 mesh) using ethyl acetate/n-hexane gradient (0/100->10/90->20/80->30/70->40/60) to obtain the title compound F1 as a pale-yellow solid (0.8 g, 48%). ¹H NMR (500 MHz, DMSO-d₆): δ 8.77 (d, 1H), 7.97 (dd, 1H), 6.40 (d, 1H), 4.14 (t, 2H), 3.54 (s, 4H), 3.10 (t, 2H), 2.04 (m, 4H), 1.57 (s, 9H). LCMS (APCI) 401.26 m/z [M+H]⁺.

### Step-5: Synthesis of 2-(6-fluoropyridin-3-yl)-6,7-dihydrothiazolo[5,4-c]pyridin-4(5H)-one (G5)

Title compound F1 (1.14 g, 3.2 mmol) was dissolved in dichloromethane (20 mL) and cooled to 0° C. in an ice bath with stirring. 4M HCl in 1,4-dioxane (10 mL) was added to the above solution and stirring was continued at room temperature for 16 hours. After completion of the reaction, solvent was removed under reduced pressure. The residue obtained was dissolved in ice cold water and basified with aqueous saturated sodium bicarbonate solution to pH 8 to 9, the precipitated solid was filtered and dried without further purification to afford the title compound G5 as a white solid (0.66 g, 80%). LCMS (ESI) 250.15 m/z [M+H]⁺.

### Step-6: Synthesis of (S)-2-(6-(3-hydroxypiperidin-1-yl)pyridin-3-yl)-6,7-dihydrothiazolo[5,4-c]pyridin-4(5H)-one (H7)

Title compound G5 (0.65 g, 2.6 mmol), (S)-piperidin-3-ol hydrochloride salt (1.07 g, 7.8 mmol), and diisopropylethylamine (2.7 mL, 15.7 mmol) were suspended in ethanol (21 mL) using a microwave vial. The sealed vial was then heated at 150° C. for 2 hours using a CEM microwave. The reaction mixture was concentrated under reduced pressure. The residue obtained was suspended in water (20 mL) and filtered over Whatmann filter paper. The solid was washed with water (20 mL) followed by n-hexane (20 mL) and dried under reduced pressure to obtain the title compound H7 as a pale-yellow solid (0.75 g, 87%). ¹H NMR (500 MHz, DMSO-d₆): δ 8.68 (d, 1H), 7.99 (dd, 1H), 7.79 (s, 1H), 6.90 (d, 1H), 4.90 (d, 1H), 4.23-3.99 (m, 2H), 3.53-3.47 (m, 3H), 3.17-3.12 (m, 1H), 2.99-2.91 (m, 3H), 1.91 (t, 1H), 1.76-1.73 (m, 1H), 1.47-1.43 (m, 2H). LCMS (ESI) 331.1 m/z [M+H]⁺.

### Step-7: Synthesis of (S)-2-(6-(3-((tert-butyldimethylsilyl)oxy)piperidin-1-yl)pyridin-3-yl)-6,7-dihydrothiazolo[5,4-c]pyridin-4(5H)-one (12)

Title compound H7 (0.2 g, 0.6 mmol) and imidazole (0.74 g, 10.9 mmol) were dissolved in dimethylformamide (15 mL) under stirring at 0° C. in an ice bath. Tert-butyl dimethyl silyl chloride (1.81 g, 4.9 mmol) was added and stirring was continued at room temperature for 24 hours. The reaction mixture was quenched with ice cold water (100 mL), the precipitated solid was filtered over Whatmann filter paper and dried to afford the title compound 12 as a pale-yellow solid (0.23 g, 86%). ¹H NMR (500 MHz, DMSO-d₆): δ 8.68 (d, 1H), 7.99 (dd, 1H), 7.80 (s, 1H), 6.90 (d, 1H), 4.09 (d, 1H), 3.87-3.85 (m, 1H), 3.75-3.70 (m, 1H), 3.5-3.47 (m, 2H), 3.2-3.17 (m, 1H), 2.98 (t, 2H), 1.88-1.87 (m, 1H), 1.77-1.75 (m, 1H), 1.55-1.43 (m, 2H), 0.82 (s, 9H), 0.07 (d, 6H). LCMS: (ESI) 445.38 m/z [M+H]⁺.

### Step-8: Synthesis of (S)-2-(6-(3-((tert-butyldimethylsilyl)oxy)piperidin-1-yl)pyridin-3-yl)-5-(1-methyl-1H-pyrazol-4-yl)-6,7-dihydrothiazolo[5,4-c]pyridin-4(5H)-one (J2)

Title compound 12 (0.24 g, 0.54 mmol), 4-iodo-1-methyl-1H-pyrazole 11 (0.34 g, 1.6 mmol), copper (I)-iodide (0.051 g, 0.27 mmol), N,N′-dimethylethylenediamine (0.056 mL, 0.54 mmol) and potassium carbonate (0.15 g, 1.1 mmol) were suspended in 1,4-dioxane (25 mL). The reaction mixture was heated at about 120° C. in an oil bath for 24 hours. The reaction mixture was concentrated under reduced pressure. The residue obtained was purified by column chromatography on basic silica gel (150 g, 100 to 200 mesh) using dichloromethane/methanol gradient (100/0->99/1->98/2->97/3) to afford the title compound J2 as a yellow solid (0.21 g, 74%). ¹H NMR (500 MHz, DMSO-d₆): δ 8.70 (d, 1H), 8.04 (s, 1H), 8.01 (dd, 1H), 7.67 (s, 1H), 6.91 (d, 1H), 4.10-4.05 (m, 3H), 3.90-3.80 (m, 4H), 3.76-3.71 (m, 1H), 3.37-3.34 (m, 1H), 3.23-3.18 (m, 3H), 1.90-1.76 (m, 2H), 1.55-1.41 (m, 1H), 1.88 (s, 1H), 0.82 (s, 9H), 0.07 (d, 6H). LCMS: (ESI) 525.34 m/z [M+H]⁺.

### Step-9: Synthesis of (S)-2-(6-(3-hydroxypiperidin-1-yl)pyridin-3-yl)-5-(1-methyl-1H-pyrazol-4-yl)-6,7-dihydrothiazolo[5,4-c]pyridin-4(5H)-one (K2)

Title compound J2 (0.21 g, 0.4 mmol) was dissolved in dichloromethane (8.4 mL) and cooled to 0° C. in an ice bath with stirring. 4M HCl in 1,4-dioxane (4.2 mL) was added to the above solution and stirring was continued at room temperature for 3 hours. After completion of the reaction, solvent was removed under reduced pressure. The residue obtained was dissolved in ice cold water and basified with aqueous saturated sodium bicarbonate solution to pH 8 to 9, the precipitated solid was filtered and dried to afford the title compound K2 as a pale-yellow solid (0.16 g, 97%). ¹H NMR (500 MHz, DMSO-d₆): δ 8.71 (d, 1H), 8.04-8.00 (m, 2H), 7.67 (s, 1H), 6.92 (d, 1H), 4.90 (d, 1H), 4.20 (d, 1H), 4.08-4.00 (m, 3H), 3.83 (s, 3H), 3.53-3.50 (m, 1H), 3.20-3.18 (m, 4H), 2.97-2.93 (m, 1H), 1.90 (s, 1H), 1.75 (s, 1H), 1.43 (s, 2H). LCMS (ESI) 411.23 m/z [M+H]⁺.

### Step-10: Synthesis of (S)-1-(5-(5-(1-methyl-1H-pyrazol-4-yl)-4-oxo-4,5,6,7-tetrahydrothiazolo [5,4-c]pyridin-2-yl)pyridin-2-yl)piperidin-3-yl methane sulfonate (L2)

Title compound K2 (0.5 g, 1.2 mmol) and triethylamine (6.9 mL, 48.7 mmol) were suspended in dichloromethane (80 mL) with stirring. The mixture was cooled to 0° C. in an ice bath. Methane sulfonyl chloride (2.9 mL, 24.4 mmol) was added and stirring was continued at room temperature for 24 hours. The reaction mixture was quenched with cold water (250 mL) and extracted with two runs of dichloromethane (250 mL). The organic phase was separated, dried over Na₂SO₄, filtered and the solvents were removed under reduced pressure. The residue obtained was purified by washing with two runs of 60% ethyl acetate in hexane (50 mL) and 60% ethyl acetate in hexane (50 mL). The resultant solid was further purified by a precipitation method by dissolving in dichloromethane (25 mL) and precipitated using n-hexane (40 mL). The solid was filtered and dried to obtain the title compound L2 as a yellow solid (0.133 g, 22%). ¹H NMR (500 MHz, DMSO-d₆): b 8.75 (d, 1H), 8.07 (dd, 1H), 8.04 (s, 1H), 7.68 (s, 1H), 6.98 (d, 1H), 4.80-4.76 (m, 1H), 4.07 (t, 2H), 4.00 (dd, 1H), 3.89-3.87 (m, 1H), 3.83 (s, 3H), 3.73-3.60 (m, 2H), 3.23 (s, 3H), 3.20 (d, 2H), 3.10 (s, 1H), 2.07-2.01 (m, 1H), 1.89-1.86 (m, 1H), 1.79-1.74 (m, 1H), 1.65-1.55 (m, 1H). LCMS (ESI) 489.37 m/z [M+H]⁺. HPLC: 86.7% [Ret. Time: 7.11 min, Eclipse plus C18].

### Example 9 Synthesis of tritiated (R)-2-(6-(3-fluoropyrrolidin-1-yl)pyridin-3-yl)-5-(1-methyl-1H-pyrazol-4-yl)-6,7-dihydrothiazolo[5,4-c]pyridin-4(5H)-one (³H Compound 1)

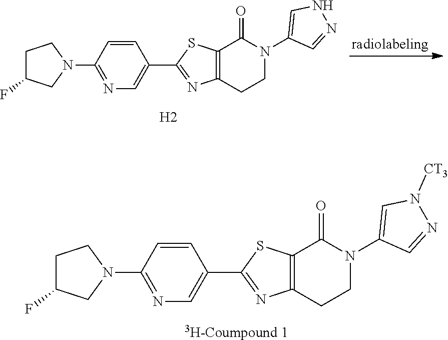

T means ³H.

Title compound H2 (0.001 g, 0.002 mmol) was added to a Tritium vessel, followed by sodium hydride (0.001 g, 0.006 mmol), dimethylformamide (0.1 mL) and iodomethane, [³H] (0.1 mL, 50 mCi). The vessel was sealed, and the solution was stirred for 3 hours at room temperature. The reaction mixture was evaporated under vacuum. The material was purified by a silica gel column. Mobile phase was removed under vacuum and the product was redissolved in 0.05% TFA in water/acetonitrile. The material was further purified by reverse phase HPLC. Mobile phase was removed under vacuum and the product was redissolved in ethanol to obtain ³H-Compound 1 (6 mCi, SA 75.83 Ci/mmol, 99% purity).

### Example 10 Synthesis of tritiated (R)-2-(6-(3-fluoropiperidin-1-yl)pyridin-3-yl)-5-(1-methyl-1H-pyrazol-4-yl)-6,7-dihydrothiazolo[5,4-c]pyridin-4(5H)-one (³H-Compound 2)

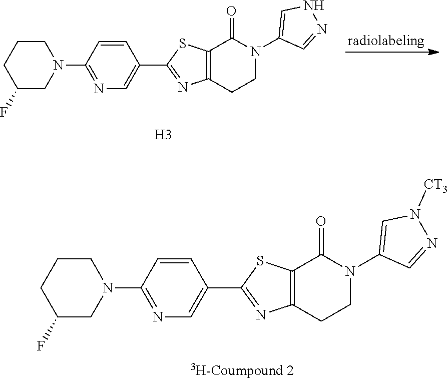

T means ³H.

Title compound H3 (0.001 g, 0.002 mmol) was added to a Tritium vessel, followed by sodium hydride (0.001 g, 0.006 mmol), dimethylformamide (0.1 mL) and iodomethane, [³H] (0.1 mL, 50 mCi). The vessel was sealed and the solution was stirred for 4 hours at room temperature. The reaction mixture was evaporated under vacuum. The material was purified by silica gel column. Mobile phase was removed under vacuum and the product was redissolved in 0.05% TFA in water/acetonitrile. The material was further purified by reverse phase HPLC. Mobile phase was removed under vacuum and the product was redissolved in ethanol to obtain ³H-Compound 2 (6.5 mCi, SA 81.5 Ci/mmol, 99% purity).

### Example 11 Synthesis of tritiated (5-(3-amino-4-fluorophenyl)-2-(6-(pyrrolidin-1-yl)pyridin-3-yl)-6,7-dihydrothiazolo[5,4-c]pyridin-4(5H)-one (³H-Compound 3)

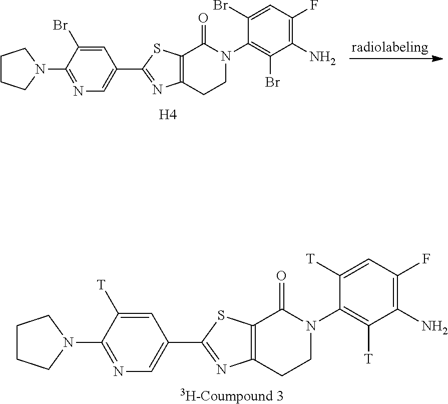

T means ³H.

10% Pd on carbon (0.002 g, 0.005 mmol) was added to a Tritium vessel, followed by a solution of H4 (0.002 g, 0.003 mmol) in dimethylformamide (3 mL) and N,N-diisopropylethylamine (DIEA) (5 μL). The vessel was attached to a Tritium line and pressurized with 0.5 atm (atmosphere) with Tritium gas at −200° C. The solution was stirred at room temperature for 3 hours and then cooled to −200° C. and excess gas was removed. The reaction mixture was evaporated under vacuum. The material was purified by silica gel column. Mobile phase was removed under vacuum and the product was redissolved in 0.05% TFA in water/acetonitrile. The material was further purified by reverse phase HPLC. Mobile phase was removed under vacuum and the product was redissolved n ethanol to obtain ³H-Compound 3 (2.2 mCi, SA 55.8 Ci/mmol, 99% purity).

### Example 12 Synthesis of fluorinated (5-(3-amino-4-fluorophenyl)-2-(6-(pyrrolidin-1-yl)pyridin-3-yl)-6,7-dihydrothiazolo[5,4-c]pyridin-4(5H)-one (¹⁸F Compound 1)

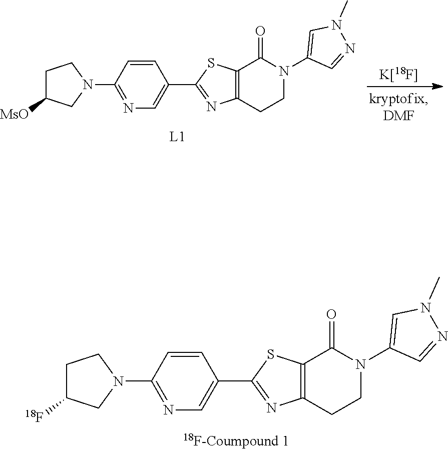

A solution of (S)-1-(5-(5-(1-methyl-1H-pyrazol-4-yl)-4-oxo-4,5,6,7-tetrahydrothiazolo [5,4-c]pyridin-2-yl)pyridin-2-yl)pyrrolidin-3-yl methanesulfonate L1 (1 mg in 1 mL anhydrous DMSO) was added to a microwave vial containing dry [¹⁸F] fluoride and the vessel was heated at 110° C. for 15 minutes. The reaction vessel was cooled to 40° C. and the mixture was diluted with HPLC buffer (4 mL). The resulting mixture was passed through a Sep-Pak® Alumina-N Light Cartridge. Crude material was purified by semi-preparative HPLC and collected purified fraction was passed through a Strata® C18-E cartridge. The final product ¹⁸F-Compound 1 was filtered through a sterilizing filter onto the final product vial inside the dispensing hot cell. The final product vial was assayed, and sample was removed for QC testing. Confirmation of the identity of the product was determined by co-injection with a sample of the ¹⁹F-reference compound.

### Synthesis of building block (S)-2-(6-(3-fluoropyrrolidin-1-yl)pyridin-3-yl)-6,7-dihydrothiazolo[5,4-c]pyridin-4(5H)-one (Prep 4)

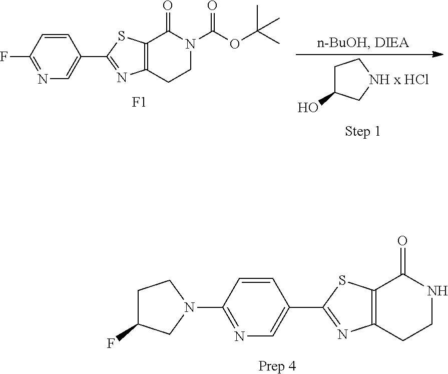

### Step-1: Synthesis of (S)-2-(6-(3-fluoropyrrolidin-1-yl)pyridin-3-yl)-6,7-dihydrothiazolo[5,4-c]pyridin-4(5H)-one (Prep 4)

Compound F1 (0.8 g, 2.29 mmol), (S)-3-fluoropyrrolidine hydrochloric acid salt (0.575 g, 4.6 mmol), and DIEA (1.6 mL, 1.18 g, 9.16 mmol) were suspended in n-butanol (15 mL) using a microwave vial. The sealed vial was then heated at 160° C. for 2 h in a CEM microwave. The reaction mixture was concentrated under reduced pressure. The residue obtained was suspended in water (20 mL) and filtered over Whatmann filter paper. The solid was washed with water (20 mL), followed by n-hexane (20 mL) and dried under reduced pressure to obtain the title compound Prep 4 as an off-white solid (0.7 g, 96%). ¹H NMR (400 MHz, DMSO-d₆): δ 8.73 (d, 1H), 8.05 (dd, 1H), 7.80 (s, 1H), 6.63 (d, 1H), 5.47 (m, 1H), 3.73 (m, 3H), 3.5 (m, 3H), 2.98 (t, 2H), 2.23 (m, 2H). LCMS (ESI) 319.00 m/z [M+H]⁺.

### Synthesis of building block (S)-2-(4-(3-fluoropyrrolidin-1-yl)phenyl)-6,7-dihydrothiazolo[5,4-c]pyridin-4(5H)-one (Prep 5)

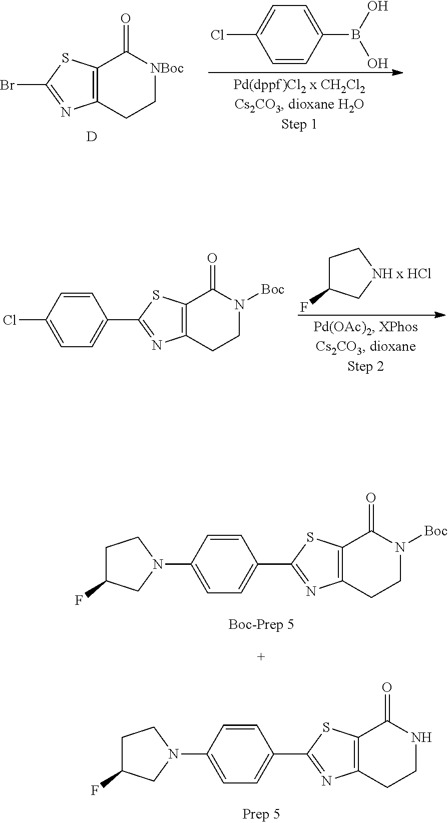

### Step-1: Synthesis of tert-butyl 2-(4-chlorophenyl)-4-oxo-6,7-dihydrothiazolo[5,4-c]pyridine-5(4H)-carboxylate

To a degassed solution of N,N′-dimethylformamide (12.5 mL) were added compound D (0.5 g, 1.5 mmol), 4-chlorophenyl boronic acid (1.17 g, 7.5 mmol), cesium carbonate (0.975 g, 3.0 mmol), copper(I)-chloride (0.15 g, 1.5 mmol), palladium acetate (0.017 g, 0.075 mmol) and 1,1′-bis(diphenylphosphino)ferrocene (0.083 g, 0.15 mmol). The reaction mixture was heated at −90° C. in a sand-bath for 5 h. The reaction mixture was diluted with ethyl acetate (200 mL) and water (120 mL). The organic phase was separated, dried over Na₂SO₄, filtered and the solvents were removed under reduced pressure. The residue was purified by column chromatography on silica gel (100-200 mesh) using ethyl acetate/n-hexane gradient (0/100->3/97->5/95) to obtain the title compound as a white solid (0.23 g, 42%). ¹H NMR (500 MHz DMSO-d₆): δ 8.05 (d, 2H), 7.62 (d, 2H), 4.08 (t, 2H), 3.15 (t, 2H), 1.49 (s, 9H). LCMS (ESI) 308.8 m/z [M+H-Boc]⁺.

### Step-2: Synthesis of (S)-2-(4-(3-fluoropyrrolidin-1-yl)phenyl)-6,7-dihydrothiazolo[5,4-c]pyridin-4(5H)-one (Prep 5)

To a solution of degassed 1,4-dioxane (10 mL) were added the title compound from Step 1 above (0.1 g, 0.275 mmol), (S)-3-fluoropyrrolidine hydrochloric acid salt (0.07 g, 0.55 mmol), palladium(II) acetate (0.006 g, 0.027 mmol), 2-dicyclohexylphosphino-2′,4′,6′-triisopropylbiphenyl (0.026 g, 0.054 mmol) and cesium carbonate (0.36 g, 1.1 mmol). The reaction mixture was heated at −120° C. in a sand-bath for 16 h. The reaction mixture was concentrated under reduced pressure. The residue was purified by column chromatography on silica gel (100-200 mesh) using ethyl acetate/n-hexane gradient (0/100->10/90->15/85->20/80) to obtain the Boc-Prep 5 as a yellow solid (0.03 g, 26%) and Prep 5 as a yellow solid (0.03 g, 34%). Boc-Prep 5: ¹H NMR (500 MHz DMSO-d₆): δ 7.81 (d, 2H), 6.64 (d, 2H), 5.45 (d, 1H), 4.0 (t, 2H), 3.49 (m, 4H), 3.03 (t, 2H), 2.2 (m, 2H), 1.45 (s, 9H). LCMS (ESI) 418 m/z [M+H]⁺. Prep 5: ¹H NMR (500 MHz DMSO-d₆): δ 7.84 (dd, 2H), 6.66 (d, 2H), 5.48 (d, 1H), 3.56 (m, 5H), 3.41 (q, 1H), 2.96 (t, 2H), 2.23 (m, 2H). LCMS (ESI) 317.9 m/z [M+H]⁺.

### Synthesis of Building Block (R)-2-(4-(3-fluoropyrrolidin-1-yl)phenyl)-6,7-dihydrothiazolo[5,4-c]pyridin-4(5H)-one (Prep 6)

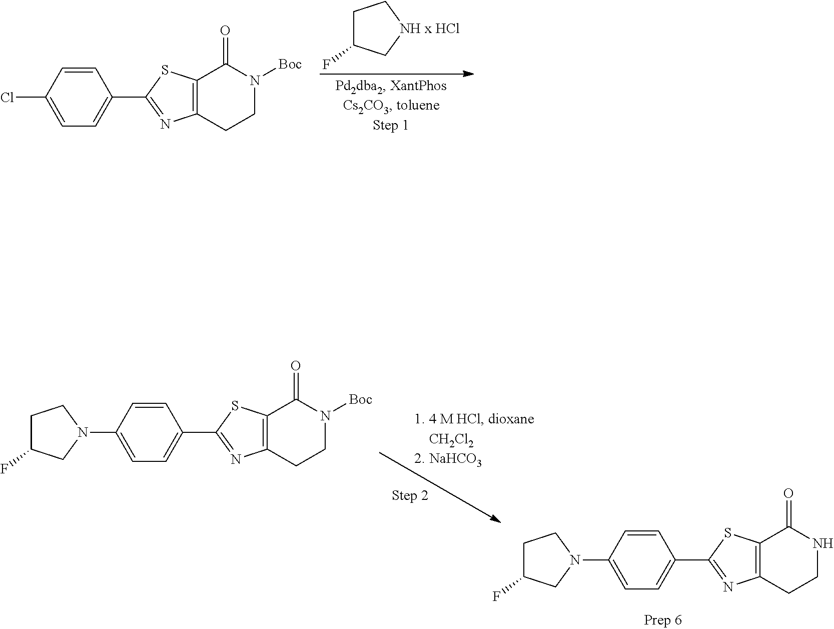

### Step-1: Synthesis of tert-butyl (R)-2-(4-(3-fluoropyrrolidin-1-yl)phenyl)-4-oxo-6,7-dihydrothiazolo[5,4-c]pyridine-5(4H)-carboxylate

To a solution of degassed toluene (5 mL) were added the title compound of Step 1 from the synthesis of building block Prep 5 (0.04 g, 0.1 mmol), (R)-3-fluoropyrrolidine hydrochloric acid salt (0.028 g, 0.21 mmol), tris(dibenzylideneacetone)dipalladium(0) (0.01 g, 0.01 mmol), 4,5-bis(diphenylphosphino)-9,9-dimethylxanthene (0.012 g, 0.02 mmol) and cesium carbonate (0.1 g, 0.3 mmol). The reaction mixture was heated at −100° C. in a sand-bath for 8 h. The reaction mixture was concentrated under reduced pressure. The residue was purified by column chromatography on silica gel (100-200 mesh) using ethyl acetate/n-hexane gradient (0/100->5/95->10/90) to obtain the title compound as a yellow solid (0.015 g, 33%). ¹H NMR (500 MHz DMSO-d₆): δ 7.85 (d, 2H), 6.68 (d, 2H), 5.49 (d, 1H), 4.04 (t, 2H), 3.59 (m, 3H), 3.43 (m, 1H), 3.07 (t, 2H), 2.28 (m, 2H), 1.49 (s, 9H). LCMS (ESI) 362.0 m/z [M+H-Boc]⁺.

### Step-2: Synthesis of (R)-2-(4-(3-fluoropyrrolidin-1-yl)phenyl)-6,7-dihydrothiazolo[5,4-c]pyridin-4(5H)-one (Prep 6)

The title compound from Step 1 above (0.04 g, 0.096 mmol) was dissolved in CH₂Cl₂ (2 mL) and cooled to 0° C. in an ice-bath with stirring. Then a 4 M solution of HCl in 1,4-dioxane (0.5 mL) was added and the reaction mixture was stirred at room temperature for 4 h. After completion of the reaction, the solvents were removed under reduced pressure. The residue was dissolved in ice cold water, basified with aqueous saturated sodium bicarbonate solution to pH 8-9, the precipitate was collected by filtration, and dried to afford Prep 6 as a pale-yellow solid (0.022 g, 73%). ¹H NMR (500 MHz DMSO-d₆): δ 7.81 (d, 2H), 7.75 (s, 1H), 6.66 (d, 2H), 5.48 (d, 1H), 3.52 (m, 6H), 2.96 (t, 2H), 2.24 (m, 2H). LCMS (ESI) 317.38 [M+H]⁺.

### Examples 13 to 28

Following the coupling procedure as reported for the preparation of Example 1 and utilizing the building blocks and halogen derivatives indicated in Table 1, the following compounds were prepared.

TABLE 1

| | | | | 1. Yield |
|---|---|---|---|---|
| | | | | 2. ¹H-NMR |
| | | Halogen | | 3. MH⁺ (ESI) |
| Example | Building block | derivative | Product | 4. Synthesis procedure |
| 13 | 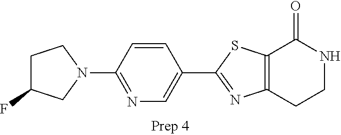 [SMILES: O=C1NCCc2nc(-c3ccc(N4CC[C@H](F)C4)nc3)sc21] | 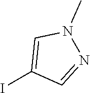 | 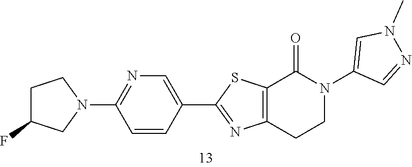 [SMILES: Cn1cc(N2CCc3nc(-c4ccc(N5CC[C@H](F)C5)nc4)sc3C2=O)cn1] | 1.25% 2. ¹H NMR (500 MHZ, DMSO-d₆): δ 8.75 (d, 2H), 8.06 (m, 2H), 7.68 (s, 1H), 6.64 (d, 1H), 5.48 (d, 1H), 4.07 (t, 2H), 3.83 (s, 1H),3.74 (m, 2H), 3.64 (dd 1H), 3.50 (m, 1H), 3.19 (m, 3H), 2.27 (m, 2H) 3. 399.37 4. Example 1 |
| 14 | 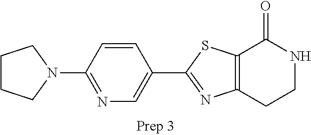 [SMILES: O=C1NCCc2nc(-c3ccc(N4CCCC4)nc3)sc21] | 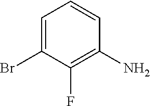 | 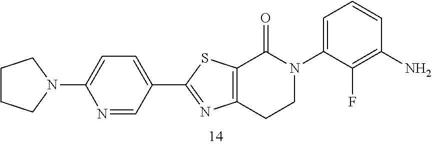 [SMILES: Nc1cccc(N2CCc3nc(-c4ccc(N5CCCC5)nc4)sc3C2=O)c1F] | 1.55% 2. ¹H NMR (500 MHZ, DMSO-d₆): δ 8.74 (d, 1H), 8.04 (dd, 1H), 6.90 (t, 1H), 6.72 (td, 1H), 6.56 (m, 2H), 5.26 (s, 2H), 3.96 (t, 2H), 3.48 (s, 4H), 3.19 (t, 2H), 1.98 (d, 4H) 3. 410.0 4. Example 1 |
| 15 | 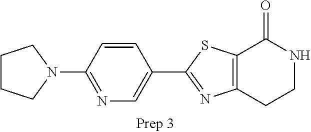 [SMILES: O=C1NCCc2nc(-c3ccc(N4CCCC4)nc3)sc21] | 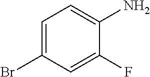 | 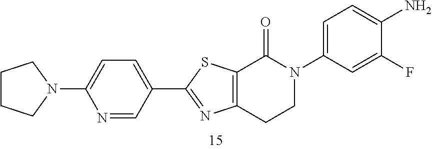 [SMILES: Nc1ccc(N2CCc3nc(-c4ccc(N5CCCC5)nc4)sc3C2=O)cc1F] | 1. 12% 2. ¹H NMR (500 MHZ, DMSO-d₆): δ 8.73 (d, 1H), 8.03 (dd, 1H), 7.07 (dd, 1H), 6.89 (dd, 1H), 6.76 (dd, 1H), 6.57 (d, 1H), 5.18 (s, 2H), 3.97 (t, 2H), 3.17 (t, 2H), 1.97 (t, 4H 3. 420.1 4. Example 1 |
| 16 | 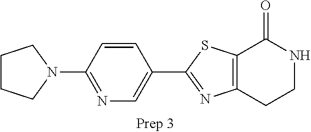 [SMILES: O=C1NCCc2nc(-c3ccc(N4CCCC4)nc3)sc21] | 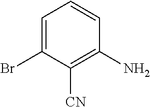 | 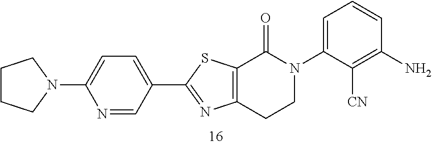 [SMILES: N#Cc1c(N)cccc1N1CCc2nc(-c3ccc(N4CCCC4)nc3)sc2C1=O] | 1. 11% 2. ¹H NMR (500 MHZ, DMSO-d₆): δ 8.75 (d, 1H), 8.05 (dd, 1H), 7.35 (t, 1H), 6.75 (d, 1H), 6.61 (m, 2H), 6.19 (s, 2H), 4.00 (s, 2H), 3.48 (s, 4H), 3.19 (m, 2H), 1.97 (t, 4H) 3. 417.2 4. Example 1 |
| 17 | 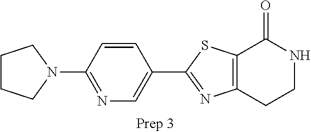 [SMILES: O=C1NCCc2nc(-c3ccc(N4CCCC4)nc3)sc21] | 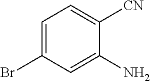 | 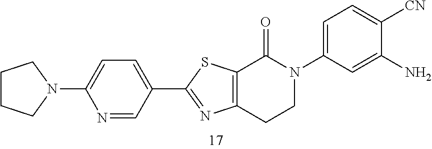 [SMILES: N#Cc1ccc(N2CCc3nc(-c4ccc(N5CCCC5)nc4)sc3C2=O)cc1N] | 1. 13.8% 2. ¹H NMR (400 MHZ, DMSO-d₆): δ 8.74 (d, 1H), 8.04 (dd, 1H), 7.42 (d, 1H), 6.82 (d, 1H),6.64 (dd, 1H), 6.58 (d, 1H), 6.10 (d, 2H), 4.05 (t, 2H), 3.48 (s, 4H), 3.19 (t, 2H), 1.97 (s, 4H) 3. 417.2 4. Example 1 |
| 18 | 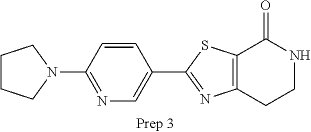 [SMILES: O=C1NCCc2nc(-c3ccc(N4CCCC4)nc3)sc21] | 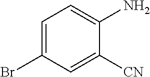 | 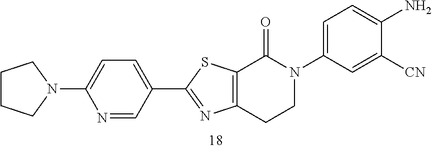 [SMILES: N#Cc1cc(N2CCc3nc(-c4ccc(N5CCCC5)nc4)sc3C2=O)ccc1N] | 1. 20% 2. ¹H NMR (500 MHZ, DMSO-d₆): δ 8.73 (d, 1H), 8.03 (dd, 1H), 7.42 (d, 1H), 7.33 (dd, 1H), 6.80 (d, 1H), 6.57 (d, 1H), 6.13 (s, 2H), 3.97 (t, 2H), 3.48 (s, 4H), 3.17 (t, 2H), 1.97 (s, 4H) 3. 417.0 4. Example 1 |
| 19 | 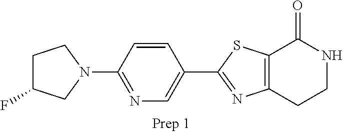 [SMILES: O=C1NCCc2nc(-c3ccc(N4CC[C@@H](F)C4)nc3)sc21] | 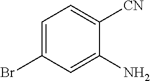 | 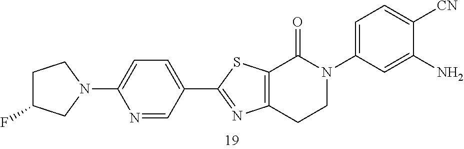 [SMILES: N#Cc1ccc(N2CCc3nc(-c4ccc(N5CC[C@@H](F)C5)nc4)sc3C2=O)cc1N] | 1. 8.6% ¹H NMR (500 MHZ, DMSO-d₆): δ 8.77 (d, 1H), 8.09 (dd, 1H), 7.42 (d, 1H), 6.83 (d,1H), 6.65 (d, 2H), 6.11 (s, 2H), 5.48 (dd, 1H), 5.06 (t, 2H), 3.75 (m, 3H), 3.51 (m, 1H), 3.19 (t, 2H), 2.24 (m, 2H) 3. 435.1 4. Example 1 |
| 20 | 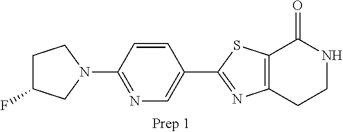 [SMILES: O=C1NCCc2nc(-c3ccc(N4CC[C@@H](F)C4)nc3)sc21] | 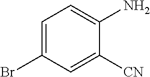 | 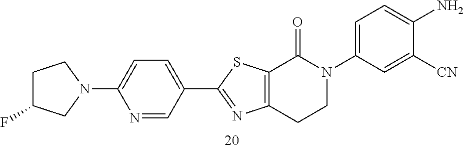 [SMILES: N#Cc1cc(N2CCc3nc(-c4ccc(N5CC[C@@H](F)C5)nc4)sc3C2=O)ccc1N] | 1. 29% 2. ¹H NMR (500 MHZ, DMSO-d₆): δ 8.76 (d, 1H), 8.08 (dd, 1H), 7.42 (d, 1H), 7.33 (dd, 1H), 6.81 (d, 1H), 6.64 (d, 2H), 6.13 (s, 2H), 5.48 (d, 1H), 3.98 (t, 2H), 3.74 (m, 3H), 3.51 (td, 1H), 3.18 (m, 2H), 2.24 (m, 2H) 3. 435.0 4. Example 1 |
| 21 | 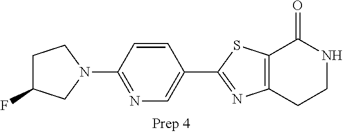 [SMILES: O=C1NCCc2nc(-c3ccc(N4CC[C@H](F)C4)nc3)sc21] | 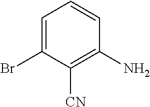 | 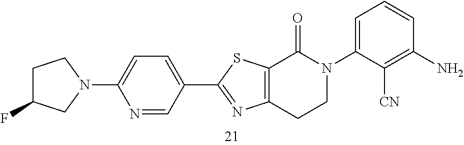 [SMILES: N#Cc1c(N)cccc1N1CCc2nc(-c3ccc(N4CC[C@H](F)C4)nc3)sc2C1=O] | 1. 7.3% 2. ¹H NMR (500 MHZ, DMSO-d₆): δ 8.77 (d, 1H), 8.09 (dd, 1H), 7.35 (t, 1H), 6.75 (d, 1H), 6.64 (t, 2H), 6.18 (s, 2H), 5.40 (d, 1H), 4.01 (s, 2H), 3.75 (m, 3H), 3.51 (td, 1H), 3.22 (t, 2H), 2.26 (m, 2H) 3. 435.28 4. Example 1 |
| 22 | 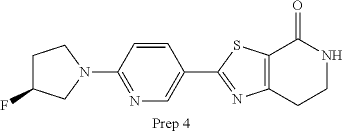 [SMILES: O=C1NCCc2nc(-c3ccc(N4CC[C@H](F)C4)nc3)sc21] | 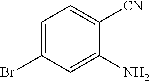 | 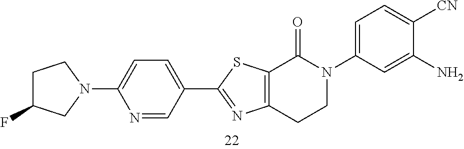 [SMILES: N#Cc1ccc(N2CCc3nc(-c4ccc(N5CC[C@H](F)C5)nc4)sc3C2=O)cc1N] | 1. 7.3% 2. ¹H NMR (500 MHZ, DMSO-d₆): δ 8.77 (d, 1H), 8.09 (dd, 1H), 7.42 (d, 1H), 6.83 (dd, 1H), 6.65 (dd, 2H), 6.11 (s, 2H), 5.49 (s, 1H), 4.06 (t, 1H), 3.74 (m, 3H), 3.51 (td, 1H), 3.20 (t, 2H), 2.24 (m, 2H) 3. 435.28 4. Example 1 |
| 23 | 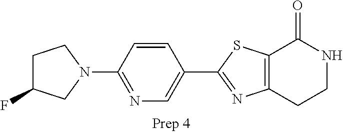 [SMILES: O=C1NCCc2nc(-c3ccc(N4CC[C@H](F)C4)nc3)sc21] | 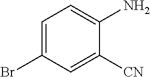 | 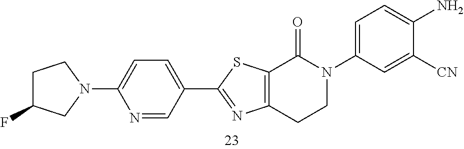 [SMILES: N#Cc1cc(N2CCc3nc(-c4ccc(N5CC[C@H](F)C5)nc4)sc3C2=O)ccc1N] | 1. 26% 2. ¹H NMR (500 MHZ, DMSO-d₆): δ 8.76 (d, 1H), 8.08 (dd, 1H), 7.42 (d, 1H), 7.33 (dd, 1H), 6.80 (d, 1H), 6.64 (d, 1H), 5.49 (d, 1H), 3.98 (t, 2H), 3.76 (m, 3H), 3.50 (td, 1H), 3.18 (m, 2H), 2.24 (m, 2H) 3. 435.0 4. Example 1 |
| 24 | 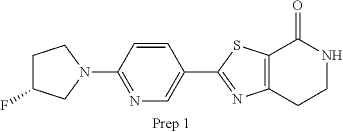 [SMILES: O=C1NCCc2nc(-c3ccc(N4CC[C@@H](F)C4)nc3)sc21] | 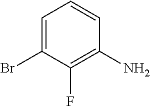 | 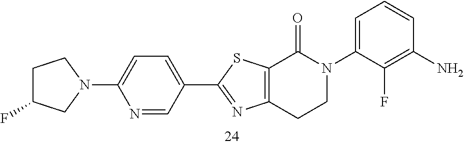 [SMILES: Nc1cccc(N2CCc3nc(-c4ccc(N5CC[C@@H](F)C5)nc4)sc3C2=O)c1F] | 1. 28% 2. ¹H NMR (500 MHZ, DMSO-d₆): δ 8.76 (d, 1H), 8.09 (dd, 1H), 6.90 (t, 1H), 6.72 (t, 1H), 6.65 (d, 1H), 6.55 (m, 1H), 5.48 (d, 1H), 5.25 (s, 2H), 3.96 (t, 2H), 3.74 (m, 3H), 3.51 (td, 1H), 3.20 (t, 2H), 2.24 (m, 2H) 3. 428.27 4. Example 1 |
| 25 | 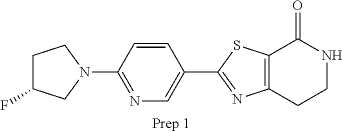 [SMILES: O=C1NCCc2nc(-c3ccc(N4CC[C@@H](F)C4)nc3)sc21] | 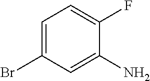 | 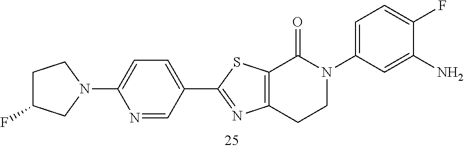 [SMILES: Nc1cc(N2CCc3nc(-c4ccc(N5CC[C@@H](F)C5)nc4)sc3C2=O)ccc1F] | 1. 60% 2. ¹H NMR (500 MHZ, DMSO-d₆): δ 8.76 (d, 1H), 8.08 (dd, 1H), 7.01 (dd, 1H), 6.76 (dd, 1H), 6.64 (d, 1H), 6.49 (m, 1H), 5.48 (d, 1H), 5.23 (s, 2H), 3.98 (t, 2H), 3.68 (dd, 3H), 3.51 (td, 1H), 3.18 (t,2H), 2.24 (m, 2H) 3. 428.22 4. Example 1 |
| 26 | 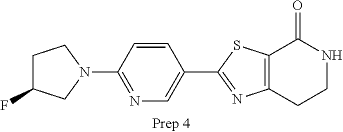 [SMILES: O=C1NCCc2nc(-c3ccc(N4CC[C@H](F)C4)nc3)sc21] | 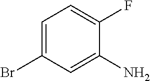 | 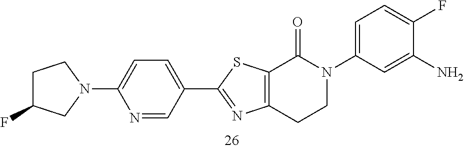 [SMILES: Nc1cc(N2CCc3nc(-c4ccc(N5CC[C@H](F)C5)nc4)sc3C2=O)ccc1F] | 1. 74% 2. ¹H NMR (500 MHZ, DMSO-d₆): δ 8.76 (d, 1H), 8.08 (dd, 1H), 7.01 (dd, 1H), 6.76 (dd, 1H), 6.64 (d, 1H), 6.49 (m, 1H), 5.48 (d, 1H), 5.23 (s, 2H), 3.98 (t, 2H), 3.73 (m, 3H), 3.51 (m, 1H), 3.18 (m, 2H), 2.22 (m, 2H) 3. 428.19 4. Example 1 |
| 27 | 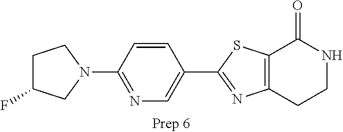 [SMILES: O=C1NCCc2nc(-c3ccc(N4CC[C@@H](F)C4)nc3)sc21] | 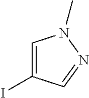 | 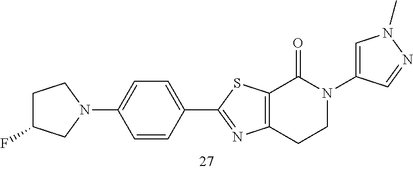 [SMILES: Cn1cc(N2CCc3nc(-c4ccc(N5CC[C@@H](F)C5)cc4)sc3C2=O)cn1] | 1. 40% 2. ¹H NMR (500 MHZ, DMSO-d₆): δ 8.03 (s, 1H), 7.84 (d, 2H), 7.67 (s, 1H), 6.67 (d, 2H), 5.48 (d, 1H), 4.05 (t, 2H), 3.84 (d, 3H), 3.63 (m, 2H), 3.54 (m, 2H), 3.43 (m, 1H), 3.18 (t, 2H), 2.24 (m, 2H), 1.23 (s, 1H) 3. 398.0 4. Example 1 |
| 28 | 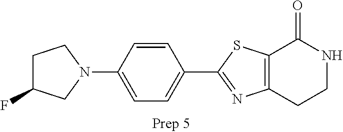 [SMILES: O=C1NCCc2nc(-c3ccc(N4CC[C@H](F)C4)cc3)sc21] | 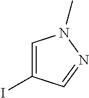 | 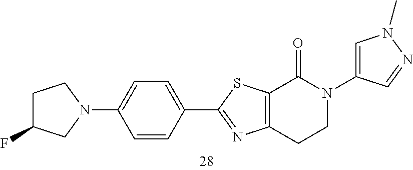 [SMILES: Cn1cc(N2CCc3nc(-c4ccc(N5CC[C@H](F)C5)cc4)sc3C2=O)cn1] | 1. 35% 2. ¹H NMR (500 MHZ, DMSO-d₆) δ 8.03 (s, 1H), 7.84 (d, 2H), 7.67 (s, 1H), 6.67 (d, 2H), 5.49 (d, 1H), 4.06 (t, 2H), 3.83 (s, 3H), 3.59 (m, 3H), 3.42 (dt, 1H), 3.19 (t, 2H), 2.25 (m, 2H) 3. 398.0 4. Example 1 |

### Synthesis of building block (S)-2-(6-(3-fluoropiperidine-1-yl)pyridin-3-yl)-6,7-dihydrothiazolo[5,4-c]pyridin-4(5H)-one (Prep 7)

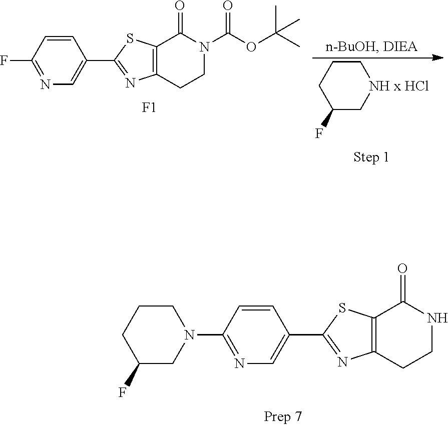

### Step-1: (S)-2-(6-(3-fluoropiperidin-1-yl)pyridin-3-yl)-6,7-dihydrothiazolo[5,4-c]pyridin-4(5H)-one (Prep 7)

Compound F1 (0.2 g, 0.57 mmol), (S)-3-fluoropiperidine hydrochloric acid salt (0.158 g, 1.177 mmol), and DIEA (0.4 mL, 0.29 g, 2.29 mmol) were suspended in n-butanol (10 mL) using a microwave vial.

The sealed vial was then heated at 170° C. for 2.5 h in a CEM microwave. The reaction mixture was concentrated under reduced pressure. The residue obtained was suspended in water (20 mL) and filtered over Whatmann filter paper. The solid was washed with water (20 mL), followed by n-hexane (20 mL) and dried under reduced pressure to obtain the title compound Prep 7 as a pale-yellow solid (0.16 g, 84%). ¹H NMR (400 MHz, DMSO-d₆): δ 8.7 (d, 1H), 8.02 (dd, 1H), 7.81 (s, 1H), 6.98 (d, 1H), 4.8 (m, 1H), 4.08 (m, 1H), 3.92 (td, 1H), 3.72 (qd, 1H), 3.49 (td, 2H), 3.42 (t, 1H), 2.98 (t, 2H), 1.92 (m, 2H), 1.76 (tt, 1H). 1.56 (m, 1H). LCMS (ESI) 333.1 m/z [M+H]⁺.

### Examples 29 to 33

Following the coupling procedure as reported in Example 1 and utilizing the building blocks and halogen derivatives indicated in Table 2, the following compounds were prepared.

TABLE 2

| | | | | 1. Yield |
|---|---|---|---|---|
| | | | | 2. ¹H-NMR |
| | | Halogen | | 3. MH⁺ (ESI) |
| Example | Building block | derivative | Product | 4. Synthesis procedure |
| 29 | 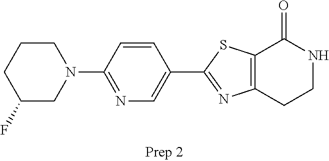 [SMILES: O=C1NCCc2nc(-c3ccc(N4CCC[C@@H](F)C4)nc3)sc21] | 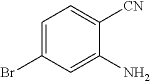 | 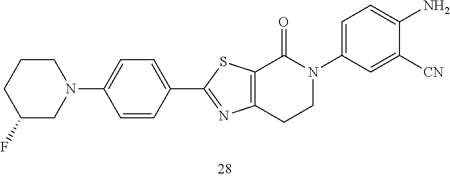 [SMILES: N#Cc1cc(N2CCc3nc(-c4ccc(N5CCC[C@@H](F)C5)cc4)sc3C2=O)ccc1N] | 1. 11% 2. ¹H NMR (500 MHZ, DMSO-d₆): δ 8.73 (d, 1H), 8.04 (dd, 1H), 7.42 (d, 1H), 7.33 (dd, 1H), 6.99 (d, 1H), 6.80 (d, 1H), 6.13 (s, 2H), 4.80 (d, 1H), 4.10 (m, 1H), 3.96 (m, 3H), 3.72 (m, 1H), 3.42 (m,2H), 3.18 (t, 2H), 1.93 (m, 2H), 1.76 (m, 1H), 1.55 (m, 1H) 3. 449.1 4. Example 1 |
| 30 | 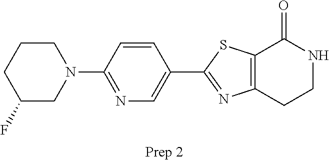 [SMILES: O=C1NCCc2nc(-c3ccc(N4CCC[C@@H](F)C4)nc3)sc21] | 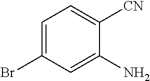 | 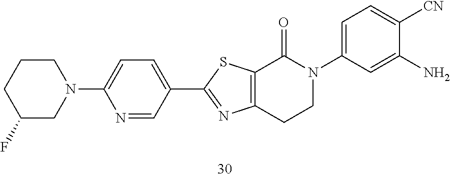 [SMILES: N#Cc1ccc(N2CCc3nc(-c4ccc(N5CCC[C@@H](F)C5)nc4)sc3C2=O)cc1N] | 1. 22% 2. ¹H NMR (500 MHZ, DMSO-d₆): δ 8.74 (d, 1H), 8.05 (dd, 1H), 7.42 (d, 1H), 6.99 (d, 1H), 6.82 (d, 1H), 6.64 (dd, 1H), 6.11 (s, 2H), 4.81 (dt, 1H), 4.09 (m, 3H), 3.95 (m, 1H), 3.71 (m, 1H), 3.42 (m,2H), 3.18 (t, 2H), 1.91 (m, 2H), 1.75 (m, 1H), 1.56 (m, 1H) 3. 449.15 4. Example 1 |
| 31 | 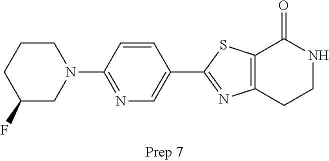 [SMILES: O=C1NCCc2nc(-c3ccc(N4CCC[C@H](F)C4)nc3)sc21] | 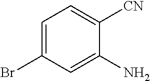 | 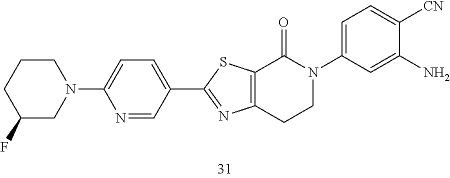 [SMILES: N#Cc1ccc(N2CCc3nc(-c4ccc(N5CCC[C@H](F)C5)nc4)sc3C2=O)cc1N] | 1. 22% 2. ¹H NMR (500 MHZ, DMSO-d₆): δ 8.74 (d, 1H), 8.05 (dd, 1H), 7.42 (d, 1H), 6.99 (d, 1H), 6.82 (d, 1H), 6.64 (dd, 1H), 6.10 (s, 2H), 4.80 (m 1H), 4.11 (dd, 1H), 4.06 (t, 2H), 3.95 (d, 1H), 3.72 (dd, 1H), 3.42 (m, 2H), 3.19 (t, 2H), 1.93 (m, 2H), 1.76 (m,1 H), 1.55 (m, 1H) 3. 449.32 4. Example 1 |
| 32 | 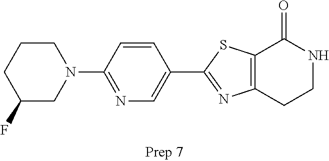 [SMILES: O=C1NCCc2nc(-c3ccc(N4CCC[C@H](F)C4)nc3)sc21] | 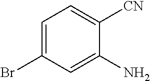 | 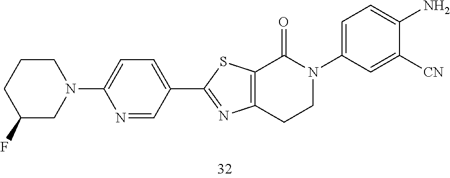 [SMILES: N#Cc1cc(N2CCc3nc(-c4ccc(N5CCC[C@H](F)C5)nc4)sc3C2=O)ccc1N] | 1. 11% 2. ¹H NMR (500 MHZ, DMSO-d₆): δ 8.73 (d, 1H), 8.04 (dd, 1H), 7.42 (d, 1H), 7.33 (dd, 1H), 6.99 (d, 1H), 6.81 (d, 1H), 6.12 (s, 2H), 4.80 (m, 1H), 4.10 (m, 1H), 3.98 (t, 3H), 3.93 (q, 1H), 3.72 (qd, 1H), 3.43 (m, 1H), 3.18 (t, 2H), 1.92 (m, 2H), 1.76 (m, 1H), 1.59 (m, 1H) 3. 449.31 4. Example 1 |
| 33 | 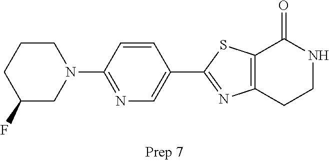 [SMILES: O=C1NCCc2nc(-c3ccc(N4CCC[C@H](F)C4)nc3)sc21] | 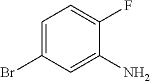 | 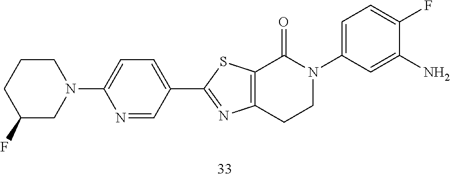 [SMILES: Nc1cc(N2CCc3nc(-c4ccc(N5CCC[C@H](F)C5)nc4)sc3C2=O)ccc1F] | 1. 27% 2. ¹H NMR (500 MHZ, DMSO-d₆): δ 8.73 (d, 1H), 8.04 (dd, 1H), 7.01 (m, 1H), 6.76 (dd, 1H), 6.49 (dt, 1H), 5.24 (s, 2H), 4.81 (d, 1H), 4.10 (m, 1H), 3.96 (dt, 3H), 3.72 (dd, 1H), 3.42 (t, 1H), 3.18 (t, 2H), 1.94 (m, 2H), 1.76 (d, 1H), 1.56 (d, 1H) 3. 442.2 4. Example 1 |

### Synthesis of building block 2-(5-(pyrrolidin-1-yl)pyridin-2-yl)-6,7-dihydrothiazolo[5,4-c]pyridin-4(5H)-one (Prep 8)

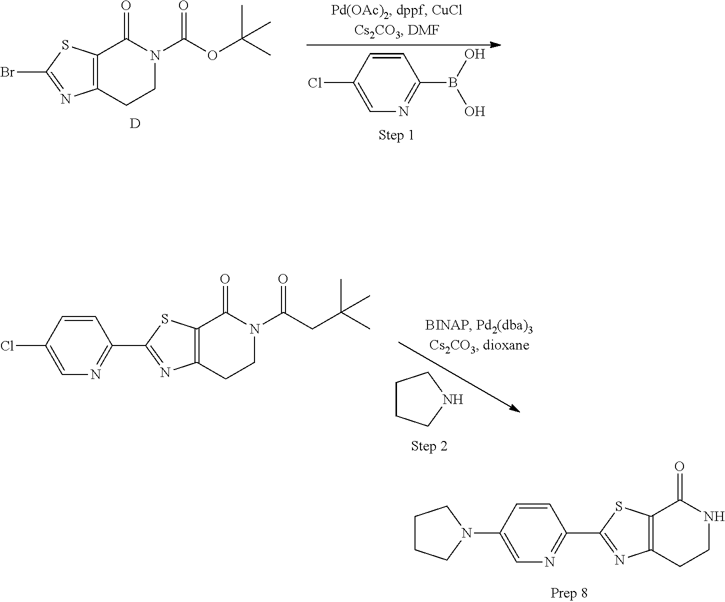

### Step-1: Synthesis of tert-butyl 2-(5-chloropyridin-2-yl)-4-oxo-6,7-dihydrothiazolo[5,4-c]pyridine-5(4H)-carboxylate

To a degassed solution of N,N′-dimethylformamide (9.2 mL) were added compound D (0.3 g, 0.9 mmol), (5-chloropyridin-2-yl)boronic acid (0.43 g, 1.8 mmol), cesium carbonate (0.588 g, 1.62 mmol), copper(I)-chloride (0.089 g, 0.9 mmol), palladium(II) acetate (0.01 g, 0.046 mmol) and 1,1′-bis(diphenylphosphino)ferrocene (0.05 g, 0.092 mmol). The reaction mixture was heated at −85° C. in a sand-bath for 5 h. The reaction mixture was diluted with ethyl acetate (200 mL) and water (80 mL). The organic phase was separated, dried over Na₂SO₄, filtered and the solvents were removed under reduced pressure. The residue was purified by chromatography on silica (50 g HP-Ultra) using a Biotage Isolera system employing an ethyl acetate/n-heptane gradient (5/95->10/90->20/80->30/70->40/60) to afford the title compound as a white solid (0.237 g, 72%).

¹H NMR (400 MHz, DMSO-d₆): δ 8.79 (d, 1H), 8.20-8.15 (m, 2H), 4.10 (t, 2H), 3.17 (t, 2H), 1.50 (s, 9H). LCMS (ESI) 309.94/311.96 m/z [M⁺H—C₄H₈]⁺.

### Step-2: Synthesis of 2-(5-(pyrrolidin-1-yl)pyridin-2-yl)-6,7-dihydrothiazolo[5,4-c]pyridin-4(5H)-one (Prep 8)

To a solution of degassed 1,4-dioxane (10 mL) were added the title compound from Step 1 above (0.170 g, 0.464 mmol), pyrrolidine (0.497 g, 0.7 mmol), 2,2′-bis(diphenylphosphino)-1,1′-binaphthyl (0.058 g, 0.093 mmol), tris(dibenzylideneacetone)dipalladium(0) (0.0425 g, 0.0464 mmol) and cesium carbonate (0.452 g, 1.39 mmol). The reaction mixture was heated at 120° C. for 3 h. The reaction mixture was concentrated under reduced pressure. The residue was purified by chromatography on silica (100-200 mesh) using a MeOH/CH₂Cl₂ gradient (1/99->2/98) to afford the title compound Prep 8 as a yellow solid (0.07 g, 50%). ¹H NMR (500 MHz, DMSO-d₆): δ 8.01 (d, 1H), 7.93 (d, 1H), 7.77 (s, 1H), 7.0 (dd, 1H), 3.49 (t, 2H), 3.36 (t, 4H), 2.97 (t, 2H), 1.99 (m, 4H). LCMS (ESI) 301.13 m/z [M+H]⁺.

### Example 34

Following the coupling procedure as reported in Example 1 using the building block and halogen derivative indicated in Table 3, the following compound was prepared.

TABLE 3

| | | | | 1. Yield |
|---|---|---|---|---|
| | | | | 2. ¹H-NMR |
| | | Halogen | | 3. MH⁺ (ESI) |
| Example | Building block | derivative | Product | 4. Synthesis procedure |
| 34 | 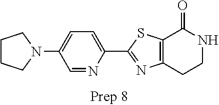 [SMILES: O=C1NCCc2nc(-c3ccc(N4CCCC4)cn3)sc21] | 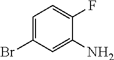 | 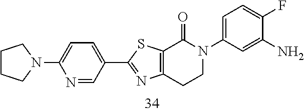 [SMILES: Nc1cc(N2CCc3nc(-c4ccc(N5CCCC5)nc4)sc3C2=O)ccc1F] | 1. 29% 2. ¹H NMR (500 MHZ, DMSO-d₆): δ 8.03 (d, 1H), 7.96 (d, 1H), 7.01 (dd, 2H), 6.76 (dd, 1H), 6.49 (td, 1H), 5.23 (s, 2H), 3.98 (t, 2H), 3.37 (t, 2H), 3.17 (m, 2H), 1.99 (t, 4H) 3. 410.25 4. Example 1 |

### Synthesis of building block (R)-2-(5-(3-fluoropyrrolidin-1-yl)pyrazin-2-yl)-6,7-dihydrothiazolo[5,4-c]pyridin-4(5H)-one (Prep 9)

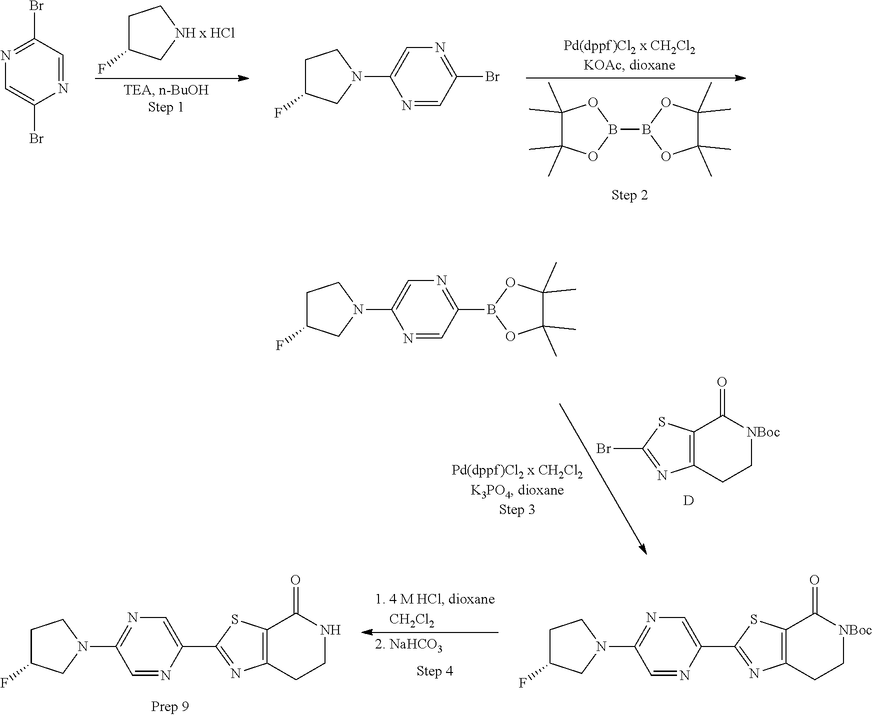

### Step-1: Synthesis of (R)-2-bromo-5-(3-fluoropyrrolidin-1-yl)pyrazine

Commercially available 2,5-dibromo pyrazine (0.2 g, 8.4 mmol), (R)-3-fluoropyrrolidine hydrochloric acid salt (2.6 g, 21.0 mmol), and Et₃N (6.0 mL, 4.35 g, 43 mmol) were suspended in n-butanol (20 mL) using a microwave vial. The sealed vial was then heated at 120° C. for 3 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (1.9 g, 93%). ¹H NMR (400 MHz DMSO-d₆): δ 8.31 (S, 1H), 7.65 (d, 1H), 5.40 (dt, 1H), 3.83 (q, 1H), 3.68 (m, 1H), 3.58 (td, 2H), 2.44 (m, 1H), 2.17 (m, 1H). LCMS (ESI) 245.70/247.7 m/z [M+H]⁺.

### Step-2: Synthesis of (R)-2-(3-fluoropyrrolidin-1-yl)-5-(4,4,5,5-tetramethyl-1,3,2-dioxaborolan-2-yl)pyrazine

The title compound from Step 1 above (1.9 g, 7.75 mmol) was dissolved in 1,4-dioxane (40 mL) and degassed by passing a stream of nitrogen through the mixture. Then [1,1′-bis(diphenyl-phosphino)ferrocene]-dichloropalladium(II), complex with dichloromethane (0.63 g, 0.77 mmol), bispinacolatodiboron (3.9 g, 15.5 mmol) and potassium acetate (1.52 g, 15.5 mmol) were added, and the reaction mixture was heated at 100° C. in an oil-bath for 5 h. The reaction mixture was washed three times with n-hexane (100 mL) and concentrated under reduced pressure to obtain the title compound as a brown solid (5 g, quant.). LCMS (ESI) 294.28 m/z [M+H]⁺.

### Step-3: Synthesis of tert-butyl (R)-2-(5-(3-fluoropyrrolidin-1-yl)pyrazin-2-yl)-4-oxo-6,7-dihydrothiazolo[5,4-c]pyridine-5(4H)-carboxylate

Compound D (0.5 g, 1.5 mmol) was dissolved in 1,4-dioxane (25 mL) and the solution was degassed by a stream of nitrogen. Then [1,1′-bis(diphenyl-phosphino)ferrocene]-dichloropalladium(II), complex with dichloromethane (0.12 g, 0.15 mmol), the title compound from Step 2 above (1.9 g, 6.6 mmol) and tripotassium phosphate (1.0 g, 4.5 mmol) were added, and the reaction mixture was heated at 80° C. in an oil-bath for 5 h. The reaction mixture was concentrated under reduced pressure. The residue was purified by column chromatography on silica gel (basic, 100-200 mesh) using ethyl acetate/n-hexane gradient (0/100->5/95->15/85->20/80->30/70) to obtain the title compound as a pale-yellow solid (0.25 g, 41%). ¹H NMR (500 MHz DMSO-d₆): δ 8.81 (S, 1H), 8.14 (s, 1H), 5.51 (d, 1H), 4.06 (t, 2H), 3.82 (m, 3H), 3.58 (m, 1H), 3.11 (t, 2H), 2.24 (m, 2H), 1.47 (s, 9H). LCMS (ESI) 363.9 m/z [M+H]⁺.

### Step-4: Synthesis of (R)-2-(5-(3-fluoropyrrolidin-1-yl)pyrazin-2-yl)-6,7-dihydrothiazolo[5,4-c]pyridin-4(5H)-one (Prep 9)

The title compound from Step 3 above (0.25 g, 0.59 mmol) was dissolved in CH₂Cl₂ (10 mL) and the mixture was cooled to 0° C. in an ice-bath. Then a 4 M solution of HCl in 1,4-dioxane (2.5 mL) was added and stirring was continued at room temperature for 3 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 9 as a pale-yellow solid (0.17 g, 89%). ¹H NMR (500 MHz DMSO-d₆): δ 8.79 (S, 1H), 8.12 (s, 1H), 7.83 (s, 1H), 5.51 (d, 1H), 3.81 (m, 3H), 3.57 (m, 1H), 3.15 (td, 2H), 3.00 (t, 2H), 2.25 (m, 2H). LCMS (ESI) 319.9 m/z [M+H].

### Synthesis of Building Block (S)-2-(5-(3-fluoropyrrolidin-1-yl)pyrazin-2-yl)-6,7-dihydrothiazolo[5,4-c]pyridin-4(5H)-one (Prep 10)

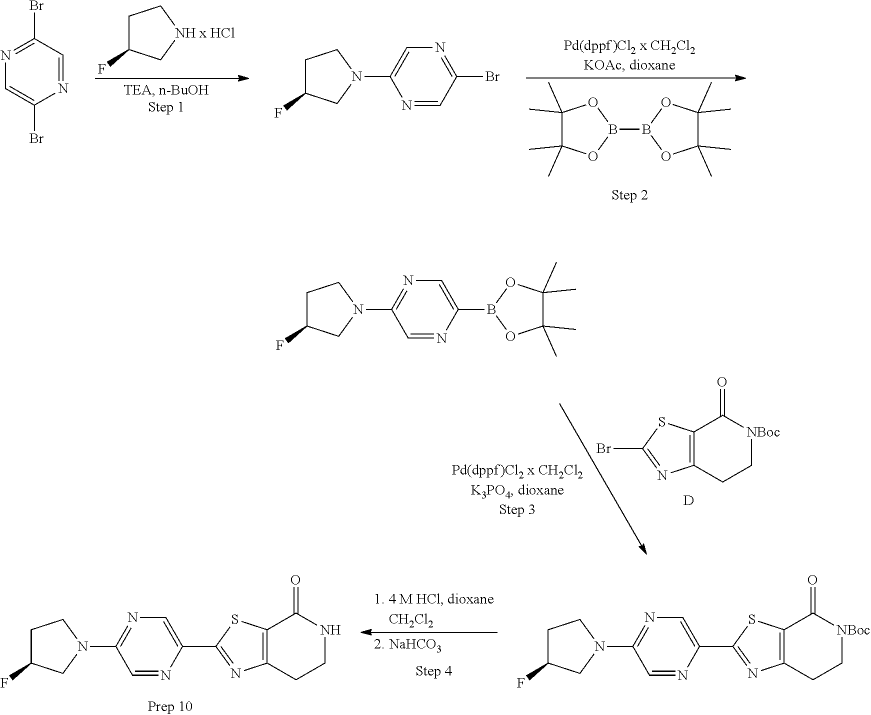

### Step-1: Synthesis of (S)-2-bromo-5-(3-fluoropyrrolidin-1-yl)pyrazine

Commercially available 2,5-dibromo pyrazine (2.0 g, 8.4 mmol), (R)-3-fluoropyrrolidine hydrochloric acid salt (2.6 g, 21.0 mmol), and Et₃N (6.0 mL, 4.35 g, 43 mmol) were suspended in n-butanol (20 mL) using a microwave vial. The sealed vial was then heated at 120° C. for 3 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (1.9 g, 95%). ¹H NMR (500 MHz DMSO-d₆): δ 8.21 (S, 1H), 7.64 (s, 1H), 5.47 (m, 1H), 3.66 (m, 3H), 3.44 (dt, 1H), 2.22 (m, 2H). LCMS (ESI) 246.05/248.05 m/z [M+H]⁺.

### Step-2: Synthesis of (S)-2-(3-fluoropyrrolidin-1-yl)-5-(4,4,5,5-tetramethyl-1,3,2-dioxaborolan-2-yl)pyrazine

The title compound from Step 1 above (0.97 g, 3.95 mmol) was dissolved in 1,4-dioxane (20 mL) and degassed by passing a stream of nitrogen through the mixture. Then [1,1′-bis(diphenyl-phosphino)ferrocene]-dichloropalladium(II), complex with dichloromethane (0.323 g, 0.39 mmol), bispinacolatodiboron (2.0 g, 7.9 mmol) and potassium acetate (0.78 g, 7.9 mmol) were added, and the reaction mixture was heated at 100° C. in an oil-bath for 5 h. The reaction mixture was washed three times with n-hexane (100 mL) and concentrated under reduced pressure to obtain the title compound as a brown solid (1.4 g, quant.). LCMS (ESI) 294.16 m/z [M+H]⁺.

### Step-3: Synthesis of tert-butyl (S)-2-(5-(3-fluoropyrrolidin-1-yl)pyrazin-2-yl)-4-oxo-6,7-dihydrothiazolo[5,4-c]pyridine-5(4H)-carboxylate

Compound D (1.0 g, 3.0 mmol) was dissolved in 1,4-dioxane (50 mL) and the solution was degassed by a stream of nitrogen. Then [1,1′-bis(diphenyl-phosphino)ferrocene]-dichloropalladium(II), complex with dichloromethane (0.24 g, 0.3 mmol), the title compound from Step 2 above (2.28 g, 7.8 mmol) and tripotassium phosphate (1.9 g, 9.0 mmol) were added, and the reaction mixture was heated at 80° C. in an oil-bath for 5 h. The reaction mixture was concentrated under reduced pressure. The residue was purified by column chromatography on silica gel (basic, 100-200 mesh) using ethyl acetate/n-hexane gradient (0/100->15/85->20/80->25/75) to obtain the title compound as a pale-yellow solid (0.13 g, 10%). ¹H NMR (400 MHz DMSO-d₆): δ 8.81 (S, 1H), 8.15 (s, 1H), 5.52 (d, 1H), 4.06 (t, 2H), 3.82 (m, 3H), 3.58 (m, 1H), 3.11 (t, 2H), 2.23 (m, 2H), 1.49 (s, 9H). LCMS (ESI) 420.1 m/z [M+H]⁺.

### Step-4: Synthesis of (S)-2-(5-(3-fluoropyrrolidin-1-yl)pyrazin-2-yl)-6,7-dihydrothiazolo[5,4-c]pyridin-4(5H)-one (Prep 10)

The title compound from Step 3 above (0.13 g, 0.31 mmol) was dissolved in CH₂Cl₂ (6.5 mL) and the mixture was cooled to 0° C. in an ice-bath. Then a 4 M solution of HCl in 1,4-dioxane (1.3 mL) was added and stirring was continued at room temperature for 3 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 10 as a pale-yellow solid (0.07 g, 71%). ¹H NMR (500 MHz DMSO-d₆): δ 8.79 (d, 1H), 8.12 (d, 1H), 7.83 (s, 1H), 5.51 (d, 1H), 3.80 (m, 3H), 3.54 (m, 3H), 3.00 (t, 2H), 2.21 (m, 2H). LCMS (ESI) 319.95 m/z [M+H]⁺.

### Synthesis of Building Block 2-(5-(4-fluoropiperidin-1-yl)pyrazin-2-yl)-6,7-dihydrothiazolo[5,4-c]pyridin-4(5H)-one (Prep 11)

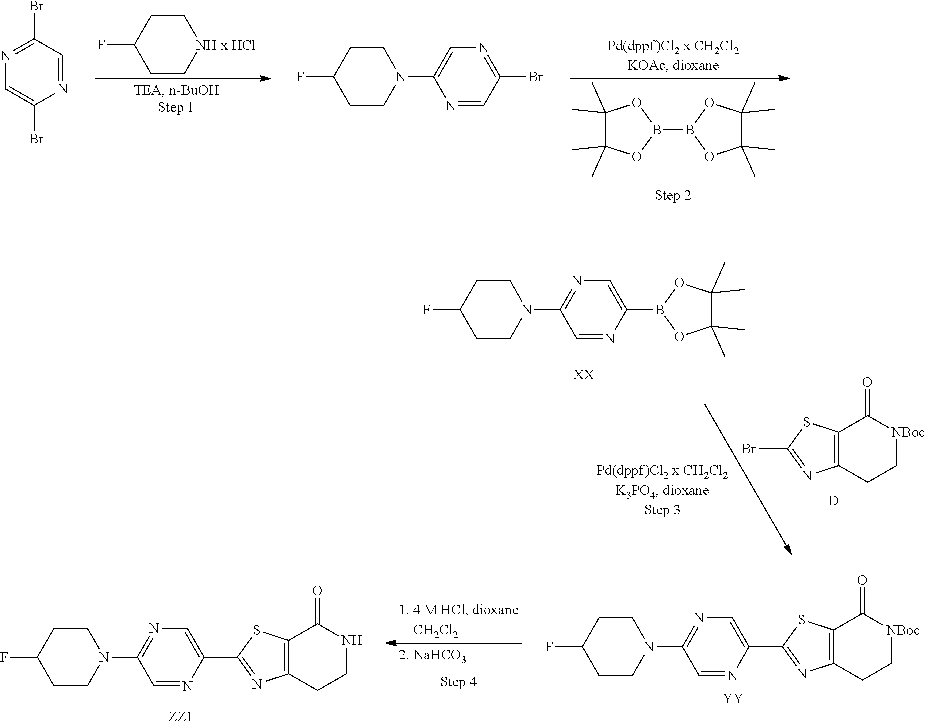

### Step-1: Synthesis of 2-bromo-5-(4-fluoropiperidin-1-yl)pyrazine

Commercially available 2,5-dibromopyrazine (2.0 g, 8.4 mmol), 4-fluoropiperidine hydrochloric acid salt (2.8 g, 21.0 mmol), and Et₃N (6.0 mL, 4.2 g, 42 mmol) were suspended in n-butanol (20 mL) using a microwave vial. The sealed vial was then heated at 120° C. for 3 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (2.15 g, 98%). ¹H NMR (500 MHz DMSO-d₆): δ 8.21 (d, 1H), 4.9 (m, 1H), 3.72 (m, 2H), 3.56 (m, 2H), 1.93 (m, 2H) 1.72 (m, 2H). LCMS (ESI) 260.15/262.15 m/z [M+H]⁺.

### Step-2: Synthesis of 2-(4-fluoropiperidin-1-yl)-5-(4,4,5,5-tetramethyl-1,3,2-dioxaborolan-2-yl)pyrazine

The title compound from Step 1 above (2.2 g, 8.5 mmol) was dissolved in 1,4-dioxane (44 mL) and degassed by passing a stream of nitrogen through the mixture. Then [1,1′-bis(diphenyl-phosphino)ferrocene]-dichloropalladium(II), complex with dichloromethane (0.704 g, 0.85 mmol), bispinacolatodiboron (4.2 g, 16.6 mmol) and potassium acetate (1.6 g, 16.6 mmol) were added, and the reaction mixture was heated at 100° C. in an oil-bath for 5 h. The reaction mixture was washed three times with n-hexane (100 mL) and concentrated under reduced pressure to obtain the title compound as a brown solid (4.3 g, quant.). LCMS (ESI) 308.15 m/z [M+H]⁺.

### Step-3: Synthesis of tert-butyl 2-(5-(4-fluoropiperidin-1-yl)pyrazin-2-yl)-4-oxo-6,7-dihydrothiazolo[5,4-c]pyridine-5(4H)-carboxylate

Compound D (0.2 g, 0.6 mmol) was dissolved in 1,4-dioxane (10 mL) and the solution was degassed by a stream of nitrogen. Then [1,1′-bis(diphenyl-phosphino)ferrocene]-dichloropalladium(II), complex with dichloromethane (0.049 g, 0.06 mmol), the title compound from Step 2 above (0.6 g, 1.98 mmol) and tripotassium phosphate (0.382 g, 1.8 mmol) were added, and the reaction mixture was heated at 80° C. in an oil-bath for 8 h. The reaction mixture was concentrated under reduced pressure. The residue was purified by column chromatography on silica gel (basic, 100-200 mesh) using ethyl acetate/n-hexane gradient (0/100->15/85->20/80) to obtain the title compound as a pale-yellow solid (0.19 g, 73%). ¹H NMR (500 MHz DMSO-d₆): δ 8.79 (S, 1H), 8.48 (s, 1H), 4.95 (d, 1H), 4.06 (t, 2H), 3.93 (s, 1H), 3.82 (m, 4H), 3.11 (t, 2H), 1.97 (m, 2H), 1.79 (d, 2H), 1.49 (s, 9H). LCMS (ESI) 434.35 m/z [M+H]⁺.

### Step-4: Synthesis of 2-(5-(4-fluoropiperidin-1-yl)pyrazin-2-yl)-6,7-dihydrothiazolo[5,4-c]pyridin-4(5H)-one (Prep 11)

The title compound from Step 3 above (0.19 g, 0.438 mmol) was dissolved in CH₂Cl₂ (9.5 mL) and the mixture was cooled to 0° C. in an ice-bath. Then a 4 M solution of HCl in 1,4-dioxane (1.9 mL) was added and stirring was continued at room temperature for 3 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 11 as a pale-yellow solid (0.12 g, 82%). ¹H NMR (500 MHz DMSO-d₆): δ 8.77 (d, 1H), 8.46 (t, 1H), 7.85 (s, 1H), 4.95 (m, 1H), 3.86 (t, 2H), 3.75 (m, 2H), 3.50 (td, 2H), 3.00 (t, 2H), 1.97 (m, 2H), 1.78 (d, 2H). LCMS (ESI) 334.3 m/z [M+H]⁺.

### Synthesis of Building Block (S)-2-(5-(3-fluoropiperidin-1-yl)pyrazin-2-yl)-6,7-dihydrothiazolo[5,4-c]pyridin-4(5H)-one (Prep 12)

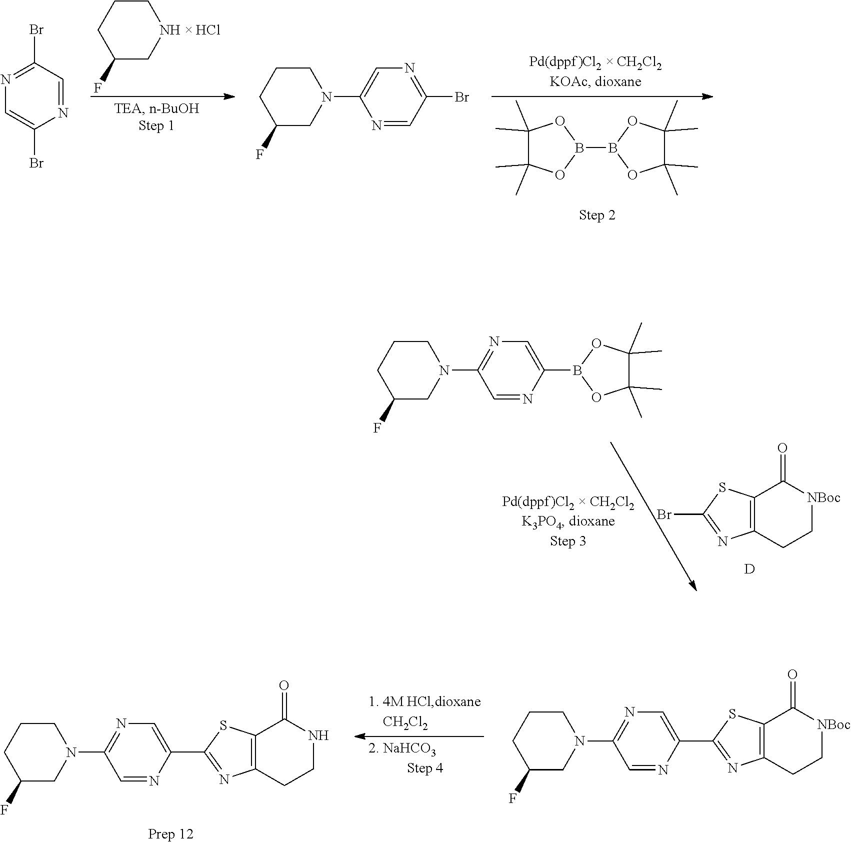

Step-1: Synthesis of (S)-2-bromo-5-(3-fluoropiperidin-1-yl)pyrazine Commercially available 2,5-dibromopyrazine (1.0 g, 4.2 mmol), (S)-3-fluoropiperidine hydrochloric acid salt (1.4 g, 10.5 mmol), and Et₃N (3.0 mL, 2.1 g, 21 mmol) were suspended in n-butanol (10 mL) using a microwave vial. The sealed vial was then heated at 120° C. for 3 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (0.98 g, 95%). ¹H NMR (500 MHz DMSO-d₆): δ 8.19 (d, 2H), 4.80 (m, 1H), 4.00 (m, 1H), 3.83 (dt, 1H), 3.60 (qd, 1H), 3.35 (d, 1H), 1.90 (m, 2H), 1.74 (m, 1H), 1.55 (m, 1H). LCMS (ESI) 260.05/262.05 m/z [M+H]⁺.

### Step-2: Synthesis of (S)-2-(3-fluoropiperidin-1-yl)-5-(4,4,5,5-tetramethyl-1,3,2-dioxaborolan-2-yl)pyrazine

The title compound from Step 1 above (2.2 g, 8.46 mmol) was dissolved in 1,4-dioxane (44 mL) and degassed by passing a stream of nitrogen through the mixture. Then [1,1′-bis(diphenyl-phosphino)ferrocene]-dichloropalladium(II), complex with dichloromethane (0.7 g, 0.846 mmol), bispinacolatodiboron (4.2 g, 16.6 mmol) and potassium acetate (1.6 g, 16.6 mmol) were added, and the reaction mixture was heated at 100° C. in an oil-bath for 5 h. The reaction mixture was washed three times with n-hexane (100 mL) and concentrated under reduced pressure to obtain the title compound as a brown solid (4.3 g, quant.). LCMS (ESI) 308.15 m/z [M+H]⁺.

### Step-3: Synthesis of tert-butyl (S)-2-(5-(3-fluoropiperidin-1-yl)pyrazin-2-yl)-4-oxo-6,7-dihydrothiazolo[5,4-c]pyridine-5(4H)-carboxylate

Compound D (0.2 g, 0.6 mmol) was dissolved in 1,4-dioxane (10 mL) and the solution was degassed by a stream of nitrogen. Then [1,1′-bis(diphenyl-phosphino)ferrocene]-dichloropalladium(II), complex with dichloromethane (0.049 g, 0.06 mmol), the title compound from Step 2 above (0.6 g, 1.98 mmol) and tripotassium phosphate (0.382 g, 1.8 mmol) were added, and the reaction mixture was heated at 80° C. in an oil-bath for 8 h. The reaction mixture was concentrated under reduced pressure. The residue was purified by column chromatography on silica gel (basic, 100-200 mesh) using ethyl acetate/n-hexane gradient (0/100->15/85->20/80) to obtain the title compound as a pale-yellow solid (0.19 g, 73%). ¹H NMR (500 MHz DMSO-d₆): δ 8.79 (S, 1H), 8.48 (s, 1H), 4.95 (d, 1H), 4.06 (t, 2H), 3.93 (s, 1H), 3.82 (m, 4H), 3.11 (t, 2H), 1.97 (m, 2H), 1.79 (d, 2H), 1.49 (s, 9H). LCMS (ESI) 434.35 m/z [M+H]⁺.

### Step-4: Synthesis of (S)-2-(5-(3-fluoropiperidin-1-yl)pyrazin-2-yl)-6,7-dihydrothiazolo[5,4-c]pyridin-4(5H)-one (Prep 12)

The title compound from Step 3 above (0.19 g, 0.438 mmol) was dissolved in CH₂Cl₂ (9.5 mL) and the mixture was cooled to 0° C. in an ice-bath. Then a 4 M solution of HCl in 1,4-dioxane (1.9 mL) was added and stirring was continued at room temperature for 3 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 12 as a pale-yellow solid (0.12 g, 82%). ¹H NMR (500 MHz DMSO-d₆): δ 8.77 (d, 1H), 8.46 (t, 1H), 7.85 (s, 1H), 4.95 (m, 1H), 3.86 (t, 2H), 3.75 (m, 2H), 3.50 (td, 2H), 3.00 (t, 2H), 1.97 (m, 2H), 1.78 (d, 2H). LCMS (ESI) 334.3 m/z [M+H]⁺.

### Examples 35 to 41

Following the coupling procedure as reported in Example 1 and using the building blocks and halogen derivatives indicated in Table 4, the following compounds were prepared.

TABLE 4

| | | | | 1. Yield |
|---|---|---|---|---|
| | | | | 2. ¹H-NMR |
| | | Halogen | | 3. MH⁺ (ESI) |
| Example | Building block | derivative | Product | 4. Synthesis procedure |
| 35 | 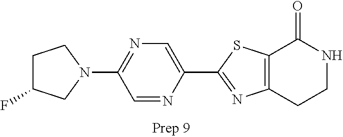 [SMILES: O=C1NCCc2nc(-c3cnc(N4CC[C@@H](F)C4)cn3)sc21] | 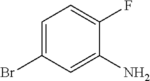 | 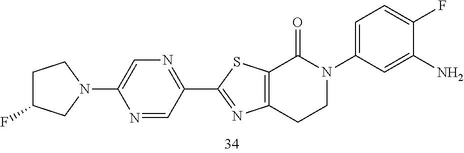 [SMILES: Nc1cc(N2CCc3nc(-c4cnc(N5CC[C@@H](F)C5)cn4)sc3C2=O)ccc1F] | 1. 47% 2. ¹H NMR (500 MHZ, DMSO-d₆): δ 8.82 (s, 1H), 8.14 (s, 1H), 7.01 (dd, 1H), 6.76 (dd, 1H), 6.50 (m, 1H), 5.52 (d, 1H), 5.24 (s, 2H), 3.99 (t, 2H), 3.82 (m, 3H), 3.58 (dd, 1H), 3.20 (t, 2H), 2.26 (m,2H) 3. 428.9 4. Example 1 |
| 36 | 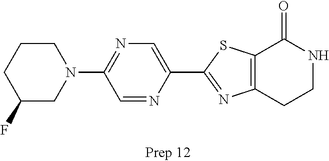 [SMILES: O=C1NCCc2nc(-c3cnc(N4CCC[C@H](F)C4)cn3)sc21] | 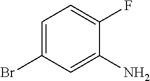 | 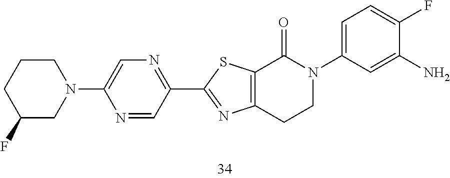 [SMILES: Nc1cc(N2CCc3nc(-c4cnc(N5CCC[C@H](F)C5)cn4)sc3C2=O)ccc1F] | 1. 66% 2. ¹H NMR (400 MHZ, DMSO-d₆): δ 8.78 (d, 1H), 8.47 (s, 1H), 7.01 (dd, 1H), 6.76 (dd, 1H), 6.50 (m, 1H), 5.23 (d, 2H), 4.88 (m, 1H), 4.26 (m, 1H), 4.08 (dd, 1H), 3.99 (t, 2H), 3.71 (m, 1H), 3.45 (m,1H), 3.20 (t, 2H), 1.94 (m, 2H), 1.78 (m, 1H), 1.60 (m, 1H) 3. 443.0 4. Example 1 |
| 37 | 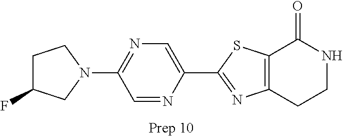 [SMILES: O=C1NCCc2nc(-c3cnc(N4CC[C@H](F)C4)cn3)sc21] | 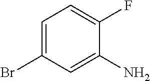 | 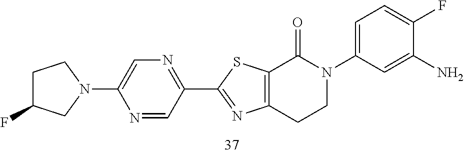 [SMILES: Nc1cc(N2CCc3nc(-c4cnc(N5CC[C@H](F)C5)cn4)sc3C2=O)ccc1F] | 1. 76% 2. ¹H NMR (500 MHZ, DMSO-d₆): δ 8.82 (s, 1H), 8.14 (s, 1H), 7.01 (dd, 1H), 6.76 (dd, 1H), 6.50 (d, 1H), 5.52 (d, 1H), 5.24 (s, 2H), 3.99 (t, 2H), 3.81 (m, 3H), 3.58 (m, 1H), 3.20 (t, 2H), 2.25 (m, 2H) 3. 429.0 4. Example 1 |
| 38 | 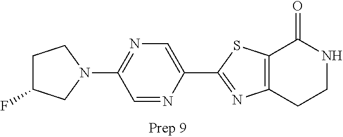 [SMILES: O=C1NCCc2nc(-c3cnc(N4CC[C@@H](F)C4)cn3)sc21] | 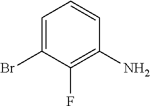 | 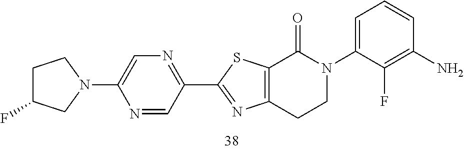 [SMILES: Nc1cccc(N2CCc3nc(-c4cnc(N5CC[C@@H](F)C5)cn4)sc3C2=O)c1F] | 1. 87% 2. ¹H NMR (500 MHZ, DMSO-d₆): δ 8.83 (d, 1H), 8.14 (d, 1H), 6.90 (t, 1H), 6.73 (td, 1H), 6.56 (m, 1H), 5.52 (d, 1H), 5.25 (s, 2H), 3.97 (t, 2H), 3.80 (m, 3H), 3.58 (dt, 1H), 3.22 (t, 2H), 2.22 (m, 2H) 3. 428.9 4. Example 1 |
| 39 | 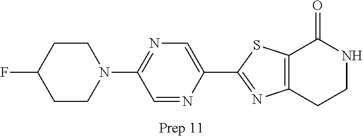 [SMILES: O=C1NCCc2nc(-c3cnc(N4CCC(F)CC4)cn3)sc21] | 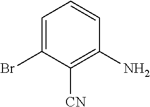 | 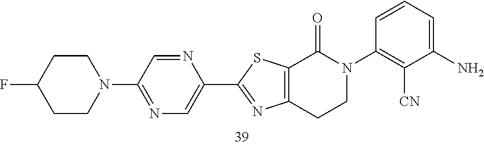 | 1. 15% 2. ¹H NMR (500 MHZ, DMSO-d₆): δ 8.81 (d, 1H), 8.49 (s, 1H), 7.35 (t, 1H), 6.76 (d, 1H), 6.64 (d, 1H), 6.18 (d, 3H), 4.96 (m, 1H), 4.02 (s, 1H), 3.87 (t, 2H), 3.78 (m, 2H), 3.24 (t, 2H), 1.98 (tq, 2H), 1.79 (m, 2H) 3. 449.9 4. Example 1 |
| 40 | 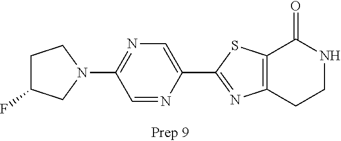 [SMILES: O=C1NCCc2nc(-c3cnc(N4CC[C@@H](F)C4)cn3)sc21] | 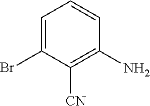 | 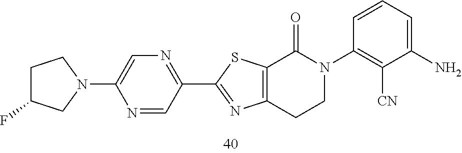 [SMILES: N#Cc1c(N)cccc1N1CCc2nc(-c3cnc(N4CC[C@@H](F)C4)cn3)sc2C1=O] | 1. 7% 2. ¹H NMR (400 MHZ, DMSO-d₆): δ 8.83 (d, 1H), 8.15 (d, 1H), 7.35 (t, 1H), 6.75 (dd, 1H), 6.64 (d, 1H), 6.19 (s, 2H), 5.52 (d, 1H), 3.85 (m, 5H), 3.59 (dt, 1H), 3.24 (t, 2H),2.24 (m, 2H) 3. 436.23 4. Example 1 |
| 41 | 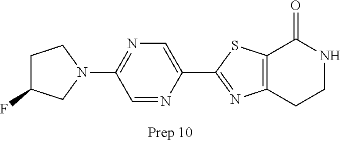 [SMILES: O=C1NCCc2nc(-c3cnc(N4CC[C@H](F)C4)cn3)sc21] | 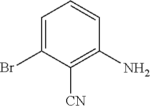 | 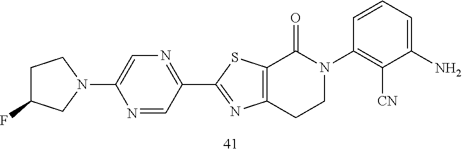 [SMILES: N#Cc1c(N)cccc1N1CCc2nc(-c3cnc(N4CC[C@H](F)C4)cn3)sc2C1=O] | 1. 18.3% 2. ¹H NMR (400 MHZ, DMSO-d₆): δ 8.83 (d, 1H), 8.15 (d, 1H), 7.35 (t, 1H), 6.75 (dd, 1H), 6.64 (d, 1H), 6.19 (s, 2H), 5.52 (d, 1H), 4.02 (s, 1H), 3.82 (m, 4H), 3.59 (dt, 1H), 3.24 (t, 2H), 2.24 (m, 2H) 3. 436.27 4. Example 1 |

### Synthesis of Building Block 2-(2-(pyrrolidin-1-yl)pyrimidin-5-yl)-6,7-dihydrothiazolo[5,4-c]pyridin-4(5H)-one (Prep 13)

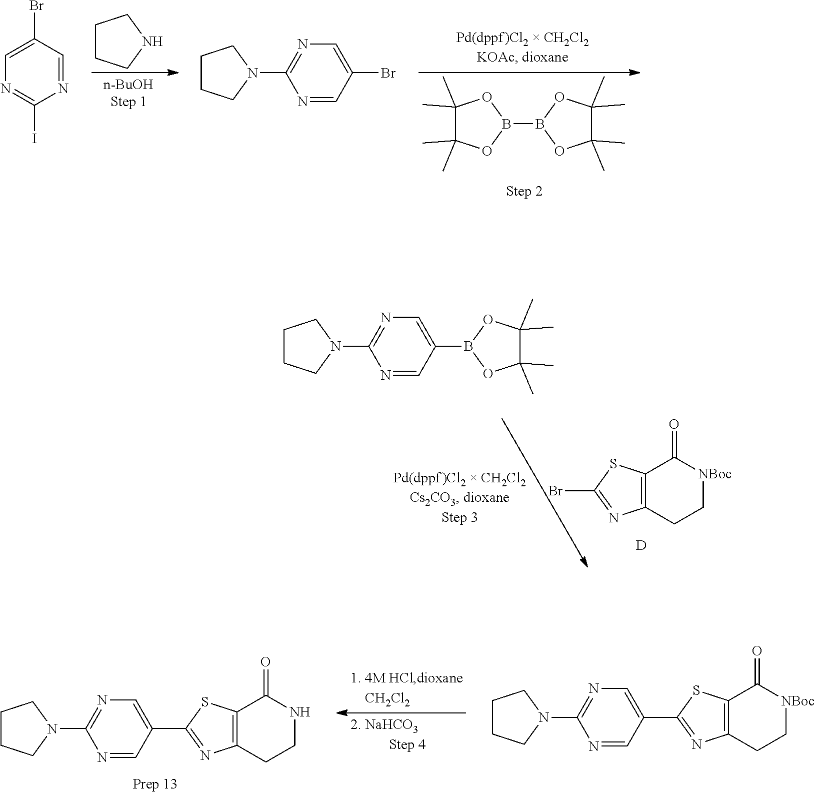

### Step-1: Synthesis of 5-bromo-2-(pyrrolidin-1-yl)pyrimidine

Commercially available 2-iodo-5-bromo pyrimidine (1.1 g, 3.8 mmol) and pyrrolidine (3 ml, 2.7 g, 3.8 mmol) were suspended in n-butanol (6 mL) using a microwave vial. The sealed vial was then heated at 120° C. for 1 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (0.88 g, quant.). ¹H NMR (500 MHz DMSO-d₆): δ ¹H NMR (500 MHz DMSO-d₆): δ 8.29 (s, 2H), 3.53 (m, 4H), 2.0 (m, 4H).

### Step-2: Synthesis of 2-(pyrrolidin-1-yl)-5-(4,4,5,5-tetramethyl-1,3,2-dioxaborolan-2-yl)pyrimidine

The title compound from Step 1 above (2.5 g, 10.9 mmol) was dissolved in 1,4-dioxane (18 mL) and degassed by passing a stream of nitrogen through the mixture. Then [1,1′-bis(diphenyl-phosphino)ferrocene]-dichloropalladium(II), complex with dichloromethane (0.89 g, 1.09 mmol), bispinacolatodiboron (5.56 g, 22 mmol) and potassium acetate (2.16 g, 22 mmol) were added, and the reaction mixture was heated at 100° C. in an oil-bath for 3 h. The reaction mixture was washed three times with n-hexane (100 mL) and concentrated under reduced pressure to obtain the title compound as a brown solid (5.0 g, quant.). LCMS (ESI) 276.1 m/z [M+H]⁺.

### Step-3: Synthesis of tert-butyl 4-oxo-2-(2-(pyrrolidin-1-yl)pyrimidin-5-yl)-6,7-dihydrothiazolo[5,4-c]pyridine-5(4H)-carboxylate

Compound D (0.7 g, 2.1 mmol) was dissolved in 1,4-dioxane (25 mL) and the solution was degassed by a stream of nitrogen. Then [1,1′-bis(diphenyl-phosphino)ferrocene]-dichloropalladium(II), complex with dichloromethane (0.17 g, 0.2 mmol), the title compound from Step 2 above (1.0 g, 3.6 mmol) and cesium carbonate (2.05 g, 6.3 mmol) were added, and the reaction mixture was heated at 95-100° C. in an oil-bath for 5 h. The reaction mixture was concentrated under reduced pressure. The residue was purified by column chromatography on silica gel (basic, 100-200 mesh) using ethyl acetate/n-hexane gradient (0/100->10/90->20/80->30/70->40/60) to obtain the title compound as a pale-yellow solid (0.32 g, 20%). ¹H NMR (500 MHz DMSO-d₆): δ 8.86 (s, 2H), 4.15 (t, 2H), 3.66 (m, 4H), 3.12 (t, 2H), 2.04 (m, 4H), 1.57 (s, 9H), 1.55 (s, 2H). LCMS (ESI) 402.28 m/z [M+H]⁺.

### Step-4: Synthesis of 2-(2-(pyrrolidin-1-yl)pyrimidin-5-yl)-6,7-dihydrothiazolo[5,4-c]pyridin-4(5H)-one (Prep 13)

The title compound from Step 3 above (0.32 g, 0.8 mmol) was dissolved in CH₂Cl₂ (10 mL) and the mixture was cooled to 0° C. in an ice-bath. Then a 4 M solution of HCl in 1,4-dioxane (3 mL) was added and stirring was continued at room temperature for 4 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 13 as a pale-yellow solid (0.2 g, 83%). ¹H NMR (400 MHz DMSO-d₆): δ 8.90 (s, 2H), 7.85 (s, 1H), 3.57 (t, 4H), 3.49 (dt, 2H), 2.99 (t, 2H), 1.96 (m, 4H). LCMS (ESI) 302.14 m/z [M+H]⁺.

### Synthesis of Building Block (R)-2-(2-(3-fluoropyrrolidin-1-yl)pyrimidin-5-yl)-6,7-dihydrothiazolo[5,4-c]pyridin-4(5H)-one (Prep 14)

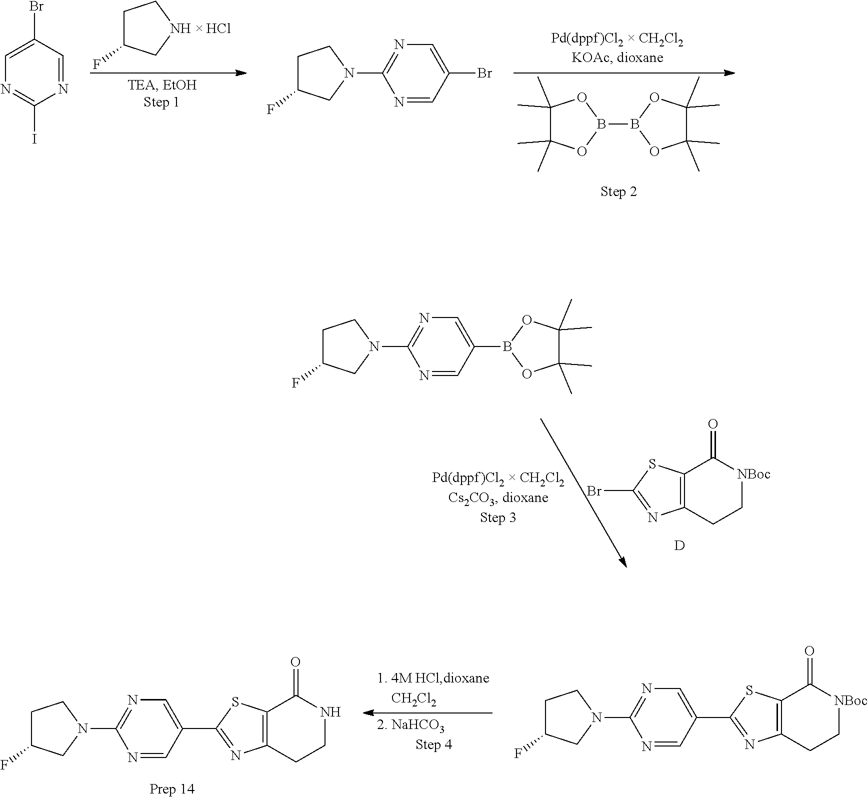

### Step-1: Synthesis of (R)-5-bromo-2-(3-fluoropyrrolidin-1-yl)pyrimidine

Commercially available 2-iodo-5-bromo pyrimidine (2.0 g, 7.02 mmol), (R)-3-flouropyrrolidine hydrochloride salt (1.76 g, 1.4 mmol), and Et₃N (3.9 mL, 2.84 g, 2.8 mmol) were suspended in ethanol (12 mL) using a microwave vial. The sealed vial was then heated at 120° C. for 1 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (1.65 g, 97%). ¹H NMR (400 MHz DMSO-d₆): δ 8.47 (s, 1H), 5.43 (m, 1H), 3.68 (m, 2H), 3.46 (dt, 1H), 2.18 (m, 2H). LCMS (ESI) 245.70/247.75 m/z [M+H]⁺.

### Step-2: Synthesis of (R)-2-(3-fluoropyrrolidin-1-yl)-5-(4,4,5,5-tetramethyl-1,3,2-dioxaborolan-2-yl)pyrimidine

The title compound from Step 1 above (1.65 g, 6.7 mmol) was dissolved in 1,4-dioxane (33 mL) and degassed by passing a stream of nitrogen through the mixture. Then [1,1′-bis(diphenyl-phosphino)ferrocene]-dichloropalladium(II), complex with dichloromethane (0.89 g, 1.09 mmol), bispinacolatodiboron (5.56 g, 22 mmol) and potassium acetate (2.16 g, 22 mmol) were added, and the reaction mixture was heated at 100° C. in an oil-bath for 5 h. The reaction mixture was washed three times with n-hexane (100 mL) and concentrated under reduced pressure to obtain the title compound as a brown solid (5.0 g, quant.). LCMS (ESI) 294.16 m/z [M+H]⁺.

### Step-3: Synthesis of tert-butyl (R)-2-(2-(3-fluoropyrrolidin-1-yl)pyrimidin-5-yl)-4-oxo-6,7-dihydrothiazolo[5,4-c]pyridine-5(4H)-carboxylate

Compound D (0.5 g, 1.5 mmol) was dissolved in 1,4-dioxane (25 mL) and the solution was degassed by a stream of nitrogen. Then [1,1′-bis(diphenyl-phosphino)ferrocene]-dichloropalladium(II), complex with dichloromethane (0.122 g, 0.15 mmol), the title compound from Step 2 above (1.8 g, 6.3 mmol) and cesium carbonate (1.4 g, 4.5 mmol) were added, and the reaction mixture was heated at 100° C. in an oil-bath for 5 h. The reaction mixture was concentrated under reduced pressure. The residue was purified by column chromatography on silica gel (basic, 100-200 mesh) using ethyl acetate/n-hexane gradient (0/100->10/90->20/80->30/70) to obtain the title compound as a pale-yellow solid (0.24 g, 38%). ¹H NMR (500 MHz DMSO-d₆): δ 8.97 (d, 2H), 5.47 (m, 1H), 4.07 (m, 2H), 3.90 (m, 2H), 3.75 (ddd, 1H), 3.59 (td, 1H), 3.16 (t, 1H), 3.11 (t, 2H), 2.23 (m, 2H), 1.49 (s, 9H). LCMS (ESI) 428.8 m/z [M+H]⁺

### Step-4: Synthesis of (R)-2-(2-(3-fluoropyrrolidin-1-yl)pyrimidin-5-yl)-6,7-dihydrothiazolo[5,4-c]pyridin-4(5H)-one (Prep 14)

The title compound from Step 3 above (0.24 g, 0.5 mmol) was dissolved in CH₂Cl₂ (5 mL) and the mixture was cooled to 0° C. in an ice-bath. Then a 4 M solution of HCl in 1,4-dioxane (2.5 mL) was added and stirring was continued at room temperature for 3 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 14 as a pale-yellow solid (0.135 g, 74%). ¹H NMR (500 MHz DMSO-d₆): δ 8.94 (d, 2H), 7.86 (s, 1H), 5.47 (d, 1H), 3.89 (m, 2H), 3.74 (ddd, 1H), 3.58 (td, 1H), 3.50 (td, 2H), 3.00 (t, 2H), 2.24 (m, 2H). LCMS (ESI) 320 m/z [M+H]⁺.

### Synthesis of Building Block (S)-2-(2-(3-fluoropyrrolidin-1-yl)pyrimidin-5-yl)-6,7-dihydrothiazolo[5,4-c]pyridin-4(5H)-one (Prep 15)

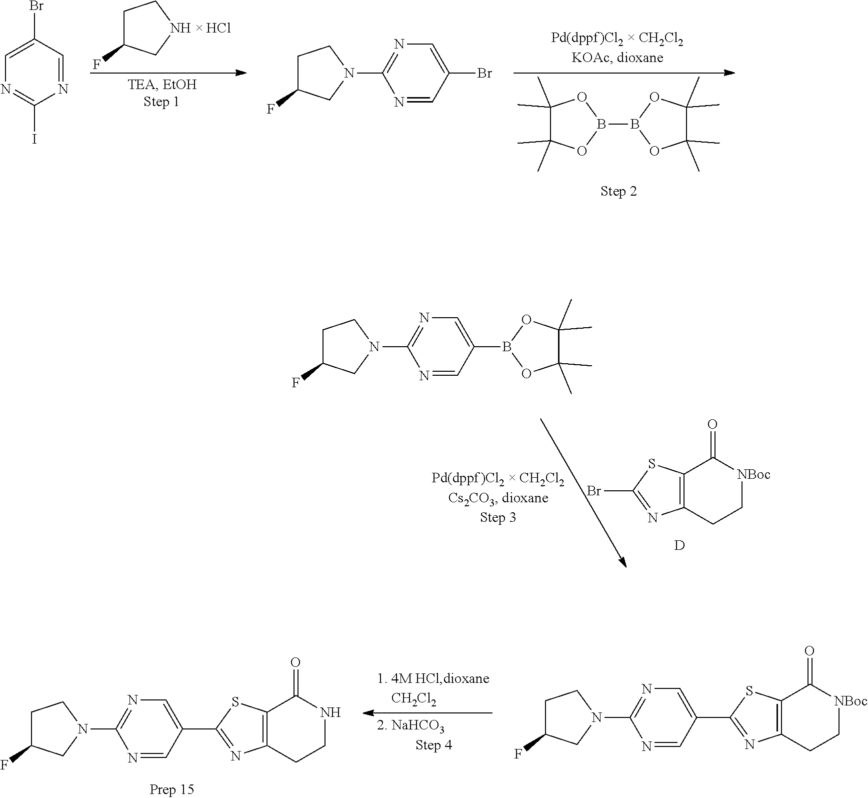

Step-1: Synthesis of (S)-5-bromo-2-(3-fluoropyrrolidin-1-yl)pyrimidine Commercially available 2-iodo-5-bromo pyrimidine (1.5 g, 5.26 mmol), (S)-3-flouro pyrrolidine hydrochloride salt (1.32 g, 1.05 mmol), and Et₃N (3.0 mL, 2.13 g, 2.1 mmol) were suspended in ethanol (9 mL) using a microwave vial. The sealed vial was then heated at 120° C. for 1 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (1.25 g, 97%). ¹H NMR (500 MHz DMSO-d₆): δ 8.47 (s, 2H), 5.43 (m, 1H), 3.68 (m, 3H), 3.46 (m, 1H), 2.20 (m, 2H). LCMS (ESI) 246.06/247.91 m/z [M+H]⁺.

### Step-2: Synthesis of (S)-2-(3-fluoropyrrolidin-1-yl)-5-(4,4,5,5-tetramethyl-1,3,2-dioxaborolan-2-yl)pyrimidine

The title compound from Step 1 above (1.3 g, 5.3 mmol) was dissolved in 1,4-dioxane (26 mL) and degassed by passing a stream of nitrogen through the mixture. Then [1,1′-bis(diphenyl-phosphino)ferrocene]-dichloropalladium(II), complex with dichloromethane (0.4 g, 0.53 mmol), bispinacolatodiboron (2.8 g, 10.6 mmol) and potassium acetate (1 g, 10.6 mmol) were added, and the reaction mixture was heated at 100° C. in an oil-bath for 5 h. The reaction mixture was washed three times with n-hexane (100 mL) and concentrated under reduced pressure to obtain the title compound as a brown solid (4.0 g, quant.). LCMS (ESI) 294.04 m/z [M+H]⁺.

### Step-3: Synthesis of tert-butyl (S)-2-(2-(3-fluoropyrrolidin-1-yl)pyrimidin-5-yl)-4-oxo-6,7-dihydrothiazolo[5,4-c]pyridine-5(4H)-carboxylate

Compound D (0.5 g, 1.5 mmol) was dissolved in 1,4-dioxane (25 mL) and the solution was degassed by a stream of nitrogen. Then [1,1′-bis(diphenyl-phosphino)ferrocene]-dichloropalladium(II), complex with dichloromethane (0.122 g, 0.15 mmol), the title compound from Step 2 above (2.0 g, 6.9 mmol) and cesium carbonate (1.4 g, 4.5 mmol) were added, and the reaction mixture was heated at 100° C. in an oil-bath for 5 h. The reaction mixture was concentrated under reduced pressure. The residue was purified by column chromatography on silica gel (basic, 100-200 mesh) using ethyl acetate/n-hexane gradient (0/100->10/90->20/80) to obtain the title compound as a pale-yellow solid (0.27 g, 42%). ¹H NMR (400 MHz DMSO-d₆): δ 8.97 (d, 2H), 5.47 (m, 1H), 4.06 (t, 2H), 3.19 (m, 2H), 3.75 (m, 2H), 3.59 (m, 1H), 3.11 (t, 2H), 2.22 (m, 2H), 1.49 (s, 9H). LCMS (ESI) 420.36 m/z [M+H]⁺.

### Step-4: Synthesis of (S)-2-(2-(3-fluoropyrrolidin-1-yl)pyrimidin-5-yl)-6,7-dihydrothiazolo[5,4-c]pyridin-4(5H)-one (Prep 15)

The title compound from Step 3 above (0.27 g, 0.62 mmol) was dissolved in CH₂Cl₂ (3 mL) and the mixture was cooled to 0° C. in an ice-bath. Then a 4 M solution of HCl in 1,4-dioxane (2.5 mL) was added and stirring was continued at room temperature for 2 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 15 as a pale-yellow solid (0.18 g, 87%). ¹H NMR (500 MHz DMSO-d₆): δ 8.94 (d, 2H), 7.86 (s, 1H), 5.47 (d, 1H), 3.89 (m, 2H), 3.75 (m, 1H), 3.58 (td, 1H), 3.50 (td, 2H), 3.00 (t, 2H), 2.21 (m, 2H), 1.19 (d, 2H). LCMS (ESI) 320.26 m/z [M+H]⁺.

### Synthesis of Building Block 2-(5-(4-fluoropiperidin-1-yl)pyrazin-2-yl)-6,7-dihydrothiazolo[5,4-c]pyridin-4(5H)-one (Prep 16)

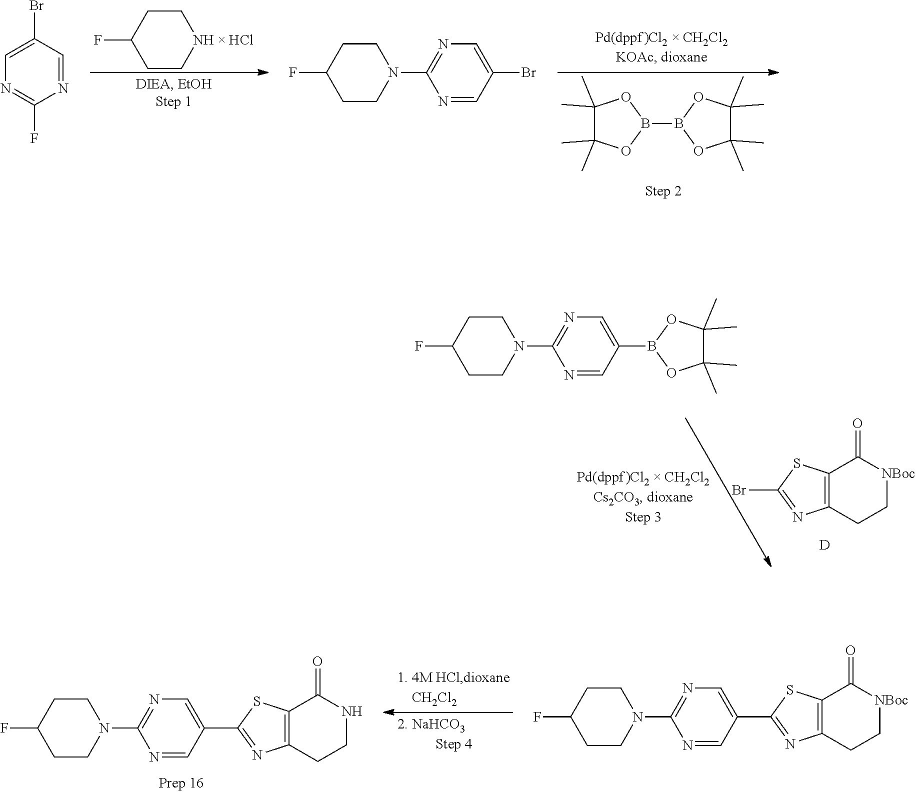

Step-1: Synthesis of 5-bromo-2-(4-fluoropiperidin-1-yl)pyrimidine)pyrazine Commercially available 2-iodo-5-bromo pyrimidine (2.0 g, 7.02 mmol), 4-fluoro piperidine hydrochloride salt (1.47 g, 10.5 mmol), and DIEA (3.4 mL, 2.64 g, 2.1 mmol) were suspended in ethanol (20 mL) using a microwave vial. The sealed vial was then heated at 120° C. for 1.5 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (1.77 g, 97%). ¹H NMR (400 MHz DMSO-d₆): δ 8.44 (s, 2H), 4.91 (m, 1H), 3.86 (m, 2H), 3.71 (m, 2H), 1.89 (m, 2H), 1.69 (m, 2H).

### Step-2: Synthesis of 2-(4-fluoropiperidin-1-yl)-5-(4,4,5,5-tetramethyl-1,3,2-dioxaborolan-2-yl)pyrimidine

The title compound from Step 1 above (1.77 g, 8.8 mmol) was dissolved in 1,4-dioxane (36 mL) and degassed by passing a stream of nitrogen through the mixture. Then [1,1′-bis(diphenyl-phosphino)ferrocene]-dichloropalladium(II), complex with dichloromethane (0.55 g, 0.68 mmol), bispinacolatodiboron (3.4 g, 13.6 mmol) and potassium acetate (1.3 g, 13.6 mmol) were added, and the reaction mixture was heated at 100° C. in an oil-bath for 5 h. The reaction mixture was washed three times with n-hexane (100 mL) and concentrated under reduced pressure to obtain the title compound as a brown solid (5.0 g, quant.). LCMS (ESI) 307.15 m/z [M+H]⁺.

### Step-3: Synthesis of tert-butyl 2-(2-(4-fluoropiperidin-1-yl)pyrimidin-5-yl)-4-oxo-6,7-dihydrothiazolo[5,4-c]pyridine-5(4H)-carboxylate

Compound D (0.2 g, 0.6 mmol) was dissolved in 1,4-dioxane (10 mL) and the solution was degassed by a stream of nitrogen. Then [1,1′-bis(diphenyl-phosphino)ferrocene]-dichloropalladium(II), complex with dichloromethane (0.05 g, 0.06 mmol), the title compound from Step 2 above (0.885 g, 2.88 mmol) and cesium carbonate (0.585 g, 1.8 mmol) were added, and the reaction mixture was heated at 100° C. in an oil-bath for 8 h. The reaction mixture was concentrated under reduced pressure. The residue was purified by column chromatography on silica gel (basic, 100-200 mesh) using ethyl acetate/n-hexane gradient (0/100->10/90->20/80) to obtain the title compound as a pale-yellow solid (0.09 g, 34%). ¹H NMR (400 MHz DMSO-d₆): δ 8.95 (s, 2H), 4.96 (dt, 1H), 4.06 (t, 2H), 3.94 (m, 5H), 3.11 (t, 2H), 1.94 (m, 2H) 1.75 (m, 2H), 1.49 (s, 9H), 1.27 (s, 1H), 1.07 (s, 4H). LCMS (ESI) 378.3 m/z [M+H-Boc]⁺.

### Step-4: Synthesis of 2-(2-(4-fluoropiperidin-1-yl)pyrimidin-5-yl)-6,7-dihydrothiazolo[5,4-c]pyridin-4(5H)-one (Prep 16)

The title compound from Step 3 above (0.09 g, 0.2 mmol) was dissolved in CH₂Cl₂ (4.5 mL) and the mixture was cooled to 0° C. in an ice-bath. Then a 4 M solution of HCl in 1,4-dioxane (0.9 mL) was added and stirring was continued at room temperature for 2 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 16 as a pale-yellow solid (0.064 g, 92%). ¹H NMR (500 MHz DMSO-d₆): δ 8.92 (s, 2H), 7.87 (s, 1H), 4.95 (m, 1H), 3.93 (m, 4H), 3.50 (td, 2H), 3.0 (t, 2H) 1.94 (qt, 2H), 1.75 (m, 2H). LCMS (ESI) 334.25 m/z [M+H]⁺.

### Synthesis of Building Block (S)-2-(2-(3-fluoropiperidin-1-yl)pyrimidin-5-yl)-6,7-dihydrothiazolo[5,4-c]pyridin-4(5H)-one (Prep 17)

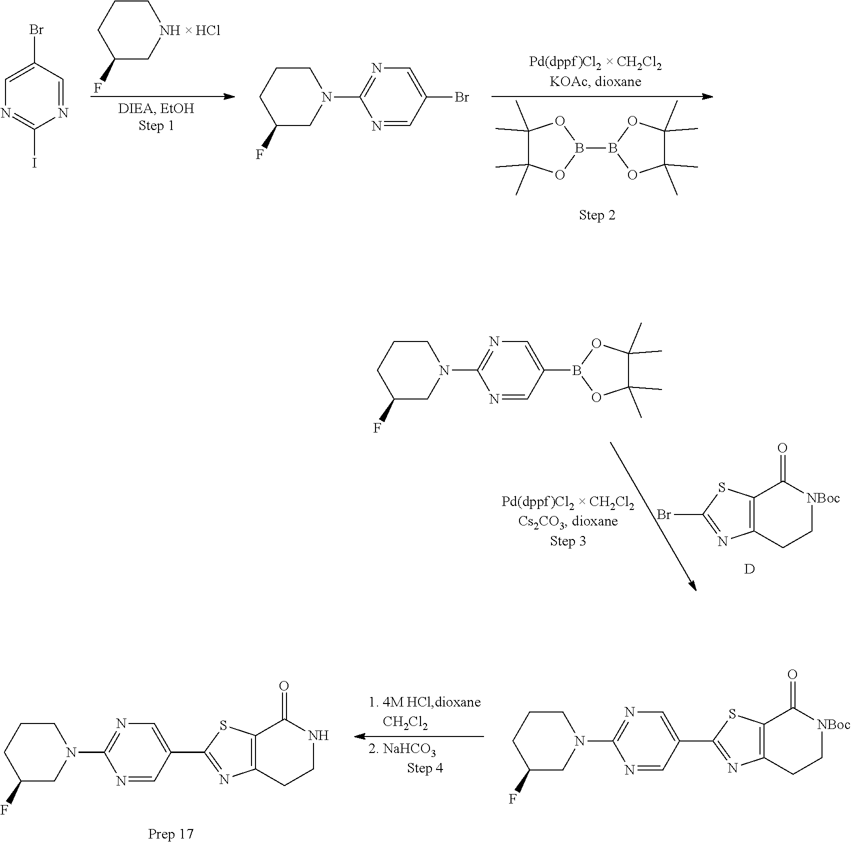

Step-1: Synthesis of (S)-5-bromo-2-(3-fluoropiperidin-1-yl)pyrimidine Commercially available 2-iodo-5-bromo pyrimidine (1.5 g, 5.28 mmol), (S)-3-fluoro piperidine hydrochloride salt (1.1 g, 7.92 mmol), and DIEA (2.6 mL, 1.98 g, 15.8 mmol) were suspended in ethanol (37 mL) using a microwave vial. The sealed vial was then heated at 120° C. for 1.5 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (1.35 g, 99%). ¹H NMR (500 MHz DMSO-d₆): δ 8.44 (s, 1H), 4.77 (m, 1H), 4.23 (m, 1H), 4.05 (dt, 1H), 3.64 (qd, 1H), 3.40 (t, 1H), 1.89 (m, 2H), 1.70 (m, 1H), 1.52 (m, 1H). LCMS (ESI) 259.7/261.75 m/z [M+H]⁺.

### Step-2: Synthesis of (S)-2-(3-fluoropiperidin-1-yl)-5-(4,4,5,5-tetramethyl-1,3,2-dioxaborolan-2-yl)pyrimidine

The title compound from Step 1 above (1.35 g, 5.23 mmol) was dissolved in 1,4-dioxane (27 mL) and degassed by passing a stream of nitrogen through the mixture. Then [1,1′-bis(diphenyl-phosphino)ferrocene]-dichloropalladium(II), complex with dichloromethane (0.42 g, 0.52 mmol), bispinacolatodiboron (2.6 g, 10. mmol) and potassium acetate (1 g, 10.4 mmol) were added, and the reaction mixture was heated at 100° C. in an oil-bath for 5 h. The reaction mixture was washed three times with n-hexane (100 mL) and concentrated under reduced pressure to obtain the title compound as a brown solid (5.0 g, quant.). LCMS (ESI) 308.28 m/z [M+H]⁺.

### Step-3: Synthesis of tert-butyl (S)-2-(2-(3-fluoropiperidin-1-yl)pyrimidin-5-yl)-4-oxo-6,7-dihydrothiazolo[5,4-c]pyridine-5(4H)-carboxylate

Compound D (1.0 g, 3 mmol) was dissolved in 1,4-dioxane (15 mL) and the solution was degassed by a stream of nitrogen. Then [1,1′-bis(diphenyl-phosphino)ferrocene]-dichloropalladium(II), complex with dichloromethane (0.245 g, 0.3 mmol), the title compound from Step 2 above (3.2 g, 12.6 mmol) and cesium carbonate (2.9 g, 9 mmol) were added, and the reaction mixture was heated at 100° C. in an oil-bath for 5 h. The reaction mixture was concentrated under reduced pressure. The residue was purified by column chromatography on silica gel (basic, 100-200 mesh) using ethyl acetate/n-hexane gradient (0/100->10/90->20/80) to obtain the title compound as a pale-yellow solid (0.98 g, 75%). ¹H NMR (400 MHz DMSO-d₆): δ 8.94 (s, 1H), 4.84 (m, 1H), 4.49 (m, 1H), 4.31 (td, 1H), 4.06 (t, 2H), 3.71 (dd, 1H) 3.47 (m, 1H), 3.11 (t, 2H), 1.92 (m, 2H), 1.73 (m, 1H), 1.60 (m, 1H), 1.49 (s, 9H). LCMS (ESI) 434.0 m/z [M+H]⁺.

### Step-4: Synthesis of (S)-2-(2-(3-fluoropiperidin-1-yl)pyrimidin-5-yl)-6,7-dihydrothiazolo[5,4-c]pyridin-4(5H)-one (Prep 17)

The title compound from Step 3 above (0.98 g, 2.26 mmol) was dissolved in CH₂Cl₂ (20 mL) and the mixture was cooled to 0° C. in an ice-bath. Then a 4 M solution of HCl in 1,4-dioxane (9.8 mL) was added and stirring was continued at room temperature for 2 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford the title compound Prep 17 as a pale-yellow solid (0.67 g, 89%). ¹H NMR (400 MHz DMSO-d₆): δ 8.91 (s, 1H), 7.86 (s, 1H), 4.84 (m, 1H), 4.45 (m, 1H), 4.26 (m, 1H), 3.73 (m, 1H) 3.48 (m, 3H), 3.00 (t, 1H), 1.91 (m, 2H), 1.75 (m, 1H), 1.57 (m, 1H). LCMS (ESI) 334.0 m/z [M+H]⁺.

### Synthesis of Building Block (S)-2-(2-(3-fluoropiperidin-1-yl)pyrimidin-5-yl)-6,7-dihydrothiazolo[5,4-c]pyridin-4(5H)-one (Prep 18)

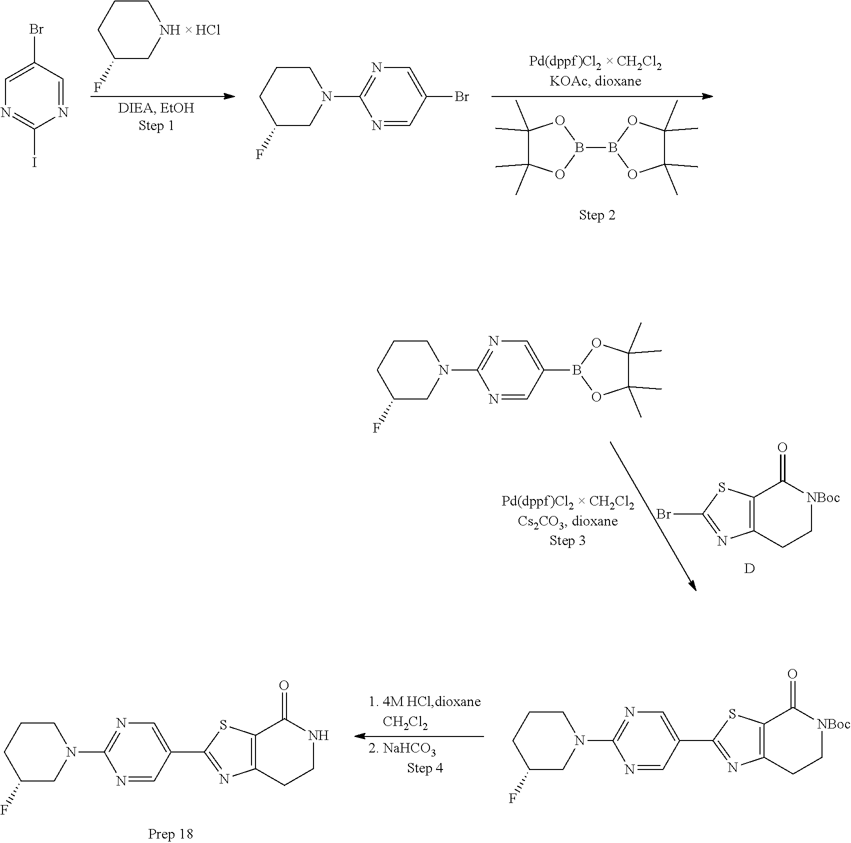

Step-1: Synthesis of (R)-5-bromo-2-(3-fluoropiperidin-1-yl)pyrimidine Commercially available 2-iodo-5-bromo pyrimidine (1 g, 3.52 mmol), (R)-3-fluoro piperidine hydrochloride salt (0.73 g, 5.28 mmol), and DIEA (1.7 mL, 1.3 g, 5.28 mmol) were suspended in ethanol (25 mL) using a microwave vial. The sealed vial was then heated at 120° C. for 1 h in a CEM microwave. The reaction mixture was concentrated under reduced pressure. The residue obtained was suspended in water (70 mL) and filtered over Whatmann filter paper. The solid was washed with water (20 mL) and dried under reduced pressure to obtain the title compound as an off-white solid (0.9 g, 99%). ¹H NMR (500 MHz DMSO-d₆): δ 8.44 (s, 2H), 4.77 (m, 1H), 4.23 (m, 1H), 4.05 (td, 1H), 3.64 (qd, 1H), 3.40 (m, 1H), 1.88 (m, 2H), 1.70 (qd, 1H), 1.53 (m, 1H). LCMS (ESI) 260.00/261.96 m/z [M+H]⁺.

### Step-2: Synthesis of (R)-2-(3-fluoropiperidin-1-yl)-5-(4,4,5,5-tetramethyl-1,3,2-dioxaborolan-2-yl)pyrimidine

The title compound from Step 1 above (0.9 g, 3.48 mmol) was dissolved in 1,4-dioxane (18 mL) and degassed by passing a stream of nitrogen through the mixture. Then [1,1′-bis(diphenyl-phosphino)ferrocene]-dichloropalladium(II), complex with dichloromethane (0.28 g, 0.34 mmol), bispinacolatodiboron (1.7 g, 6.97. mmol) and potassium acetate (0.68 g, 6.97 mmol) were added, and the reaction mixture was heated at 100° C. in an oil-bath for 5 h. The reaction mixture was washed three times with n-hexane (100 mL) and concentrated under reduced pressure to obtain the title compound as a brown solid (2.7 g, quant.). LCMS (ESI) 308.44 [M+H]⁺.

### Step-3: Synthesis of tert-butyl (R)-2-(2-(3-fluoropiperidin-1-yl)pyrimidin-5-yl)-4-oxo-6,7-dihydrothiazolo[5,4-c]pyridine-5(4H)-carboxylate

Compound D (0.5 g, 1.5 mmol) was dissolved in 1,4-dioxane (25 mL) and the solution was degassed by a stream of nitrogen. Then [1,1′-bis(diphenyl-phosphino)ferrocene]-dichloropalladium(II), complex with dichloromethane (0.12 g, 0.15 mmol), the title compound from Step 2 above (1.8 g, 6 mmol) and cesium carbonate (1.46 g, 4.5 mmol) were added, and the reaction mixture was heated at 100° C. in an oil-bath for 5 h. The reaction mixture was concentrated under reduced pressure. The residue was purified by column chromatography on silica gel (basic, 100-200 mesh) using ethyl acetate/n-hexane gradient (05/95->15/85->30/70) to obtain the title compound as a pale-yellow solid (0.474 g, 73%). ¹H NMR (400 MHz DMSO-d₆): δ 8.94 (s, 2H), 4.84 (m, 1H), 4.49 (m, 1H), 4.31 (m, 1H), 4.06 (t, 2H), 3.71 (dd, 1H), 3.47 (m, 1H), 3.11 (t, 2H), 1.92 (m, 2H), 1.73 (m, 1H), 1.60 (m, 1H), 1.49 (s, 9H). LCMS (ESI) 434.1 [M+H]⁺.

### Step-4: Synthesis of (R)-2-(2-(3-fluoropiperidin-1-yl)pyrimidin-5-yl)-6,7-dihydrothiazolo[5,4-c]pyridin-4(5H)-one (Prep 18)

The title compound from Step 3 above (0.47 g, 1.08 mmol) was dissolved in CH₂Cl₂ (10 mL) and the mixture was cooled to 0° C. in an ice-bath. Then a 4 M solution of HCl in 1,4-dioxane (4.7 mL) was added and stirring was continued at room temperature for 2 h. After completion of the reaction, the solvent was removed under reduced pressure. The residue was dissolved in ice cold water and basified with an aqueous saturated sodium bicarbonate solution to pH 8-9, the precipitate was collected by filtration, and dried to afford title compound Prep 18 as a pale-yellow solid (0.3 g, 83%). ¹H NMR (400 MHz DMSO-d₆): δ 7.85 (s, 1H), 4.83 (m, 1H), 4.45 (m, 1H), 4.26 (m, 1H), 3.72 (dd, 1H), 3.48 (m, 3H), 3.00 (t, 2H), 1.90 (m, 2H), 1.73 (m, 1H), 1.58 (dd, 1H). LCMS (ESI) 334.0 [M+H]⁺.

### Examples 42 to 50

Following the coupling procedure as reported in Example 1 and using the building blocks and halogen derivatives indicated in Table 5, the following compounds were prepared.

TABLE 5

| | | | | 1. Yield |
|---|---|---|---|---|
| | | | | 2. ¹H-NMR |
| | | Halogen | | 3. MH⁺ (ESI) |
| Example | Building block | derivative | Product | 4. Synthesis procedure |
| 42 | 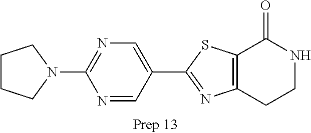 [SMILES: O=C1NCCc2nc(-c3cnc(N4CCCC4)nc3)sc21] | 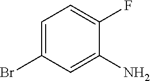 | 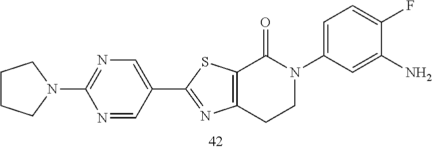 [SMILES: Nc1cc(N2CCc3nc(-c4cnc(N5CCCC5)nc4)sc3C2=O)ccc1F] | 1. 38% 2. ¹H NMR (400 MHZ, DMSO-d₆): δ 8.93 (s, 2H), 7.01 (dd, 1H), 6.76 (dd, 1H), 6.49 (dd, 1H), 5.25 (s, 2H), 3.99 (t, 2H), 3.58 (t, 4H), 3.19 (t, 2H), 1.97 (m, 4H) 3. 411.2 4. Example 1 |
| 43 | 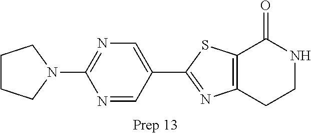 [SMILES: O=C1NCCc2nc(-c3cnc(N4CCCC4)nc3)sc21] | 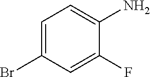 | 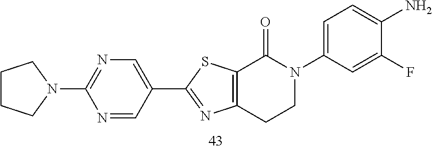 [SMILES: Nc1ccc(N2CCc3nc(-c4cnc(N5CCCC5)nc4)sc3C2=O)cc1F] | 1. 30% 2. ¹H NMR (400 MHZ, DMSO-d₆): δ 8.92 (s, 2H), 7.07 (dd, 1H), 6.90 (dd, 1H), 6.76 (m, 1H), 5.18 (d, 2H), 3.98 (t, 2H), 3.57 (t, 4H), 3.19 (t, 2H), 1.97 (m, 4H) 3. 411.0 4. Example 1 |
| 44 | 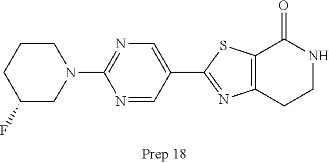 [SMILES: O=C1NCCc2nc(-c3cnc(N4CCC[C@@H](F)C4)nc3)sc21] | 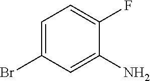 | 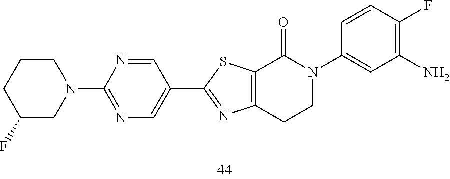 [SMILES: Nc1cc(N2CCc3nc(-c4cnc(N5CCC[C@@H](F)C5)nc4)sc3C2=O)ccc1F] | 1. 91% 2. ¹H NMR (400 MHZ, DMSO-d₆): δ 8.94 (s, 2H), 7.01 (dd, 1H), 6.76 (dd, 1H), 6.49 (dd, 1H), 5.25 (s, 2H), 4.85 (dt, 1H), 4.48 (m, 1H), 4.30 (m, 1H), 3.99 (t, 2H), 3.72 (dd, 1H), 3.48 (m,1H), 3.20 (t, 2H), 1.92 (m, 2H), 1.67 (m, 2H) 3. 443.0 4. Example 1 |
| 45 | 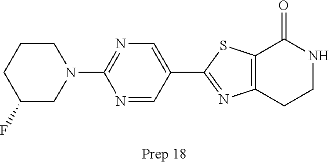 [SMILES: O=C1NCCc2nc(-c3cnc(N4CCC[C@@H](F)C4)nc3)sc21] | 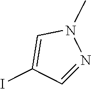 | 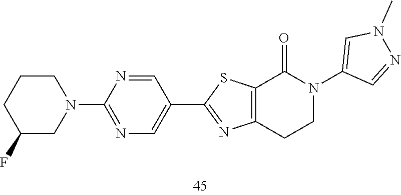 [SMILES: Cn1cc(N2CCc3nc(-c4cnc(N5CCC[C@H](F)C5)nc4)sc3C2=O)cn1] | 1. 30% 2. ¹H NMR (400 MHZ, DMSO-d₆): δ 8.94(s, 2H), 8.06 (s, 1H), 7.68 (s, 1H), 4.85 (m, 1H), 4.48 (m, 1H), 4.30 (m, 1H), 4.08 (t, 2H), 3.84 (s,3H), 3.71 (dd,1H), 3.49 (s, 1H), 3.23 (t, 2H), 1.92 (m, 2H), 1.67 (m, 2H) 3. 414.0 4. Example 1 |
| 46 | 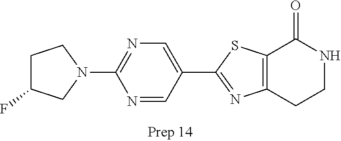 [SMILES: O=C1NCCc2nc(-c3cnc(N4CC[C@@H](F)C4)nc3)sc21] | 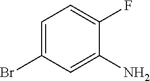 | 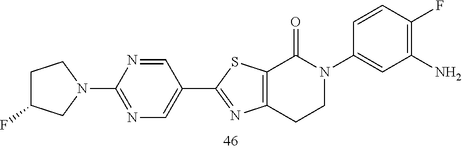 [SMILES: Nc1cc(N2CCc3nc(-c4cnc(N5CC[C@@H](F)C5)nc4)sc3C2=O)ccc1F] | 1. 87% 22. ¹H NMR (500 MHZ, DMSO-d₆): δ 8.97 (d, 2H), 7.01 (dd, 1H), 6.76 (dd, 1H), 6.50 (td, 1H), 5.48 (d, 1H), 5.25 (s, 2H), 4.00 (t, 2H), 3.90 (m, 2H), 3.75 (dd, 1H), 3.59 (td, 1H), 3.20 (t,2H), 2.24 (m,2H) 3. 429.1 4. Example 1 |
| 47 | 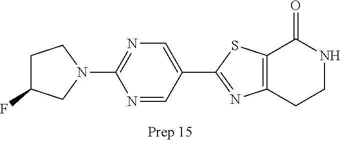 [SMILES: O=C1NCCc2nc(-c3cnc(N4CC[C@H](F)C4)nc3)sc21] | 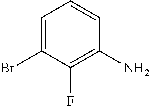 | 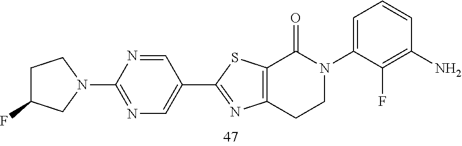 [SMILES: Nc1cccc(N2CCc3nc(-c4cnc(N5CC[C@H](F)C5)nc4)sc3C2=O)c1F] | 1. 72% 2. ¹H NMR (400 MHZ, DMSO-d₆): δ 8.98 (d, 2H), 6.91 (t, 1H), 6.73 (td, 1H), 6.56 (m, 1H), 6.48 (m, 1H), 5.26 (d, 2H), 3.97 (t, 2H), 3.89 (m, 2H), 3.75 (dd, 1H), 3.59 (td, 1H), 3.22 (t, 2H), 2,23 (m, 2H) 3. 428.95 4. Example 1 |
| 48 | 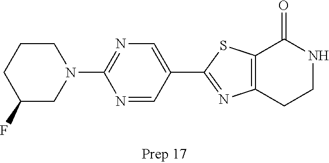 [SMILES: O=C1NCCc2nc(-c3cnc(N4CCC[C@H](F)C4)nc3)sc21] | 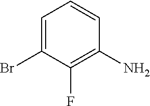 | 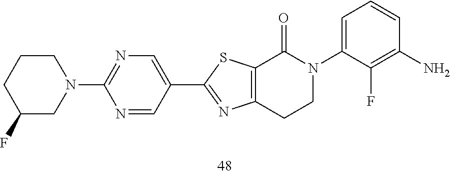 [SMILES: Nc1cccc(N2CCc3nc(-c4cnc(N5CCC[C@H](F)C5)nc4)sc3C2=O)c1F] | 1. 51% 2. ¹H NMR (500 MHZ, DMSO-d₆): δ 8.95 (s, 2H), 6.90 (t, 1H), 6.73 (t, 1H), 6.56 (t, 1H), 5.26 (d, 2H), 4.85 (d, 1H), 4.48 (m, 1H), 4.30 (q, 1H), 3.97 (t, 2H), 3.72 (dd, 1H), 3.48 (t, 1H), 3.22 (t, 2H), 1.92 (m, 2H), 1.75 (m, 1H), 1.59 (m, 1H) 3. 443.25 4. Example 1 |
| 49 | 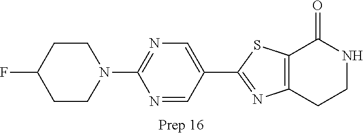 [SMILES: O=C1NCCc2nc(-c3cnc(N4CCC(F)CC4)nc3)sc21] | 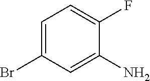 | 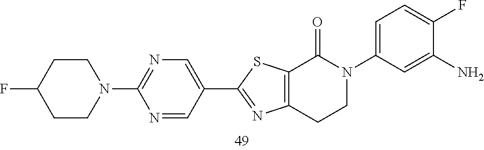 [SMILES: Nc1cc(N2CCc3nc(-c4cnc(N5CCC(F)CC5)nc4)sc3C2=O)ccc1F] | 1. 96% 2. ¹H NMR (400 MHZ, DMSO-d₆): δ 8.95 (s, 2H), 7.01 (dd, 1H), 6.76 (dd, 1H), 6.50 (dd, 1H), 5.24 (s, 2H), 4.96 (m, 1H), 3.99 (m, 4H), 3.90 (m, 2H), 3.20 (t, 2H), 1.86 (m, 4H) 3. 443.3 4. Example 1 |
| 50 | 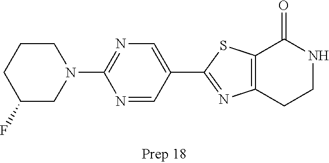 [SMILES: O=C1NCCc2nc(-c3cnc(N4CCC[C@@H](F)C4)nc3)sc21] | 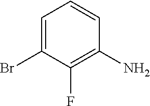 | 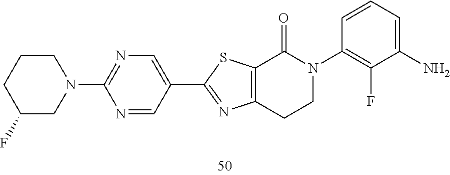 [SMILES: Nc1cccc(N2CCc3nc(-c4cnc(N5CCC[C@@H](F)C5)nc4)sc3C2=O)c1F] | 1. 77% 2. ¹H NMR (400 MHZ, DMSO-d₆): δ 8.95 (s, 2H), 6.90 (t, 1H), 6.73 (dt, 1H), 6.56 (m, 1H), 5.25 (d, 2H), 4.85 (dt, 1H), 4.48 (m, 1H), 4.29 (td, 1H), 3.97 (t,2H), 3.72 (dd, 1H), 3.48 (m, 1H), 3.22 (t, 2H), 3.16 (m, 1H), 1.92 (m, 2H), 1.74 (m, 1H), 1.59 (m, 1H) 3. 442.85 4. Example 1 |

### Example 51 Synthesis scheme of (R)-2-(5-bromo-6-(3-fluoropyrrolidin-1-yl)pyridin-3-yl)-5-(5-bromopyridin-3-yl)-6,7-dihydrothiazolo[5,4-c]pyridin-4(5H)-one (precursor of ³H Compound 52)

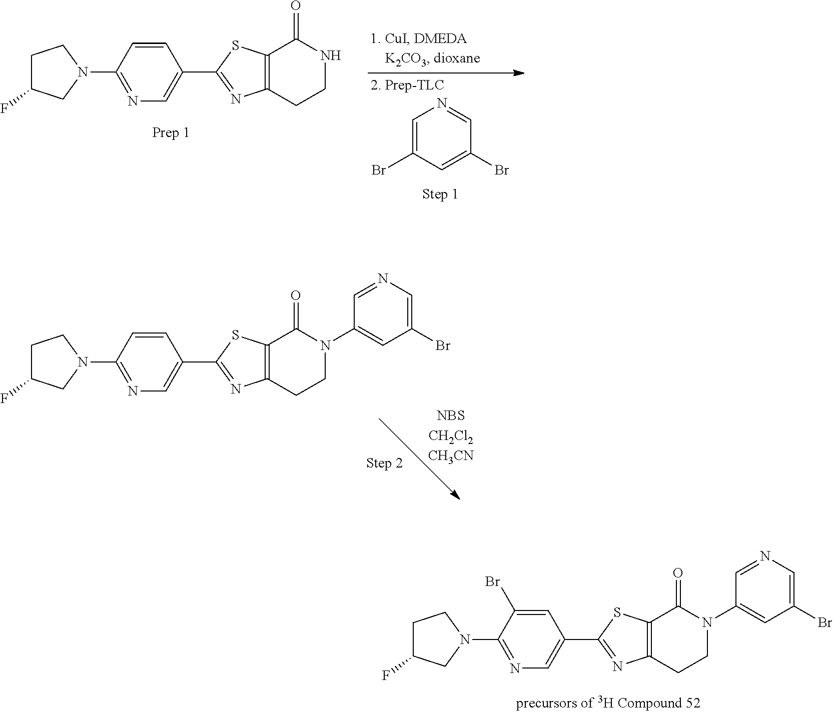 [SMILES: O=C1NCCc2nc(-c3ccc(N4CC[C@@H](F)C4)nc3)sc21 ; Brc1cncc(Br)c1 ; O=C1c2sc(-c3ccc(N4CC[C@@H](F)C4)nc3)nc2CCN1c1cncc(Br)c1 ; O=C1c2sc(-c3cnc(N4CC[C@@H](F)C4)c(Br)c3)nc2CCN1c1cncc(Br)c1]

### Step-1: Synthesis of (R)-5-(5-bromopyridin-3-yl)-2-(6-(3-fluoropyrrolidin-1-yl)pyridin-3-yl)-6,7-dihydrothiazolo[5,4-c]pyridin-4(5H)-one

Compound Prep 1 (0.046 g, 0.144 mmol), 3,5-dibromopyridine (0.112 g, 0-472 mmol), copper(I)iodide (0.003 g, 0.0157 mmol), N,N′-dimethylethylenediamine (0.025 mL, 0.27 mmol) and potassium carbonate (0.043 g, 0.314 mmol) were suspended in 1,4-dioxane (9 mL). The reaction mixture was heated at −120° C. in a sand-bath for 24 h. The reaction mixture of two runs was diluted with ethyl acetate (200 mL) and washed with water (80 mL) and brine (80 mL). The organic phase was separated, dried over Na₂SO₄, filtered and the solvents were removed under reduced pressure. The residue was purified by chromatography on silica (25 g HP-Ultra) using a Biotage Isolera system employing a CH₂Cl₂/MeOH (100/0->98/2->93/7->96/4->95/5->90/10) to obtain the precursor of ³H Compound 52 as a yellow solid (0.0814 g). The title compound was further purified by preparative TLC plates using CH₂Cl₂/MeOH (95/5) as a mobile phase to obtain the title compound as a yellow solid (0.0677 g, 49%). ¹H NMR (400 MHz, DMSO-d₆): δ 8.79 (d, 1H), 8.68 (d, 1H), 8.59 (d, 1H), 8.18 (t, 1H), 8.10 (dd, 1H), 6.66 (d, 1H), 5.49 (d, 1H), 4.18 (t, 2H), 3.88-3.62 (m, 3H), 3.55-3.49 (m, 1H), 3.25 (t, 2H), 2.33-2.25 (m, 2H). LCMS (ESI) 474.01/475.97 m/z [M+H]⁺

### Step-2: Synthesis of (R)-2-(5-bromo-6-(3-fluoropyrrolidin-1-yl)pyridin-3-yl)-5-(5-bromopyridin-3-yl)-6,7-dihydrothiazolo[5,4-c]pyridin-4(5H)-one (precursor of ³H Compound 52)

The title compound from Step 1 above (0.0677 g, 0.143 mmol) was dissolved in CH₂Cl₂ (3.5 mL) and acetonitrile (3.5 mL). The mixture was cooled to 0° C., N-bromosuccinimide (0.101 g, 0.574 mmol) was added, and the reaction mixture was stirred at 0° C. for 3 h. The reaction mixture was diluted with ethyl acetate (80 mL) and washed with water/brine (40 mL; 1/1). The organic phase was separated, dried over Na₂SO₄, filtered and the solvents were removed under reduced pressure. The residue was purified by chromatography on silica (25 g Interchim) using a Biotage Isolera system employing an ethyl acetate/n-heptane gradient (5/95->80/20->80/20) to obtain the precursor of ³H Compound 52 as a yellow solid. The precursor of ³H Compound 52 was treated with methanol (3 mL), sonicated for 1 min, and the solvent was evaporated under reduced pressure to obtain the precursor of ³H Compound 52 as a yellow solid (0.0481 g, 60%). ¹H NMR (400 MHz, DMSO-d₆): δ 8.77 (d, 1H), 8.68 (d, 1H), 8.60 (d, 1H), 8.37 (d, 1H), 8.18 (t, 1H), 5.45 (d, 1H), 4.19 (t, 2H), 4.07 (ddd, 1H), 3.98-3.84 (m, 3H), 3.26 (t, 2H), 2.28-2.07 (m, 2H). LCMS (ESI) 552.09/554.06/556.01 m/z [M+H]⁺

### Example 52 Synthesis of tritiated (R)-2-(6-(3-fluoropyrrolidin-1-yl)pyridin-3-yl)-5-(pyridin-3-yl)-6,7-dihydrothiazolo[5,4-c]pyridin-4(5H)-one (³H-Compound 52)

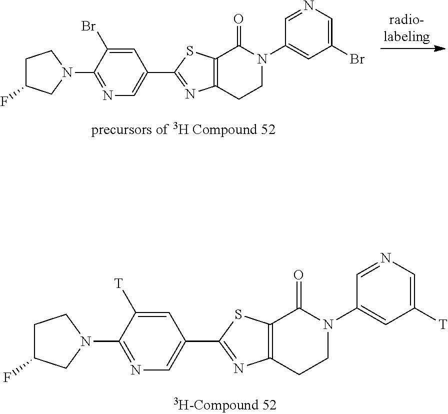

Experimental Procedure for the Halogen Vs. Tritium Exchange:

10% Palladium on charcoal (0.5 mg) was added to a tritium reaction vessel, followed by a solution of precursors of ³H Compound 52 (0.5 mg) in DMF (0.3 mL), then DIEA (0.005 mL). The vessel was attached to the tritium line and pressurized to 0.5 atm with tritium gas at −200° C. The solution was stirred for 2 h at room temperature, cooled to −200° C. and excess gas was removed. The reaction flask was rinsed with 4×1 mL methanol passing each of the methanol washes through a celite pad. The combined methanol was removed under vacuum. Crude yield: 30 mCi. The material was purified by HPLC, mobile phase was removed under vacuum and compound ³H-Compound 52 was redissolved in absolute ethanol. Yield 15 mCi, purity >99%. The specific activity was determined to be 47.8 Ci/mmol by MS.

### Example 53 Synthesis of 2-amino-3,5-dibromo-6-(2-(5-bromo-6-(pyrrolidin-1-yl)pyridin-3-yl)-4-oxo-6,7-dihydrothiazolo[5,4-c]pyridin-5(4H)-yl)benzonitrile (precursor of ³H Compound 16)

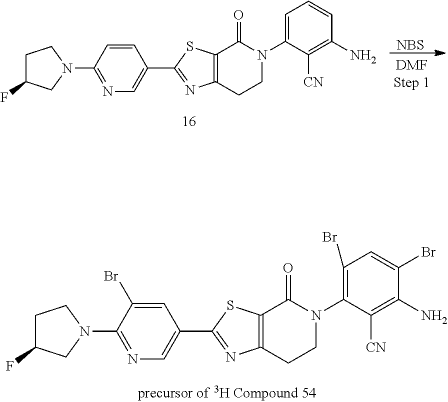

Compound 16 (0.15 g, 0.36 mmol) was dissolved in DMF (10 mL). The mixture was cooled to −50° C. and N-bromosuccinimide (0.224 g, 1.26 mmol) was added in portions. The reaction mixture was allowed to warm to 0° C. and then to ambient temperature over a period of 1 h. The reaction mixture was diluted with ethyl acetate (80 mL) and water (100 mL). The organic phase was separated, dried over Na₂SO₄, filtered and the solvents were removed under reduced pressure. The residue was further purified by preparative TLC plates using CH₂Cl₂/MeOH (98/2) as a mobile phase to obtain the precursor of ³H Compound 16 (0.033 g, 15%). ¹H NMR (500 MHz, DMSO-d₆): δ 8.73 (d, 1H), 8.32 (d, 1H), 8.11 (s, 1H), 6.55 (d, 2H), 3.97 (m, 2H), 3.73 (s, 4H), 3.25 (m, 2H), 3.26 (m, 2H), 1.91 (s, 4H).

### Example 54 Synthesis scheme of tritium labeled 2-amino-6-(4-oxo-2-(6-(pyrrolidin-1-yl)pyridin-3-yl)-6,7-dihydrothiazolo[5,4-c]pyridin-5(4H)-yl)benzonitrile (³H-Compound 16)

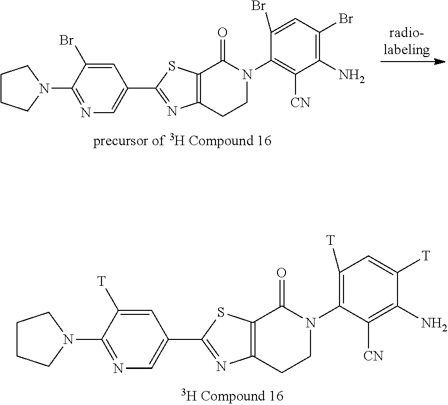

Experimental Procedure for the Halogen Vs. Tritium Exchange:

Precursor of ³H Compound 16 (1.82 mg), 8.53 mg of Pd/C (10% metal) and 10 μl DIEA were suspended in 0.4 ml DMF. The suspension was degassed three times at the high vacuum manifold and stirred under an atmosphere of tritium gas (5.9 Ci) for 1 h at room temperature. The pressure started at 620 mbar and ended at 516 mbar at room temperature. The solvent was removed in vacuo, and the labile tritium was exchanged by adding methanol/CH₂Cl₂ (0.3 mL, 1/1), stirring the solution, and removing the solvent again under reduced pressure. This process was repeated three times. Finally, the well dried solid was extracted with methanol/CH₂Cl₂ (5 mL, 1/1) and the suspension was filtered through a 0.2 μm nylon membrane, obtaining a clear yellow solution. The desired product was isolated from the HPLC solvent mixture by solid phase extraction. The HPLC-solution was neutralized with an aqueous solution of NaHCO₃, and the volumes of the fractions were partially reduced at the rotary evaporator. Then the product was extracted with a Phenomenex StrataX cartridge which was eluted with 5 ml of ethanol. The extracted ³H Compound 16 showed a radiochemical purity of >99% and the specific activity was determined to be 72.5 Ci/mmol by MS.

### Example 55 Synthesis of (S)-2-amino-3,5-dibromo-6-(2-(5-bromo-6-(3-fluoropyrrolidin-1-yl)pyridin-3-yl)-4-oxo-6,7-dihydrothiazolo[5,4-c]pyridin-5(4H)-yl)benzonitrile (precursor of ³H Compound 21)

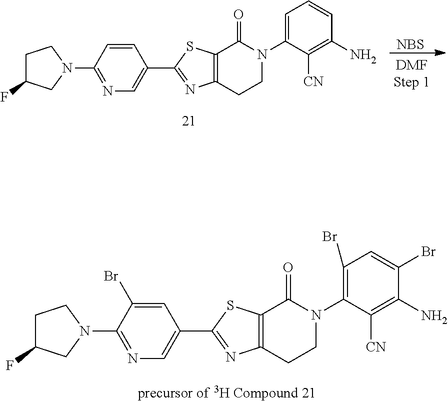

### Step-1: Synthesis of (S)-2-amino-3,5-dibromo-6-(2-(5-bromo-6-(3-fluoropyrrolidin-1-yl)pyridin-3-yl)-4-oxo-6,7-dihydrothiazolo[5,4-c]pyridin-5(4H)-yl)benzonitrile (precursor of ³H Compound 21)

Compound 21 (0.08 g, 0.18 mmol) was dissolved in DMF (10 mL), and the solution was cooled to −50° C. After the addition of N-bromosuccinimide (0.131 g, 0.737 mmol) in small portions, the reaction mixture was allowed to warm up to 20° C. over a period of 1 h. The reaction mixture was diluted with cold water (100 mL) and the precipitate was collected by filtration. The solid was purified by preparative TLC plates using CH₂Cl₂/MeOH/(98.5/1.5) as a mobile phase to obtain the precursor of ³H Compound 21 as a pale-yellow solid (0.038 g, 32%). ¹H NMR (500 MHz DMSO-d₆): δ 8.72 (d, 1H), 8.33 (d, 1H), 8.07 (s, 1H), 6.5 (s, 2H), 5.4 (d, 1H), 3.95 (m, 6H), 3.21 (m, 2H), 2.13 (m, 2H).

### Example 56 Synthesis scheme of tritium labeled (S)-2-amino-6-(2-(6-(3-fluoropyrrolidin-1-yl)pyridin-3-yl)-4-oxo-6,7-dihydrothiazolo[5,4-c]pyridin-5(4H)-yl)benzonitrile (³H Compound 21)

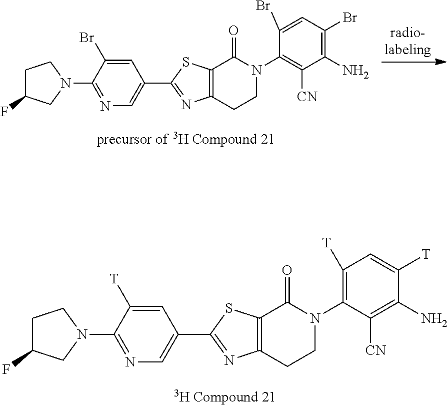

Experimental Procedure or the Halogen Vs. Tritium Exchange:

The precursor of ³H Compound 21 (3.94 mg), 45.1 mg of Lindlar's catalyst (10% metal) and 20 μl DIEA were suspended in 0.5 ml DMF. The suspension was degassed three times at the high vacuum manifold and stirred under an atmosphere of tritium gas (8.7 Ci) for 16 h at room temperature. The pressure started at 640 mbar and ended at 428 mbar at room temperature. The solvent was removed in vacuo, and the labile tritium was exchanged by adding methanol/CH₂Cl₂ (0.3 mL, 1/1), stirring the solution, and removing the solvent again under reduced pressure. This process was repeated three times. Finally, the well dried solid was extracted with methanol/CH₂Cl₂ (5 mL, 1/1) and the suspension was filtered through a 0.2 μm nylon membrane, obtaining a clear yellow solution. The desired product was isolated from the HPLC solvent mixture by solid phase extraction. The HPLC-solution was neutralized with an aqueous solution of NaHCO₃, and the volumes of the fractions were partially reduced at the rotary evaporator. Then the product was extracted with a Phenomenex StrataX cartridge which was eluted with 5 ml of ethanol. The extracted ³H Compound 21 showed a radiochemical purity of >99% and the specific activity was determined to be 64.4 Ci/mmol by MS.

### Example 57 Synthesis of (R)-1-(5-(5-(3-(bis(tert-butoxycarbonyl)amino)-4-fluorophenyl)-4-oxo-4,5,6,7-tetrahydrothiazolo[5,4-c]pyridin-2-yl)pyridin-2-yl)pyrrolidin-3-yl methanesulfonate (precursor of ¹⁸F Compound 26)

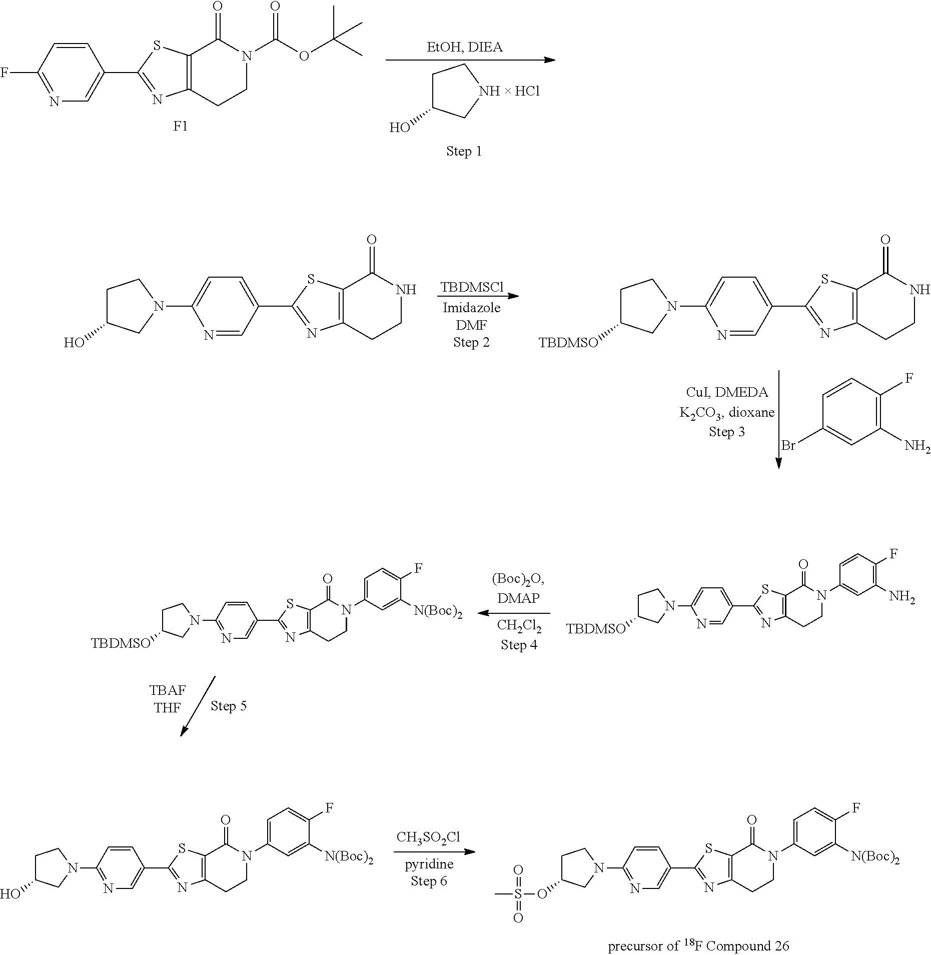

### Step-1: Synthesis of (R)-2-(6-(3-hydroxypyrrolidin-1-yl)pyridin-3-yl)-6,7-dihydrothiazolo[5,4-c]pyridin-4(5H)-one

Compound F1 (0.4 g, 1.14 mmol), (R)-pyrrolidin-3-ol hydrochloric acid salt (0.424 g, 3.4 mmol), and DIEA (1.2 mL, 0.88 g, 6.8 mmol) were suspended in n-butanol (20 mL) using a microwave vial. The sealed vial was then heated at 160° C. for 2 h in a CEM microwave. The reaction mixture was concentrated under reduced pressure. The residue obtained was suspended in water (20 mL) and filtered over Whatmann filter paper. The solid was washed with water (20 mL), followed by n-hexane (20 mL) and dried under reduced pressure to obtain the title compound as an off-white solid (0.29 g, 80%). ¹H NMR (400 MHz, DMSO-d₆): δ 8.7 (d, 1H), 8.01 (dd, 1H), 7.79 (s, 1H), 6.58 (d, 1H), 4.59 (s, 1H), 3.64 (dd, 1H), 3.52 (m, 4H), 3.3 (m, 1H), 2.98 (t, 2H), 2.02 (m, 2H), 0.86 (s, 9H), 0.2 (d, 2H). LCMS (ESI) 317.33 m/z [M+H]⁺

### Step-2: Synthesis of (R)-2-(6-(3-((tert-butyldimethylsilyl)oxy)pyrrolidin-1-yl)pyridin-3-yl)-6,7-dihydrothiazolo[5,4-c]pyridin-4(5H)-one

The title compound from Step 1 above (0.33 g, 1.04 mmol) and imidazole (0.75 g, 10.4 mmol) were dissolved in DMF (16.5 mL). The reaction mixture was cooled to 0° C. in an ice bath and tert-butyl dimethylsilyl chloride (0.8 g, 5.2 mmol) was added in portions. The reaction mixture was stirred at ambient temperature for 16 h. The reaction mixture was diluted with cold water (150 mL), the precipitate was collected by filtration and dried to obtain the title compound as a white solid (0.4 g, 83%). ¹H NMR (400 MHz, DMSO-d₆): δ 8.7 (d, 1H), 8.0 (dd, 1H), 7.79 (s, 1H), 6.58 (d, 1H), 4.59 (s, 1H), 3.64 (dd, 1H), 3.52 (m, 4H), 3.33 (m, 1H), 2.98 (t, 1H), 2.02 (m, 2H), 0.86 (s, 9H), 0.1 (d, 6H). LCMS (ESI) 431.41 m/z [M+H]⁺

### Step-3: Synthesis of (R)-5-(3-amino-4-fluorophenyl)-2-(6-(3-((tert-butyldimethylsilyl)oxy)pyrrolidin-1-yl)pyridin-3-yl)-6,7-dihydrothiazolo[5,4-c]pyridin-4(5H)-one

The title compound form Step 2 above (0.4 g, 0.92 mmol), 5-bromo-2-fluoroaniline (0.44 g, 2.32 mmol), copper(I)iodide (0.089 g, 0.46 mmol), N,N′-dimethylethylenediamine (0.1 mL, 0.92 mmol) and potassium carbonate (0.52 g, 3.7 mmol) were suspended in 1,4-dioxane (40 mL). The reaction mixture was heated at −120° C. in a sand-bath for 24 h. The reaction mixture was concentrated under reduced pressure to remove the solvent. The residue was purified by column chromatography on silica gel (100-200 mesh) using MeOH/CH₂Cl₂ gradient (0/100->1/99->2/98) to obtain the title compound as a pale-yellow solid (0.45 g, 90%). ¹H NMR (500 MHz, DMSO-d₆): δ 8.73 (d, 1H), 8.04 (dd, 1H), 7.0 (dd, 1H), 6.76 (dd, 1H), 6.6 (d, 1H), 6.49 (t, 1H), 5.22 (d, 1H), 4.59 (s, 1H), 3.98 (t, 2H), 3.59 (m, 3H), 3.3 (m, 1H), 3.17 (t, 2H), 2.11 (m, 1H), 1.92 (s, 1H), 0.86 (s, 9H), 0.1 (d, 6H). LCMS (ESI) 540.1 m/z [M+H]⁺

### Step-4: Synthesis of tert-butyl (R)-(tert-butoxycarbonyl)(5-(2-(6-(3-((tert-butyldimethylsilyl)oxy)pyrrolidin-1-yl)pyridin-3-yl)-4-oxo-6,7-dihydrothiazolo[5,4-c]pyridin-5(4H)-yl)-2-fluorophenyl)carbamate

The title compound from Step 3 above (0.45 g, 0.83 mmol), 4-(dimethylamino)pyridine (0.2 g, 1.6 mmol), and di-tert-butyl dicarbonate (0.86 mL, 0.81 g, 3.75 mmol) were suspended in CH₂Cl₂ (72 mL) at 0 C. Then the reaction mixture was stirred at room temperature for 24 h. The reaction mixture was diluted with water (30 mL) and MecOH (10 mL). The organic phase was separated, dried over Na₂SO₄, filtered and the solvents were removed under reduced pressure. The residue was purified by chromatography silica gel (basic 100-200 mesh) using a MeOH/CH₂Cl₂ gradient (0/100->0.5/99.5) to obtain the title compound as a pale-yellow solid (0.28 g, 36%). ¹H NMR (500 MHz, DMSO-d₆): δ 8.75 (d, 1H), 8.05 (m, 1H), 7.43 (m, 2H), 7.35 (t, 1H), 6.6 (d, 1H), 4.6 (s, 1H), 4.06 (t, 2H), 3.37 (m, 4H), 3.22 (t, 2H), 2.1-1.9 (m, 2H), 1.4 (s, 18H), 0.86 (s, 9H), 0.1 (d, 6H). LCMS (ESI) 740.57 m/z [M+H]⁺

### Step-5: Synthesis of tert-butyl (R)-(tert-butoxycarbonyl)(2-fluoro-5-(2-(6-(3-hydroxypyrrolidin-1-yl)pyridin-3-yl)-4-oxo-6,7-dihydrothiazolo[5,4-c]pyridin-5(4H)-yl)phenyl)carbamate

The title compound from Step 4 above (0.28 g, 0.37 mmol) was dissolved in THF (6 mL) and cooled to −20° C. in a dry ice-bath with stirring. Then tetrabutylammonium fluoride (0.196 g, 0.75 mmol) was added and stirring was continued at room temperature for 2.5 h. The reaction mixture was diluted with water (20 mL) and extracted with 10% MeOH in CH₂Cl₂ (3×20 mL). The organic phase was separated, dried over Na₂SO₄, filtered and the solvents were removed under reduced pressure. The residue was purified by chromatography silica gel (basic 100-200 mesh) using a MeOH/CH₂Cl₂ gradient (0/100->1/99->2/98) to obtain the title compound as a pale-yellow solid (0.2 g, 87%). ¹H NMR (500 MHz, DMSO-d₆): δ 8.70 (d, 1H), 8.0 (dd, 1H), 7.39 (m, 2H), 7.3 (d, 1H), 6.53 (d, 1H), 5.0 (d, 1H), 4.37 (s, 1H), 4.02 (t, 2H), 3.49 (m, 3H), 3.3 (m, 1H), 1.9 (m, 2H), 1.36 (s, 18H). LCMS (ESI) 626.48 m/z [M+H]⁺.

### Step-6: Synthesis of (R)-1-(5-(5-(3-(bis(tert-butoxycarbonyl)amino)-4-fluorophenyl)-4-oxo-4,5,6,7-tetrahydrothiazolo[5,4-c]pyridin-2-yl)pyridin-2-yl)pyrrolidin-3-yl methanesulfonate (precursor of ¹⁸F Compound 26)

The title compound from Step 5 above (0.19 g, 0.30 mmol) was dissolved in pyridine (7 mL) and cooled to −50° C. in a dry ice-bath with stirring. Then methanesulfonyl chloride (0.514 g, 4.5 mmol) was added and stirring was continued at room temperature for 1.5 h. The reaction mixture was diluted with water (35 mL), the precipitate was collected by filtration, washed with water (2×15 mL, and dried to obtain the precursor of ¹⁸F Compound 26 as a pale-yellow solid (0.2 g, 94%). ¹H NMR (500 MHz, DMSO-d₆): δ 8.77 (d, 1H), 8.1 (dd, 1H), 7.43 (m, 2H), 7.3 (t, 1H), 6.56 (d, 1H), 5.45 (s, 1H), 4.06 (t, 2H), 3.77 (m, 3H), 3.53 (m, 1H), 3.27 (s, 3H), 3.23 (t, 2H), 2.35 (d, 2H), 1.4 (s, 18H). LCMS (ESI) 704.3 m/z [M+H]⁺.

### Example 58 Synthesis of fluorinated (S)-5-(3-amino-4-fluorophenyl)-2-(6-(3-fluoropyrrolidin-1-yl)pyridin-3-yl)-6,7-dihydrothiazolo[5,4-c]pyridin-4(5H)-one (¹⁸F Compound 26)

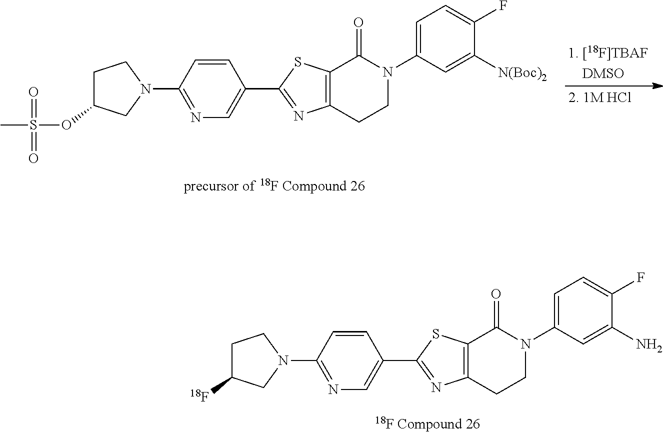

To dried [¹⁸F]tetrabutylammonium fluoride was added the precursor of ¹⁸F Compound 26 (1 mg in 0.7 mL), and the reaction mixture was heated at 100° C. for 10 min, followed by Boc-deprotection with 1 M HCl at 90° C. for 8 min. The crude reaction mixture was diluted with HPLC mobile phase (MeOH/NH₄OAc solution (20 mM, 55/45, v/v)), injected onto HPLC and purified by reverse phase HPLC. The product fraction was collected, diluted with ascorbic acid in water and passed through an tC18 cartridge. The cartridge was washed with ascorbic acid in water, followed by product elution with ethanol and rinsing of the cartridge with a formulation of ascorbic acid in saline. The resulting mixture was passed through a sterilizing filter and further diluted with saline to afford the formulated final product ¹⁸F-Compound 26. Confirmation of the identity of the product was determined by co-injection with a sample of the ¹⁹F-reference compound.

### Biological Assay Description

### 1. General Methods

Human brain material for these studies was obtained from Prof. William Seeley at the Neurodegenerative Disease Brain Bank UCSF (funding support from NIH grants P01AG019724 and P50AG023501, the Consortium for Frontotemporal Dementia Research, and the Tau Consortium), and also from Prof. Tammaryn Lashley at the Queen Square Brain Bank for Neurological Disorders, UCL. All material has been collected from donors from whom a written informed consent for brain autopsy and the use of the material and clinical information for research purposes has been obtained by the brain bank.

### 1.1 Radioligands

[³H]-Compound 1, [³H]-Compound 52, and [³H]-Compound 16 having specific activities of 75.83 Ci/mmol (1.0 mCi/mL), 52.4 or 47.8 Ci/mmol (1.0 mCi/mL) and 72.5 Ci/mmol (1.0 mCi/mL), respectively were used in the assays described below.

### 1.2. Preparation of Recombinant TDP-43 Aggregates

The TDP-43 aggregation protocol (Shimonaka et al., 2016) was implemented to generate TDP-43 aggregates. The protein preparation of high purity without any degradation was used for the aggregation procedure.

### 1.3. Preparation of Human Frontotemporal Dementia (FTD) Sarkosyl-Insoluble Brain Extracts

Human brain extracts were prepared as described in Laferriere et al., 2019, Nature Neurosc. A sample of brain tissue (frontal or temporal cortex) was homogenized at 1:4 (w/v) ratio in the homogenization-solubilization (HS) buffer at 4° C. using tissue homogenizer (Precellys) with CKmix homogenization tubes. The following sequence was used for homogenization: 3 cycles of 30 seconds at 5000 rpm (with 15 seconds pause between each cycle). Homogenized samples were aliquoted and stored at −80° C. in 1.5 mL low protein binding tubes.

Brain homogenates were thawed on ice and resuspended in HS buffer to obtain a final concentration of 2% sarkosyl, 1 unit/μL Benzonase and 1 mM MgCl₂. The samples were then incubated at 37° C. under constant shaking at 600 rpm on a thermomixer for 45 minutes (min). The supernatants were collected in a new tube (sarkosyl-soluble fraction, S1). The pellet was resuspended in 1000 μL of myelin floatation buffer and centrifuged at 20,000 g for 60 min at 4° C. The supernatant was carefully removed to remove all the floating lipids. This step was repeated if all the lipids could not be removed in a single step. The pellet was subsequently washed with Phosphate-Buffered Saline (PBS) and centrifuged for 30 min at 20,000 g at 4° C. The final pellet was resuspended in 200 μL PBS and stored at −80° C. (sarkosyl-insoluble fraction). The samples were analyzed by immunoblotting in denaturing conditions.

### 2. Assays

2.1 Kd Determination on Recombinant TDP-43 Aggregates with Filter-Binding Assay of [³H]-Compound 1

Recombinant flag-tagged TDP-43 aggregates (1.3 μM) were incubated in the presence of [³H]-Compound 1 with increasing concentrations of radioligand from 0 to 150 nM in a total reaction volume of 100 μL, for two hours at room temperature. 45 μL of sample (corresponding to each concentration) in duplicate was filtered under vacuum on a UniFilter-96 GF/C plate. The filters were washed 5 times with ice-cold buffer (50 mM Tris pH 7.4) and then dried overnight. 30 μL scintillation liquid was added in each well for 30 minutes before quantifying the signal on a Microbeta Trilux device (Perkin Elmer). The non-specific signal was determined with an excess of unlabeled Compound 1 (10 μM) and the specific binding was calculated by subtracting the non-specific signal from the total signal. The Kd (Dissociation constant) and R2 (parameter ranging between 0.0 and 1.0 that quantifies the goodness of fit, and the best curve fit obtained with value of 1.0) were obtained by fitting the specific binding data with non-linear regression analysis, using a one-site specific binding model in GraphPad Prism.

### Results:

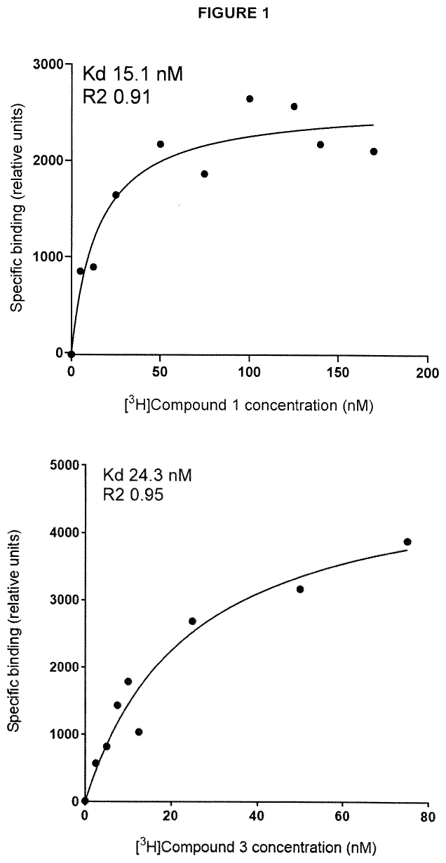

The dissociation constant (Kd) for [³H]-Compound 1 was determined on recombinant TDP-43 aggregates in a filter-binding assay. [³H]-Compound 1 had a high specific binding resulting in a high dynamic range and showed a Kd value of 15 nM on recombinant flag-tagged TDP-43 aggregates (FIG. 1). Data from three independent experiments resulted in the average Kd of 18.4±4 nM.

The dissociation constant (Kd) for [³H]-Compound 3 was determined on recombinant TDP-43 aggregates in a filter-binding assay. [³H]-Compound 3 had a high specific binding resulting in a high dynamic range and showed a Kd value of 24.3 nM on recombinant flag-tagged TDP-43 aggregates (FIG. 1).

2.2. Micro-Autoradiography Staining with [³H]-Compound 1 in Brain Sections from Patients with FTD

All tissues were collected from donors for or from whom a written informed consent for a brain autopsy and the use of the material and clinical information for research purposes had been obtained by the respective brain banks. All samples were anonymized and coded. Frozen brain tissue blocks with confirmed TDP-43 pathology upon autopsy were processed using a cryotome to generate sections of 10 μm in thickness and mounted on glass slides. Sections were kept at −80° C. until use.

Brain sections were immunostained using a commercially available antibody, specific for phosphorylated serine at amino acid 409/410 (anti-pTDP-43 pS409/410, Cosmobio, TIP-PTD-P02). Sections were fixed for 15 minutes at 4° C. with 4% formaldehyde (Sigma, 252549) and washed three times for five minutes with 1×PBS (Dulbecco's phosphate buffered saline, Sigma D1408) at room temperature. Next, sections were saturated and permeabilized in blocking buffer (PBS, 10% normal goat serum (NGS), 0.25% Triton X-100) for one hour at room temperature and incubated overnight at 4° C. with primary antibody against pTDP-43 (diluted at 1/250 in PBS, 5% NGS, 0.25% Triton X-100). The following day, sections were washed three times for five minutes with 1×PBS before incubation with a secondary, AlexaFluor647-labeled goat-anti-rabbit antibody (Abcam, ab150079, diluted at 1/500 in PBS) for 45 minutes at room temperature. Following incubation with primary antibodies the sections were washed three times in PBS before being processed further.

For micro-autoradiography, [³H]-Compound 1 was incubated on the sections at 60 nM in 50 mM Tris buffer pH 7.4 for 45 minutes at room temperature. Sections were then washed as follows: once in ice-cold 50 mM Tris-HCl pH 7.4 buffer for one minute, twice in ice-cold 70% ethanol for one minute, once in ice-cold 50 mM Tris-HCl pH 7.4 buffer for one minute and finally rinsed briefly in ice-cold distilled water. Sections were subsequently dried for one hour under a stream of air and then exposed to Ilford Nuclear Emulsion Type K5 (Agar Scientific, AGP9281) for five days at 4° C. in a light-proof slide storage box. After seven days, the sections were developed by immersing them successively in the following solutions: 1.) Ilford Phenisol Developer (1:5 dilution in H₂O, Agar Scientific, AGP9106) for four minutes, 2.) Ilfostop solution (1:20 dilution in H₂O, Agar Scientific, AGP9104) for two minutes, 3.) Ilford Hypam Fixer (1:5 dilution in H₂O, Agar Scientific, AGP9183) for four minutes and finally rinsed with H₂O for 10 minutes.

For image acquisition, sections were mounted using ProLong Gold Antifade reagent (Invitrogen P36930) and imaged on a Panoramic250 Slide Scanner (3DHistech) with a 40× objective capturing brightfield and fluorescent images separately or on a Panoramic Scan II (3DHistech) with a 20× objective. The Visiopharm image analysis software suite was used to align the fluorescent image with the brightfield image.

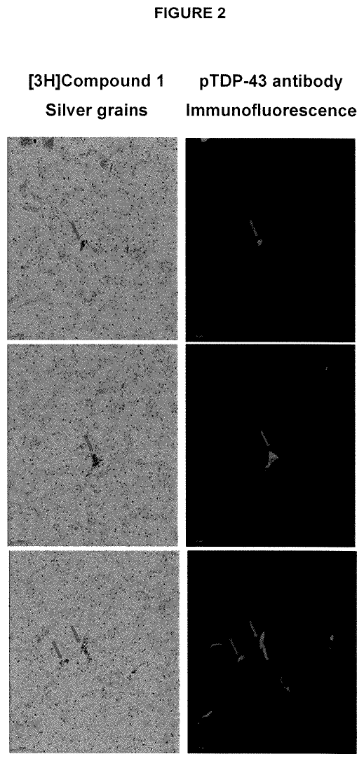

Micro-autoradiography signal from [³H]-Compound 1, incubated on human brain sections, was detected in the form of accumulating silver grains that co-localize with immunofluorescence signal from pTDP-43 antibody suggesting target engagement of [³H]-Compound 1 on TDP-43 aggregates. Incubation of brain sections from FTLD-TDP donors with [³H]-Compound 1 (60 nM) showed co-localization with pTDP-43 aggregates (FIG. 2). To our knowledge, this is the first compound showing target engagement to TDP-43 aggregates on human brain sections by an autoradiographic technique.

### 2.3. Micro-Radiobinding Competition Assay for the Determination of Binding Affinity

Human FTD sarkosyl-insoluble brain extracts were spotted onto microarray slides. The slides were incubated with [³H]-Compound 52 or [³H]-Compound 16 ligands at 75 nM or 40 nM, respectively, and the example compounds (non-radiolabelled) at 2 μM and 250 nM. In some cases, the non-radiolabelled example compounds were further assessed for a range of different concentrations, varying from 0.24 nM to 2 μM for determination of the inhibition constant (Ki). After incubation, slides were washed and scanned by a real-time autoradiography system (BeaQuant, ai4R). Quantification of the signal was performed by using the Beamage image analysis software (ai4R). Non-specific signal was determined with an excess of non-radiolabelled compound 52 or compound 16 (2 μM) and specific binding was calculated by subtracting the non-specific signal from the total signal. Competition was calculated as percent, where 0% was defined as the specific binding in the presence of vehicle and 100% as the values obtained in the presence of excess of the non-radiolabelled compound 52 or compound 16. Kᵢ values were calculated in GraphPad Prism7 by applying a nonlinear regression curve fit using a one site, specific binding model. Measurements were performed with at least two technical replicates in the two-concentration competition experiment and with one technical replicate in the experiments including a range of concentrations. For compounds tested in more than one experiment, the mean of the replicates or Kᵢ values in independent experiments is reported.

Results: Example compounds were assessed for their potency to compete with the binding of [³H]-ligands ([³H]-Compound 52 or [³H]-Compound 16 [) to FTD patient brain-derived TDP-43 aggregates. Results of the micro-radiobinding competition assay for the example compounds tested are shown in Table 6 below as: % competition at 2 μM and 250 nM. Kᵢ values are also shown when available.

### Examples 1-3 and 13 to 50

Following the screening methods described above, the following Compounds were characterized.

TABLE 6

| | | Human brain | Human brain | |
|---|---|---|---|---|
| | | derived TDP-43 | derived TDP-43 | |
| | | aggregates | aggregates | Human brain |
| | | Mean% | Mean% | derived TDP- |
| | | competition at | competition at | 43 aggregates |
| Example | Product | 2 μM | 250 nM | Ki [nM] |
| 1 | 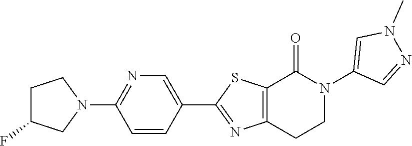 [SMILES: Cn1cc(N2CCc3nc(-c4ccc(N5CC[C@@H](F)C5)nc4)sc3C2=O)cn1] | n.d.^{b} | n.d.^{b} | 18.7ª |
| 2 | 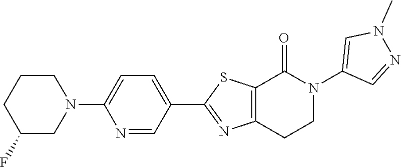 [SMILES: Cn1cc(N2CCc3nc(-c4ccc(N5CCC[C@@H](F)C5)nc4)sc3C2=O)cn1] | 92.5ª | 61.0ª | 71.7 |
| 3 | 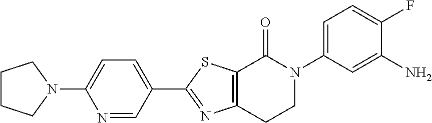 [SMILES: Nc1cc(N2CCc3nc(-c4ccc(N5CCCC5)nc4)sc3C2=O)ccc1F] | 91.2ª | 62.2ª | 73.1ª |
| 13 | 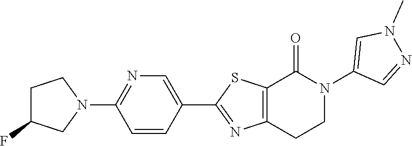 [SMILES: Cn1cc(N2CCc3nc(-c4ccc(N5CC[C@H](F)C5)nc4)sc3C2=O)cn1] | 105.0ª | 69.4ª | n.d.^{b} |
| 14 | 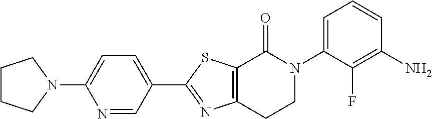 [SMILES: Nc1cccc(N2CCc3nc(-c4ccc(N5CCCC5)nc4)sc3C2=O)c1F] | 85.7ª | 44.0ª | 58.8ª |
| 15 | 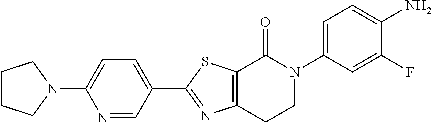 [SMILES: Nc1ccc(N2CCc3nc(-c4ccc(N5CCCC5)nc4)sc3C2=O)cc1F] | 96.4ª | 64.6ª | 139.3ª |
| 16 | 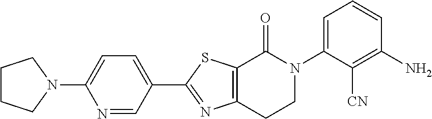 [SMILES: N#Cc1c(N)cccc1N1CCc2nc(-c3ccc(N4CCCC4)nc3)sc2C1=O] | 83.2ª | 46.0ª | 81.0ª |
| 17 | 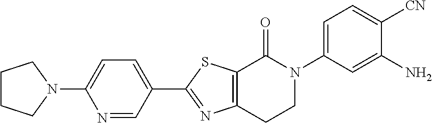 [SMILES: N#Cc1ccc(N2CCc3nc(-c4ccc(N5CCCC5)nc4)sc3C2=O)cc1N] | 95.0ª | 82.0ª | n.d.^{b} |
| 18 | 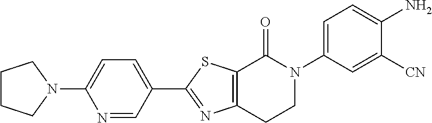 [SMILES: N#Cc1cc(N2CCc3nc(-c4ccc(N5CCCC5)nc4)sc3C2=O)ccc1N] | 102.0ª | 76.2ª | 68.6ª |
| 19 | 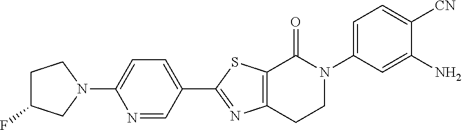 [SMILES: N#Cc1ccc(N2CCc3nc(-c4ccc(N5CC[C@@H](F)C5)nc4)sc3C2=O)cc1N] | 92.1ª | 76.9ª | 54.2ª |
| 20 | 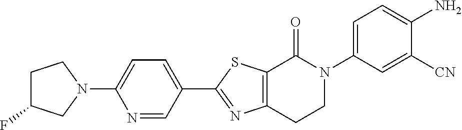 [SMILES: N#Cc1cc(N2CCc3nc(-c4ccc(N5CC[C@@H](F)C5)nc4)sc3C2=O)ccc1N] | 88.3ª | 66.7ª | 161ª |
| 21 | 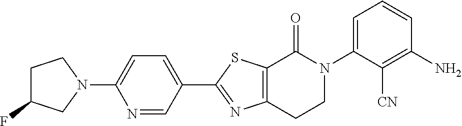 [SMILES: N#Cc1c(N)cccc1N1CCc2nc(-c3ccc(N4CC[C@H](F)C4)nc3)sc2C1=O] | 40.1ª | 22.7ª | n.d.^{b} |
| 22 | 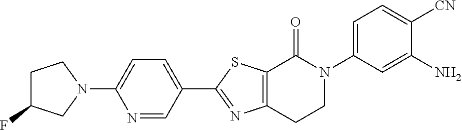 [SMILES: N#Cc1ccc(N2CCc3nc(-c4ccc(N5CC[C@H](F)C5)nc4)sc3C2=O)cc1N] | 80.3ª | 64.2ª | n.d.^{b} |
| 23 | 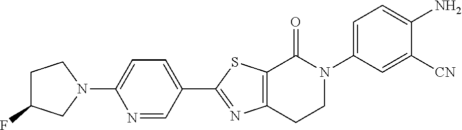 [SMILES: N#Cc1cc(N2CCc3nc(-c4ccc(N5CC[C@H](F)C5)nc4)sc3C2=O)ccc1N] | 76.1ª | 35.5ª | 100.1ª |
| 24 | 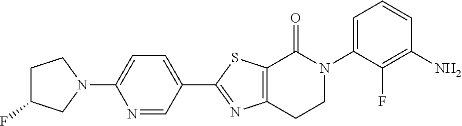 [SMILES: Nc1cccc(N2CCc3nc(-c4ccc(N5CC[C@@H](F)C5)nc4)sc3C2=O)c1F] | 84.1ª | 56.9ª | 109.6ª |
| 25 | 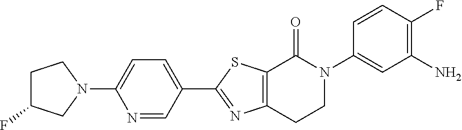 [SMILES: Nc1cc(N2CCc3nc(-c4ccc(N5CC[C@@H](F)C5)nc4)sc3C2=O)ccc1F] | 83.0ª | 66.6ª | n.d.^{b} |
| 26 | 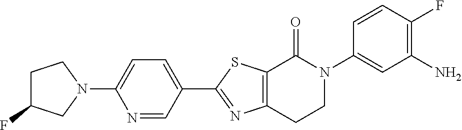 [SMILES: Nc1cc(N2CCc3nc(-c4ccc(N5CC[C@H](F)C5)nc4)sc3C2=O)ccc1F] | 70.8ª | 40.8ª | 9.9^{a} |
| 27 | 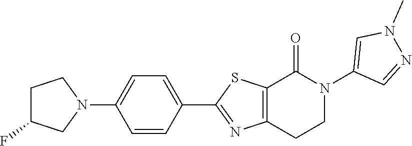 [SMILES: Cn1cc(N2CCc3nc(-c4ccc(N5CC[C@@H](F)C5)cc4)sc3C2=O)cn1] | 80.2ª | 54.3ª | 128.0ª |
| 28 | 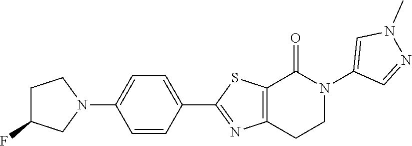 [SMILES: Cn1cc(N2CCc3nc(-c4ccc(N5CC[C@H](F)C5)cc4)sc3C2=O)cn1] | 79.7ª | 56.5ª | 111.0ª |
| 29 | 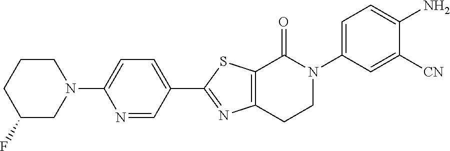 [SMILES: N#Cc1cc(N2CCc3nc(-c4ccc(N5CCC[C@@H](F)C5)nc4)sc3C2=O)ccc1N] | 73.9ª | 42.6ª | 27.7ª |
| 30 | 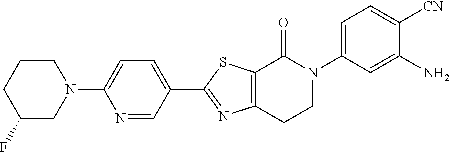 [SMILES: N#Cc1ccc(N2CCc3nc(-c4ccc(N5CCC[C@@H](F)C5)nc4)sc3C2=O)cc1N] | 75.7ª | 55.9ª | 86.6ª |
| 31 | 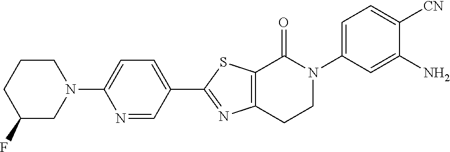 [SMILES: N#Cc1ccc(N2CCc3nc(-c4ccc(N5CCC[C@H](F)C5)nc4)sc3C2=O)cc1N] | 79.7ª | 67.6ª | 100.8ª |
| 32 | 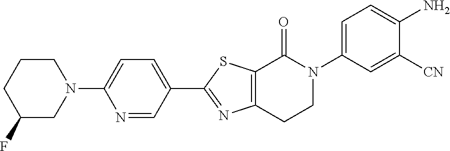 [SMILES: N#Cc1cc(N2CCc3nc(-c4ccc(N5CCC[C@H](F)C5)nc4)sc3C2=O)ccc1N] | 80.9ª | 44.0ª | n.d.^{b} |
| 33 | 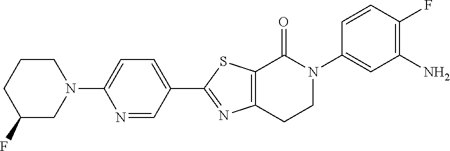 [SMILES: Nc1cc(N2CCc3nc(-c4ccc(N5CCC[C@H](F)C5)nc4)sc3C2=O)ccc1F] | 81.0ª | 52.1ª | 144.1ª |
| 34 | 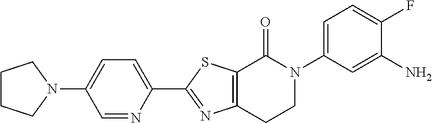 [SMILES: Nc1cc(N2CCc3nc(-c4ccc(N5CCCC5)cn4)sc3C2=O)ccc1F] | 67.9ª | 72.8ª | n.d.^{b} |
| 35 | 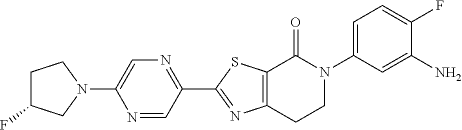 [SMILES: Nc1cc(N2CCc3nc(-c4cnc(N5CC[C@@H](F)C5)cn4)sc3C2=O)ccc1F] | 74.6ª | 53.0ª | n.d.^{b} |
| 36 | 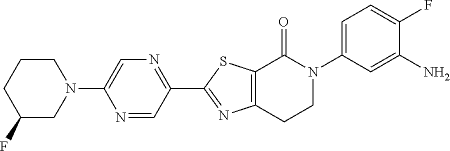 [SMILES: Nc1cc(N2CCc3nc(-c4cnc(N5CCC[C@H](F)C5)cn4)sc3C2=O)ccc1F] | n.d.b | 51.0ª | n.d.^{b} |
| 37 | 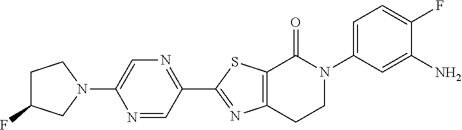 [SMILES: Nc1cc(N2CCc3nc(-c4cnc(N5CC[C@H](F)C5)cn4)sc3C2=O)ccc1F] | 74.9ª | 54.3ª | 79.9ª |
| 38 | 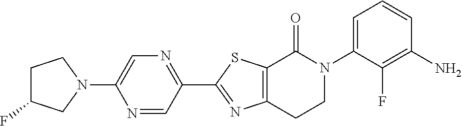 [SMILES: Nc1cccc(N2CCc3nc(-c4cnc(N5CC[C@@H](F)C5)cn4)sc3C2=O)c1F] | 74.0ª | 46.3ª | 114.5ª |
| 39 | 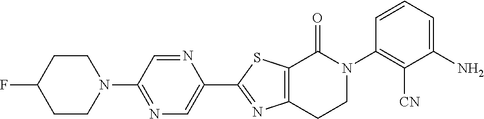 [SMILES: N#Cc1c(N)cccc1N1CCc2nc(-c3cnc(N4CCC(F)CC4)cn3)sc2C1=O] | 77.4^{c} | 64.5^{c} | 147.9^{c} |
| 40 | 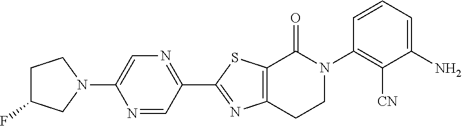 [SMILES: N#Cc1c(N)cccc1N1CCc2nc(-c3cnc(N4CC[C@@H](F)C4)cn3)sc2C1=O] | 77.9^{c} | 54.6^{c} | 104.3^{c} |
| 41 | 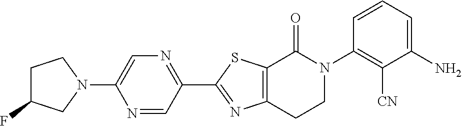 [SMILES: N#Cc1c(N)cccc1N1CCc2nc(-c3cnc(N4CC[C@H](F)C4)cn3)sc2C1=O] | 67.7^{c} | 47.2^{c} | n.d.^{b} |
| 42 | 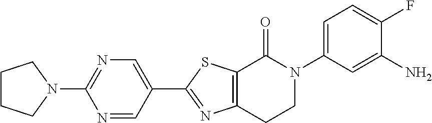 [SMILES: Nc1cc(N2CCc3nc(-c4cnc(N5CCCC5)nc4)sc3C2=O)ccc1F] | 79.6ª | 43.8ª | n.d.^{b} |
| 43 | 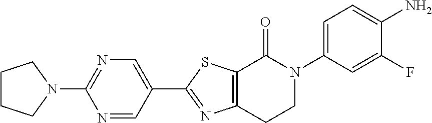 [SMILES: Nc1ccc(N2CCc3nc(-c4cnc(N5CCCC5)nc4)sc3C2=O)cc1F] | 69.4ª | 45.0ª | n.d.^{b} |
| 44 | 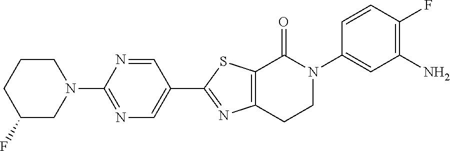 [SMILES: Nc1cc(N2CCc3nc(-c4cnc(N5CCC[C@@H](F)C5)nc4)sc3C2=O)ccc1F] | 77.4ª | 60.0ª | 126.0ª |
| 45 | 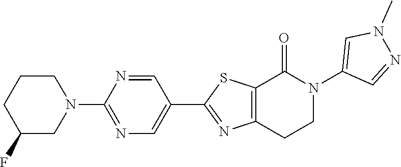 [SMILES: Cn1cc(N2CCc3nc(-c4cnc(N5CCC[C@H](F)C5)nc4)sc3C2=O)cn1] | 64.3ª | 47.0ª | n.d.^{b} |
| 46 | 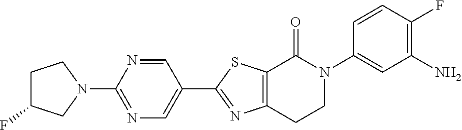 [SMILES: Nc1cc(N2CCc3nc(-c4cnc(N5CC[C@@H](F)C5)nc4)sc3C2=O)ccc1F] | 85.9ª | 62.5ª | 93.0ª |
| 47 | 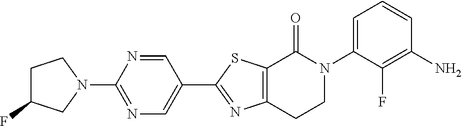 [SMILES: Nc1cccc(N2CCc3nc(-c4cnc(N5CC[C@H](F)C5)nc4)sc3C2=O)c1F] | 81.7ª | 50.2ª | 151.8ª |
| 48 | 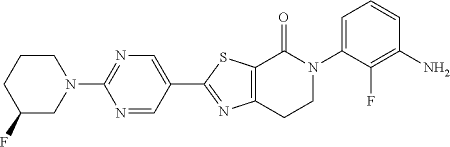 [SMILES: Nc1cccc(N2CCc3nc(-c4cnc(N5CCC[C@H](F)C5)nc4)sc3C2=O)c1F] | 77.9ª | 46.9ª | 73.6ª |
| 49 | 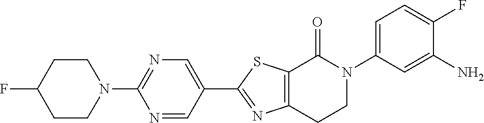 [SMILES: Nc1cc(N2CCc3nc(-c4cnc(N5CCC(F)CC5)nc4)sc3C2=O)ccc1F] | 65.5^{c} | 63.1^{c} | 117.5^{c} |
| 50 | 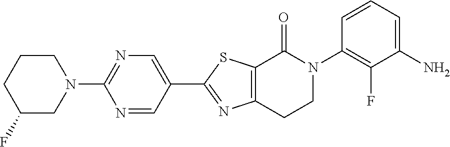 [SMILES: Nc1cccc(N2CCc3nc(-c4cnc(N5CCC[C@@H](F)C5)nc4)sc3C2=O)c1F] | 63.4^{c} | 52.8^{c} | n.d.^{b} |

^{a}([³H]-Compound 52
n.d.^{b}: not determined
^{c3}H]-Compound 16

1. A compound having the formula (I) 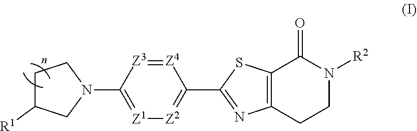
or a detectably labelled compound, stereoisomer, polymorph, racemic mixture, tautomer, pharmaceutically acceptable salt, prodrug, hydrate, or solvate thereof, or mixtures thereof;
wherein
Z¹, Z², Z³ and Z⁴ are each selected from CH and N, wherein
if Z¹ is N, Z² is CH;
if Z² is N, Z¹ is CH;
if Z³ is N, Z⁴ is CH;
if Z⁴ is N, Z³ is CH;
n is 1 or 2;
R¹ is H or F;
R² is selected from
a 5- or 6-membered carbocyclic ring which can be optionally substituted with F, NH₂, CN and/or CH₃,
a 5- or 6-membered heterocycloalkyl ring which can be optionally substituted with F, NH₂, CN and/or CH₃, wherein the 5- or 6-membered heterocycloalkyl ring contains one or more heteroatoms selected from N, O and S,
a 5-membered heteroaryl ring which can be optionally substituted with F, NH₂, CN and/or CH₃, wherein the 5-membered heteroaryl ring contains one or more heteroatoms selected from N, O and S, or
a 6-membered heteroaryl ring which can be optionally substituted with F, NH₂, CN and/or CH₃, wherein the 6-membered heteroaryl ring contains one heteroatom selected from O and S, or two or more heteroatoms selected from O, N and S.

2. The compound according to claim 1, having the formula (Ia), (Ib), (Ic), (Id) or (Ie) 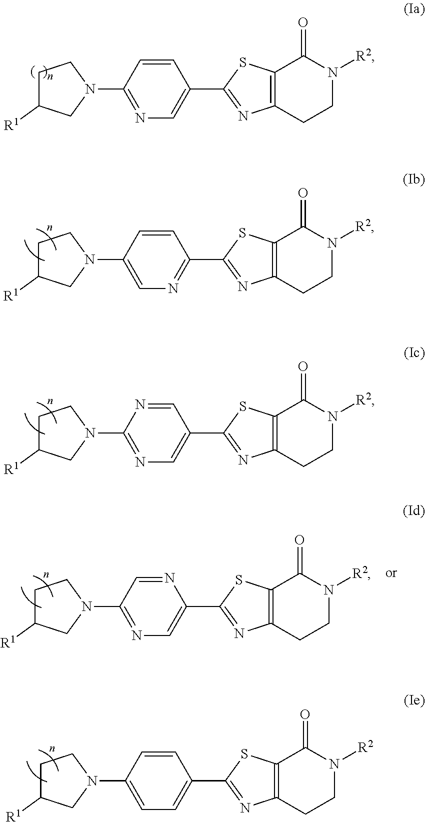
or a detectably labelled compound, stereoisomer, polymorph, racemic mixture, tautomer, pharmaceutically acceptable salt, prodrug, hydrate, or solvate thereof, or mixtures thereof;
wherein
n is 1 or 2;
R¹ is H or F;
R² is
a 5- or 6-membered carbocyclic ring which can be optionally substituted with F, NH₂, CN and/or CH₃,
a 5- or 6-membered heterocycloalkyl ring which can be optionally substituted with F, NH₂, CN and/or CH₃, wherein the 5- or 6-membered heterocycloalkyl ring contains one or more heteroatoms selected from N, O and S,
a 5-membered heteroaryl ring which can be optionally substituted with F, NH₂, CN and/or CH₃, wherein the 5-membered heteroaryl ring contains one or more heteroatoms selected from N, O and S, or
a 6-membered heteroaryl ring which can be optionally substituted with F, NH₂, CN and/or CH₃, wherein the 6-membered heteroaryl ring contains one heteroatom selected from O and S, or two or more heteroatoms selected from O, N and S.

3-6. (canceled)

7. The compound according to claim 1, wherein
R² is 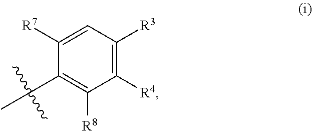
wherein
R³ is F, R⁴ is NH₂, R⁷ is H, and R⁸ is H,
R³ is NH₂, R⁴ is F, R⁷ is H, and R⁸ is H,
R³ is CN, R⁴ is NH₂, R⁷ is H, and R⁸ is H,
R³ is H, R⁴ is NH₂, R7 is H, and R⁸ is CN, or
R³ is H, R⁴ is NH₂, R⁷ is H, and R⁸ is F;
or
R² is 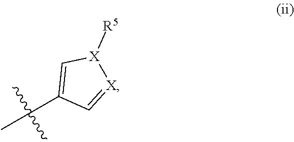
wherein X is N and R⁵ is CH₃ or H.

8. The compound according to claim 1, which comprises a detectable label, wherein the detectable label is preferably ³H or ¹⁸F.

9. (canceled)

10. The compound according to claim 8, having the formula (I-T) 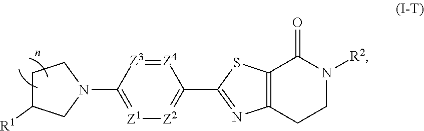
or a stereoisomer, polymorph, racemic mixture, tautomer, pharmaceutically acceptable salt, prodrug, hydrate, or solvate thereof, or mixtures thereof,
wherein
Z¹, Z², Z³ and Z⁴ are each selected from CH and N, wherein
if Z¹ is N, Z² is CH;
if Z² is N, Z is CH;
if Z³ is N, Z⁴ is CH;
if Z⁴ is N, Z³ is CH;
n is 1 or 2;
R¹ is H or F;
R² is selected from
a 5- or 6-membered carbocyclic ring which can be optionally substituted with F, NH₂, CN and/or CH₃,
a 5- or 6-membered heterocycloalkyl ring which can be optionally substituted with F, NH₂, CN and/or CH₃, wherein the 5- or 6-membered heterocycloalkyl ring contains one or more heteroatoms selected from N, O and S,
a 5-membered heteroaryl ring which can be optionally substituted with F, NH₂, CN and/or CH₃, wherein the 5-membered heteroaryl ring contains one or more heteroatoms selected from N, O and S, or
a 6-membered heteroaryl ring which can be optionally substituted with F, NH₂, CN and/or CH₃, wherein the 6-membered heteroaryl ring contains one heteroatom selected from O and S, or two or more heteroatoms selected from O, N and S.
wherein
at least one of Z¹, Z², Z³, Z⁴ is selected from C-T; or
R² is substituted by at least one CT₃ or at least one of the hydrogen atoms in R² is replaced by T.

11. The compound according to claim 10, having the formula (I-Ta), (I-Tb), (I-Td) or (I-Te) 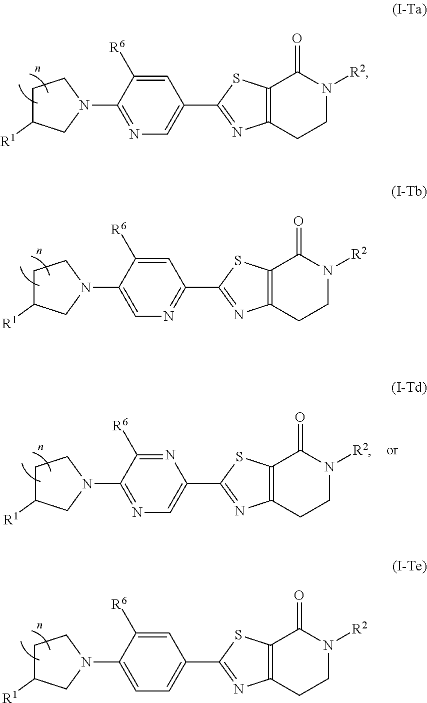
or a stereoisomer, polymorph, racemic mixture, tautomer, pharmaceutically acceptable salt, prodrug, hydrate, or solvate thereof, or mixtures thereof,
wherein
n is 1 or 2;
R¹ is H or F;
R⁶ is T or H;
T is ³H; and
wherein
R² is 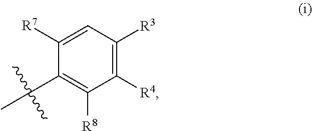
wherein
R³ is F, R⁴ is NH₂, and at least one of R⁷ and R¹ is T,
R³ is CN, R⁴ is NH₂, and at least one of R⁷ and R¹ is T,
R⁴ is NH₂, R¹ is —CN, and at least one of R³ and R⁷ is T, or
R⁴ is NH₂, R⁸ is F, and at least one of R³ and R⁷ is T and, if applicable, the other is H;
or wherein
R² is 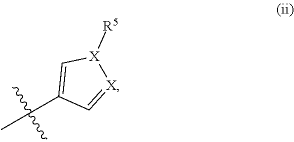
wherein X is N and R⁵ is CT₃; and
R⁶ is H.

12. The compound according to claim 10, wherein the compound is selected from 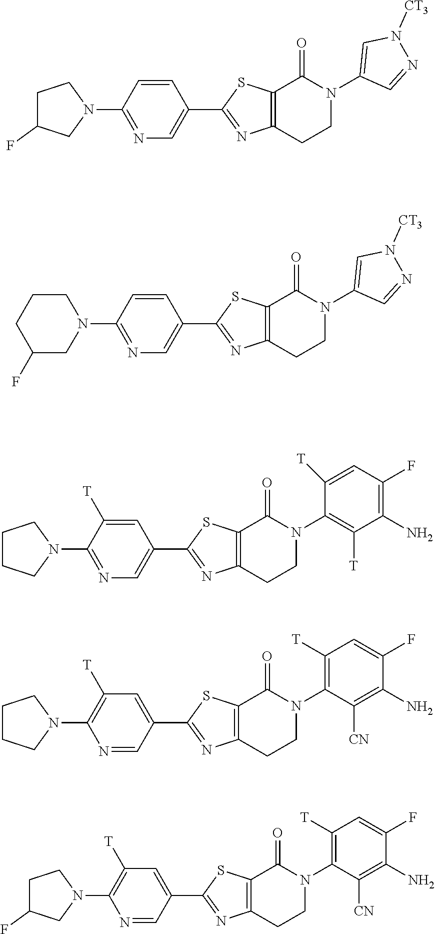
or a detectably labelled compound, stereoisomer, polmorph, racemic mixture, tautomer, pharmaceutically acceptable salt, prodrug hydrate, or solvate thereof or mixtures thereof wherein T is ³H.

13. The compound according to claim 8, having the formula (I-F) 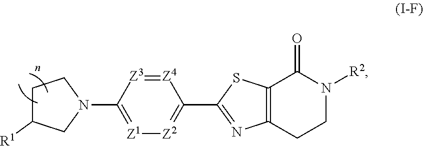
or a stereoisomer, polymorph, racemic mixture, tautomer, pharmaceutically acceptable salt, prodrug, hydrate, or solvate thereof, or mixtures thereof,
wherein
n, Z¹, Z², Z³, Z⁴, R¹ and R² are as defined in claim 1, wherein at least one F is ¹⁸F, preferably wherein R¹ is ¹⁸F.

14. The compound according to claim 13, which is 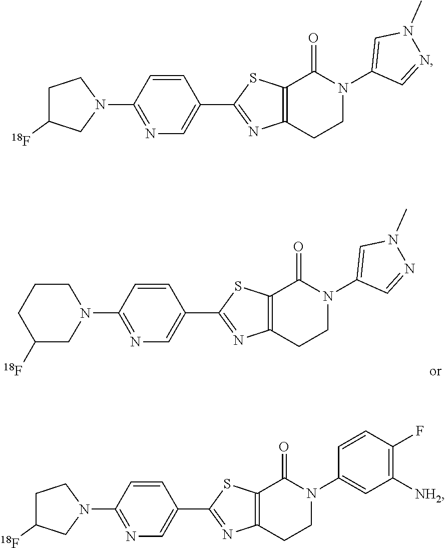 [SMILES: Cn1cc(N2CCc3nc(-c4ccc(N5CCC([18F])C5)nc4)sc3C2=O)cn1 ; Cn1cc(N2CCc3nc(-c4ccc(N5CCCC([18F])C5)nc4)sc3C2=O)cn1 ; "or" ; Nc1cc(N2CCc3nc(-c4ccc(N5CCC([18F])C5)nc4)sc3C2=O)ccc1F]
or a detectably labelled compound, stereoisomer, polymorph, racemic mixture, tautomer, pharmaceutically acceptable salt, prodrug, hydrate, or solvate thereof, or mixtures thereof.

15. A diagnostic composition comprising a compound according to claim 1 or a detectably labelled compound, stereoisomer, polymorph, racemic mixture, tautomer, pharmaceutically acceptable salt, prodrug, hydrate, or solvate thereof, or mixtures thereof, and at least one physiologically acceptable carrier, diluent, adjuvant and/or excipient.

16. (canceled)

17. A method of imaging of TDP-43 aggregates, wherein the compound according to claim 1, or a detectably labelled compound, stereoisomer, polymorph, racemic mixture, tautomer, pharmaceutically acceptable salt, prodrug, hydrate, or solvate thereof, or mixtures thereof, is employed, particularly wherein the imaging is conducted by positron emission tomography.

18. (canceled)

19. A method selected from
(A) a method of imaging a disease, disorder or abnormality associated with TDP-43 aggregates in a subject, the method comprising the steps:
(a) Administering a compound according to claim 1 or a detectably labelled compound, stereoisomer, polymorph, racemic mixture, tautomer, pharmaceutically acceptable salt, prodrug, hydrate, or solvate thereof, or mixtures thereof to the subject;
(b) Allowing the compound to bind to the TDP-43 aggregates;
(c) Detecting the compound bound to the TDP-43 aggregates; and
(d) Optionally generating an image representative of the location and/or amount of the compound bound to the TDP-43 aggregates;
or
(B) a method of positron emission tomography (PET) imaging of TDP-43 aggregates in a tissue of a subject, the method comprising the steps:
(a) Administering a compound according to claim 1 or a detectably labelled compound, stereoisomer, polymorph, racemic mixture, tautomer, pharmaceutically acceptable salt, prodrug, hydrate, or solvate thereof, or mixtures thereof to the subject;
(b) Allowing the compound to bind to the TDP-43 aggregates; and
(c) Detecting the compound bound to the TDP-43 aggregates by collecting a positron emission tomography (PET) image of the tissue of the subject;
wherein the tissue is preferably a tissue of the central nervous system (CNS), an eye tissue, or a brain tissue, preferably wherein the tissue is brain tissue;
or
(C) a method for the detection and optionally quantification of TDP-43 aggregates in a tissue of a subject, the method comprising the steps:
(a) Bringing a sample or a specific body part or body area suspected to contain TDP-43 aggregates into contact with a compound according to claim 1 or a detectably labelled compound, stereoisomer, polymorph, racemic mixture, tautomer, pharmaceutically acceptable salt, prodrug, hydrate, or solvate thereof, or mixtures thereof;
(b) Allowing the compound to bind to the TDP-43 aggregates;
(c) Detecting the compound bound to the TDP-43 aggregates using positron emission tomography; and
(d) Optionally quantifying the amount of the compound bound to the TDP-43 aggregates;
or
(D) a method of diagnosing of a disease, disorder or abnormality associated with TDP-43 aggregates or for diagnosing a TDP-43 proteinopathy, the method comprising the steps:
(a) Bringing a sample or a specific body part or body area suspected to contain TDP-43 aggregates into contact with a compound according to claim 1 or a detectably labelled compound, stereoisomer, polymorph, racemic mixture, tautomer, pharmaceutically acceptable salt, prodrug, hydrate, or solvate thereof, or mixtures thereof;
(b) Allowing the compound to bind to the TDP-43 aggregates;
(c) Detecting the compound bound to the TDP-43 aggregates; and
(d) Optionally correlating the presence or absence of the compound bound with the TDP-43 aggregates with the presence or absence of TDP-43 aggregates in the sample or specific body part or body area;
or
(E) A method of determining a predisposition to a disease, disorder or abnormality associated with TDP-43 aggregates or a TDP-43 proteinopathy, the method comprising the steps:
(a) Bringing a sample or a specific body part or body area suspected to contain TDP-43 aggregates into contact with a compound according to claim 1 or a detectably labelled compound, stereoisomer, polymorph, racemic mixture, tautomer, pharmaceutically acceptable salt, prodrug, hydrate, or solvate thereof, or mixtures thereof;
(b) Allowing the compound to bind to the TDP-43 aggregates;
(c) Detecting the compound bound to the TDP-43 aggregates; and
(d) Optionally correlating the presence or absence of the compound bound with the TDP-43 aggregates with the presence or absence of TDP-43 aggregates in the sample or specific body part or body area;
or
(F) a method of monitoring the progression of a disease, disorder or abnormality associated with TDP-43 aggregates or for monitoring the progression of a TDP-43 proteinopathy in a patient, the method comprising the steps:
(a) Bringing a sample, a specific body part or body area suspected to contain TDP-43 aggregates into contact with the compound according to claim 1 or a detectably labelled compound, stereoisomer, polymorph, racemic mixture, tautomer, pharmaceutically acceptable salt, prodrug, hydrate, or solvate thereof, or mixtures thereof;
(b) Allowing the compound to bind to the TDP-43 aggregates;
(c) Detecting the compound bound to the TDP-43 aggregates;
(d) Optionally correlating the presence or absence of the compound bound with the TDP-43 aggregates with the presence or absence of TDP-43 aggregates in the sample or specific body part or body area; and
(e) Optionally repeating steps (a) to (c) and, if present, optional step (d) at least one time;
or
(G) a method of collecting data for predicting responsiveness of a patient suffering from a disease, disorder or abnormality associated with TDP-43 aggregates to a treatment with a medicament, the method comprising the steps:
(a) Bringing a sample, a specific body part or body area suspected to contain TDP-43 aggregates into contact with a compound according to claim 1 or a detectably labelled compound, stereoisomer, polymorph, racemic mixture, tautomer, pharmaceutically acceptable salt, prodrug, hydrate, or solvate thereof, or mixtures thereof;
(b) Allowing the compound to bind to the TDP-43 aggregates;
(c) Detecting the compound bound to the TDP-43 aggregates;
(d) Optionally correlating the presence or absence of the compound bound with the TDP-43 aggregates with the presence or absence of TDP-43 aggregates in the sample or specific body part or body area; and
(e) Optionally repeating steps (a) to (c) and, if present, optional step (d) at least one time.

20-27. (canceled)

28. The method according to claim 19, wherein the step of optionally correlating the presence or absence of the compound bound to the TDP-43 aggregates with the presence or absence of TDP-43 aggregates in the sample or specific body part or body area; comprises
Determining the amount of the compound bound to the TDP-43 aggregates;
Correlating the amount of the compound bound to the TDP-43 aggregates with the amount of TDP-43 aggregates in the sample or specific body part or body area; and
Optionally comparing the amount of the compound bound with the TDP-43 aggregates in a sample or specific body part or body area to a normal control value in a healthy control subject.

29. A TDP-43 aggregates' biomarker or a TDP-43 proteinopathy biomarker or a TDP-43 proteinopathy diagnostic agent or TDP-43 proteinopathy diagnostic tool or an in vitro analytical reference or an in vitro screening tool comprising the compound according to claim 1 or a detectably labelled compound, stereoisomer, polymorph, racemic mixture, tautomer, pharmaceutically acceptable salt, prodrug, hydrate, or solvate thereof, or mixtures thereof.

30-31. (canceled)

32. The method according to claim 19, wherein the disease, disorder or abnormality associated with TDP-43 aggregates or the TDP-43 proteinopathy is selected from Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) including Frontotemporal lobar dementia TDP-43 or Frontotemporal lobar degeneration with ubiquitin-positive TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (such as nfvPPA), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy, Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (Sporadic inclusion body myositis, Inclusion body myopathy with a mutation in the valosin-containing protein (VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES), Traumatic Brain Injury (TBI), Dementia with Lewy Bodies (DLB) and Parkinson's disease (PD), preferably, the disease, the disorder or the abnormality associated with TDP-43 aggregates or the TDP-43 proteinopathy is selected from Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

33-36. (canceled)

37. A compound having the formula (II) 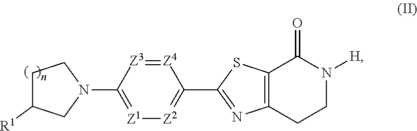
or a stereoisomer, polymorph, racemic mixture, tautomer, pharmaceutically acceptable salt, prodrug, hydrate, or solvate thereof, or mixtures thereof,
wherein
Z¹, Z², Z³ and Z⁴ are each selected from C—Br, C—I, C—H and N, wherein
if Z¹ is N, Z² is C—Br, C—I, or C—H;
if Z² is N, Z¹ is C—Br, C—I, or C—H;
if Z³ is N, Z⁴ is C—Br, C—I, or C—H;
if Z⁴ is N, Z³ is C—Br, C—I, or C—H;
n is 1 or 2; and
R¹ is H or F.

38. A compound having the formula (III) 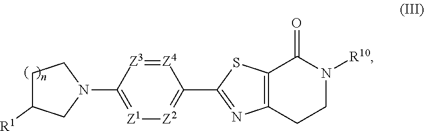
or a stereoisomer, polymorph, racemic mixture, tautomer, pharmaceutically acceptable salt, prodrug, hydrate, or solvate thereof, or mixtures thereof,
wherein
Z¹, Z², Z³ and Z⁴ are each selected from C—Br, C—I, C—H and N, wherein
if Z¹ is N, Z² is C—Br, C—I, or C—H;
if Z² is N, Z¹ is C—Br, C—I, or C—H;
if Z³ is N, Z⁴ is C—Br, C—I, or C—H;
if Z⁴ is N, Z³ is C—Br, C—I, or C—H;
n is 1 or 2;
R¹ is H or F;
R¹⁰ is
a 5- or 6-membered carbocyclic ring which can be optionally substituted with Br, I, F, NH₂, CN and/or CH₃,
a 5- or 6-membered heterocycloalkyl ring which can be optionally substituted with Br, I, F, NH₂, CN and/or CH₃, wherein the 5- or 6-membered heterocycloalkyl ring contains one or more heteroatoms selected from N, O and S,
a 5-membered heteroaryl ring which can be optionally substituted with Br, I, F, NH₂, CN and/or CH₃, wherein the 5-membered heteroaryl ring contains one or more heteroatoms selected from N, O and S, or
a 6-membered heteroaryl ring which can be optionally substituted with Br, I, F, NH₂, CN and/or CH₃, wherein the 6-membered heteroaryl ring contains one heteroatom selected from O and S, or two or more heteroatoms selected from O, N and S;
at least one of Z¹, Z², Z³ or Z⁴ is selected from C—Br or C—I, and/or
R¹⁰ comprises Br or I.

39. A method for preparing a compound having the formula (I) as defined in claim 8, the method comprising the step of radiolabeling a compound having the formula (III) with ³H 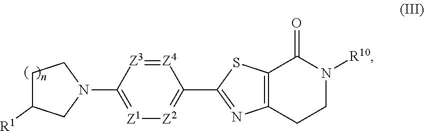
or a stereoisomer, polymorph, racemic mixture, tautomer, pharmaceutically acceptable salt, prodrug, hydrate, or solvate thereof, or mixtures thereof,
wherein
Z¹, Z², Z³ and Z⁴ are each selected from C—Br, C—I, C—H and N, wherein
if Z¹ is N, Z² is C—Br, C—I, or C—H;
if Z² is N, Z¹ is C—Br, C—I, or C—H;
if Z³ is N, Z⁴ is C—Br, C—I, or C—H;
if Z⁴ is N, Z³ is C—Br, C—I, or C—H;
n is 1 or 2;
R¹ is H or F;
R¹⁰ is
a 5- or 6-membered carbocyclic ring which can be optionally substituted with Br, I, F, NH₂, CN and/or CH₃,
a 5- or 6-membered heterocycloalkyl ring which can be optionally substituted with Br, I, F, NH₂, CN and/or CH₃, wherein the 5- or 6-membered heterocycloalkyl ring contains one or more heteroatoms selected from N, O and S,
a 5-membered heteroaryl ring which can be optionally substituted with Br, I, F, NH₂, CN and/or CH₃, wherein the 5-membered heteroaryl ring contains one or more heteroatoms selected from N, O and S, or
a 6-membered heteroaryl ring which can be optionally substituted with Br, I, F, NH₂, CN and/or CH₃, wherein the 6-membered heteroaryl ring contains one heteroatom selected from O and S, or two or more heteroatoms selected from O, N and S;
at least one of Z¹, Z², Z³ or Z⁴ is selected from C—Br or C—I, and/or
R¹⁰ comprises Br or I,
wherein the at least one Br or I is replaced by CT₃, or T,
and wherein T is ³H.

40. A compound having the formula (IV) 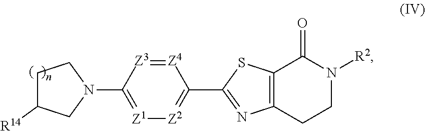
or a detectably labelled compound, stereoisomer, polymorph, racemic mixture, tautomer, pharmaceutically acceptable salt, prodrug, hydrate, or solvate thereof, or mixtures thereof
wherein
n, Z¹, Z², Z³, Z⁴ and R² are as defined in claim 1; and R¹⁴ is a leaving group (LG).

41. A method for preparing a compound having the formula (I) as defined in claim 98, the method comprising the step of:
radiolabeling a compound having the formula (IV) with ¹⁸F 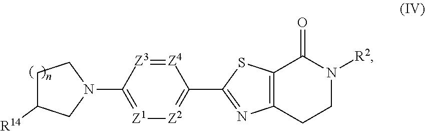
wherein
Z², Z², Z³ and Z⁴ are each selected from CH and N, wherein
if Z¹ is N, Z² is CH;
if Z² is N, Z is CH;
if Z³ is N, Z⁴ is CH;
if Z⁴ is N, Z³ is CH;
n is 1 or 2;
R² is
a 5- or 6-membered carbocyclic ring which can be optionally substituted with F, NH₂, CN and/or CH₃,
a 5- or 6-membered heterocycloalkyl ring which can be optionally substituted with F, NH₂, CN and/or CH₃, wherein the 5- or 6-membered heterocycloalkyl ring contains one or more heteroatoms selected from N, O and S,
a 5-membered heteroaryl ring which can be optionally substituted with F, NH₂, CN and/or CH₃, wherein the 5-membered heteroaryl ring contains one or more heteroatoms selected from N, O and S, or
a 6-membered heteroaryl ring which can be optionally substituted with F, NH₂, CN and/or CH₃, wherein the 6-membered heteroaryl ring contains one heteroatom selected from O and S, or two or more heteroatoms selected from O, N and S; and
R¹⁴ is a leaving group which is replaced by ¹⁸F in the radiolabeling step.

42. A kit for preparing a radiopharmaceutical preparation comprising a compound of formula (II) as defined in claim 37 or a stereoisomer, polymorph, racemic mixture, tautomer, pharmaceutically acceptable salt, prodrug, hydrate, or solvate thereof, or mixtures thereof.

43. A kit for preparing a radiopharmaceutical preparation comprising a compound of formula (III) as defined in claim 38 or a stereoisomer, polymorph, racemic mixture, tautomer, pharmaceutically acceptable salt, prodrug, hydrate, or solvate thereof, or mixtures thereof.

44. A kit for preparing a radiopharmaceutical preparation comprising a compound of formula (IV) as defined in claim 40 or a stereoisomer, polymorph, racemic mixture, tautomer, pharmaceutically acceptable salt, prodrug, hydrate, or solvate thereof, or mixtures thereof.
